(19) United States
(12) Patent Application Publication
(10) Pub. No.: US 2008/0108694 A1
(43) Pub. Date: May 8, 2008

(54) Novel Macrocycles and Uses Thereof

(76) Inventors: Samuel J Danishefsky, Englewood, NJ (US); Zhi-Qiang Yang, Schwenksville, PA (US); Xudong Geng, Cambridge, MA (US); Ting-Chao Chou, Paramus, NJ (US); Neal Rosen, Englewood, NJ (US)

Correspondence Address: CHOATE, HALL & STEWART LLP;SLOAN-KETTERING INSTITUTE FOR CANCER RESEARCH, TWO INTERNATIONAL PLACE, BOSTON, MA, 02110, US

(21) Appl. No.: 10/583,002

(22) PCT Filed: Dec. 17, 2004
(86) PCT No.: PCT/US04/42887

§ 371 Date: Aug. 2, 2007

Related U.S. Application Data
(60) Provisional application No. 60/531,092, filed on Dec. 19, 2003.

(51) Int. Cl. A61K 31/335; A61K 31/395; A61P 35/00; C07D 225/04; C07D 313/06; C07F 15/06

(52) U.S. Cl. 514/450; 514/183; 540/452; 540/461; 549/208; 549/268; 556/142

(57) ABSTRACT

The present invention relates to compounds having the structure (and pharmaceutically acceptable derivatives thereof)

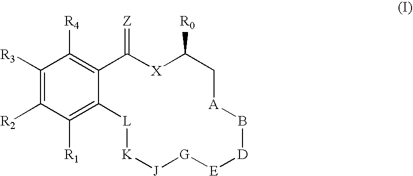

wherein Rₒ-R₄, Z, X, A-B, D-E, G-J, and K-L are as defined herein, the synthesis thereof and the use of these compounds as therapeutic agents.

### GOVERNMENT SUPPORT

The present invention was made with support from a grant from the National Institutes of Health (Number CA-28824; Samuel J. Danishefsky). Additionally, the present invention was made with support from a grant from the United States Army Breast Cancer Research Program (Xudong Geng). Therefore, the government may have certain rights in the invention.

### BACKGROUND OF THE INVENTION

Many natural products, usually bacterial metabolites, feature a macrolide fused to a monocyclic benzenoid matrix, bearing a resorcinol-like substitution pattern. Not infrequently, the resorcinol moiety carries additional functionality, resulting in higher levels of oxidation. Natural products in this family (cf. inter alia radicicol (Delmotte, P.; Delmotte-Plaquee, J. Nature 1953, 171, 344; incorporated herein by reference), LL-Z-1640s (McGahren, W. J. J. Org. Chem. 1978, 43, 2339-2343; which is incorporated herein by reference), monocillins (Ayer, W. A.; Lee, S. P.; Tsuneda, A.; Hiratsuka, Y. Can. J. Microb. 1980, 26, 766-773; incorporated herein by reference), nordinone (Ayer, W. A.; Pena-Rodriguez, L. Phytochemistry 1987, 26, 1353-1355; incorporated herein by reference) and zearelenone (Sugawara, F.; Kim, K. W.; Kobayashi, K.; Uzawa, J.; Yoshida, S.; Murofushi, N.; Takahashi, N.; Strobel, G. A. Phytochemistry 1992, 31, 1987-1990; incorporated herein by reference) possess potentially exploitable patterns of antitumor, antibiotic, and antimalarial activity.

Radicicol (Delmotte et al. Nature 1953, 171, 344; Ayer et al. Canad. J. Microbiol. 1980, 26, 766; incorporated herein by reference) (1) and monocillin I (Ayer et al. Canad. J. Microbiol. 1980, 26, 766) (2) are resorcylic macrolides which can both be isolated from Monocillium nordinii (Ayer et al. Canad. J. Microbiol. 1980, 26, 766; incorporated herein by reference) (FIG. 1). While the skeletal structure of radicicol was determined in 1964, (McCapra et al. Tetrahedron Lett. 1964, 869; Mirrington et al. Tetrahedron Lett. 1964, 365; incorporated herein by reference) its relative and absolute stereochemical configuration was not unambiguously established until 1987 (Cutler et al. Agric. Biol. Chem. 1987, 51, 3331; incorporated herein by reference). The structure of monocillin I was confirmed by its direct conversion into radicicol. Affirmation of these structures was achieved by their only total synthesis through the efforts of Lett and Lampilas (Lampilas et al. Tetrahedron Lett. 1992, 33, 773 and 777; incorporated herein by reference).

Both radicicol (1) and monocillin I (2) (see FIG. 1) exhibit a variety of antifungal and antibiotic properties not shared by other members of this class of natural products. Recently, the antitumor properties of radicicol have come into focus as its ability to suppress the transformed phenotype caused by various oncogenes such as src, ras, and raf has been linked to its tight binding (20 nM) and inhibition of the Hsp90 molecular chaperone (Roe et al. J. Med. Chem. 1999, 42, 260-266; incorporated herein by reference). This ‘anti-chaperone’ activity may stimulate depletion of oncogenic proteins, and could therefore be of clinical interest. Specifically, occupancy of the ATP binding pocket of Hsp90 is believed to lead to the degradation in the proteasome of a subset of proteins involved in signal transduction that require Hsp90 for conformational maturation (see, Schneider et al. Proc. Natl. Acad. Sci. USA 93: 14536-14541, 1996; Mimnaugh et al J. Biol. Chem. 271: 22796-22801, 1996; Whitesell et al. Mol. Endocrinol. 10: 705-712, 1996; each of which is incorporated herein by reference). These proteins include the HER and insulin receptor families of tyrosine kinases, Raf-1 serine kinase and steroid receptors to name a few. Downregulation of any of these would be expected to have positive antiproliferative effects, so that Hsp90 is an attractive target for the development of antitumor drugs.

More recently, five new 14-membered resorcyclic macrolides, termed aigailomycins A-E, were isolated from the marine mangrove fungus Aigialus parvus BCC5311 (Isalca, M.; Suyarnsestakorn, C.; Tanticharoen, M.; Kongsaeree, P.; Thebtaranonth, Y. J. Org. Chem. 2002, 67, 1561-1566; incorporated herein by reference). Among the aigailomycins, aigailomycin D exhibits potent antimalarial activity (IC₅₀: 6.6 μg/mL against P. falciparum) and antitumor activity (IC₅₀: 3.0 μg/mL against KB cells) (Isaka, M.; Suyarnsestakorn, C.; Tanticharoen, M.; Kongsaeree, P.; Thebtaranonth, Y. J. Org. Chem. 2002, 67, 1561-1566; incorporated herein by reference).

The demonstrated ability of radicicol to bind to and inhibit the activity of Hsp90 has generated an interest in further exploring the biological and pharmacological activity of radicicol and analogues thereof. Significantly, to date, only one synthesis of radicicol itself has been recorded (Lampilas et al. Tetrahedron Lett. 1992, 33, 773 and 777; incorporated herein by reference). Other groups have accessed a variety of analogues from the natural product itself (see, U.S. Pat. No. 5,650,430; U.S. Pat. No. 5,731,343; U.S. Pat. No. 6,239,168; U.S. Pat. No. 5,977,165; and U.S. Pat. No. 5,597,846; each of which is incorporated herein by reference), but have been limited in the range of analogues that can be generated. Thus, there remains a need to develop a practical synthesis of radicicol and other resorcyclic macrolides to generate novel analogs and conjugates to explore novel biological and pharmacological activities, and to improve the stability and therapeutic efficacy of radicicol, monocillin, and aigialomycins in the treatment of cancer.

### DESCRIPTION OF THE DRAWING

FIG. 1 depicts structures of Monocillin I (2), Radicicol (1) and Geldanamycin (3).

FIG. 2 depicts two strategies for the synthesis of radicicol (1) and monocillin (2).

FIG. 3 depicts the synthetic strategy for the construction of the chiral alkylic alcohol.

FIG. 4 depicts the synthetic strategy for the construction of intermediate (18).

FIG. 5 depicts the synthetic strategy for the construction of intermediate (7) via a Mitsunobou esterification.

FIG. 6 depicts a synthetic strategy for the synthesis of radicicol (1) and monocillin (2).

FIG. 7 depicts the synthesis of a variety of chiral components (28), (30) and (32).

FIG. 8 depicts the synthesis of dithiane fragment (34).

FIG. 9 depicts the synthesis of a variety of benzoic acid components (35), (36), (37) and (38).

FIG. 10 depicts the generation of diversity at aromatic positions in the macrocycle.

FIG. 11 depicts the synthesis of a variety of analogues (40), (42), (44) and (46).

FIG. 12 depicts the synthesis of a variety of analogues (48), (50), (52) and (54).

FIG. 13 depicts the synthesis of the chiral cyclopropyl moiety (30) and generation of intermediate (39).

FIG. 14 depicts the synthetic scheme for cyclopropyl-monocillin I (2c) and cyclopropyl-radicicol (40).

FIG. 15 depicts the synthesis of a variety of inventive conjugates.

FIG. 16 depicts the synthesis of a variety of inventive conjugates.

FIG. 17 depicts the results of MCF7 cells (HER2 overexpressed, Rb positive) treated with radicicol and analogues.

FIG. 18 depicts the results of BT474 cells (HER2 overexpressed, Rb positive) treated with radicicol and analogues. The gels demonstrate reduction of HER2 levels over a range of concentrations.

FIG. 19 depicts the growth of MCF7 cells (HER2 overexpressed, Rb positive) treated with radicicol and analogues.

FIG. 20 depicts the ability of radicicol and cyclopropyl radicicol to inhibit breast cancer cells with wild type Rb and small cell lung cancer cells with defective Rb function.

FIG. 21 depicts the growth curve for N417 cells (Rb negative cell line) for radicicol and analogues thereof.

FIG. 22 shows the effect of cycloproparadicicol in nude mice bearing human mammary carcinoma MX-1 xenograft (Q2Dx7, iv injection).

FIG. 23 shows the effect of cycloproparadicicol treatment (Q2Dx6, iv injection) on body weight of nude mice bearing mammary carcinoma MX-1 xenograft.

FIG. 24 shows the therapeutical effect of radicicol and cycloproparadicicol in nude mice bearing human colon carcinoma (HCT-116) xenograft (QDx7, 4 hr. iv-infusion).

FIG. 25 shows body weight changes of nude mice bearing human colon carcinoma (HCT-116) xenografts following treatment with radicicol and cycloproparadicicol (QDx7, 4 hr. iv-infusion).

FIG. 26 shows the effect of cycloproparadicicol on HER2 degradation.

FIG. 27 lists the IC₅₀s of various radidicicol analogues on growth inhibition in different tumor cell lines.

FIG. 28 shows the effect of four different cycloproparadicicol analogues on the degradation of HER2

FIG. 29 depicts the structure of difluoro-cycloproparadicicol.

FIG. 30 is a table showing the cytotoxic effect of radicicol and cycloproparadicicol analogs on CCR_{F}-CEM cell growth.

FIG. 31 shows Her2 degradation assays.

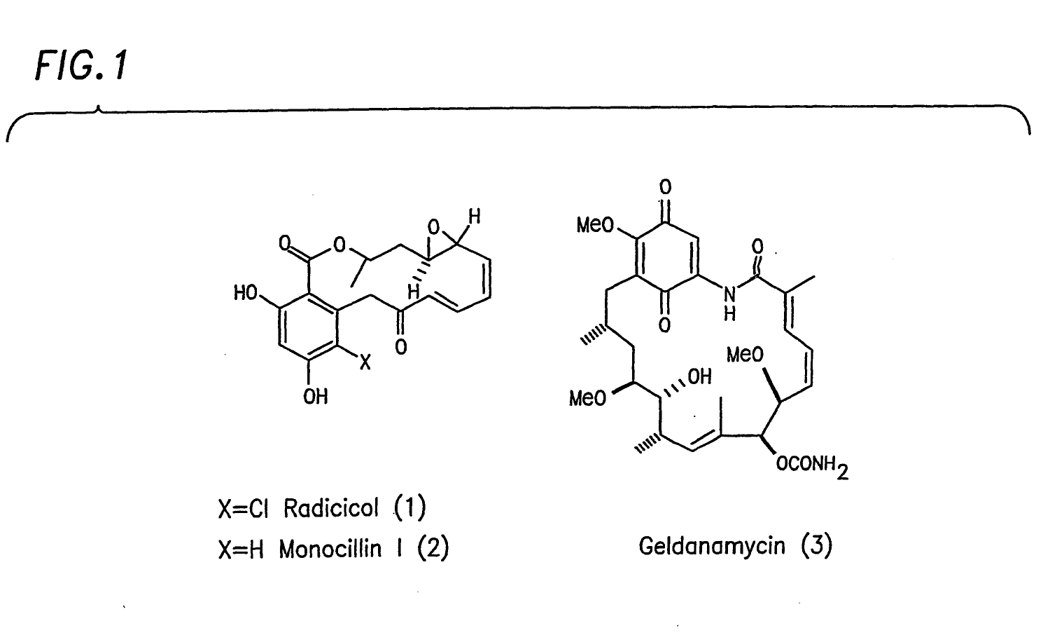

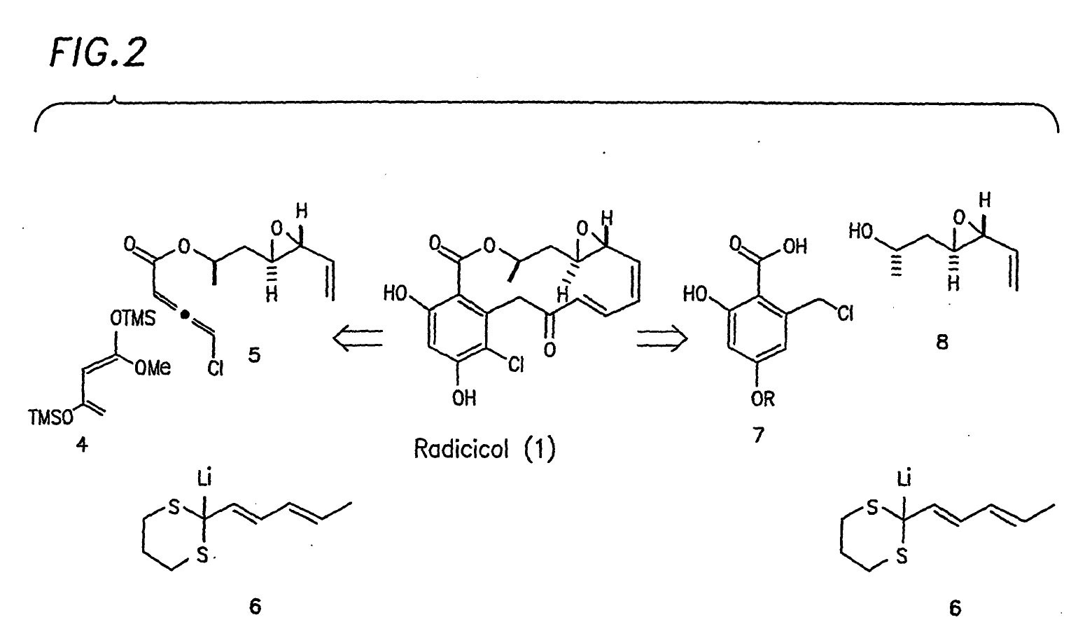

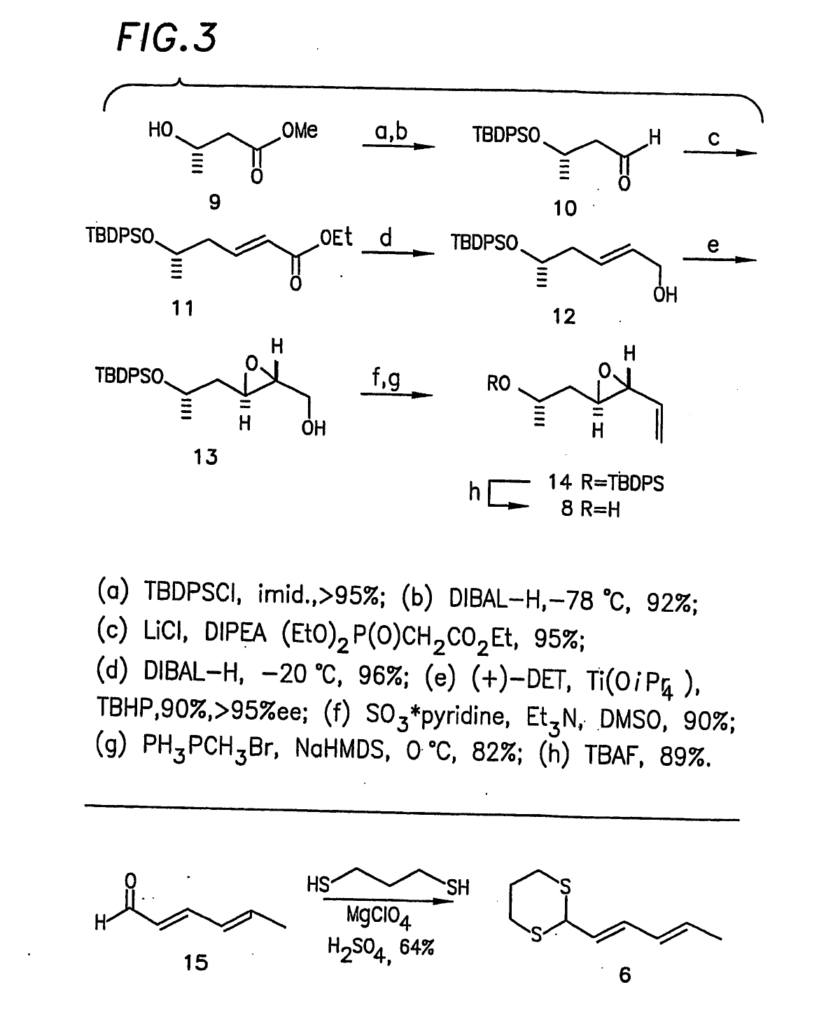

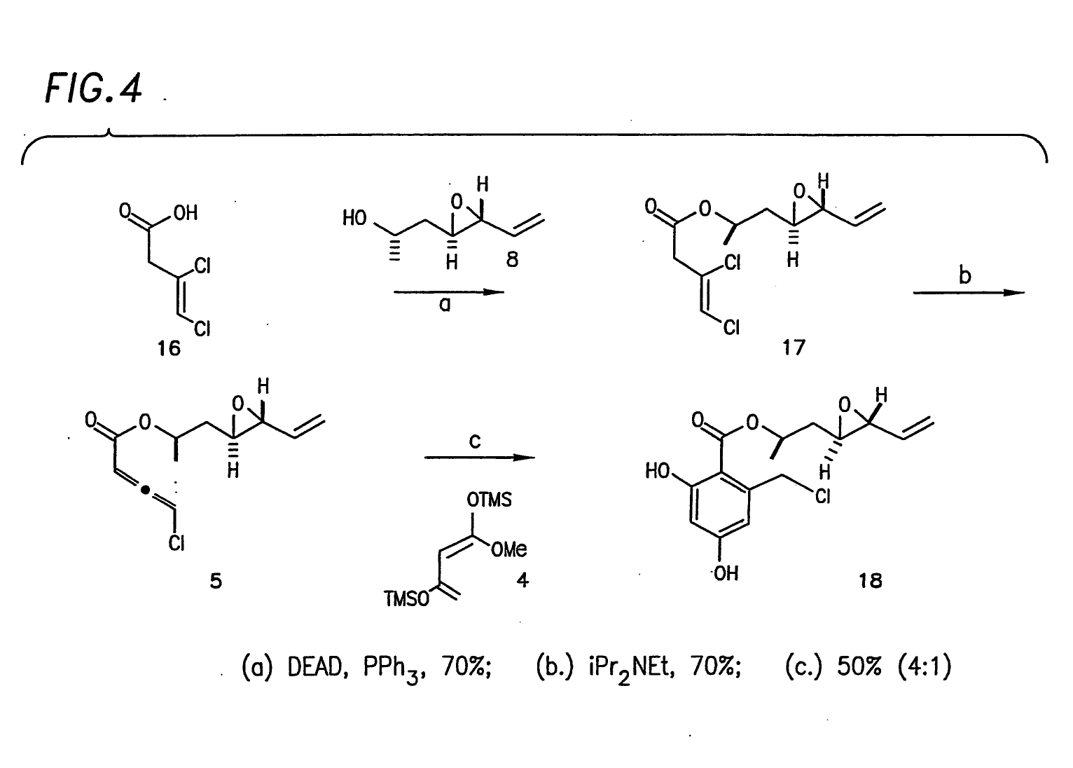

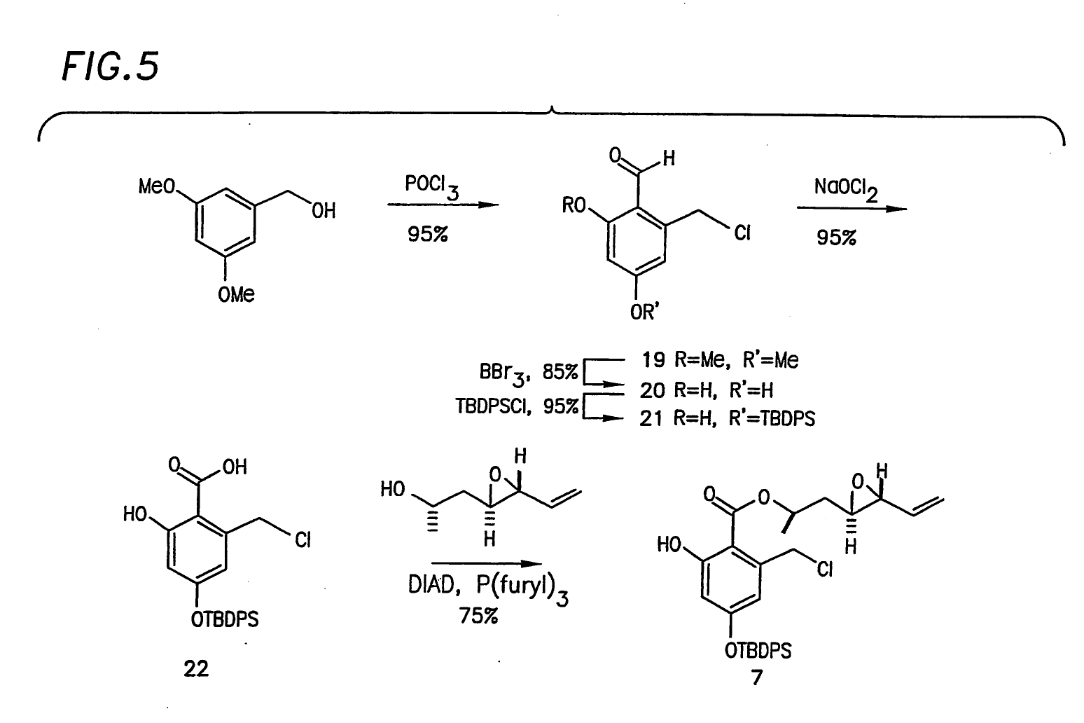

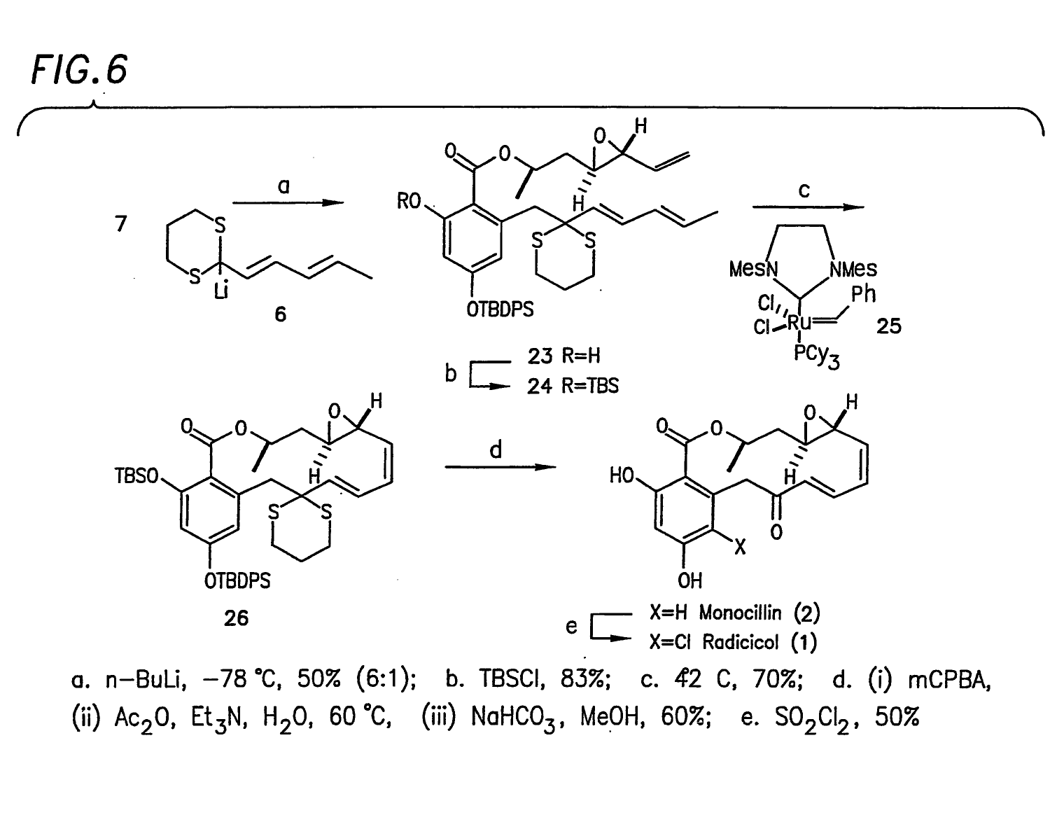

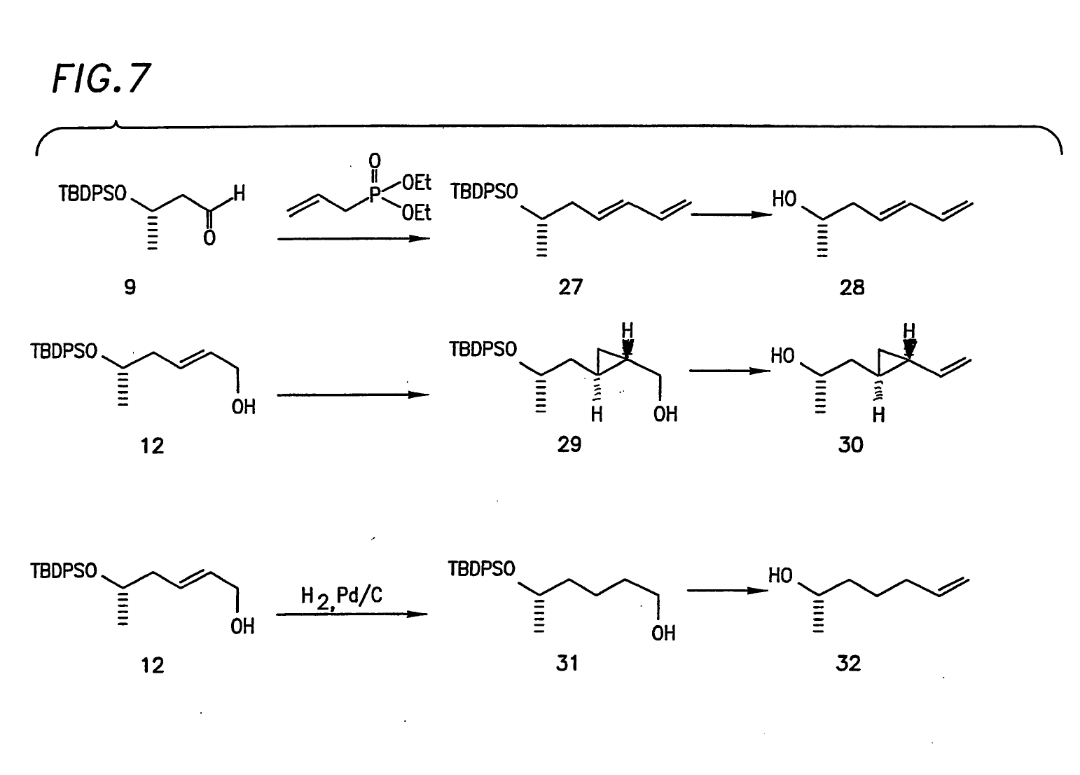

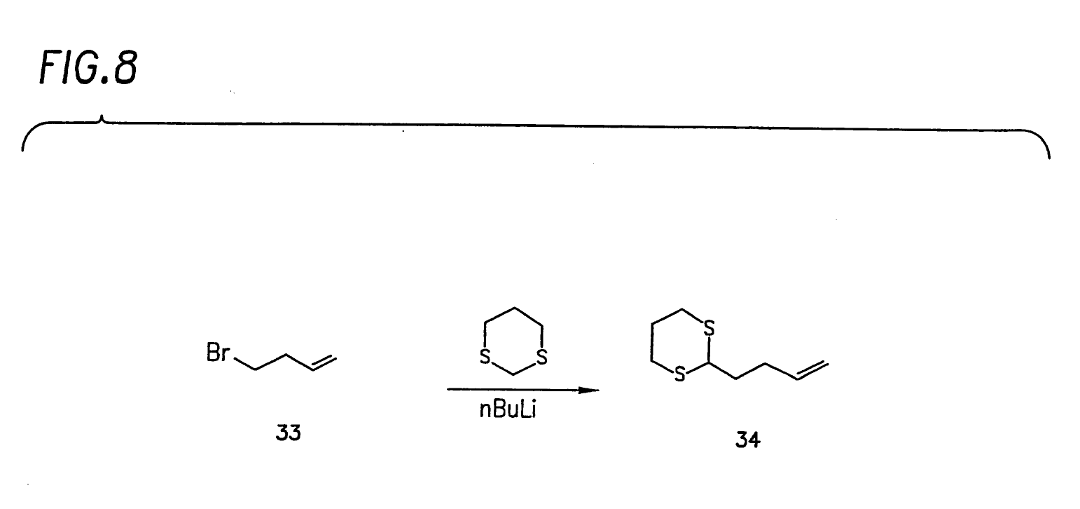

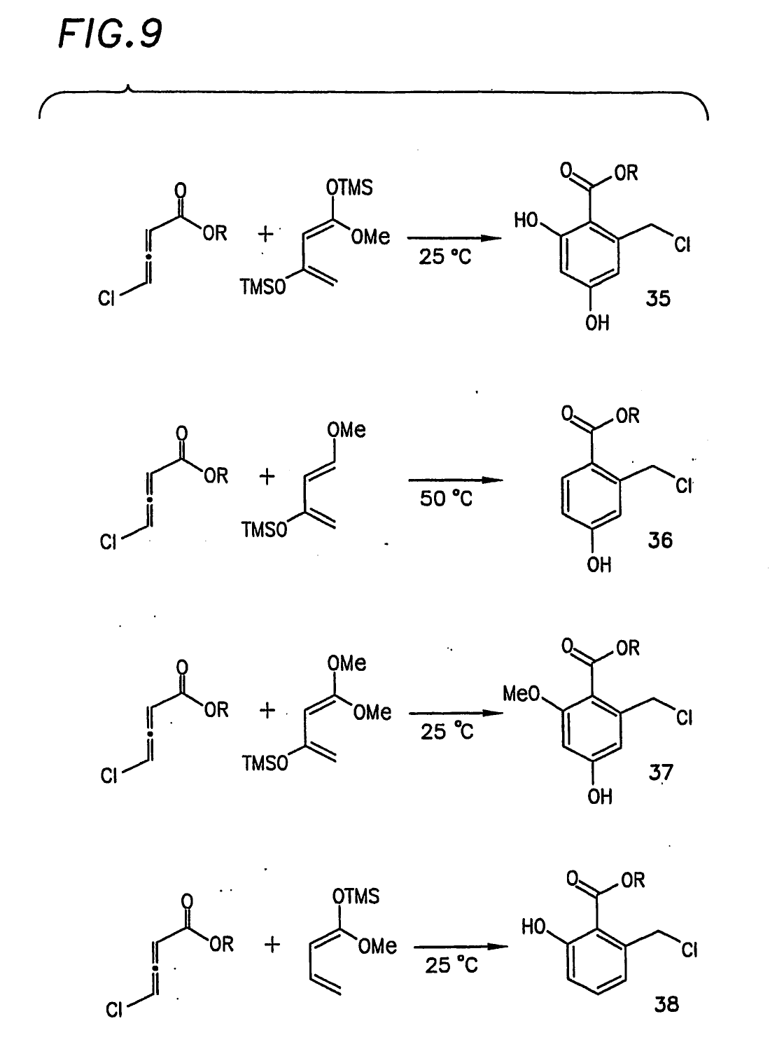

### DESCRIPTION OF THE INVENTION

In recognition of the need to develop novel and effective cancer therapies, the present invention provides novel synthetic methodologies enabling access to macrocycles having a broad range of biological and pharmacological activity. In certain embodiments, the inventive compounds are useful in the treatment of cancer. In certain other embodiments of special interest, the compounds are useful for the treatment of cancers comprising Rb negative cancer cells.

### 1) General Description of Compounds of the Invention

The compounds of the invention include compounds of the general formula (II) as further defined below:

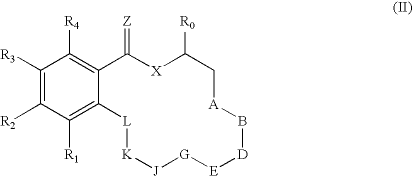

wherein

R₀ is hydrogen, halogen, cyano, —OR_{Z}, —N(R_{Z})₂, —SR_{Z}, —O(C═O)R_{Z}, —N(R_{Z})(C═O)(R_{Z}), —C(O)R_{Z}, —C(O)OR_{Z}, —CON(R_{Z})₂, —OCO₂R_{Z}, or an aliphatic, heteroaliphatic, aryl, heteroaryl, alkylaryl, or alkylheteroaryl moiety, wherein each occurrence of R_{Z} is independently hydrogen, a protecting group, or an aliphatic, heteroaliphatic, aryl, heteroaryl, alkylaryl, or alkylheteroaryl moiety

R₁ is hydrogen, halogen, cyano, —OR_{A}, —N(R_{A})₂, —SR_{A}, —O(C═O)R_{A}, —N(R_{A})(C═O)(R_{A}),

—C(O)R^{A}, —C(O)OR_{A}, —CON(R_{A})₂, —OCO₂R_{A}, or an aliphatic, heteroaliphatic, aryl, heteroaryl, alkylaryl, or alkylheteroaryl moiety, wherein each occurrence of R_{A} is independently hydrogen, a protecting group, or an aliphatic, heteroaliphatic, aryl, heteroaryl, alkylaryl, or alkylheteroaryl moiety;

R₂ is hydrogen, halogen, cyano, —OR_{B}, —N(R_{B})₂, —SR_{B}, —O(C═O)R_{B}, —N(R_{B})(C═O)(R_{B}),

—C(O)R_{B}, —C(O)OR_{B}, —CON(R_{B})₂, —OCO₂R_{B}, or an aliphatic, heteroaliphatic, aryl, heteroaryl, alkylaryl, or alkylheteroaryl moiety, wherein each occurrence of R_{B} is independently hydrogen, a protecting group or an aliphatic, heteroaliphatic, aryl, heteroaryl, alkylaryl, or alkylheteroaryl moiety;

R₃ is hydrogen, halogen, cyano, —OR_{C}, —N(R_{C})₂, —SR_{C}, —O(C—O)R_{C}, —N(R_{C})(C═O)(R_{C}),

—C(O)R_{C}, —C(O)OR_{C}, —CON(R_{C})₂, —OCO₂R_{C}, or an aliphatic, heteroaliphatic, aryl, heteroaryl, alkylaryl, or alkylheteroaryl moiety, wherein each occurrence of R_{C} is independently hydrogen, a protecting group, or an aliphatic, heteroaliphatic, aryl, heteroaryl, alkylaryl, or alkylheteroaryl moiety;

R₄ is hydrogen, halogen, cyano, —OR_{D}, —N(R_{D})₂, —SR_{D}, —O(C═O)R_{D}, —N(R_{D})(C═O)(R_{D}),

—C(O)R_{D}, —C(O)OR_{D}, —CON(R_{D})₂, —OCO₂R_{D}, or an aliphatic, heteroaliphatic, aryl, heteroaryl, alkylaryl, or alkylheteroaryl moiety, wherein each occurrence of R_{D} is independently hydrogen, a protecting group or an aliphatic, heteroaliphatic, aryl, heteroaryl, alkylaryl, or alkylheteroaryl moiety;

Z is O, S, or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;

X is O, S or NR_{G}, wherein R_{G} is hydrogen or lower alkyl;

A and B together represent

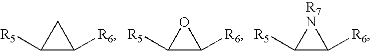

### —CHR₅—CHR₆—, —CR₅═CR₆—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},

—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring;

D and E together represent

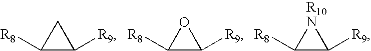

### —CHR₈—CHR₉—, —CR₈═CR₉—, wherein R₈ and R₉ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(—O)OR_{J},

—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₀ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K} or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₈—CHR₉—, R₉ and R₉ taken together represent a substituted or unsubstituted 3-7 membered aliphatic, heteroaliphatic, aryl or heteroaryl ring;

G and J together represent

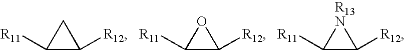

### —CHR₁₁—CHR₁₂—, —CR₁₁═CR₁₂—, wherein R₁₁ and R₁₂ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(—O)OR_{J},

—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₃ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₁—CHR₁₂—, R₁₁ and R₁₂ taken together represent a substituted or unsubstituted 3-7 membered aliphatic, heteroaliphatic, aryl or heteroaryl ring;

K and L together represent

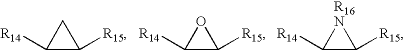

### —CHR₁₄—CHR₁₅—, —CR₁₄═CR₁₅—, wherein R₁₄ and R₁₅ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(—O)OR_{J},

—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₆ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{C} or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₄—CHR₁₅—, R₁₄ and R₁₅ taken together represent a substituted or unsubstituted 3-7 membered aliphatic, heteroaliphatic, aryl or heteroaryl ring;

whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted; and

pharmaceutically acceptable derivatives thereof.

In certain other embodiments of the invention the compounds are subject to one or more, or all of the following limitations:

if Z is O; if X is O; if R₀ is methyl; if A and B together are —CR₄═CR₅— and R₅ and R₆ are each hydrogen; if D and E together are —COH═COH—; if G and J together are —CH₂—CH₂—; if K and L together are —CH═CH—; if R₁ is hydrogen; and if R₃ is hydrogen;

then R₂ and R₄ are each not —OR_{B}, wherein R_{B} is hydrogen or an alkyl, alkoxy, alkenyl, alkenyloxy, alkynyl, aryl, aryloxy, heterocycle, cycloalkyl, cycloalkenyl, or cycloalkenyl fused to an aryl group.

In certain embodiments of the invention, the genuses or subclasses of compounds of the invention exclude aigailomycins A-E.

### 2) Featured Classes of Compounds

It will be appreciated that for compounds as generally described above, certain classes of compounds are of special interest. For example, one class of compounds of special interest includes those compounds having the structure of formula (II) in which Z and X are each O, and the compound has the structure:

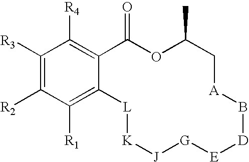

and R₁, R₂, R₃, R₄, A-B, D-E, G-J, and K-L are as defined above and in subclasses herein.

Another class of compounds of special interest consists of compounds having the structure of formula (I) in which Z is O and X is NR_{G}, and the compound has the structure:

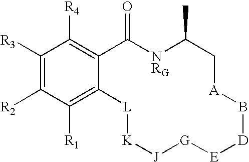

and R₁, R₂, R₃, R₄, R_{G}, A-B, D-E, G-J, and K-L are as defined above and in subclasses herein.

Another class of compounds of special interest consists of compounds having the structure of formula (I) in which G and J together represent —CH₂—CH₂— and the compound has the structure:

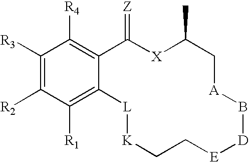

and R₁, R₂, R₃, R₄, Z, X, A-B, D-E, and K-L are as defined above and in subclasses herein.

Another class of compounds of special interest consists of compounds having the structure of formula (I) in which A and B together represent —CH═CH— and the compound has the structure:

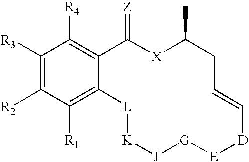

and R₁, R₂, R₃, R₄, R₅, R₆, Z, X, D-E, G-J, and K-L are as defined above and in subclasses herein.

Another class of compounds of special interest consists of compounds having the structure of formula (I) in which K and L together represent —CH═CH— and the compound has the structure:

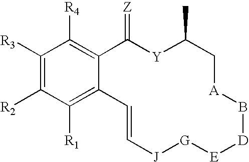

and R₁, R₂, R₃, R₄, Z, X, A-B, D-E, and G-J are as defined above and in subclasses herein.

Another class of compounds of special interest consists of compounds having the structure of formula (I) in which D and E together represent —CHOH═CHOH— and the compound has the structure:

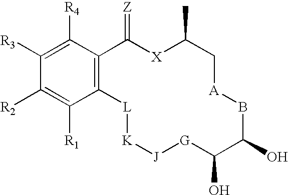

and R₁, R₂, R₃, R₄, R₇, Z, X, A-B, G-J, and K-L are as defined above and in subclasses herein.

Another class of compounds of special interest consists of compounds having the structure of formula (I) in which the compound has the structure:

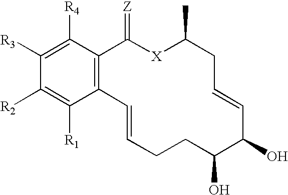

and R₁, R₂, R₃, R₄, Z, and X are as defined above and in subclasses herein. In certain embodiments, Z is oxygen and X is NR_{G}, wherein R_{G} is defined above. In other embodiments, Z is oxygen an X is NH.

Another class of compounds of special interest consists of compounds having the structure of formula (I) in which the compound has the structure:

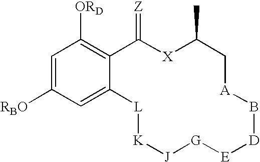

and Z, X, A-B, D-E, G-J, and K-L are as defined above and in subclasses herein. In certain embodiments, R_{D} and R_{B} are each hydrogen.

Another class of compounds of special interest consists of compounds having the structure of formula (I) in which the compound has the structure:

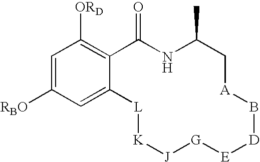

and R_{D}, R_{B}, A-B, D-E, G-J, and K-L are as defined above and in subclasses herein.

Another class of compounds of special interests consists of compounds having the structure of formula (I) in which the compound has the structure:

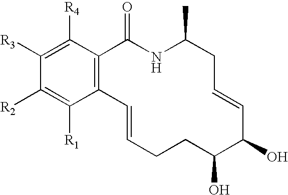

and R₁, R₂, R₃, and R₄ are as defined above and in subclasses herein.

Another class of compounds of special interest consists of compounds having the structure of formula (I) in which the compound has the structure:

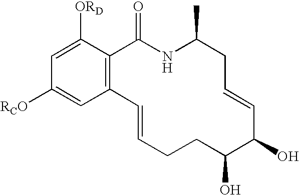

and R_{C} and R_{D} are as defined above and in subclasses herein. In certain embodiments, R_{D} and R_{C} are each hydrogen.

Another class of compounds of special interest consists of compounds having the structure of formula (I) in which the compound has the stricture:

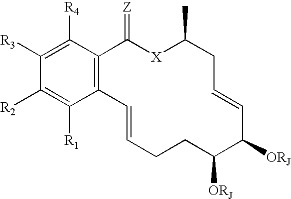

and R₁, R₂, R₃, R₄, R_{J}, Z, and X are as defined above and in subclasses herein. In certain embodiments, Z is oxygen and X is oxygen. In other embodiments, Z is oxygen and X is NH.

The following structures illustrate several exemplary types of compounds of these classes. Others will be readily apparent to the reader.

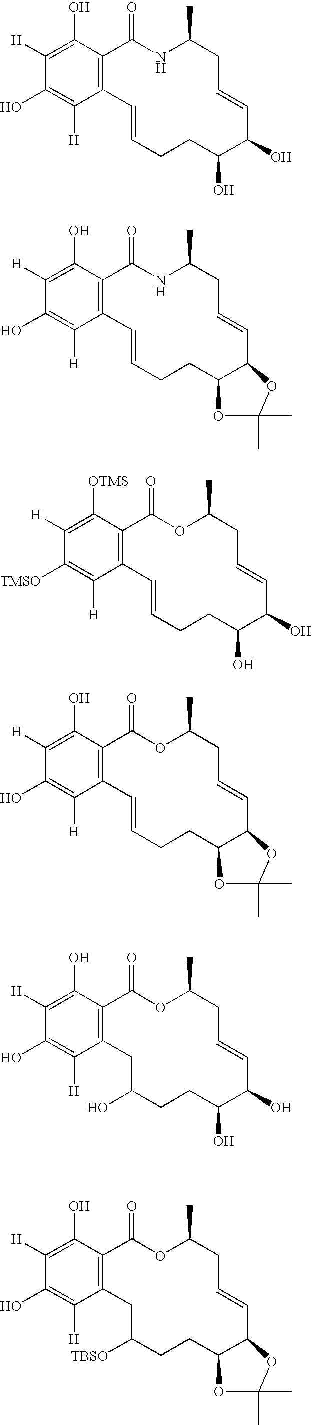

A number of important subclasses of each of the foregoing classes deserve separate mention; these subclasses include subclasses of the foregoing classes in which:

i) Z and X are each O;

ii) Z is o and X is NH;

iii) A and B together are a trans carbon-carbon double bond;

iv) A and B together are a carbon-carbon single bond;

v) D and E together are —CHOH—CHOH—;

vi) J and G together are a carbon-carbon single bond;

vii) K and L together are a trans carbon-carbon double bond;

viii) R₂ and R₄ are each independently hydrogen, halogen, cyano, —OR_{J}, —N(R_{J})₂, —SR_{J},

—O(C═O)R_{J}, —O(S═O)R_{J}, —N(R_{J})(C═O)(R_{J}), or —OCO₂R_{J}, —OSO₂R_{J}, and each occurrence of R_{J} is independently hydrogen, a protecting group or an aliphatic, heteroaliphatic, aryl, heteroaryl, alkylaryl, or alkylheteroaryl moiety;

ix) R₂ and R₄ are each independently hydroxy;

x) D and E together are —CHR₈—CHR₉—;

xi) D and E together are —CR₈═CR₉—;

xii) R₁ and R₃ are each hydrogen;

xiii) G and J together are —CHR₁₀—CHR₁₁—;

xiv) G and J together are —CR₁₀═CR₁₁—;

xv) R₁₀ and R₁₁ are each hydrogen;

xvi) Z is O;

xvii) Z is S;

xviii) X is S;

xix) X is NR_{G};

xx) X is O;

xxi) R₁ is hydrogen, halogen, lower alkyl, lower heteroalkyl, lower alkylaryl, lower alkylheteroaryl, or N(R_{A})₂, wherein each occurrence of R_{A} is independently hydrogen, a protecting group, or an aliphatic, heteroaliphatic, aryl, heteroaryl, alkylaryl, or alkylheteroaryl moiety and R₃ is hydrogen, halogen, lower alkyl, lower heteroalkyl, lower alkylaryl, lower alkylheteroaryl, or —N(R_{C})₂, wherein each occurrence of R_{C} is independently hydrogen, a protecting group, or an aliphatic, heteroaliphatic, aryl, heteroaryl, alkylaryl, or alkylheteroaryl moiety;

xxii) R₁ and R₃ are each independently halogen, hydrogen, or lower alkyl;

xxiii) R₂ is hydrogen, halogen, —OR_{B}, —N(R_{B})₂, —SR_{B}, —O(C═O)R_{B}, —N(R_{B})(C═O)(R_{B}),

—C(O)R_{B}, —C(O)OR_{B}, —CON(R_{B})₂, —OCO₂R_{B}, or lower alkyl, lower heteroalkyl, lower alkylaryl, lower alkylheteroaryl, wherein each occurrence of R_{B} is independently hydrogen, a protecting group or an aliphatic, heteroaliphatic, aryl, heteroaryl, alkylaryl, or alkylheteroaryl moiety, and R₄ is hydrogen, halogen, —OR_{D}, —N(R_{D})₂, —SR_{D}, —O(C═O)R_{D}, —N(R_{D})(C═O)(R_{D}),
—C(O)R_{D}, —C(O)OR_{D}, —CON(R_{D})₂, —OCO₂R_{D}, or lower alkyl, lower heteroalkyl, lower alkylaryl, lower alkylheteroaryl, wherein each occurrence of R_{D} is independently hydrogen, a protecting group or an aliphatic, heteroaliphatic, aryl, heteroaryl, alkylaryl, or alkylheteroaryl moiety;

xxiv) R₂ is hydrogen or —OR_{B}, wherein each occurrence of R_{B} is independently hydrogen, a protecting group or an aliphatic, heteroaliphatic, aryl, heteroaryl, alkylaryl, or alkylheteroaryl moiety, and R₄ is hydrogen or —OR_{D}, wherein each occurrence of R_{D} is independently hydrogen, a protecting group or an aliphatic, heteroaliphatic, aryl, heteroaryl, alkylaryl, or alkylheteroaryl moiety;

xxv) R₀ is hydrogen or aliphatic;

xxvi) R₀ is methyl;

xxvii) K and L together are a cis carbon-carbon double bond.

In certain embodiments of the compounds described above, R₁ and R₃ are each independently halogen, hydrogen, or lower alkyl; R₂ is hydrogen or —OR_{B}, wherein each occurrence of R_{B} is independently hydrogen, a protecting group or an aliphatic, heteroaliphatic, aryl, heteroaryl, alkylaryl, or alkylheteroaryl moiety, and R₄ is hydrogen or —OR_{D}, wherein each occurrence of R_{D} is independently hydrogen, a protecting group or an aliphatic, heteroaliphatic, aryl, heteroaryl, alkylaryl, or alkylheteroaryl moiety.

Some of the foregoing compounds can exist in various isomeric forms. The invention encompasses the compounds as individual isomers substantially free of other isomers and alternatively, as mixtures of various isomers, e.g., racemic mixtures of stereoisomers, cis and trans isomers. The invention also encompasses tautomers of specific compounds as described above. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of this invention which are of particular interest include those which:
exhibit cytotoxic or growth inhibitory effect on cancer cell lines maintained in vitro or in animal studies using a scientifically acceptable cancer cell xenograft model;
exhibit cytotoxic or growth inhibitory effect on cancer cell lines comprising Rb negative cells;
exhibit antimalarial activity;
exhibit antibacterial activity;
bind to and/or inhibit the Hsp90 family of chaperones;
exhibit cytotoxic or growth inhibitory effect on cancer cell lines comprising Rb positive cells.

This invention also provides a pharmaceutical preparation comprising at least one of the compounds as described above and herein, or a pharmaceutically acceptable derivative thereof, which compounds are capable of inhibiting the growth of or killing cancer cells, and, in certain embodiments of special interest are capable of inhibiting the growth of or killing cancer cells.

The invention further provides a method for inhibiting tumor growth and/or tumor metastasis. The method involves the administration of a therapeutically effective amount of the compound or a pharmaceutically acceptable derivative thereof to a subject (including, but not limited to a human or animal) in need of it. In certain embodiments, the inventive compounds are useful for the treatment of solid tumors. In still other embodiments of interest, the inventive compounds are useful for the treatment of glioblastoma, retinoblastoma or small cell lung cancer.

### 3) Compounds and Definitions

As discussed above, this invention provides novel compounds with a range of biological properties. Compounds of this invention have biological activities relevant for the treatment of diseases or other disorders such as proliferative diseases, including, but not limited to cancer. More generally, the compounds are useful in the regulation of the cell cycle pathway.

Compounds of this invention include those specifically set forth above and described herein, and are illustrated in part by the various classes, subgenera and species disclosed elsewhere herein.

It will be appreciated by one of ordinary skill in the art that asymmetric centers may exist in the compounds of the present invention. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, a mixtures of stereoisomers or diastereomers are provided.

Additionally, the present invention provides pharmaceutically acceptable derivatives of the inventive compounds, and methods of treating a subject using these compounds, pharmaceutical compositions thereof, or either of these in combination with one or more additional therapeutic agents. The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety which is susceptible to removal in vivo yielding the parent molecule as the pharmacologically active species. An example of a pro-drug is an ester which is cleaved in vivo to yield a compound of interest. Pro-drugs of a variety of compounds, and materials and methods for derivatizing the parent compounds to create the pro-drugs, are known and may be adapted to the present invention. Certain exemplary pharmaceutical compositions and pharmaceutically acceptable derivatives will be discussed in more detail herein below.

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in “Organic Chemistry”, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups. By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other funcational groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. Exemplary protecting groups are detailed herein, however, it will be appreciated that the present invention is not intended to be limited to these protecting groups; rather, a variety of additional equivalent protecting groups can be readily identified using the above criteria and utilized in the method of the present invention. Additionally, a variety of protecting groups are described in “Protective Groups in Organic Synthesis” Third Ed. Greene, T. W. and Wuts, P. G., Eds., John Wiley & Sons, New York: 1999, the entire contents of which are hereby incorporated by reference.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative disorders, including, but not limited to cancer. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “aliphatic”, as used herein, includes both saturated and unsaturated, straight chain (i.e., unbranched), branched, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties. Thus, as used herein, the term “alkyl” includes straight, branched and cyclic alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (cyclic, acyclic, substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, cyclopropyl, —CH₂-cyclopropyl, alkyl, n-butyl, sec-butyl, isobutyl, tert-butyl, cyclobutyl, —CH₂-cyclobutyl, n-pentyl, sec-pentyl, isopentyl, tert-pentyl, cyclopentyl, —CH₂-cyclopentyl, n-hexyl, sec-hexyl, cyclohexyl, —CH₂-cyclohexyl moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alkoxy”, or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 14 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 14 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)R^{x}; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the terms “aryl” and “heteroaryl”, as used herein, refer to stable mono- or polycyclic, heterocyclic, polycyclic, and polyheterocyclic unsaturated moieties having preferably 3-14 carbon atoms, each of which may be substituted or unsubstituted. Substituents include, but are not limited to, any of the previously mentioned substitutents, i.e., the substituents recited for aliphatic moieties, or for other moieties as disclosed herein, resulting in the formation of a stable compound. In certain embodiments of the present invention, “aryl” refers to a mono- or bicyclic carbocyclic ring system having one or two aromatic rings including, but not limited to, phenyl, naphthyl, tetrahydronaphthyl, indanyl, indenyl and the like. In certain embodiments of the present invention, the term “heteroaryl”, as used herein, refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one, two or three of the hydrogen atoms thereon independently with any one or more of the following moieties including, but not limited to: aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or heterocyclic moieties, may optionally be substituted with substituents including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R^{x}; —CO₂(Rₓ); —CON(Rₓ)₂; —OC(O)R^{x}; —OCO₂Rₓ; —OCON(R^{x})₂; —N(Rₓ)₂; —S(O)₂R^{x}; —NRₓ(CO)Rₓ wherein each occurrence of Rₓ independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The term “heteroaliphatic”, as used herein, refers to aliphatic moieties which contain one or more oxygen, sulfur, nitrogen, phosphorus or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of R^{x} independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “heterocycloalkyl” or “heterocycle”, as used herein, refers to a non-aromatic 5-, 6- or 7-membered ring or a polycyclic group, including, but not limited to a bi- or tri-cyclic group comprising fused six-membered rings having between one and three heteroatoms independently selected from oxygen, sulfur and nitrogen, wherein (i) each 5-membered ring has 0 to 1 double bonds and each 6-membered ring has 0 to 2 double bonds, (ii) the nitrogen and sulfur heteroatoms may be optionally be oxidized, (iii) the nitrogen heteroatom may optionally be quaternized, and (iv) any of the above heterocyclic rings may be fused to a benzene ring. Representative heterocycles include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl. In certain embodiments, a “substituted heterocycloalkyl or heterocycle” group is utilized and as used herein, refers to a heterocycloalkyl or heterocycle group, as defined above, substituted by the independent replacement of one, two or three of the hydrogen atoms thereon with but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(x); —CON(Rₓ)₂; —OC(O)Rₓ; —OCO₂Rₓ; —OCON(Rₓ)₂; —N(R^{x})₂; —S(O)₂R^{x}; —NR^{x}(CO)R^{x} wherein each occurrence of R^{x} independently includes, but is not limited to, aliphatic, heteroaliphatic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples which are described herein.

### 4) Synthetic Methodology

Access to resorcyclic macrolides such as radicicol and monocillin was previously limited to compounds accessed via the natural products (see, for example, U.S. Pat. No. 5,650,430; U.S. Pat. No. 5,731,343; U.S. Pat. No. 6,239,168; U.S. Pat. No. 5,977,165; and U.S. Pat. No. 5,597,846; each of which is incorporated herein by reference). An efficient and practical synthesis of radicicol and monocillin was previously described in U.S. Ser. No. 09/938,754, filed Aug. 25, 2001, which is incorporated herein by reference. In recognition of the need for a more efficient and practical route to this class of therapeutic agents, the present invention also provides novel synthetic methodologies for the synthesis of radicicol, monocillin, and analogues thereof as well as aigialomycins and analogues thereof. Although the synthesis of radicicol and aigialomycin D is described specifically herein directly below (and in the Examples), it will be appreciated that this methodology is generally applicable to the generation of analogues and conjugates as discussed in more detail after the discussion of the synthesis of radicicol and aigialomycin D.

a) Synthesis of Radicicol and Monocillin:

As described in more detail below, a novel synthetic route to radicicol and monocillin has been developed, which methodology allows for the efficient generation of a variety of analogues of radicicol, monocillin, homodimers, heterodimers, and conjugates thereof.

The underlying general scheme for this new synthesis of radicicol is shows in the scheme below, which is directed to the synthesis of cycloproparadicicol. The central element of the synthesis is the production of an “ynolide” intermediate, which is prepared by olefin metathesis, and its reaction with a diene via a Diel-Alder cycloaddition to form the benzo-fused macrolide.

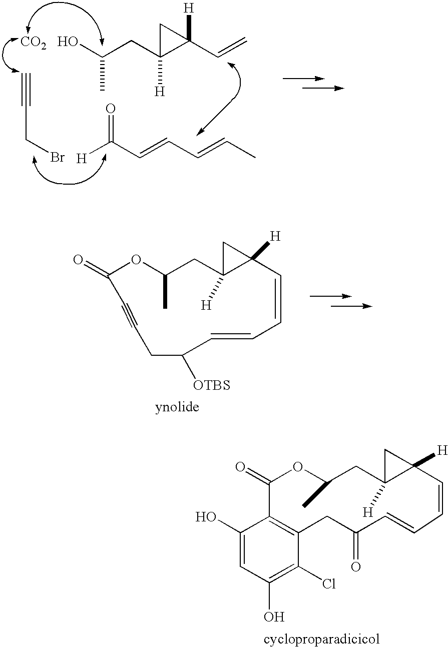

The same general approach has also been used to synthesize compounds of the invention including aigialomycin D as shown below.

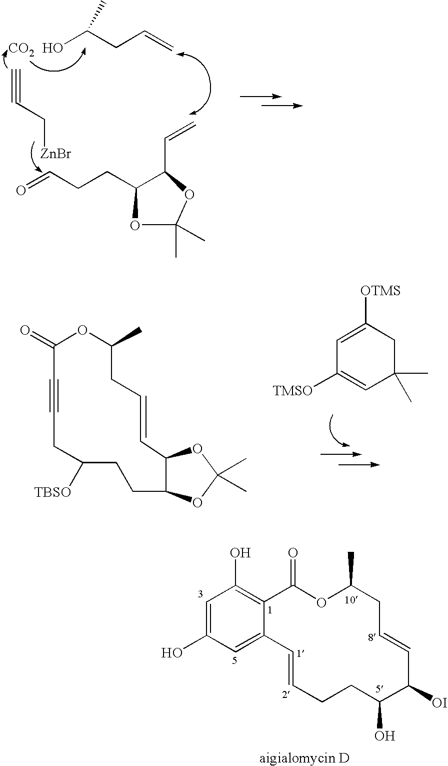

b) General Synthetic Methodology:

This new synthetic methodology provides for the rapid synthesis of a variety of analogues of radicicol, monocillin, dimers, and conjugates thereof. It will be appreciated that the ability to rapidly generate a range of analogues is important because it is believed that in vivo activity is lost due to certain structural characteristics of radicicol and monocillin. For example, it is postulated that in vivo activity is lost due to the nucleophilic action of cellular thiols (i.e. glutathione) on either the epoxide or the α,β unsaturated ketone of radicicol. This nucleophilic addition changes the overall conformation of radicicol, and results in an inability to bind to Hsp90. A second likely pathway of deactivation involves the conjugation of the aromatic ring, or perhaps cytochrome P-450 oxidation.

Without wishing to be bound by any particular theory, one strategy to restore in vivo activity would be to reduce the affinity of radicicol to nucleophiles such as thiols, specifically to deactivate electrophilic sites in radicicol. Here care must be taken not to dramatically change the overall conformation of the natural product. Thus these analogues have been designed to attenuate electrophilicity with simple alterations to the structure that should not affect the overall conformation. It should be emphasized that the analogues as described using the methodology herein cannot be made from the natural product. The three component nature of the synthetic process described above and described more generally below emphasizes the ability to generate numerous analogues by the modification of one component and its incorporation into a short and efficient process.

In general, the method involves the synthesis of analogues from four easily obtainable and diversifiable components. Depicted below is a general retrosynthetic strategy for the synthesis of analogues:

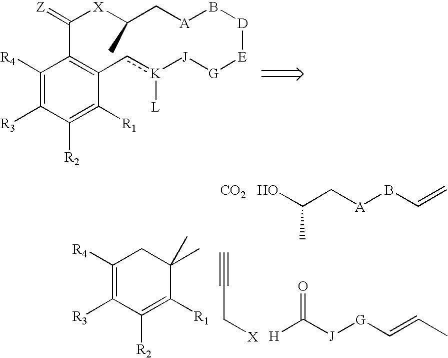

It should be noted that these analogs can be generated either with a single modification, or they may be combined in a single entity to maximize their benefit if they are found to be synergistic. It will also be appreciated, as described in more detail herein, that each of the components can be diversified prior to formation of the macrocycle, or alternatively or additionally, can be diversified after formation of the macrocycle.

As depicted generally below, the synthesis of analogues can be carried out in a similar fashion to the synthesis of radicicol and monocillin as described above. The synthesis of the acyclic alkynoic ester is shown below:

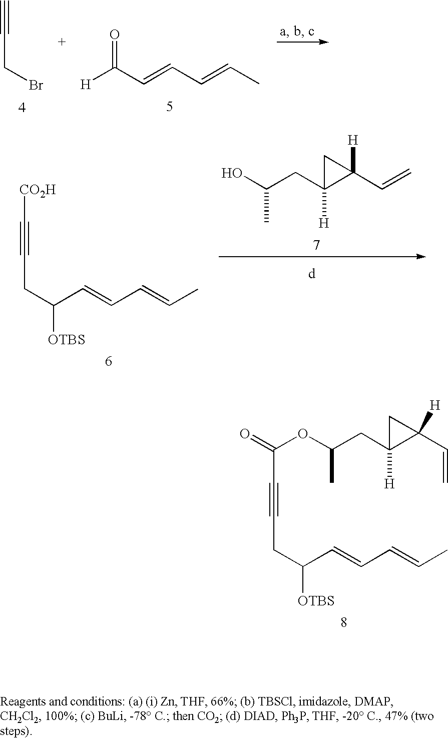

The resulting triene 8 is then treated with dicobalt carbonyl to complex with the acetylene. The combalt complex 14 is then cyclized under ring closing metathesis conditions to form 15. The cobalt is then removed to give 16.

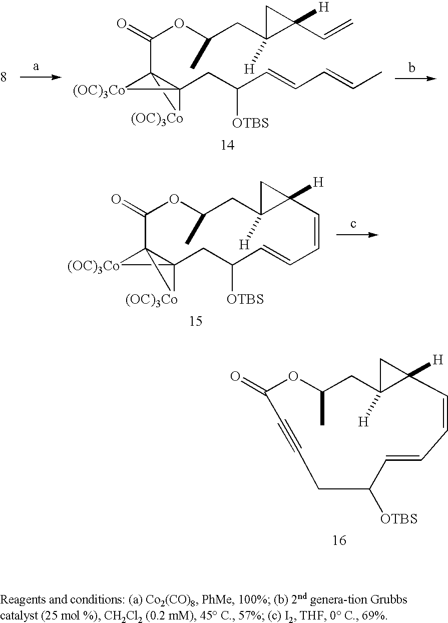

The resulting ynolide 16 is then reacted with a diene using a Diels-Alder cycloaddition to yield 17, which is then oxidized and chlorinated to give the desired product, cycloproparadicicol (2).

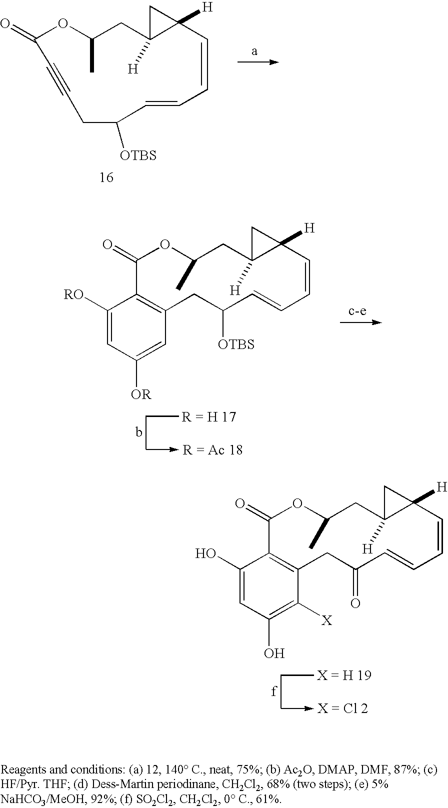

This basic approach can be used to prepare a variety of benofused macrolactones, including aigialomycin D. First the ring closing metathesis precurusor is made starting from D-2-deoxyribose.

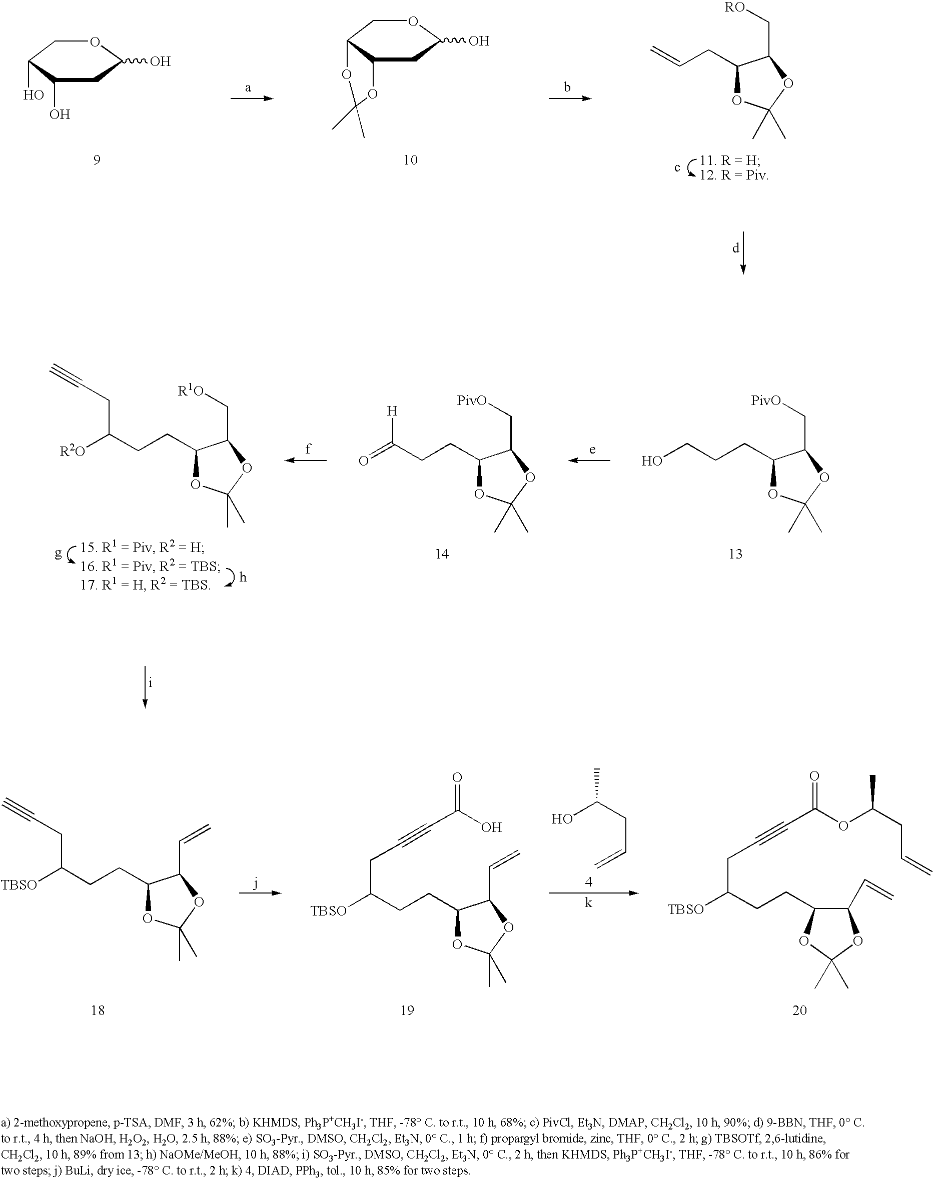

The precursor is treated with dicobalt hexacarbonyl, and the resulting cobalt-complex is cyclized using a ring closing metathesis reaction.

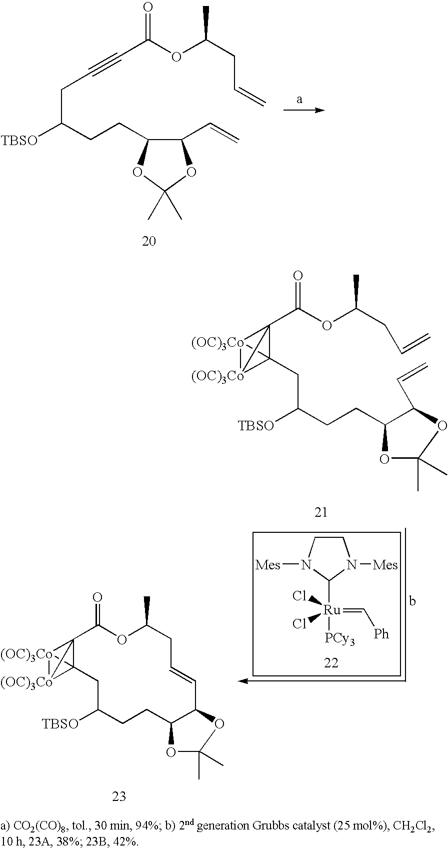

The marocycle was then decomplexed and reacted with disiloxydiene to yield a benzo-fused macrocycle. The styrene double bond was installed, and the protecting groups were removed to yield aigialomycin D.

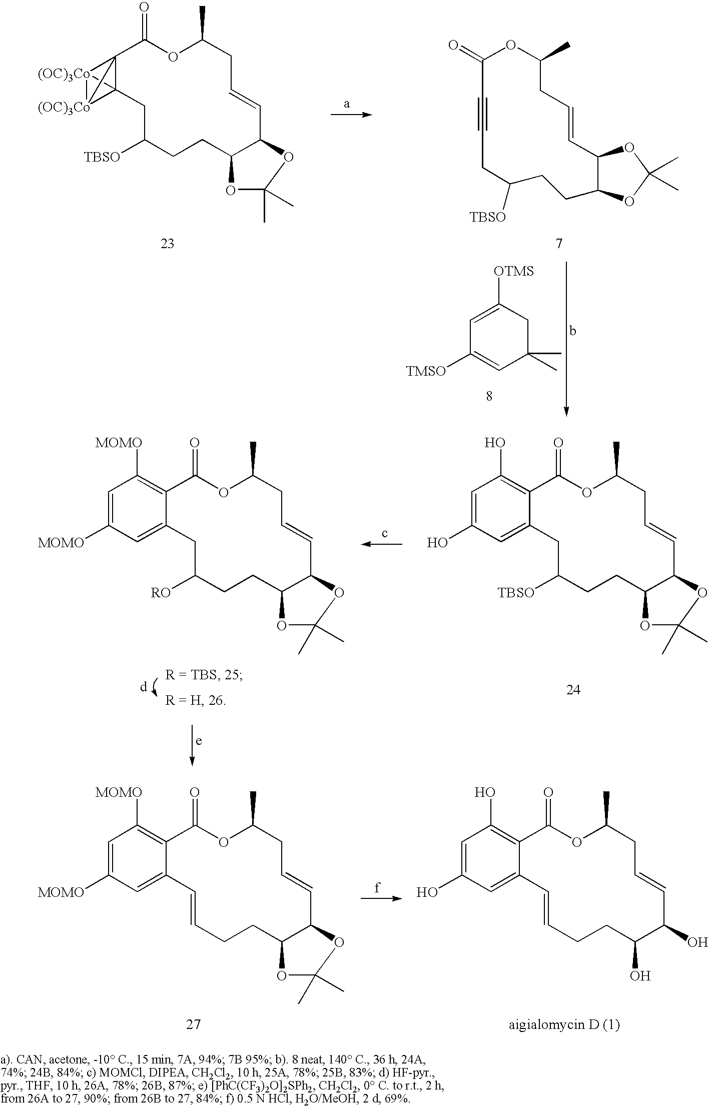

Thus, in addition to providing cycloproparadicicol as described above and herein, the present invention additionally provides a method for the synthesis of compounds having the general structure (I):

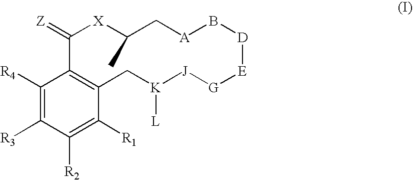

wherein

R₁ is hydrogen, halogen, an aliphatic, heteroaliphatic, aryl, heteroaryl, alkylaryl, or alkylheteroaryl moiety, or N(R_{A})₂, wherein each occurrence of R_{A} is independently hydrogen, a protecting group, or an aliphatic, heteroaliphatic, aryl, heteroaryl, alkylaryl, or alkylheteroaryl moiety;

R₂ is hydrogen, halogen, cyano, —OR_{B}, —N(R_{B})₂, —SR_{B}, —O(C═O)R_{B}, —N(R_{B})(C═O)(R_{B}),

—C(O)R_{B}, —C(O)OR_{B}, —CON(R_{B})₂, —OCO₂R_{B}, or an aliphatic, heteroaliphatic, aryl, heteroaryl, alkylaryl, or alkylheteroaryl moiety, wherein each occurrence of R_{B} is independently hydrogen, a protecting group or an aliphatic, heteroaliphatic, aryl, heteroaryl, alkylaryl, or alkylheteroaryl moiety;

R₃ is hydrogen, halogen, an aliphatic, heteroaliphatic, aryl, heteroaryl, alkylaryl, or alkylheteroaryl moiety, or —N(R_{C})₂, wherein each occurrence of R_{C} is independently hydrogen, a protecting group, or an aliphatic, heteroaliphatic, aryl, heteroaryl, alkylaryl, or alkylheteroaryl moiety;

R₄ is hydrogen, halogen, cyano, —OR_{D}, —N(R_{D})₂, —SR_{D}, —O(C—O)R_{D}, —N(R_{D})(C═O)(R_{D}),

—C(O)R_{D}, —C(O)OR_{D}, —CON(R_{D})₂, —OCO₂R_{D} or an aliphatic, heteroaliphatic, aryl, heteroaryl, alkylaryl, or alkylheteroaryl moiety, wherein each occurrence of R_{D} is independently hydrogen, a protecting group or an aliphatic, heteroaliphatic, aryl, heteroaryl, alkylaryl, or alkylheteroaryl moiety;

Z is O, S or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;

X is O, S or NR_{G}, wherein R_{G} is hydrogen or lower alkyl;

A and B together represent

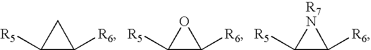

### —CHR₅—CHR₆—, —CR₅═CR₆—, wherein R₅ and R₆ are each independently hydrogen, halogen, cyano, —OR_{J}, —N(R_{J})₂, —SR_{J}, O(C═O)R_{J}, —O(S═O)R_{J}, —N(R_{J})(C═O)(R_{J}), —C(═O)R_{J}, —C(═O)OR_{J},

—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring,

D and E together represent —CHR₈—CHR₉—, —CR₈═CR₉—, wherein R₈ and R₉ are each independently hydrogen or lower alkyl;

G and J together represent —CHR₁₀—CHR₁₁—, —CR₁₀═CR₁₁—, wherein R₁₀ and R₁₁ are each independently hydrogen or lower alkyl;

K and L together represent C═O, C═S, CH—CH₃, CH—CH(R_{L})₂, C═C(R_{L})₂, —CH₂, —C(—S(CH₂)₃S—)—, CH—OR_{L}, CH—SR_{L}, CH—N(R_{L})₂, CH—N(R_{L})(C═O)(R_{L}), C═N—O—R_{L}, CH—N═O, C═C(R_{L})—N(R_{L})₂, C═N—R_{L}, C═N—N(R_{L})₂, or, if the dotted line - - - represents a bond, whereby a double bond is present, then K and L together represent C—N(R_{L})₂, wherein each occurrence of R_{L} is independently hydrogen, a protecting group, an aliphatic, heteroaliphatic, aryl, heteroaryl, alkylaryl, or alkylheteroaryl moiety, or two occurrences of R_{L} taken together represent a 3 to 7-membered cyclic aliphatic, heteroaliphatic, aromatic or heteroaromatic moiety;

whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted;

wherein one or any two of R₁, R_{A}, R₂, R_{B}, R₃, R_{C}, R₄, R_{D}, R₅, R₆, R_{J}, or R_{L} are optionally a linker covalently bonded to a compound selected from the group consisting of radicicol, monocillin, analogues of radicicol and monocillin, geldanamycin, analogues of geldanamycin, and steroids,

said method comprising:

(1) reacting an acidic component having the structure:

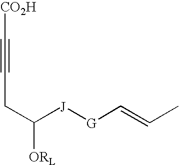

wherein R_{L}, J, and G are as defined above, with a chiral component having the structure:

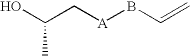

wherein A and B are as defined above, in the presence of an esterification reagent to generate an intermediate having the structure:

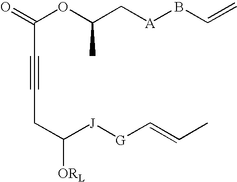

(2) complexing the intermediate with a cobalt, such as dicobalt hexcarbonyl, to yield a structure:

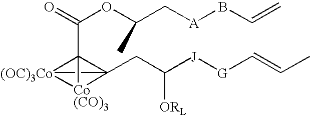

(3) cyclizing the combalt complex in the presence of an olefin metathesis catalyst to generate the compound:

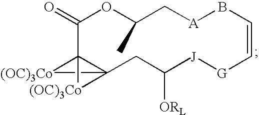

(4) removing the cobalt to form a ynolide;

(5) reacting the alkyne moiety of the ynolide with a diene under cyclcoaddition conditions to generate the compound:

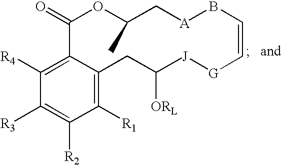

(6) optionally further reacting the macrocycle with one or more reagents to diversify and optionally deprotecting the macrocycle to generate a compound having the formula (I).

Thus, in addition to providing aigialomycin D as described above and herein, the present invention additionally provides a method for the synthesis of compounds having the general structure (IIa):

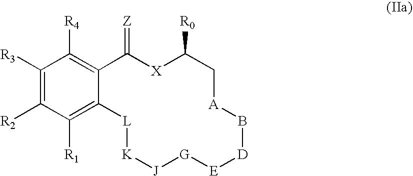

wherein

R₀ is hydrogen, halogen, cyano, —OR_{Z}, —N(R_{Z})₂, —SR_{Z}, —O(C═O)R_{Z}, —N(R_{Z})(C═O)(R_{Z}), —C(O)R_{Z}, —C(O)OR_{Z}, —CON(R_{Z})₂, —OCO₂R_{Z}, or an aliphatic, heteroaliphatic, aryl, heteroaryl, alkylaryl, or alkylheteroaryl moiety, wherein each occurrence of R_{Z} is independently hydrogen, a protecting group, or an aliphatic, heteroaliphatic, aryl, heteroaryl, alkylaryl, or alkylheteroaryl moiety

R₁ is hydrogen, halogen, cyano, —OR_{A}, —N(R_{A})₂, —SR_{A}, —O(C═O)R_{A}, —N(R_{A})(C═O)(R_{A}),

—C(O)R_{A}, —C(O)OR_{A}, —CON(R_{A})₂, —OCO₂R_{A}, or an aliphatic, heteroaliphatic, aryl, heteroaryl, alkylaryl, or alkylheteroaryl moiety, wherein each occurrence of R_{A} is independently hydrogen, a protecting group, or an aliphatic, heteroaliphatic, aryl, heteroaryl, alkylaryl, or alkylheteroaryl moiety;

R₂ is hydrogen, halogen, cyano, —OR_{B}, —N(R_{B})₂, —SR_{B}, —O(C═O)R_{B}, —N(R_{B})(C═O)(R_{B}),

—C(O)R_{B}, —C(O)OR_{B}, —CON(R_{B})₂, —OCO₂R_{B}, or an aliphatic, heteroaliphatic, aryl, heteroaryl, alkylaryl, or alkylheteroaryl moiety, wherein each occurrence of R_{B} is independently hydrogen, a protecting group or an aliphatic, heteroaliphatic, aryl, heteroaryl, alkylaryl, or alkylheteroaryl moiety;

R₃ is hydrogen, halogen, cyano, —OR_{C}, —N(R_{C})₂, —SR_{C}, —O(C═O)R_{C}, —N(R_{C})(C═O)(R_{C}),

—C(O)R_{C}, —C(O)OR_{C}, —CON(R_{C})₂, —OCO₂R_{C}, or an aliphatic, heteroaliphatic, aryl, heteroaryl, alkylaryl, or alkylheteroaryl moiety, wherein each occurrence of R_{C} is independently hydrogen, a protecting group, or an aliphatic, heteroaliphatic, aryl, heteroaryl, alkylaryl, or alkylheteroaryl moiety;

R₄ is hydrogen, halogen, cyano, —OR_{D}, —N(R_{D})₂, —SR_{D}, —O(C═O)R_{D}, —N(R_{D})(C═O)(R_{D}),

—C(O)R_{D}, —C(O)OR_{D}, —CON(R_{D})₂, —OCO₂R_{D}, or an aliphatic, heteroaliphatic, aryl, heteroaryl, alkylaryl, or alkylheteroaryl moiety, wherein each occurrence of R_{D} is independently hydrogen, a protecting group or an aliphatic, heteroaliphatic, aryl, heteroaryl, alkylaryl, or alkylheteroaryl moiety;

Z is O, S, or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;

X is O, S or NR_{G}, wherein R_{G} is hydrogen or lower alkyl;

A and B together represent

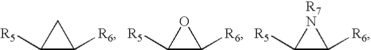

### —CHR₅—CHR₆—, —CR₅═CR₅—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},

—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring,

D and E together represent

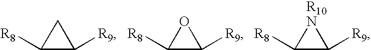

### —CHR₈—CHR₉—, —CR₈═CR₉—, wherein R₈ and R₉ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},

—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₀ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₈—CHR₉—, R₉ and R₉ taken together represent a substituted or unsubstituted 3-7 membered aliphatic, heteroaliphatic, aryl or heteroaryl ring;

G and J together represent

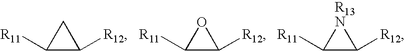

### —CHR₁₁—CHR₁₂—, —CR₁₁═CR₁₂—, wherein R₁₁ and R₁₂ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(—O)R_{J}, —C(—O)OR_{J},

—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₃ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₁—CHR₁₂—, R₁₁ and R₁₂ taken together represent a substituted or unsubstituted 3-7 membered aliphatic, heteroaliphatic, aryl or heteroaryl ring;

K and L together represent

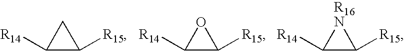

### —CHR₁₄—CHR₁₅—, —CR₁₄═CR₁₅—, wherein R₁₄ and R₁₅ are each independently hydrogen, halogen, cyano, —OR^{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},

—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₆ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₄—CHR₁₅—, R₁₄ and R₁₅ taken together represent a substituted or unsubstituted 3-7 membered aliphatic, heteroaliphatic, aryl or heteroaryl ring;

whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted,

said method comprising:

(1) reacting a component having the structure:

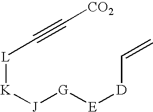

wherein R_{L}, J, and G are as defined above, with a chiral component having the structure:

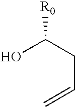

wherein A, B, D, E, G, J, K, and L are as defined above, in the presence of an esterification reagent to generate an intermediate having the structure:

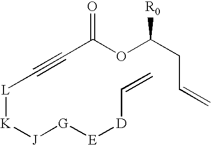

(2) complexing the intermediate with a cobalt, such as dicobalt hexcarbonyl, to yield a structure:

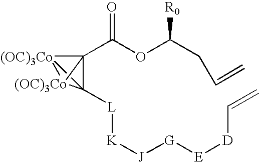

(3) cyclizing the combalt complex in the presence of an olefin metathesis catalyst to generate the compound:

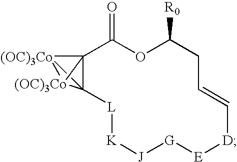

(4) removing the cobalt to form a ynolide;

(5) reacting the alkyne moiety of the ynolide with a diene under cyclcoaddition conditions to generate the compound:

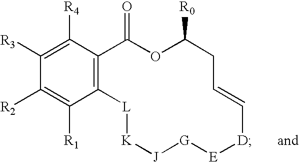

(6) optionally further reacting the macrocycle with one or more reagents to diversify and optionally deprotecting the macrocycle to generate a compound having the formula (IIa).

In addition to the general method described and depicted above, the present invention provides additional synthetic methods, and compounds, as described herein, in which each of the intermediate steps and intermediate compounds are provided, as described below and generically herein. It will be appreciated that the classes, and subclasses, as described above for the inventive compounds are also intended to encompass the inventive methods and intermediate compounds as described above and herein. Thus, certain classes and subclasses of interest in which the moieties R₀-R₄, Z, X, A-B, D-E, G-J and K-L are specifically defined (and moieties defined within those definitions) also apply to the inventive methods and intermediate compounds. It will be appreciated that certain exemplary species of the compounds of formula (I) and (IIa) and intermediates thereto are described herein, but are not limited to those species.

In certain embodiments, if any one or more of R₀-R₄ is a protected thio, amino or hydroxyl group, the method further comprises optionally deprotecting said unprotected group.

In still other embodiments, the method optionally further comprises reacting the intermediates with one or more reagents to diversify the macrocycle and generate a compound having the structure (I) or (IIa). In one embodiment, the one or more positions on the benzene ring are halogenated (e.g., chlorinated). In yet other embodiments, the method further comprises optionally deprotecting the compound having the structure (I) or (IIa), to generate a deprotected compound having the structure (I) or (IIa).

It will also be appreciated that each of the steps as described above can be carried out using reagents and conditions as described for the synthesis of radicicol or aigialomycins (as described below in the Examples), or they may be modified using other available reagents. For example, a variety of esterification conditions and olefin metathesis conditions are well-known in the art and can be utilized in the method of the invention. See, generally, March, Advanced Organic Chemistry, John Wiley & Sons, 1992.

As mentioned above, it will also be appreciated that each of the components used in the synthesis of analogues can be diversified either before synthesis or alternatively after the construction of the macrocycle. As used herein, the term “diversifying” or “diversify” means reacting an inventive compound or intermediate, as defined herein, at one or more reactive sites to modify a functional moiety or to add a functional moiety. For example, the aromatic ring can be diversified to either add functionality (e.g., where hydrogen is present, a halogen, e.g., Cl, can be added) or to modify functionality (e.g., where a hydroxyl group is present on the aromatic ring, the aromatic ring can be diversified by reacting with a reagent to protect the hydroxyl group, or in another example, by reacting with a reagent to add a linker moiety that has a conjugate (e.g., geldanamycin, etc.) attached thereto). Described generally below are a variety of schemes to assist the reader in the synthesis of a variety of analogues, either by diversification of the intermediate components or by diversification of the macrocyclic structures.

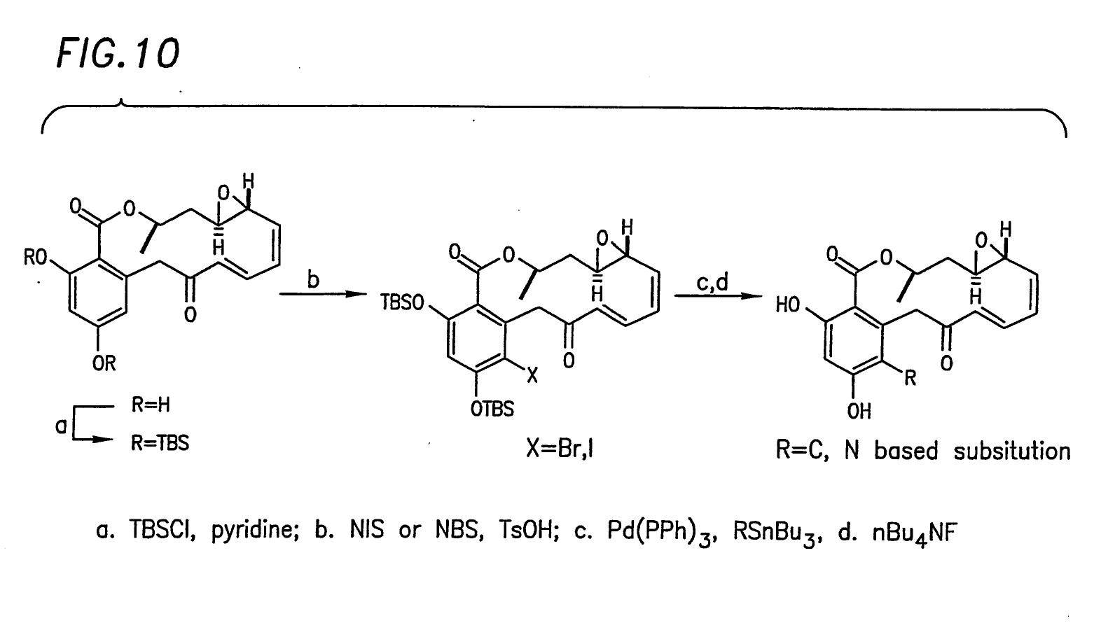

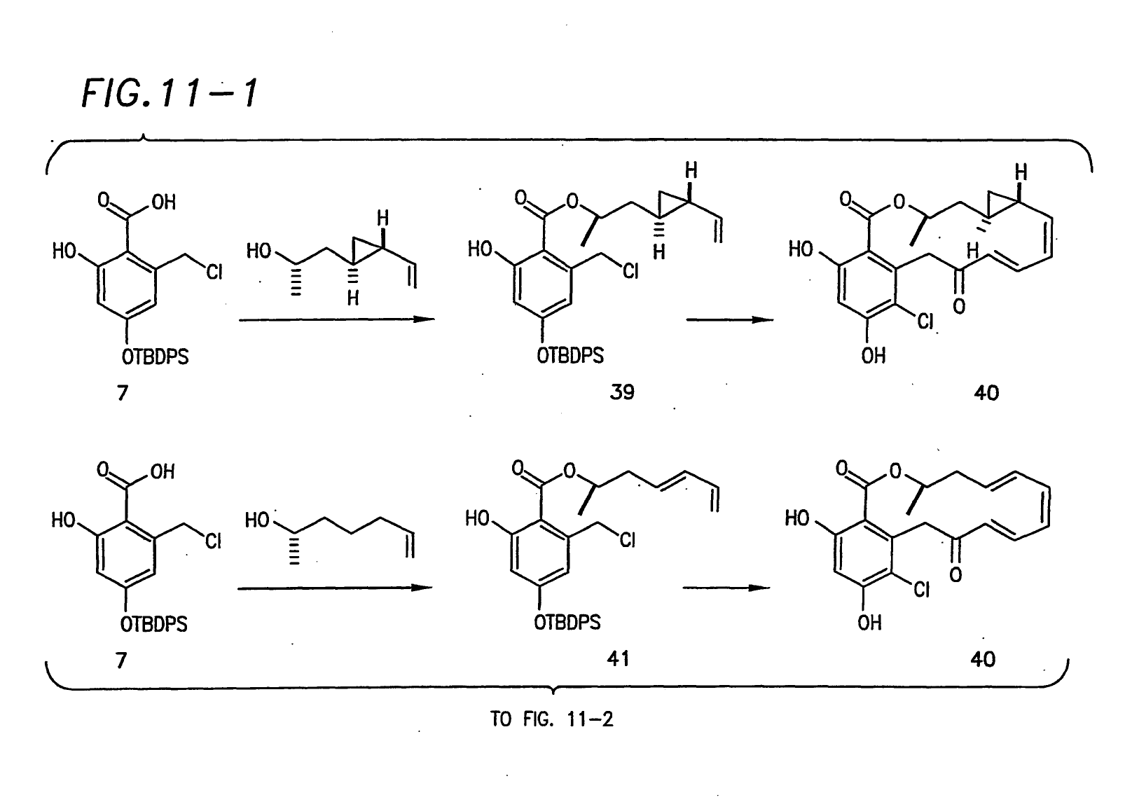

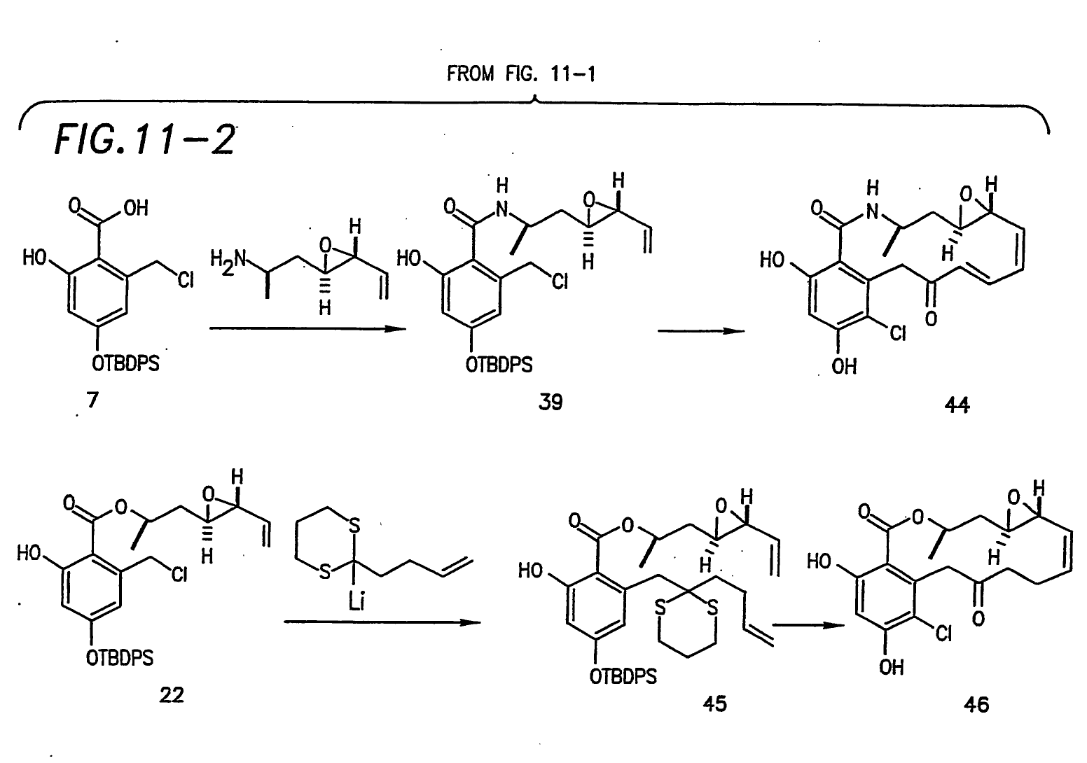

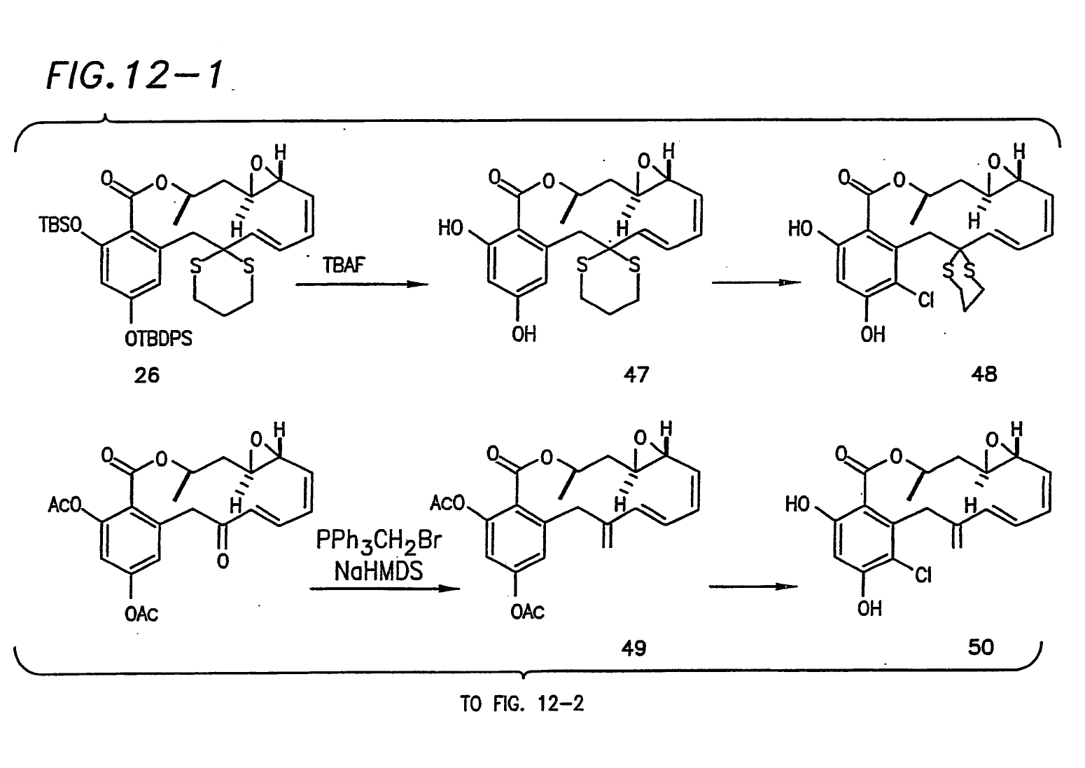

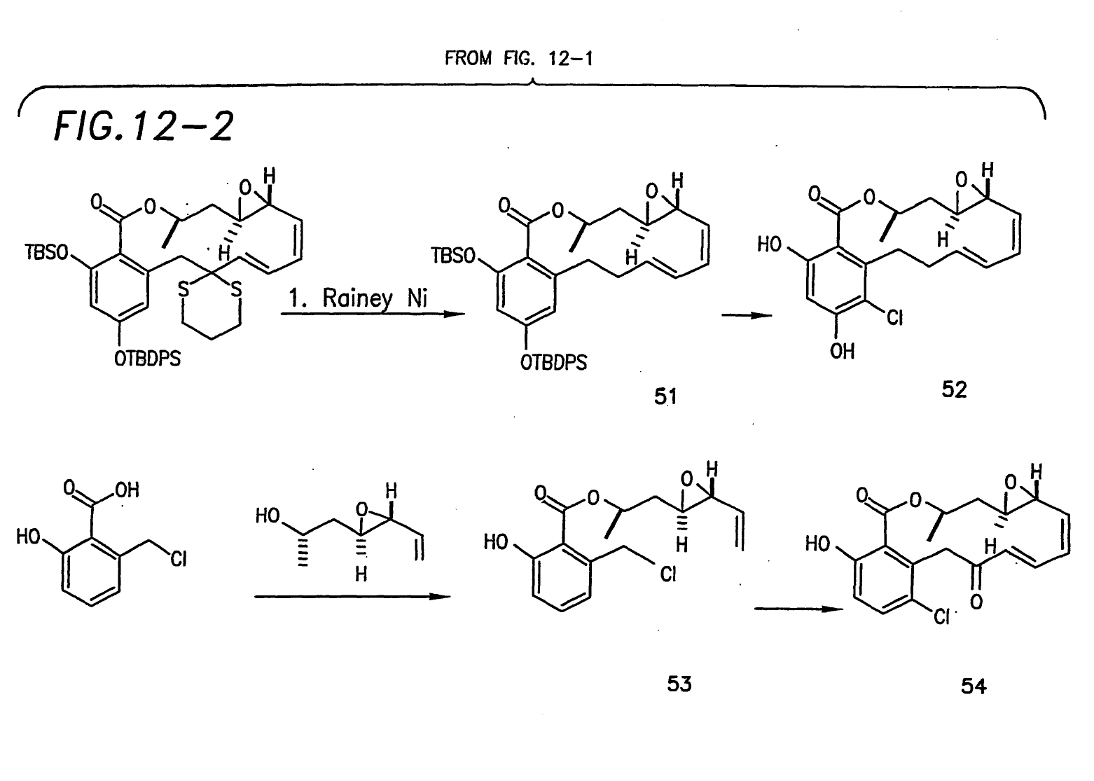

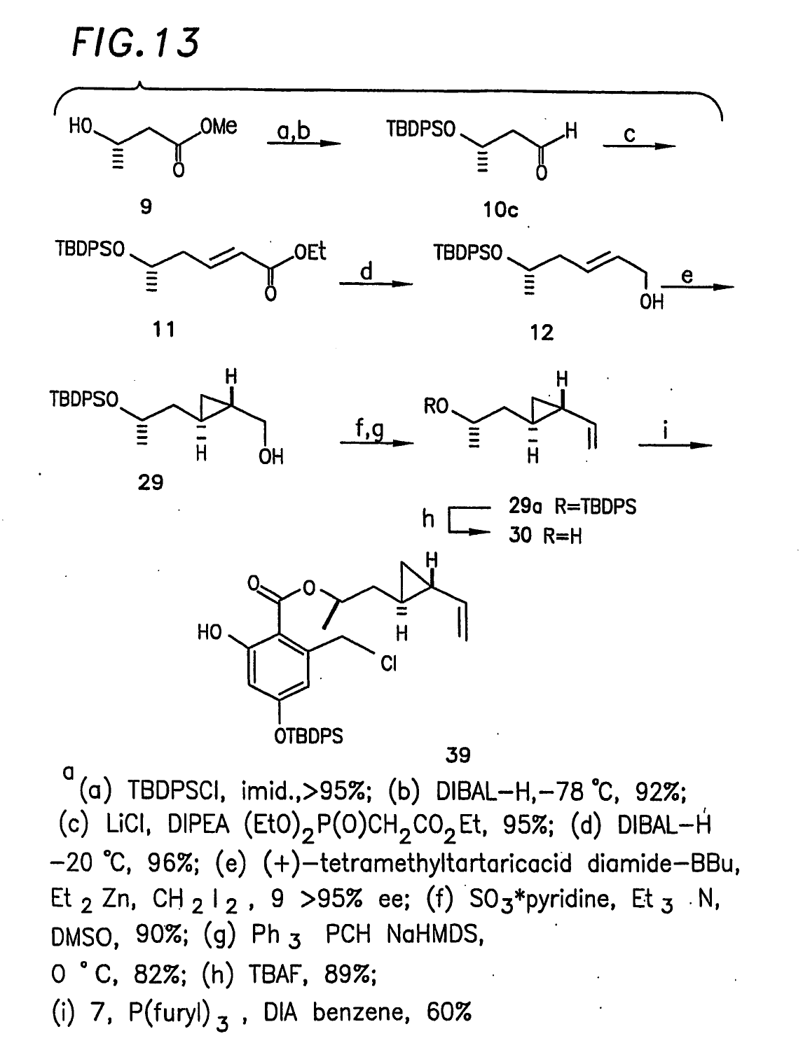

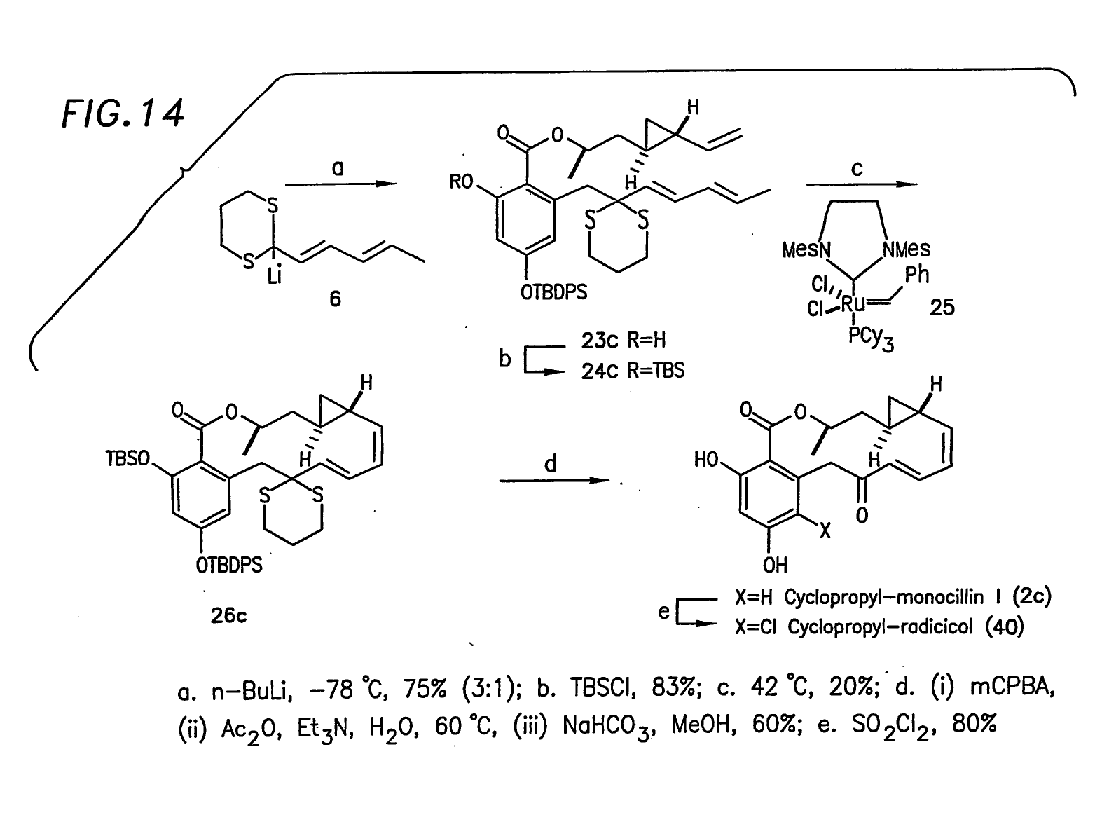

The aromatic ring can incorporate numerous changes. Novel Diels Alder methodology can be utilized to present different substitution patterns around the benzene ring. It will be appreciated that traditional aromatic synthesis can also be utilized to access permutations on the aromatic core. For example, as shown in FIG. 10, diversity can be generated at aromatic positions, in one embodiment, after generation of the core structure. Specifically, R₁ and R₃ include substitution patterns arising from a cross-coupling strategy as depicted to enable introduction of amino, aliphatic, heteroaliphatic, aryl, heteroaryl, and alkylheteroaryl moieties.

Another analog serves to replace the labile ester functionality with a stronger amide component. Esterase activity is responsible for the premature metabolism of many potential therapeutic agents, and therefore this analogs aims to prevent this activity. Simple modification of the chiral component and incorporation into this process provides a facile route to an amide analog.

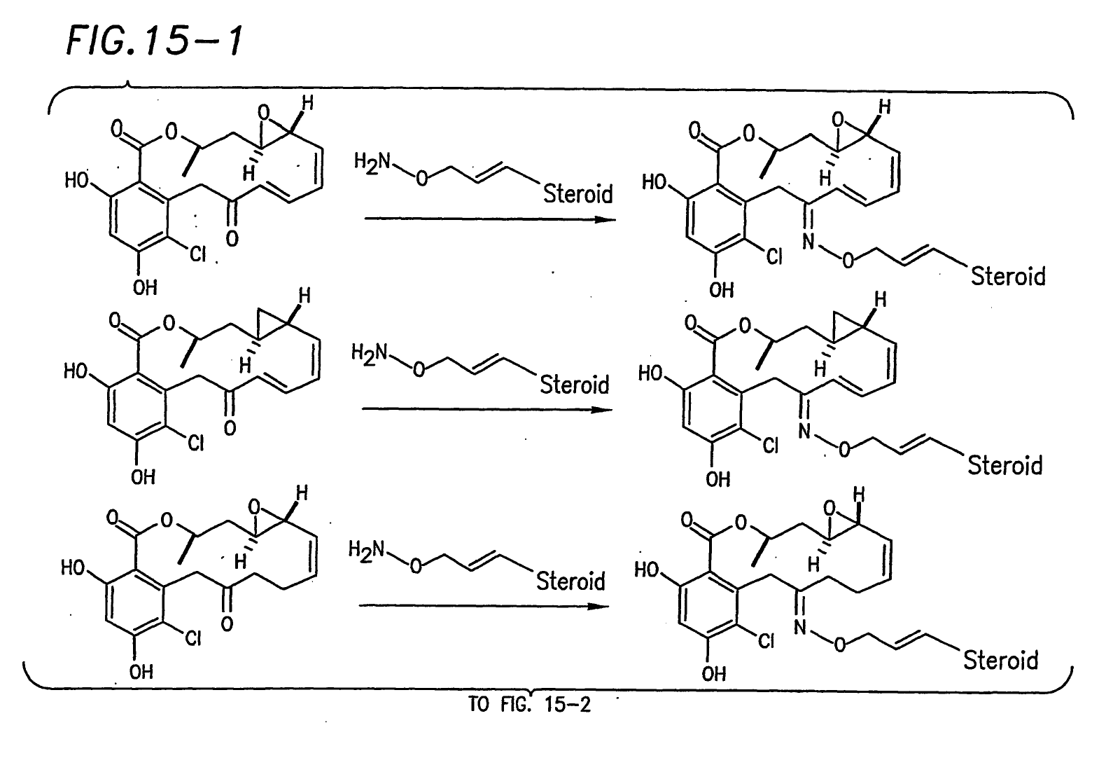

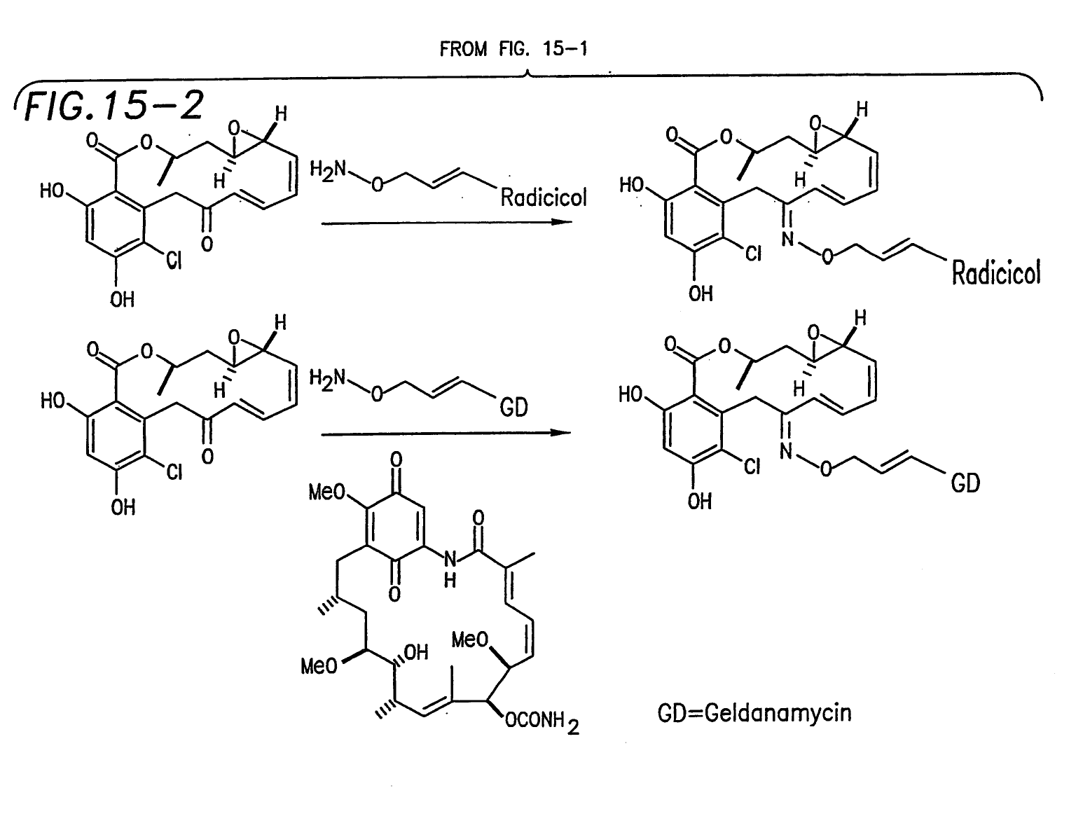

As detailed above, a variety of reactions can be utilized to diversify the radicicol and/or monocillin and aigialomycin core structures either during assembly or after assembly of the macrocycle. An alternative strategy endeavours not only to restore in vivo activity and achieve diversity, but also to enhance it. In a fashion similar to that produced with geldanamycin, a number of known steroid hormones can linked to radicicol and analogs via an oxime technology that has already been shown to restore in vivo activity (see U.S. Pat. No. 6,239,168; U.S. Pat. No. 5,977,165; Soga et al. Cancer Res. 1999, 59, 2931-2938; each of which is incorporated herein by reference). The steroid hormones bind specifically to receptors found in important Hsp90 complexes. In addition, dimers of radicicol or heterodimers of radicicol and geldanamycin will be used to probe the activity of bifunctional binding agents on Hsp90 activity (see FIG. 15).

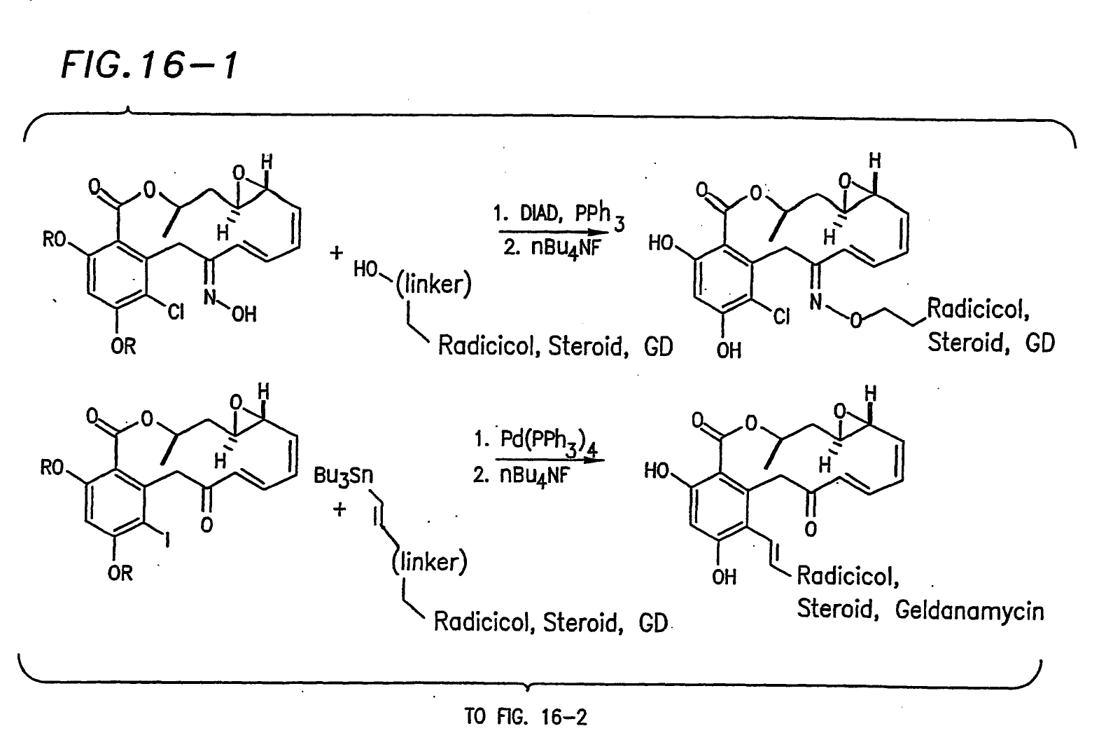

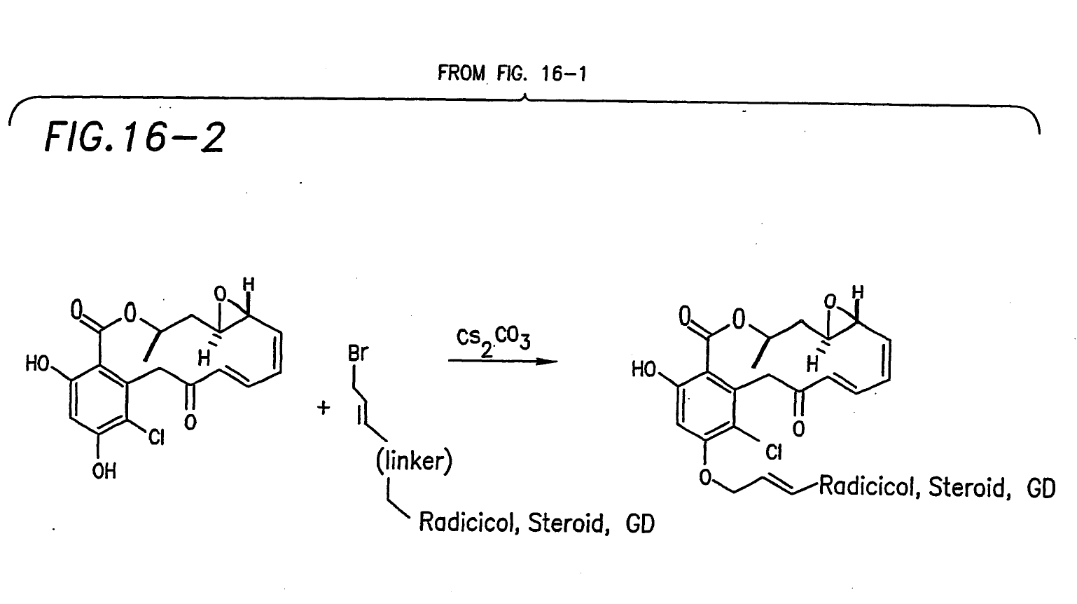

In addition to conjugation via oxime technology as described above and depicted in FIG. 15, it will be appreciated that conjugation can be effected through available functionality on the aromatic ring or through the A-B moiety, as described generally herein. FIG. 16 depicts a variety of methods that can be utilized to effect conjugation.

It will be appreciated that a variety of linkers can be utilized to effect conjugation of geldanamycin, radicicol, monocillin and analogues thereof and steroids to the inventive compounds. As described above, any one or any two of R₁, R_{A}, R₂, R_{B}, R₃, R_{C}, R₄, R_{D}, R₅, R₆, R_{J}, or R_{L} may potentially be a site for conjugation and conjugation can be effected through a linker covalently bonded to a compound to be conjugated. The term “linker” as used herein, is intended to encompass a chemical moiety that is capable of effecting a stable (e.g., sufficiently unhindered that the conjugation can be performed) covalent linkage between an inventive compound as described herein, and another conjugate (geldanamycin, radicicol, monocillin, analogues thereof as described herein and elsewhere) and steroids. It will be appreciated that a variety of linkers can be utilized, including, but not limited to heteroatom linkages (e.g.,

O—S(═O)O, —O—(C═O)—O—, heteroalkyl, etc.), and aliphatic or heteroaliphatic linkages, in which the aliphatic and heteroaliphatic linkages may be substituted or unsubstituted, branched or unbranched, or cyclic or acyclic. In certain embodiments for the compounds as described above, the linker is an aliphatic or heteroaliphatic moiety, whereby said aliphatic or heteroaliphatic moiety is substituted or unsubstituted, branched or unbranched, or cyclic or acyclic. For example, it will be appreciated that this linker may be of varying length, and that altering the length of the linker may, in certain circumstances, confer a therapeutic benefit. In general, the linker may be 1-12 carbon atoms in length, and may also be 1-10, 1-6, or 1-4 carbon atoms in length, in other embodiments of special interest. As described above, the linker may be a linear chain or a substituted chain, for example incorporating double or triple bonds, an aryl group or a secondary or tertiary amine. In certain other embodiments for the compounds as described above, the linker is a moiety having one of the structures —(CH₂)ₙ—CH═CH—(CH₂)ₘ—, —(CH₂)ₚ—C≡C—(CH₂)_{q}—, or —CH₂(CH₂)ₛCH₂—, wherein each occurrence of n, m, p, q and s is independently an integer from 0-10. As described generally above, it will be appreciated that one or more of the hydrogen atoms may be replaced with a substituent including, but not limited to alkyl, heteroalkyl, secondary or tertiary amine, hydroxyl, thiol, aryl, heteroaryl, alkylaryl, or alkylheteroaryl. Similar dimers, trimers, and conjugates and linkers used for the conjugation thereof are described in more detail in Kuduk et al., Bioorg. Med. Chem. Lett. 2000, 10, 1303-1306; Kuduk et al. Bioorg. Med. Chem. Lett., 1999, 9, 1233-1238; Zheng et al. Cancer Res. 2000, 60, 2090-2094; and WO00/61578; each of which is incorporated herein by reference). Additional guidance for the preparation of conjugates can be found in U.S. Pat. No. 5,977,165 (which is incorporated herein by reference) (in which, for example, two radiciciol derivatives are linked via —O—S(═O)—O—).

In certain embodiments, the compound to be conjugated is selected from the group consisting of radicicol, monocillin, analogues of radicicol and monocillin, geldanamycin, analogues of geldanamycin, and steroids. In particular, any of the compounds of the present invention may be conjugated with one or two compounds of the same structure or with one or two compounds of different structures, such as geldanamycin, analogues thereof and steroids. The term “analogues”, as used herein, is intended to encompass radiciciol analogues as described herein and elsewhere (see, e.g., U.S. Pat. Nos. 5,977,165, 5,731,343, and 5,597,846; each of which is incorporated herein by reference) and geldanamycin analogues generally described in the art (see, e.g., WO00/61578; incorporated herein by reference). It will be appreciated that a variety of steroids can be utilized in the method of the present invention. In certain embodiments, steroids are utilized to develop selective cytotoxic agents directed towards cancer cells that express steroid receptors (e.g., estrogen receptor). Suitable steroids for use in the present invention include, but are not limited to, estradiol, estradiol valerate, estradiol cypionate, ethinyl estradiol, mestranol, quinestrol, estrone, estrone sulfate, equilin, testosterone, androstenedione, dehydroepiandrosterone, estriol 16α-hydroxydehydro-epiandrosterone, and 16α-hydroxyandrostenedione, to name a few.

### 5) Uses, Formulation and Administration

### Pharmaceutical Compositions

As discussed above this invention provides novel compounds which have biological properties which make them of interest for the treatment of cancer, in particular those cancers characterized in that they comprise Rb negative cancer cells. Accordingly, in another aspect of the present invention, pharmaceutical compositions are provided, which comprise any one of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be a cytotoxic agent or anticancer agent approved for the treatment of cancer, as discussed in more detail herein, or it may be any one of a number of agents undergoing approval in the Food and Drug Administration that ultimately obtain approval for the treatment of cancer (e.g., epothilones, geldanamycin, to name a few). It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a prodrug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, S. M. Berge, et al. describe pharmaceutically acceptable salts in detail in J. Pharmaceutical Sciences, 66: 1-19 (1977), incorporated herein by reference. The salts can be prepared in situ during the final isolation and purification of the compounds of the invention, or separately by reacting the free base function with a suitable organic acid. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters includes formates, acetates, propionates, butyrates, acrylates and ethylsuccinates.

Furthermore, the term “pharmaceutically acceptable prodrugs” as used herein refers to those prodrugs of the compounds of the present invention which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Fifteenth Edition, E. W. Martin (Mack Publishing Co., Easton, Pa., 1975) discloses various carriers used in formulating pharmaceutical compositions and known techniques for the preparation thereof. Except insofar as any conventional carrier medium is incompatible with the anti-viral compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

### Uses of Compounds of the Invention

As described in more detail herein, in general, the present invention provides compounds useful for their ability to inhibit the growth of or kill cancer cells and thus are useful in the treatment of cancer. The compounds of the invention are also useful as inhibitors of Hsp90 and thus are useful, more generally as inhibitors of proteins such as transmembrane receptors (e.g., HER2, androgen receptor, erbB, EGFR, etc.), tyrosine kinases, serine and/or threonine kinases, transcriptional regulators, or of proteins that regulate them. In certain embodiments, the inventive compounds are also useful for the inhibition of the growth of or for the killing of Rb negative cancer cells and thus are useful in the treatment of cancers comprising Rb negative cancer cells. In certain other embodiments, the inventive compounds are also useful for the destruction of cells expressing a HER-family tyrosine kinase. In still other embodiments, the inventive compounds are useful as inhibitors of the androgen receptor.

In general, the unregulated growth characteristic of cancer cells typically results from disruption of a mitogenic signal transduction pathway. Such pathways can be disrupted at any of a number of points, through activation or inhibition of proteins such as transmembrane receptors (e.g., HER2, which is often overexpressed in breast cancers; steroid receptors such as the androgen receptor, which is often overexpressed in prostate cancers; erbB; EGFR; etc.), tyrosine kinases (including those that are domains of transmembrane receptors), serine and/or threonine kinases (e.g., Akt; Raf; Src; etc.), transcriptional regulators (e.g., Rb; STATs; etc.), or of proteins that regulate them. For instance, the molecular chaperone Hsp90 is required for proper folding of a variety of signal transduction proteins, including, for example, steroid receptors, HER2, met, Akt, Raf, etc. When Hsp90 activity is blocked, these proteins are degraded, and mitogenic signal transduction is attenuated.

It has been previously demonstrated that geldanamycin acts as an Hsp90 inhibitor; administration of this compound to tumor cells results in degradation of Hsp90-regulated proteins and arrest in the G1 phase of the cell cycle. Tumors that express high levels of HER2 are particularly sensitive to such agents. For example, treatment of tumors with geldamycin leads to a dose dependent reduction in HER2 levels as well as visible cellular differentiation. Unfortunately, the cell cycle arrest observed after treatment with these compounds is dependent upon the retinoblastoma (Rb) protein. Geldanamycin is currently in Phase II clinical trials, however, geldanamycin has been shown to be ineffective for the treatment of tumor cells with defective Rb function.

Unexpectedly, the present invention demonstrates that radicicol and radicicol analogs function as Hsp90 inhibitors independent of Rb function. Tumor cells treated with these compounds arrest in G1 in the presence of Rb, and arrest in the prometaphase stage of mitosis in the absence of Rb. Unlike geldanamycin, such compounds are therefore useful for the treatment of Rb-positive and Rb-negative cancers. Importantly, there are cancers which currently lack sufficient treatment, and are comprised of Rb negative cells. These include, but are not limited to, small-cell lung carcinoma, glioblastoma (brain) and retinoblastoma (eye). Small-cell lung carcinoma does not have an effective treatment, results in a high mortality rate, and represents 25% of lung cancers. In some embodiments, it may be desirable to combine administration of the inventive compounds described herein with proapoptotic chemotherapeutic agents and/or with radiation therapy in order to encourage arrested cells to enter apoptosis.

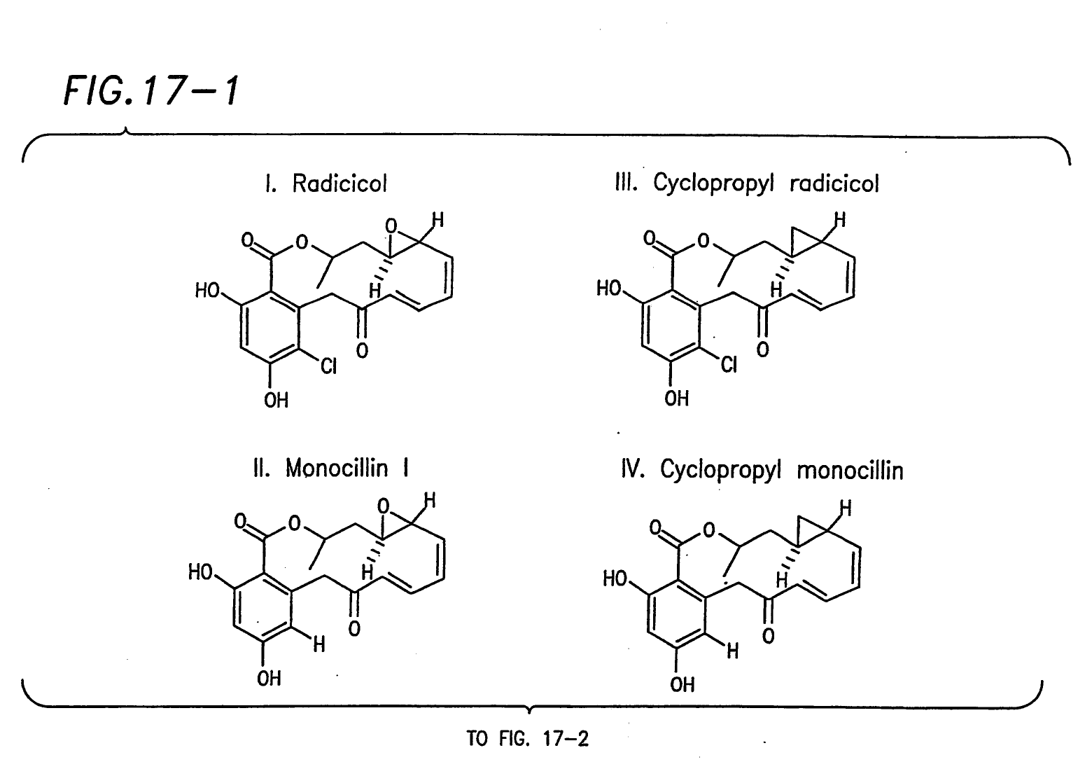

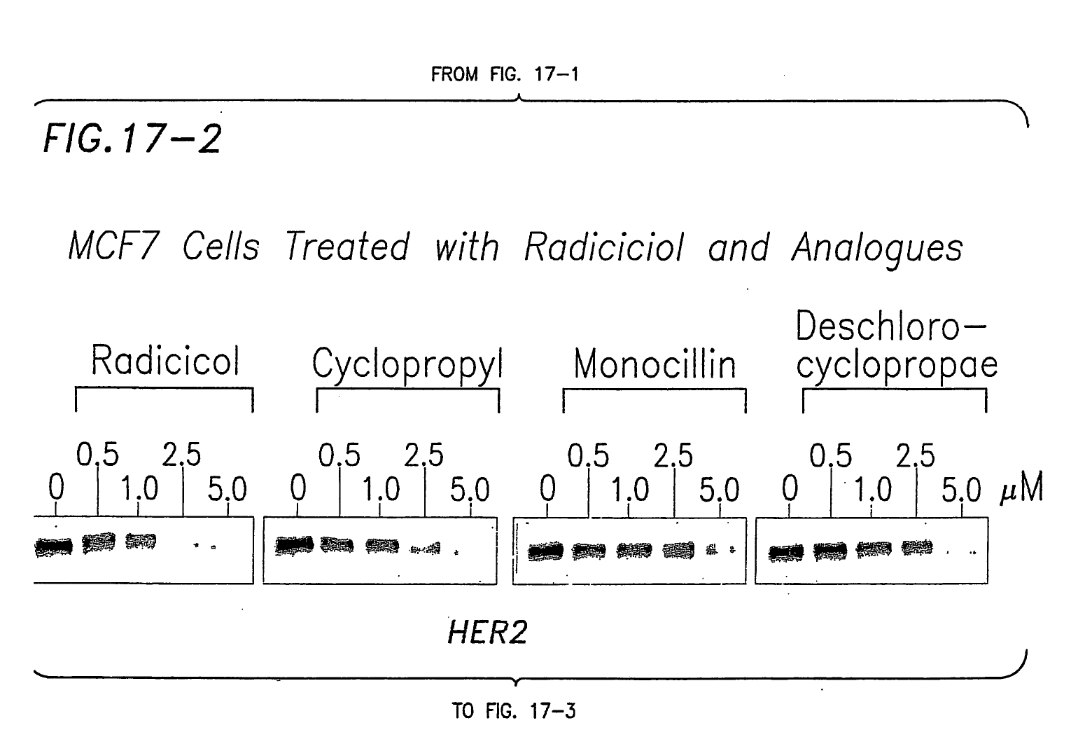

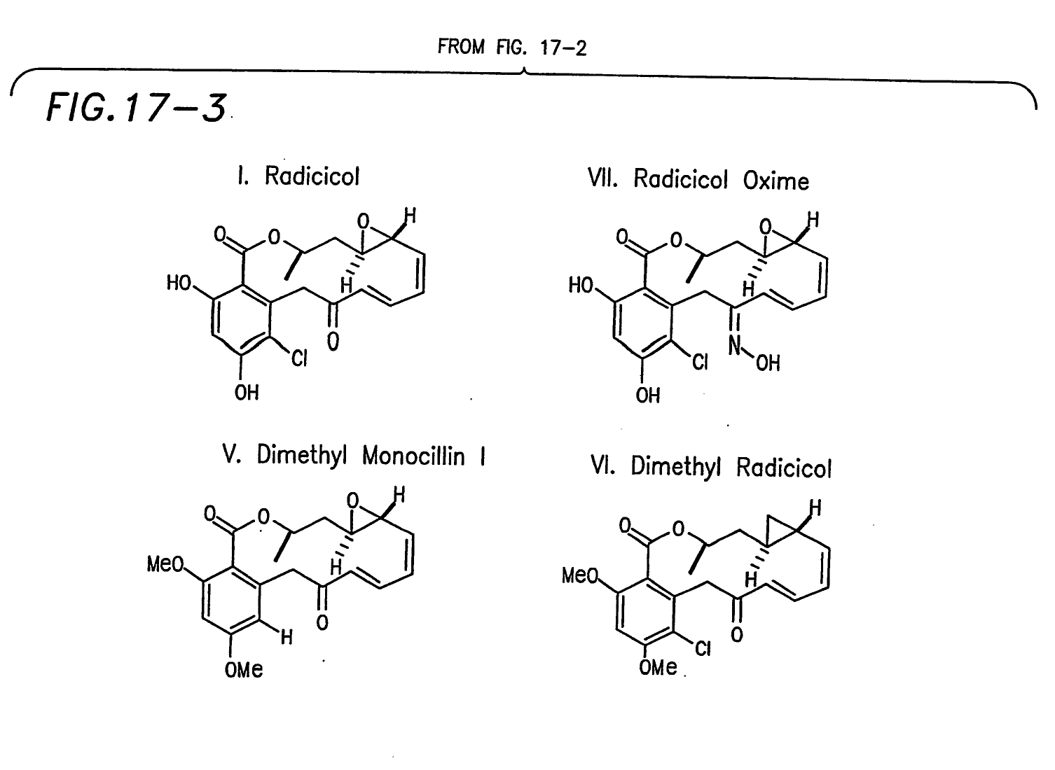

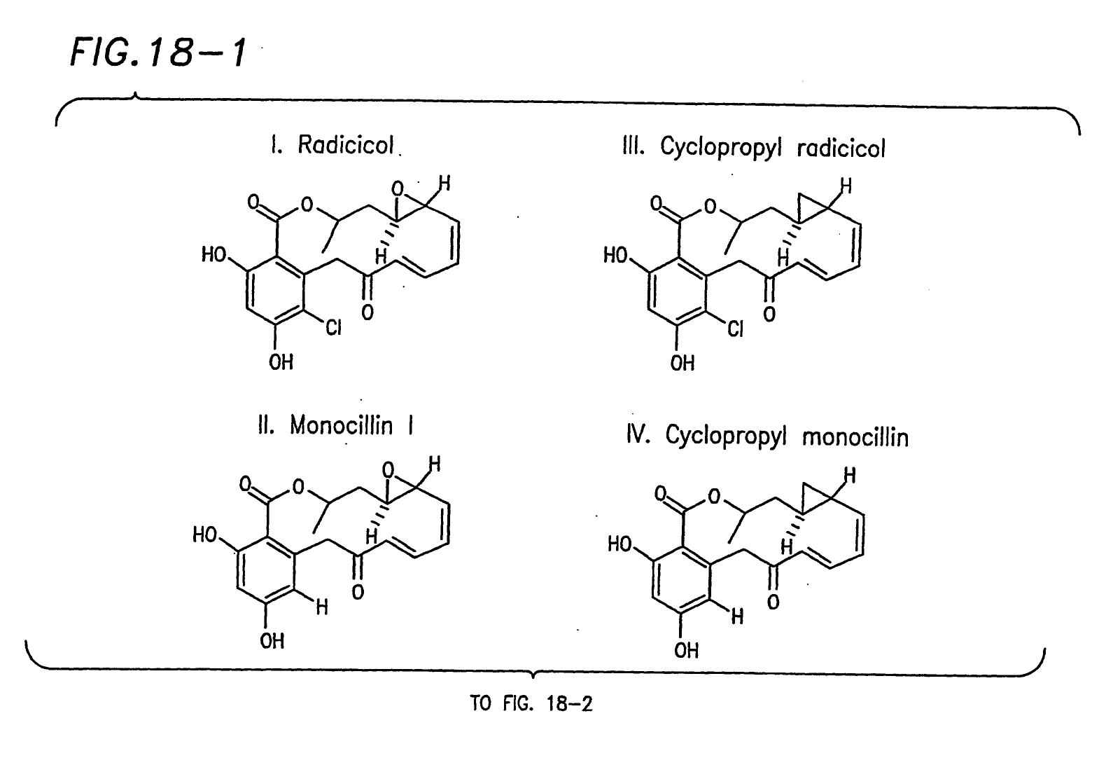

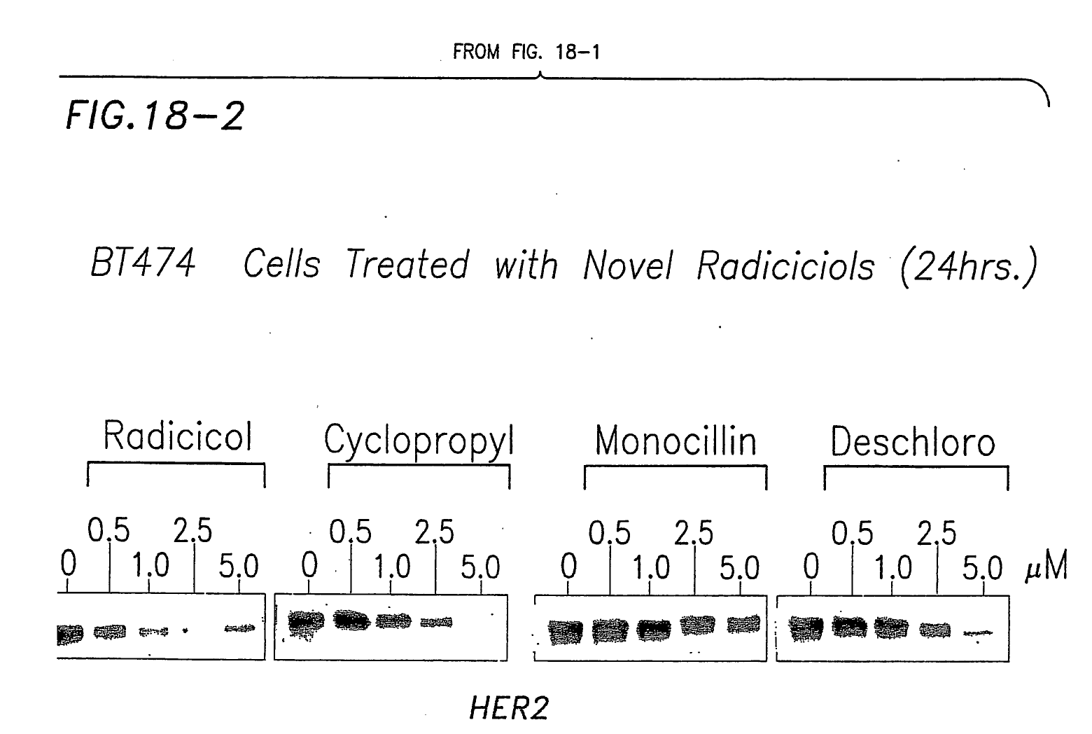

As demonstrated herein (see FIGS. 17-21), radicicol and certain of its analogues have been tested for their cytotoxicity in a panel of cancer cell lines, and importantly, for their ability to lead to the reduction of HER2. As depicted in FIGS. 17 and 18, four analogues have been tested for their ability to reduce HER2 in MCF7 and BT474 cells. Radicicol (1), monocillin I (II), cyclopropyl radicicol (III) and cyclopropyl monocillin I (IV) were each tested at similar concentrations (0.5 μM through 5 μM). These results demonstrate two previously unknown properties with respect to structure-activity relationships: the aromatic chloride contributes to efficacy, and the oxygen of the epoxide does not, despite its binding implication in the known crystal structure. Radicicol is approximately 10× superior in effecting destruction of Her-2 than monocillin I, which only lacks the aromatic chloride. In addition, the cyclopropane analog (II) is at least as effective, if not better than radicicol merely by replacing the oxygen of the epoxide with a CH₂ group. Significantly, currently, the cyclopropane analog can only be made by the route as detailed herein. Furthermore, while the vinyl epoxide, a reactive moiety, may be responsible for undesired cytotoxicity, the cyclopropane is far more stabile to non-productive cellular nucleophiles.

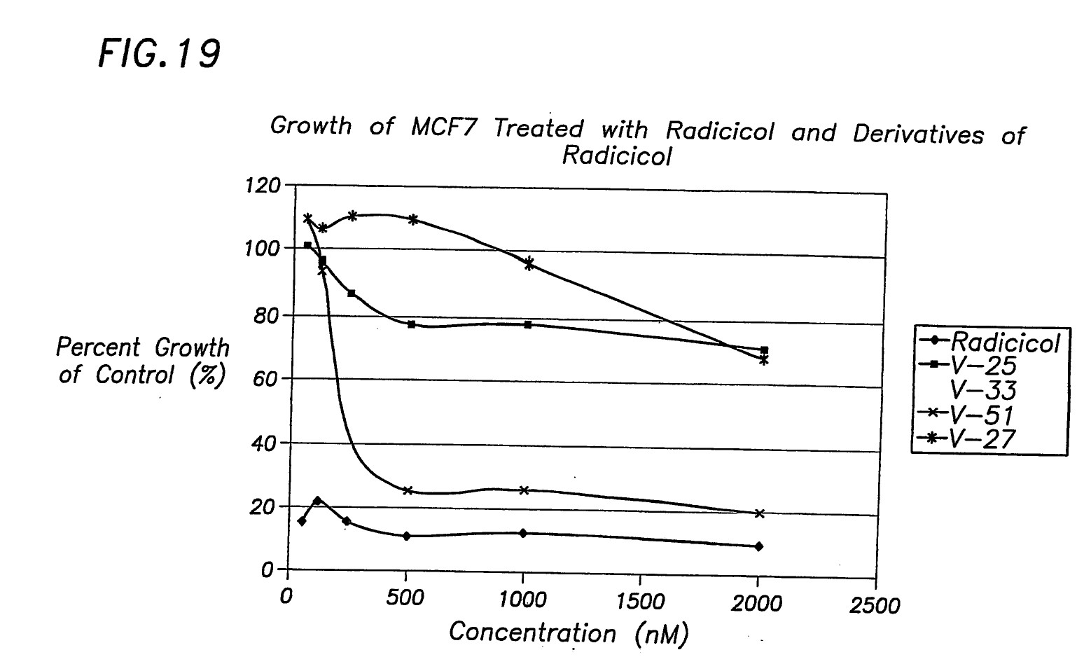

Additionally, the dimethyl ethers of both radicicol and monocillin (V and VI respectively) have been tested, as well as the oxime disclosed by Kyowa Hakko (VII) in a side by side experiment with radicicol. Radicicol again demonstrated efficacy, as did the oxime analog VII which has been reported to also be active in vivo. As shown in FIG. 19, MCF7 cells (HER2 overexpressed, Rb positive) were treated with radicicol (V-27), radicicol oxine (VI-51), dimethyl monocillin (V-25) and dimethyl radicicol (V-33) and the growth of the MCF7 cells was monitored.

As discussed above, the action of geldanamycin and radicicol on Hsp90 in Rb (retinoblastoma) positive cells results in a clear G1 (growth) phase block of the cell cycle, degradation of HER2, and eventual reversion, apoptosis or necrosis. Interestingly, and without a clear explanation, when radicicol is applied to Rb negative cells, the cells are blocked in the M (mitosis) phase. While geldanamycin and its derivatives are demonstrating success in Rb positive cell lines, they are much less effective in their ability to halt growth in the corresponding Rb negative cells. Radicicol and derivatives appear to have an advantage in these Rb negative cell lines as they demonstrate superior efficacy blocking these cells in mitosis.

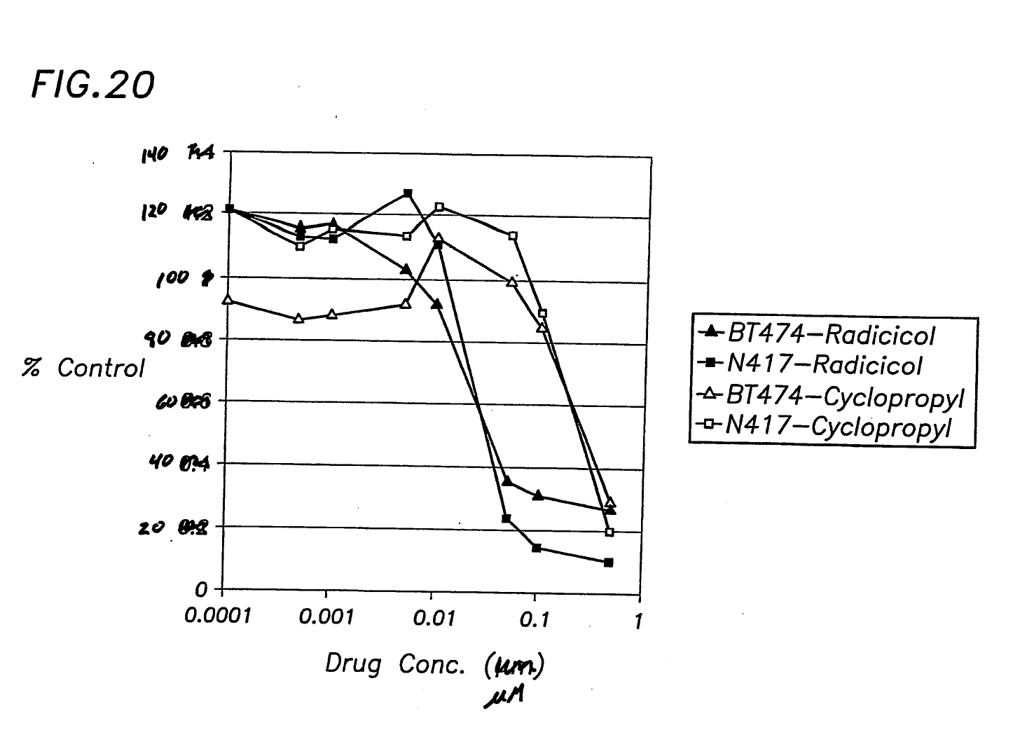

In addition, it has also been shown that 17-alkylaminogeldanamycin (17-AGG), an ansamycin derivative, is much more potent against cells with wild type Rb than in cells with defective Rb function. It has also been shown that radicicol and cyclopropyl radicicol are potent inhibitors of both a breast cancer cell with wild type Rb and a small cell lung cancer cell line with defective Rb function (See FIG. 20). Significantly, they have been found to be much more potent than 17-AGG in the latter cell type. These data suggest that radicicol derivatives are useful in the treatment of the 15-20% of human tumors with mutated Rb gene and especially in small lung cell cancer, in which the gene is almost always mutated.

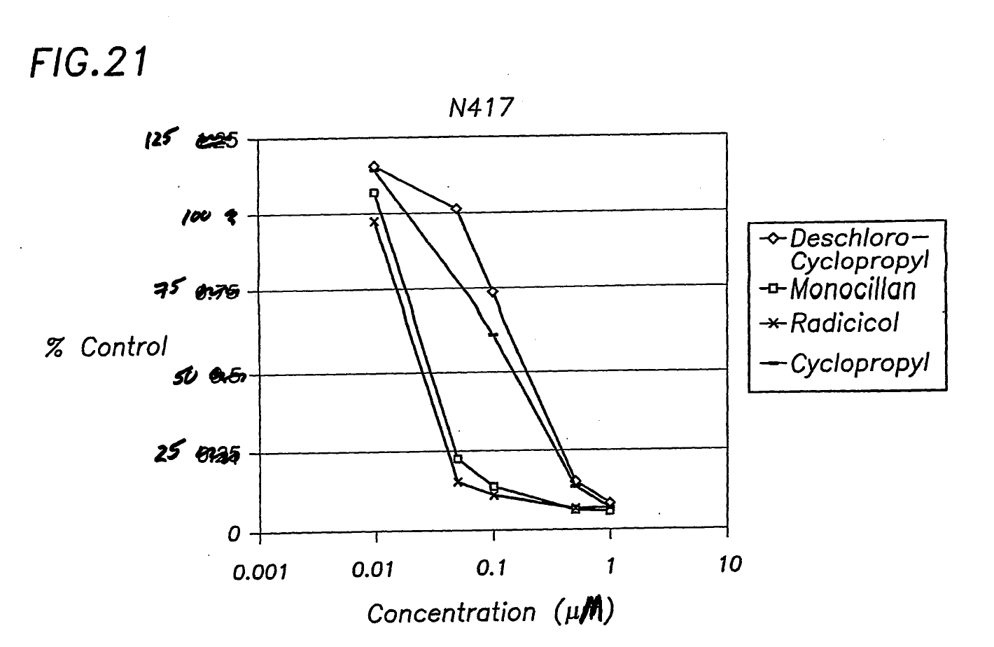

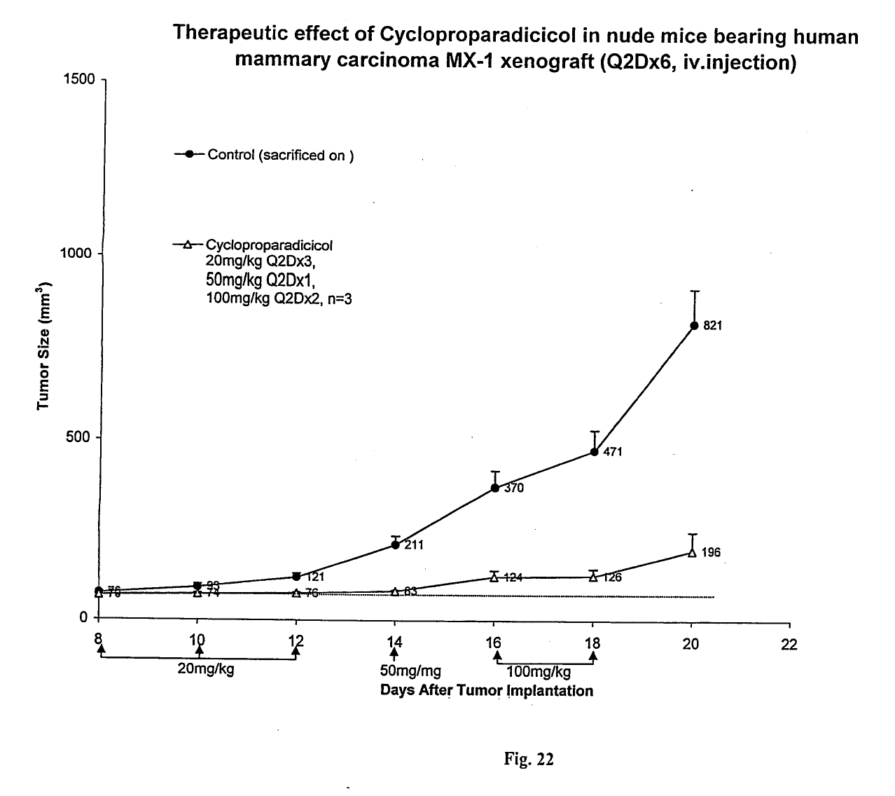

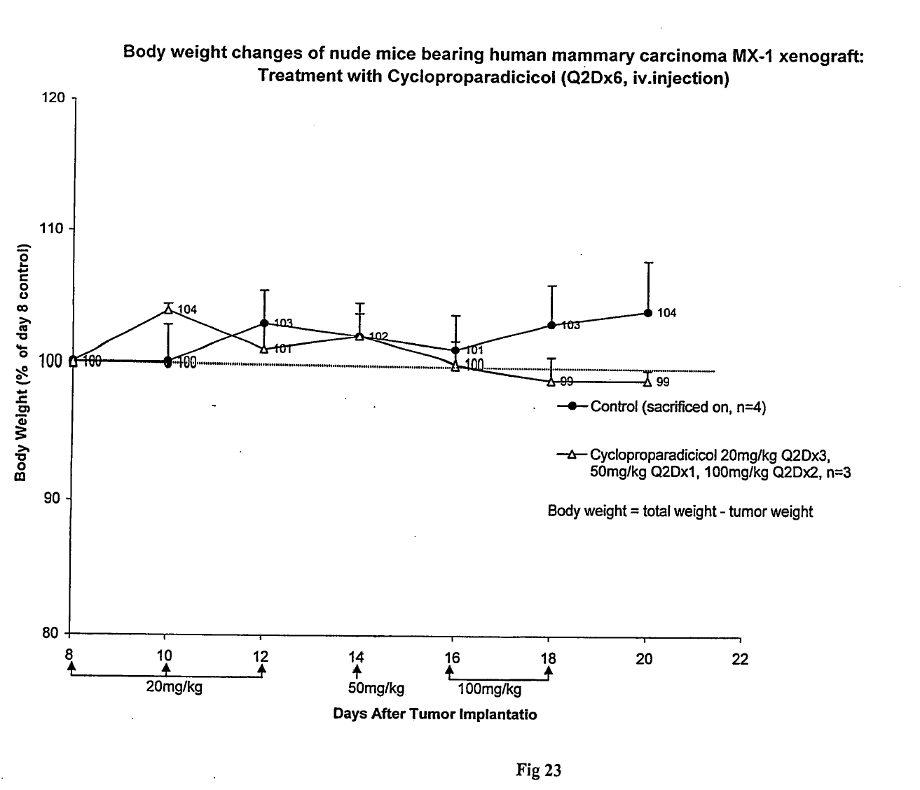

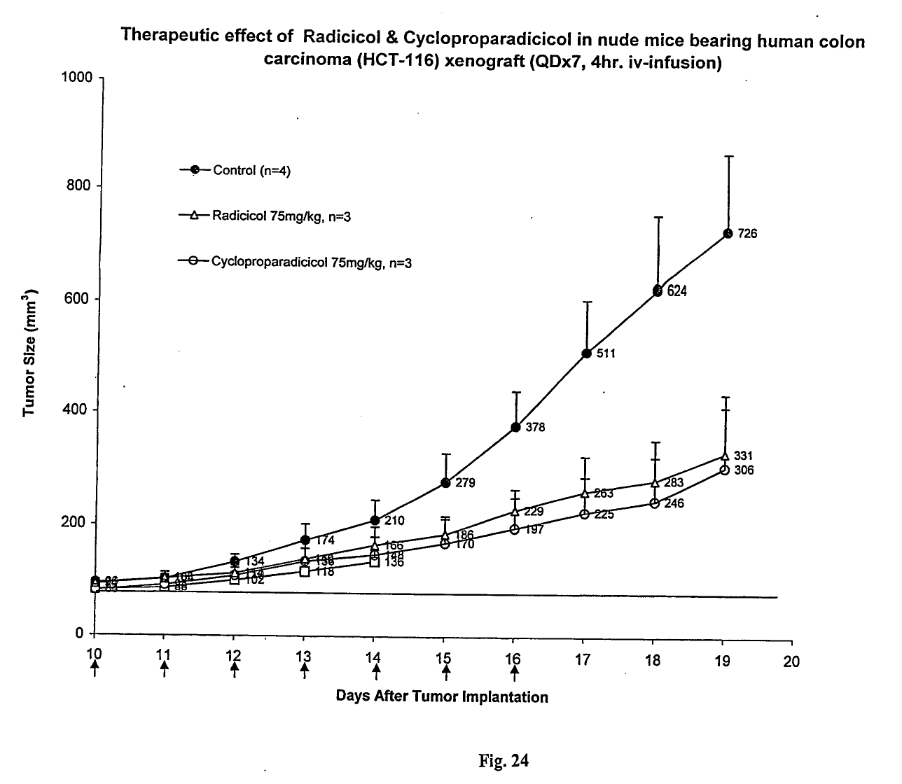

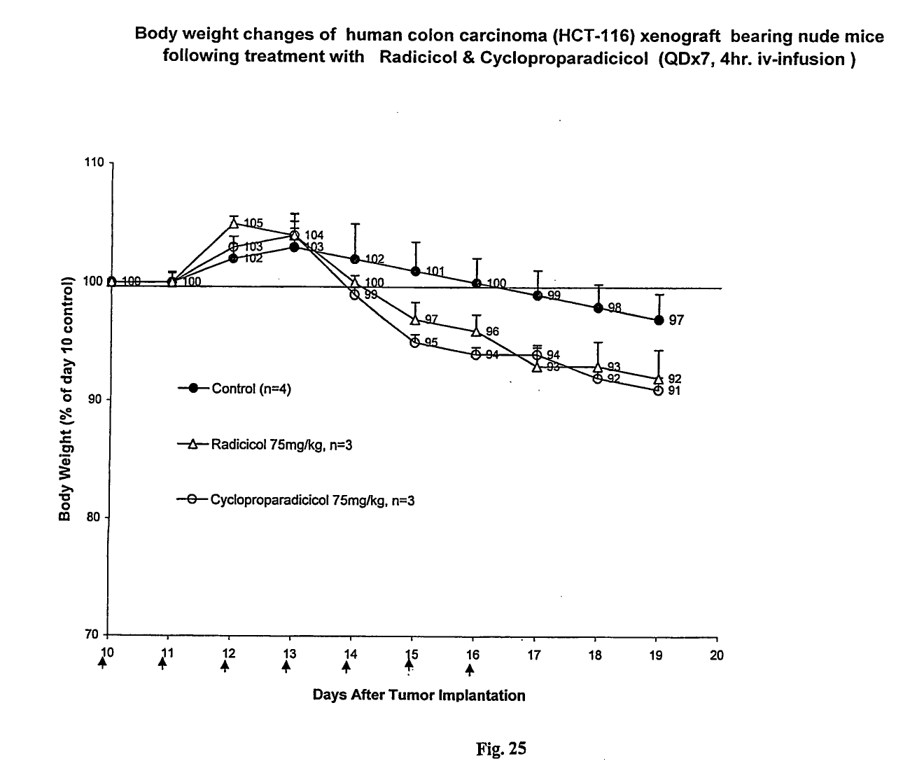

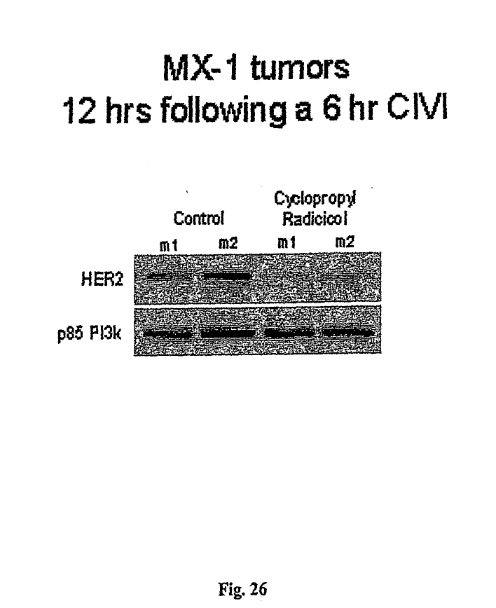

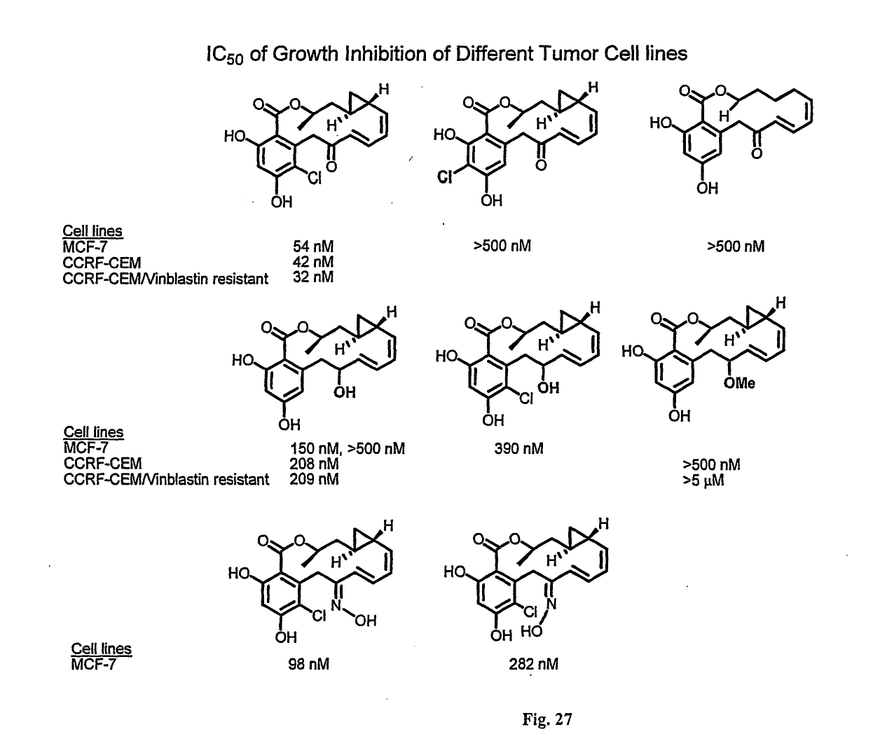

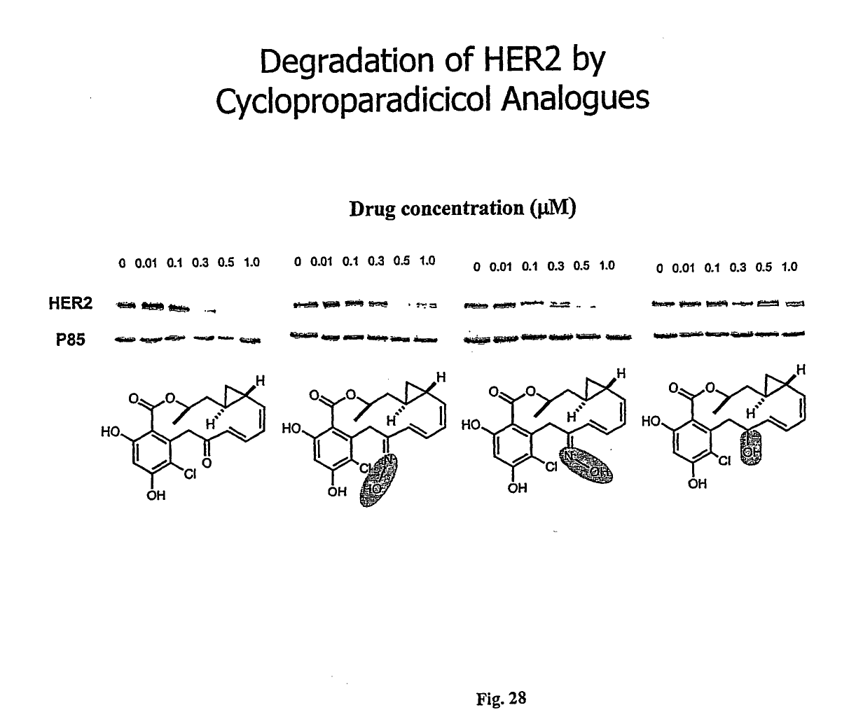

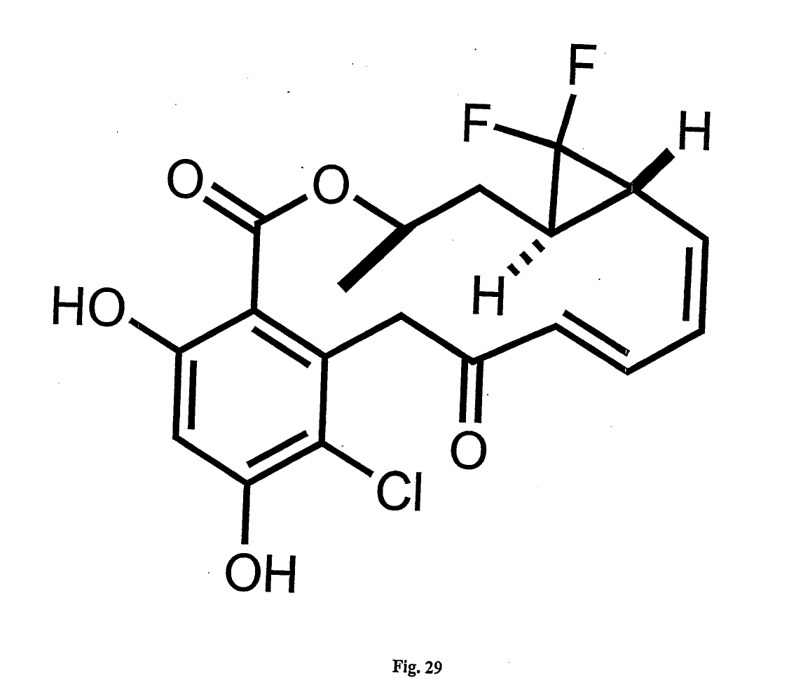

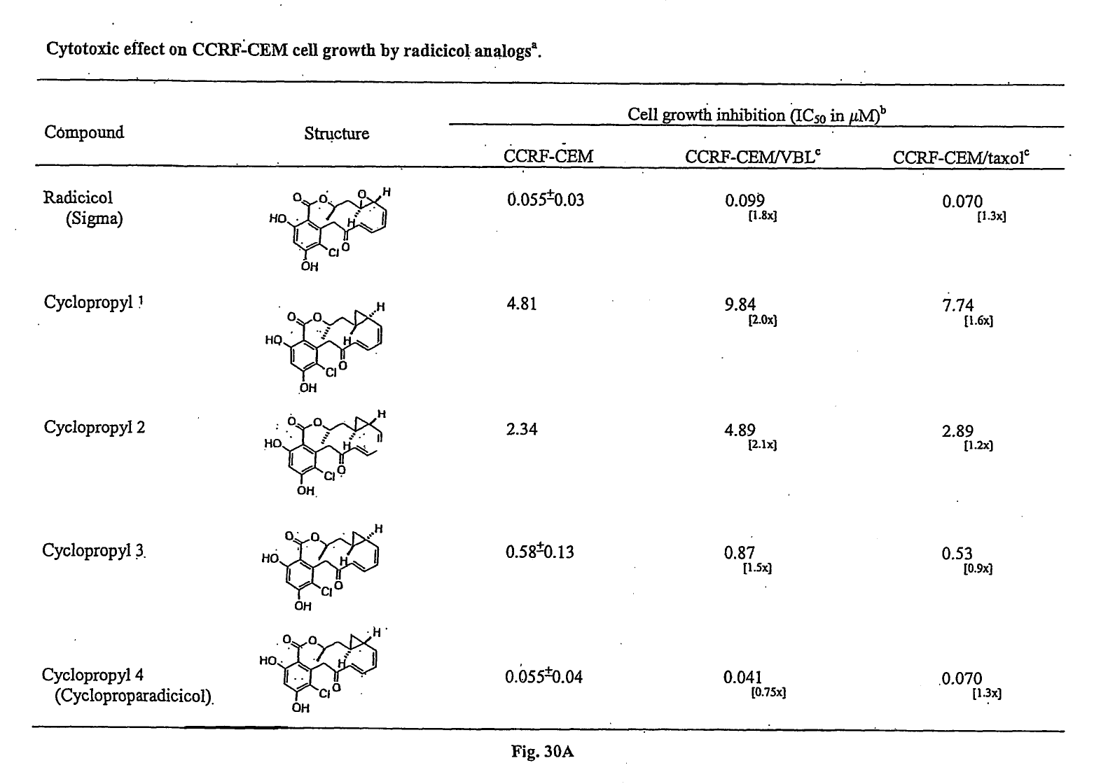

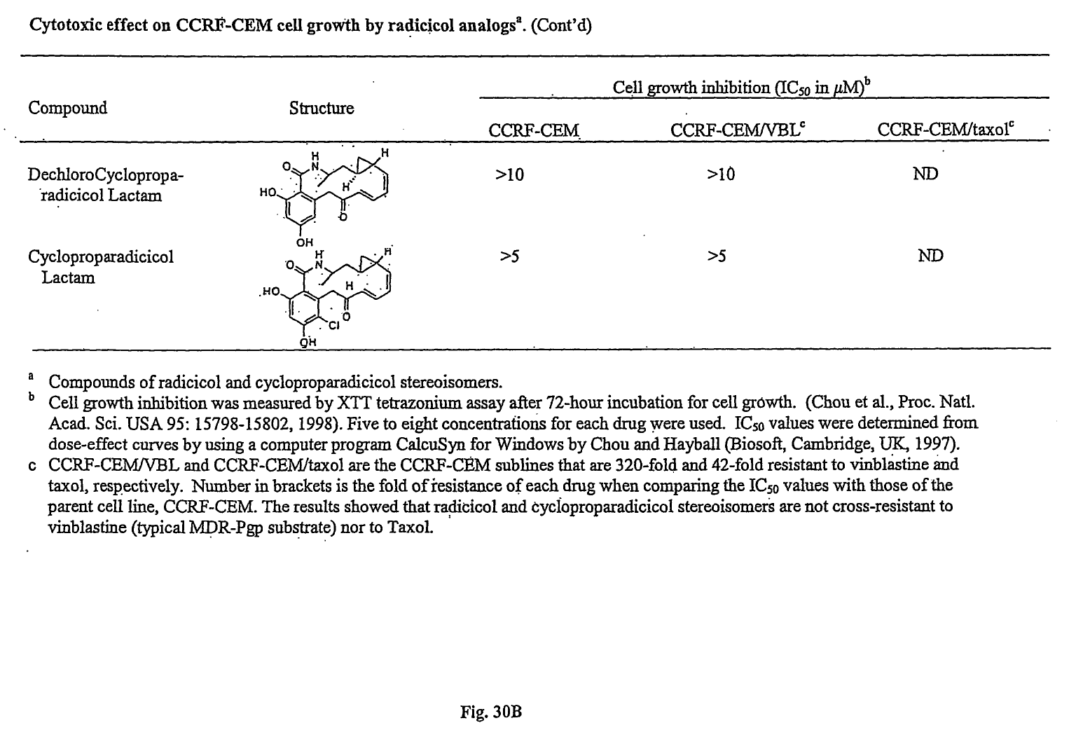

As discussed herein, the compounds of the present invention have been shown to reduce HER2 in MCF-7 and BT474 cells, and are cytotoxic against a panel of cancer cell lines (see exemplification herein and FIGS. 19, 20 and 21), in particular against Rb negative cancer cell lines (FIGS. 20 and 21). The inventive compounds are thus useful for the inhibition of the growth of or for killing cancer cells and are useful in the treatment of cancer (or more generally useful in the treatment of proliferative disorders). In addition, compounds as described herein have been found to act as potent inhibitors of cancer cell lines comprising Rb negative cells, and thus are useful in the treatment of cancers comprising Rb negative cells. Currently, there is no effective treatment for many of these cancers comprising Rb negative cells. The method of the invention comprises administering to a subject in need thereof a therapeutically effective amount of a compound of the invention.

In certain embodiments of the present invention a “therapeutically effective amount” of the inventive compound or pharmaceutical composition is that amount effective for detectable killing or inhibiting the growth of cancer cells, and in certain embodiments of special interest an amount for detectable killing or inhibiting the growth of cancer cells comprising Rb negative cancer cells.

The compounds and compositions, according to the method of the present invention, may be administered using any amount and any route of administration effective for killing or inhibiting the growth of tumor cells. Thus, the expression “effective amount” as used herein, refers to a sufficient amount of agent to kill or inhibit the growth of tumor cells. The exact amount required will vary from subject to subject, depending on the species, age, and general condition of the subject, the severity of the infection, the particular anticancer agent, its mode of administration, and the like. The anticancer compounds of the invention are preferably formulated in dosage unit form for ease of administration and uniformity of dosage. The expression “dosage unit form” as used herein refers to a physically discrete unit of anticancer agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. I will also be appreciated that dosages smaller than 0.001 mg/kg or greater than 50 mg/kg (for example 50-100 mg/kg) can be administered to a subject. In certain embodiments, compounds are administered orally or parenterally.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

As discussed above, in one aspect, the compounds of the present invention are useful as anticancer agents, and thus may be useful in the treatment of cancer, by effecting tumor cell death or inhibiting the growth of tumor cells. In general, the inventive anticancer agents are useful in the treatment of cancers and other proliferative disorders, including, but not limited to glioblastoma, retinoblastoms, breast cancer, cervical cancer, colon and rectal cancer, leukemia, lung cancer (including, but not limited to small cell lung cancer), melanoma, multiple myeloma, non-Hodgkin's lymphoma, ovarian cancer, pancreatic cancer, prostate cancer, and gastric cancer, to name a few. In certain embodiments, the inventive anticancer agents are active against cancers comprising Rb negative cells, including, but not limited to small cell lung cancer, retinoblastoma and glioblastoma. In certain other embodiments, the inventive anticancer agents are active against breast cancer cells, leukemia cells and melanoma cells, and thus are useful for the treatment of breast cancer, leukemias (e.g., myeloid, lymphocytic, myelocytic and lymphoblastic leulcemias) and malignant melanomas. In still other embodiments, the inventive anticancer agents are active against solid tumors and also kill and/or inhibit the growth of multidrug resistant cells (MDR cells).

It will also be appreciated that the compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

For example, other therapies or anticancer agents that may be used in combination with the inventive anticancer agents of the present invention include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. Additionally, the present invention also encompasses the use of certain cytotoxic or anticancer agents currently in clinical trials and which may ultimately be approved by the FDA (including, but not limited to, epothilones and analogues thereof and geldanamycins and analogues thereof). For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference.

### Treatment Kits

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the substituted purine dosages, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### Equivalents

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

The following examples contain important additional information, exemplification and guidance that can be adapted to the practice of this invention in its various embodiments and the equivalents thereof.

### EXEMPLIFICATION

### Example 1

### Total Synthesis of Aigialomycin D

There are many natural products, usually bacterial metabolites, featuring a macrolide fused to a monocyclic benzenoid matrix, bearing a resorcinol-like substitution pattern. Not infrequently, the resorcinol moiety carries additional functionality, resulting in higher levels of oxidation. Natural products in this family (cf. inter alia radicicol (Delmotte, P.; Delmotte-Plaquee, J. Nature 1953, 171, 344; incorporated herein by reference), LL-Z-1640s (McGahren, W. J. J. Org. Chem. 1978, 43, 2339-2343; incorporated herein by reference), monocillins (Ayer, W. A.; Lee, S. P.; Tsuneda, A.; Hiratsuka, Y. Can. J. Microb. 1980, 26, 766-773; incorporated herein by reference), nordinone (Ayer, W. A.; Pena-Rodriguez, L. Phytochemistry 1987, 26, 1353-1355; incorporated herein by reference), and zearelenone (Sugawara, F.; Kim, K. W.; Kobayashi, K.; Uzawa, J.; Yoshida, S.; Murofushi, N.; Takahashi, N.; Strobel, G. A. Phytochemistry 1992, 31, 1987-1990; incorporated herein by reference) possess potentially exploitable patterns of antitumor, antibiotic and antimalarial activity. Indeed, we were first attracted to this structural series by radicicol (Delmotte, P.; Delmotte-Plaquee, J. Nature 1953, 171, 344; Sharma, S. V.; Agatsuma, T.; Nakano, H. Oncogene 1998, 16, 2639; each of which is incorporated herein by reference)—a non quinoidal inhibitor of the key molecular chaperone HSP90. Using radicicol (2) as a lead compound, we were soon led to cycloproparadicicol (3) (Yamamoto, K.; Garbaccio, R. M.; Stachel, S. J.; Solit, D. B.; Chiosis, G.; Rosen, N.; Danishefsky, S. J. Angew. Chem., Int. Ed. 2003, 42, 1280-1284; incorporated herein by reference) as a potentially valuable analog structure, wherein the cyclopropane simulates the conformational consequences of the epoxide, without the liabilities associated with a potentially labile alkylation site. Indeed, xenograft studies suggest that cycloproparadicicol (3) may well be a superior drug relative to radicicol (2). The promise of cycloproparadicicol, albeit in an early preclinical setting, as well as the structural diversity encountered in this family of bioactive molecules, prompted us to explore new strategies for building such compounds in the laboratory. Indeed, a new strategy was described to reach cycloproparadicicol (Yang, Z.-Q., Danishefsky, S. J. J. Am. Chem. Soc. 2003, 125, 9602-9603; incorporated herein by reference).

Recently, five new 14-membered resorcyclic macrolides, termed aigailomycins A-E, were isolated from the marine mangrove fungus Aigialus parvus BCC5311 (Isalca, M.; Suyarnsestakorn, C.; Tanticharoen, M.; Kongsaeree, P.; Thebtaranonth, Y. J. Org. Chem. 2002, 67, 1561-1566; incorporated herein by reference). Among those compounds, aigialomycin D (1) (shown below) exhibited potent antimalarial activity (IC₅₀: 6.6 μg/mL against P. falciparum) and antitumor activity (IC₅₀: 3.0 μg/mL against KB cells) (Isaka, M.; Suyarnsestakorn, C.; Tanticharoen, M.; Kongsaeree, P.; Thebtaranonth, Y. J. Org. Chem. 2002, 67, 1561-1566; incorporated herein by reference).

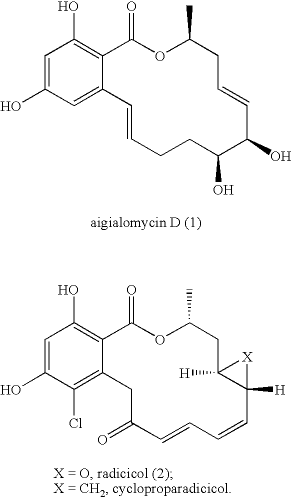

Resorcinylic Macrolides: Aigialomycin D, Radicicol, and Cycloproparadicicol.

Not surprisingly, our first thought was to use the synthetic paradigm developed for cyclproparadicicol (Yang, Z.-Q., Danishefsky, S. J. J. Am. Chem. Soc. 2003, 125, 9602-9603; incorporated herein by reference). However, unlike 3, 1 does not, in the end, contain benzylic oxygen functionality. Rather, it contains a 1′, 2′ styrene-like double bond, ortho to the acyloxyl group of the lactone. It was our plan to install this double bond by β-elimination of a C2′ leaving group toward the benzo domain (vide infra). To bring about a pre-elimination setup, the initial bond formation would be between future carbons 1′ and 2′. The more serious incremental complexity in the aigialomycin series arises from the two hydroxy-bearing stereogenic centers at C5′ and C6′ in allylic and homoallylic relationships respectively to the C7′-C8′ double bond. The thought was to construct this double bond by ring forming olefin metatheses via extrusion of carbons 7″ and 8″. Scheme 1-1 (below) sets forth the thinking that led to a remarkably straightforward total synthesis of agialomycin D.

It was recognized that, if properly managed, the functionality present in the readily available D-2-deoxyribose (9) could lead to a functional equivalent of the key proposed formal building block 6. Compound 6 would not be used as such (vide infra).

In the event, the secondary hydroxyl groups at C3 and C4 of 9, were engaged in an isopropylidene linkage (see 10 (Barbat, J.; Gelas, J.; Horton, D. Carbohydrate Res. 1983, 116, 312-316; incorporated herein by reference), Scheme 1-2). The masked aldehyde character of Cl of the pentose could be exploited in the context of a Wittig protocol. The primary alcohol in the resultant product, 11, was protected as its pivaloyl derivative (see 12). In this compound, the primary methylene group bearing the pivaolyloxy group would emerge as C7′ of the ring closing metathesis (RCM) precursor (vide infra). Hydroboration of 12 followed by oxidation, as shown, led us to 13 which, following oxidation of its primary alcohol function, delivered 14.

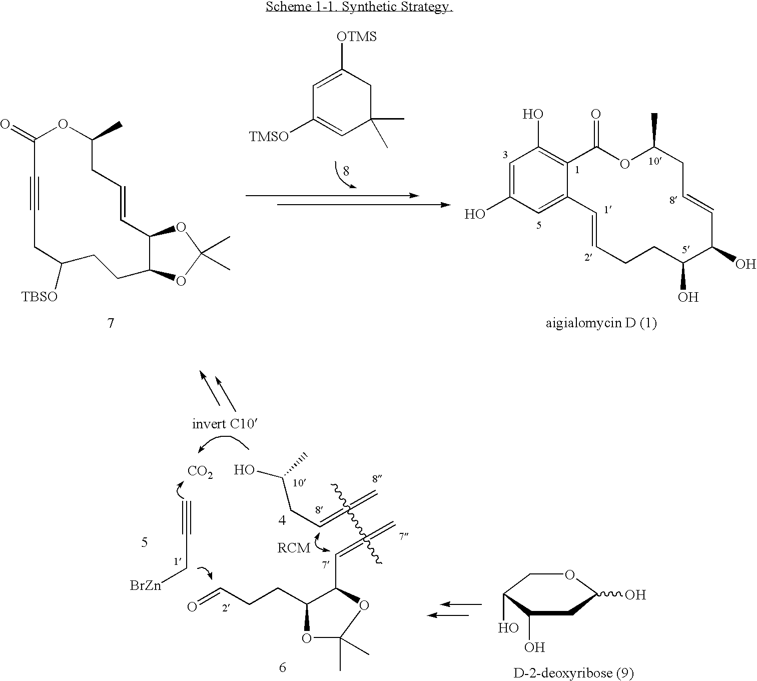

Chain extension of the aldehyde by propargylation afforded 15 as a mixture of stereoisomers. In this compound, as well as in subsequent seco intermediates, these stereoisomers manifested nearly identical chromatographic characteristics. Thus, the mixtures were treated as single entities in the progression until compound 23. The secondary hydroxyl groups in epimers 15 were protected as t-butyldimethyl silyl ethers (see 16), thus enabling installation of a vinyl group, destined to serve as the C7′-C7″ moiety in the eventual RCM (see steps leading to 18).

Carboxylation of the ethynyl group in 18 occurred smoothly to afford carboxylic acid 19. The latter reacted with R alcohol 4, giving rise, through a Mitsunobu protocol, to the S-ester 20, still bearing epimeric OTBS ethers at the future carbon 2′.

Our previous experience (Yang, Z.-Q., Danishefsky, S. J. J: Am. Chem. Soc. 2003, 125, 9602-9603; incorporated herein by reference) had prepared us well for accomplishing the much needed ring closing metathesis reaction. First, it would be necessary to immobilize the ethynyl linkage in 20 as its dicobalt hexacarbonyl complex (Scheme 1-3) (Yang, Z.-Q., Danishefsky, S. J. J. Am. Chem. Soc. 2003, 125, 9602-9603; Nicholas, K. M.; Pettit, R. Tetrahedron Lett. 1971, 37, 3475-3478; Young, D. G. J.; Burlison, J. A.; Peters, U. J. Org. Chem. 2003, 68, 3494-3497; each of which is incorporated herein by reference). This step accomplishes two objectives. First, the acetylene function is insulated from diversion to an ene-yne metathesis format. Furthermore, it is likely that the formation of the complex modifies the angles of the ethynyl sector (Sternberg, H. W.; Greenfield, H.; Friedel, R. A.; Wotiz, J.; Markby, R.; Wender, I. J. Am. Chem. Soc. 1954, 76, 1457-1458; incorporated herein by reference), such as to bring carbons 7′ and 8′ into closer proximity. In the event, the hexacarbonyl complex 21 was obtained in 94% yield. Ring closing metathesis was easily accomplished, using the recently published catalysis methodology from the Grubbs group (Scholl, M.; Ding, S.; Lee, C. W.; Grubbs, R. H. Org. Lett. 1999, 1, 953-956; incorporated herein by reference). The 14 membered macrolide (23) was thus in hand. At this stage the two stereoisomeric products were easily separated by

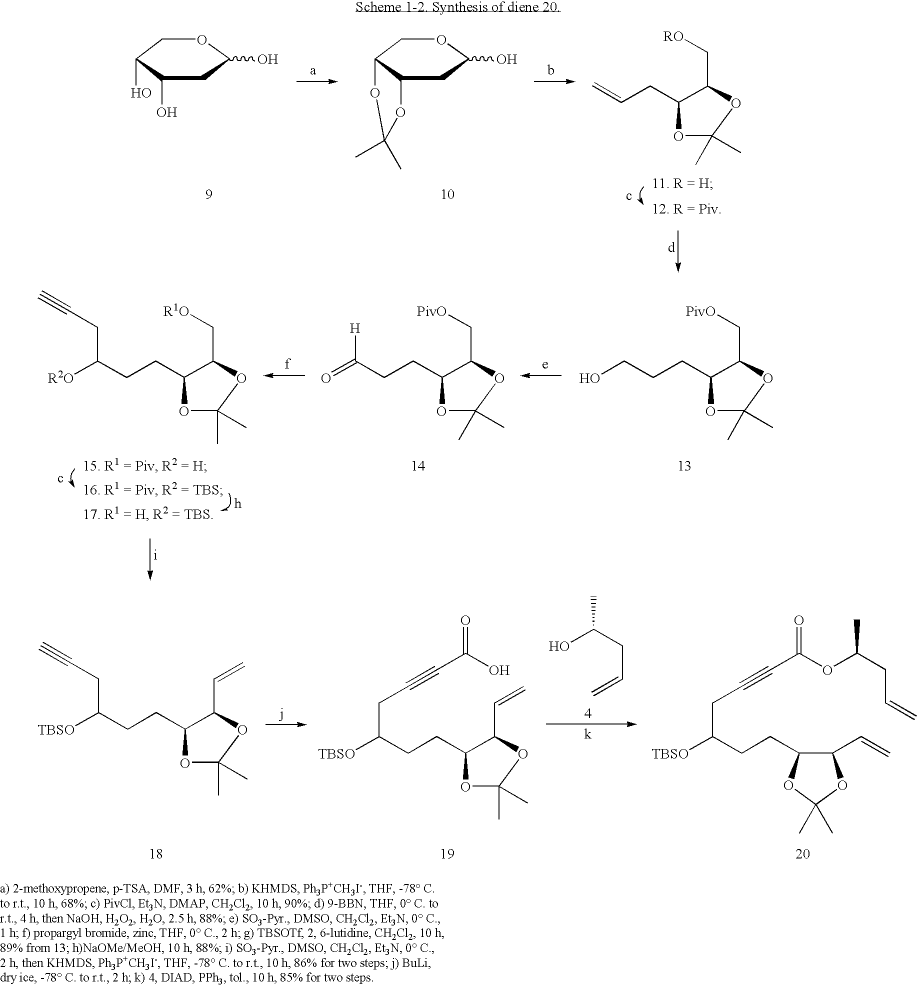

chromatography to provide the individual compound(s) at a 1.2:1 ratio (stereochemistry not rigorously assigned). We note that in each diastereomer, only the E configured double bond was obtained (J≅15.2 Hz).

Decomplexation of the two separated compounds 23a and 23b, using standard conditions, thereby afforded “ynolide” epimers 7 (Scheme 4). Each stereoisomer was subjected to Diels Alder reaction with the disiloxydiene 8, following previously developed conditions (Yang, Z.-Q., Danishefsky, S. J. J. Am. Chem. Soc. 2003, 125, 9602-9603; incorporated herein by reference). In the event, cycloaddition followed by extrusion of isobutylene occurred smoothly affording macrolides 24a and 24b. It proved useful to protect the two resorcycilic hydroxyl groups in the form of their MOM derivatives (see 25a and 25b) before proceeding with installation of the styrene like double bond. At this stage, deprotection of the silyl group was accomplished through the agency of HF-pyridine. Indeed, dehydration of the resulting alcohol functions in 26a and 26b, each using Martin's sulfurane conditions (Martin, J. C.; Arhart, R. J. J. Am. Chem. Soc. 1971, 93, 4327-4329; incorporated herein by reference), resulted in installation of the C1′-C2′ double

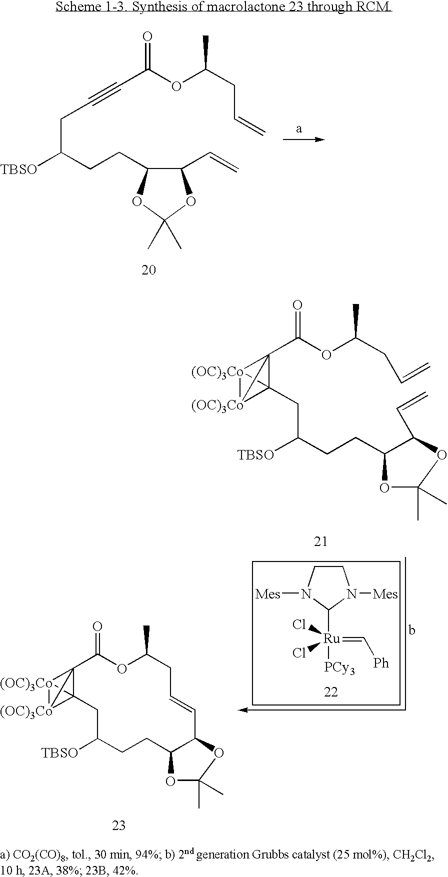

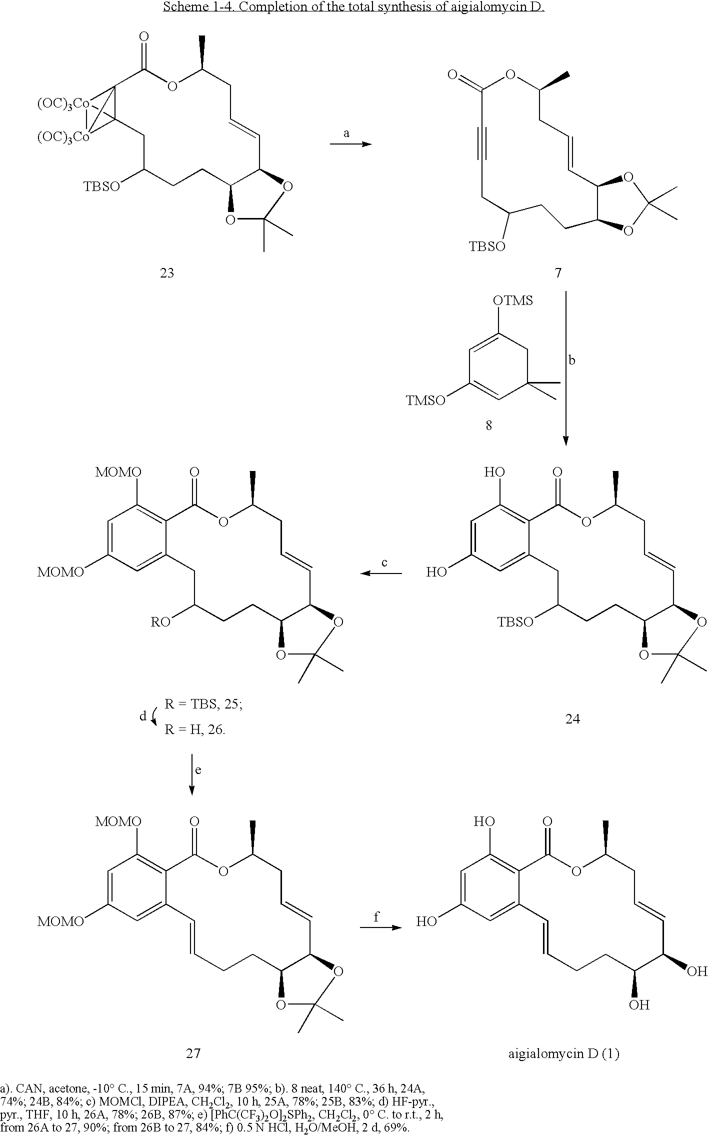

bond with the formation of the identical product, 27. Global acidic deprotection of the two MOM functions and the acetonide (0.5 N HCl) served to complete the first total synthesis of aigialomycin D (1). The assignment of structural and relative configuration could well have been rigorously accomplished based on our measurements (proton NMR, carbon NMR, mass spec and IR) accumulated on the fully synthetic material. In the case at hand, further support comes from the very close correspondence of our data with those previously reported for the target structure aigialomycin D (Isaka, M.; Suyarnsestakorn, C.; Tanticharoen, M.; Kongsaeree, P.; Thebtaranonth, Y. J. Org. Chem. 2002, 67, 1561-1566; incorporated herein by reference).

The synthesis described above, serves to further demonstrate the adaptability and generalizability of the basic protocol (“seco ylolide”→“ylolide”→resorcinylic macrolide, see Scheme 1-5). The total synthesis of 1 was accomplished in 18 steps in an overall yield of approximately 8%. We note in passing that compound 1 does bind to HSP90 (though significantly less so than radicicol (2)). It will be interesting to attempt to utilize this newly acquired and highly concise route to matrices resembling radicicol for the purposes of discovering new and superior agents based on the theme of HSP90 inhibition.

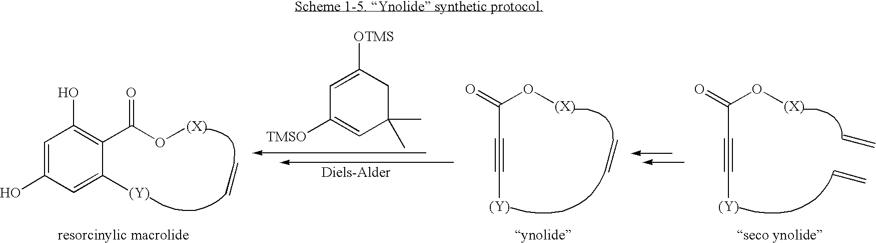

### Experimentals:

General Methods Reagents obtained from commercial suppliers were used without further purification unless otherwise noted. THF, toluene, and methylene chloride was obtained from a dry solvent system (passed through a prepacked column of alumina) and used without further drying. All air and water sensitive reactions were performed in flame-dried glassware under a positive pressure of prepurified argon gas. NMR (¹H and ¹³C) spectra were recorded on Bruker AMX-400 MHz or Bruker Advance DRX-500 MHz as noted individually, referenced to CDCl₃ (7.27 ppm for ¹H and 77.0 ppm for ¹³C) or CD₃COCD₃ (2.09 ppm for ¹H and 30.6 and 205.9 ppm for ¹³C). Infrared spectra (IR) were obtained on a Perkin-Elmer FT-IR model 1600 spectrometer. Melting point was tested on a electrothermal series IA9100 digital melting point apparatus. Optical rotations were obtained on a JASCO model DIP-370 digital polarimeter. Analytical thin-layer chromatography was performed on E. Merck silica gel 60 F254 plates. Compounds which were not UV active were visualized by dipping the plates in para-anisaldehyde solution and heating. Preparative thin layer chromatography was performed using the indicated solvent on Whatman® (LK6F Silica gel 60 Å 250 μM or Pk6F Silica Gel 60 Å 1000 μM) TLC plate.

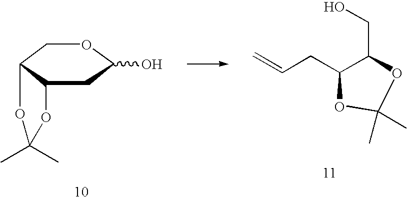

### (2R,3S)-Hex-5-ene-1,2,3-triol 2,3-acetonide (11):

To a stirring suspension of Ph₃P⁺CH₃I⁻ (11.2 g, 27.7 mmol) in 30 mL THF was added KHMDS (0.5 M in toluene, 46.0 mL, 23.0 mmol) at −78° C. The solution was warmed up to 0° C. and stirred for 30 min before cooled down to −78° C. Acetonide 10 (Barbat, J.; Gelas, J.; Horton, D. Carbohydrate Res. 1983, 116, 312-316; incorporated herein by reference) (1.6 g, 9.2 mmol) in 5 mL THF was added via cannula and the solution was warmed up to r.t. overnight (10 h) before quenched with saturated aqueous NH₄Cl solution, extracted with EtOAC (100 mL×3). The organic layers were combined and dried with anhydrous MgSO₄, filtered, and concentrated under reduced pressure vacuum. The residue was purified on a silica gel column using petroleum ether/EtOAc (4/1) as the eluant to afford 11 as a colorless oil (1.02 g, 68%). [α]_{D}²⁵ 54.8 (c 0.26, CHCl₃). ¹H NMR (400 MHz, CDCl₃) δ 1.34 (s, 3H), 1.46 (s, 3H), 2.02 (b, 1H), 2.25-2.32 (m, 1H), 2.37-2.44 (m, 1H), 3.65 (m, 1H), 4.16-4.21 (m, 1H), 4.24-4.28 (m, 1H), 5.10-5.18 (m, 2H), 5.79-5.89 (m, 1H). ¹³C NMR (100 MHz, CDCl₃) δ 25.4, 28.1, 33.6, 61.6, 76.2, 77.8, 108.3, 117.3, 134.2. LRMS (ESI) calcd for C₉H₁₆O₃Na⁺ [M+Na]⁺: 195.1, found 194.9. LRMS (ESI) calcd for C₉H₁₆O₃Cl⁻ [M+Cl]⁻: 207.1, found 207.1.

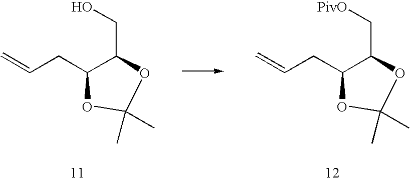

### Pivalate (12):

To a solution of 11 (752 mg, 4.37 mmol), DMAP (106 mg, 0.874 mmol) and triethylamine (2.5 ml, 17.5 mmol) in CH₂Cl₂ (8 ml) was added PivCl (1.1 ml, 8.74 mmol) at 0° C. The reaction mixture was warmed up to r.t. overnight (10 h) before quenched with saturated aqueous NaHCO₃ solution, extracted with EtOAC (100 mL×3). The organic layers were combined and dried with anhydrous MgSO₄, filtered, and concentrated under reduced pressure vacuum. The residue was purified on a silica gel column using hexanes/EtOAc (4/1) as the eluant to afford 12 as a colorless oil (984 mg, 90%). [α]_{D}²⁵ 15.6 (c 0.32, CHCl₃). ¹H NMR (400 MHz, CDCl₃) δ 1.22 (s, 9H), 1.33 (s, 3H), 1.47 (s, 3H), 2.28-2.41 (m, 2H), 4.10-4.13 (m, 2H), 4.22-4.29 (m, 2H), 5.10-5.17 (m, 2H), 5.81-5.91 (m, 1H). ¹³C NMR (100 MHz, CDCl₃) δ 25.9, 27.5, 28.4, 39.1, 63.2, 75.5, 77.8, 108.8, 117.7, 134.6, 178.5. LRMS (ESI) calcd for C₁₄H₂₄O₄Na⁺ [M+Na]⁺ 279.2, found 278.9.

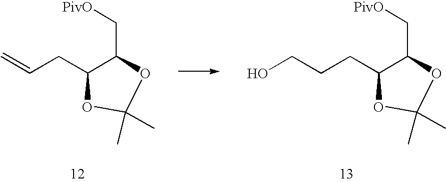

### Alcohol 13:

To a solution of 12 (166 mg, 0.648 mmol) in THF (1.5 mL) was added 9-BBN (0.5 M in THF, 2.8 mL, 1.425 mmol) at 0° C. The reaction mixture was warmed up to r.t. over 4 h and H₂O (0.1 mL), NaOH (3 M, 0.7 mL) and H₂O₂ (30%, 0.2 mL) were added. The reaction mixture was diluted with H₂O 2.5 h later and acidified with citric acid (5%) till pH=7. The mixture was extracted with EtOAc, washed with saturated aqueous Na₂S₂O₃, H₂O and brine. The organic layers were combined and dried with anhydrous MgSO₄, filtered, and concentrated under reduced pressure vacuum. The residue was purified on a silica gel column using petroleum ether/EtOAc (1/1) as the eluant to afford 13 as a colorless oil (156 mg, 88%). [α]_{D}²⁵ 82.4 (c 0.07, CHCl₃). ¹H NMR (400 MHz, CDCl₃) δ 1.21 (s, 9H), 1.36 (s, 3H), 1.46 (s, 3H), 1.59-1.80 (m, 4H), 2.30 (b, 1H), 3.54 (m, 2H), 4.08 (dd, J=6.1, 11.5 Hz, 1H), 4.12 (dd, J=5.6, 11.5 Hz, 1H), 4.19 (m, 1H), 4.25 (m, 1H). ¹³C NMR (100 MHz, CDCl₃) δ 25.5, 25.9, 27.1, 28.0, 29.9, 38.7, 62.3, 62.9, 75.3, 77.0, 108.3, 178.2. HRMS (ESI) calcd for C₁₄H₂₆O₅Na⁺ [M+Na]⁺ 297.1678, found 297.1660, Δ=−6.0 ppm.

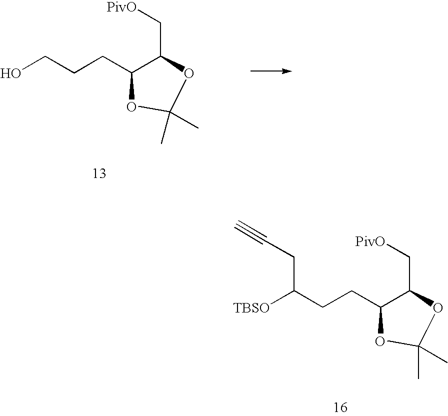

### Alkyne 16:

To a solution of 13 (682 mg, 2.485 mmol) in CH₂Cl₂ (5 mL), DMSO (5 mL) and Et₃N (3 mL) was added SO₃-pyridine complex (1.6 g, 10.2 mmol) at 0° C. The reaction mixture was stirred for 1 h before diluted with EtOAc and washed with HCl (0.5 N), H₂O, saturated aqueous NaHCO₃ solution and brine. The organic layers were dried with anhydrous MgSO₄, filtered, and concentrated under reduced pressure vacuum. The crude aldehyde 14 was used directly for next step without any further purification.

To a suspension of Zn (Nano-size power, pre-activated, 390 mg, 5.949 mmol) in THF (10 mL) was added propargyl bromide (80% in toluene, 0.53 mL, 4.759 mmol) at 0° C. The reaction mixture was stirred for 1 h at 0° C. and a solution of aldehyde 14 thus obtained in THF (5 mL) was added and reaction mixture was warmed up to r.t. over 2 h before quenched with saturated aqueous NH₄Cl solution and extracted with EtOAc. The organic layers were dried with anhydrous MgSO₄, filtered, and concentrated under vacuum. To a solution of crude alcohol 15 thus obtained and 2,6-lutidine (0.6 mL, 4.76 mmol) in CH₂Cl₂ (8 mL) was added TBSOTf (0.82 mL, 3.57 mmol) and the reaction mixture was stirred for 10 h before quenched with saturated aqueous NH₄Cl solution, extracted with EtOAc (100 mL×3). The organic layers were combined and dried with anhydrous MgSO₄, filtered, and concentrated under reduced pressure vacuum. The residue was purified on a silica gel column using petroleum ether/EtOAc (20/1) as the eluant to afford 16 as a colorless oil (896 mg, 89% from 13). ¹H NMR (400 MHz, CDCl₃) δ −0.02-0.05 (m, 6H), 0.80 (s, 9H), 1.13 (s, 9H), 1.26 (s, 3H), 1.36 (s, 3H), 1.42-1.74 (m, 4H), 1.81 (m, 1H), 2.22-2.29 (m, 2H), 3.78 (m, 1H), 4.01-4.08 (m, 3H), 4.12-4.17 (m, 1H). ¹³C NMR (100 MHz, CDCl₃) δ 18.0, 18.1, 24.3, 24.7, 25.6, 25.8, 27.0, 27.1, 27.2, 28.06, 28.07, 33.2, 33.3, 38.7, 62.8, 62.9, 70.1, 70.2, 70.4, 70.5, 75.2, 76.1, 77.1, 81.2, 108.20, 108.24, 178.1. HRMS (FAB) calcd for C₂₃H₄₂O₅SiH⁺ [M+H]⁺: 427.2880, found 427.2880, Δ=−0.1 ppm.

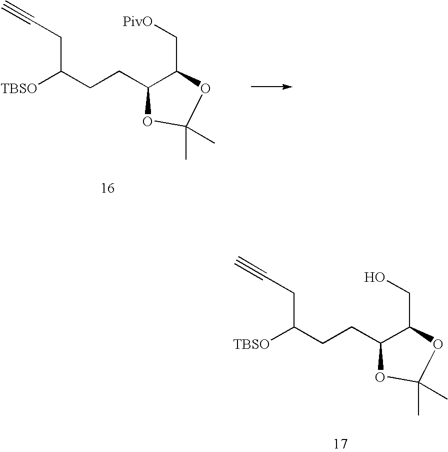

### Alcohol 17:

To a solution of 16 (124 mg, 0.291 mmol) in MeOH (6 mL) was added NaOMe/MeOH (25%, 0.2 mL) and the reaction mixture was stirred for 10 h before quenched with saturated aqueous NH₄Cl solution, extracted with EtOAc (100 mL×3). The organic layers were combined and dried with anhydrous MgSO₄, filtered, and concentrated under reduced pressure vacuum. The residue was purified on a silica gel column using petroleum ether/EtOAc (8/1) as the eluant to afford 17 as a colorless oil (87 mg, 88%). ¹H NMR (400 MHz, CDCl₃) δ −0.02-0.00 (m, 6H), 0.80 (s, 9H), 1.32 (s, 3H), 1.38 (s, 3H), 1.32-1.91 (m, 4H), 1.90 (m, 1H), 1.96 (b, 1H), 2.23-2.31 (m, 2H), 3.53 (m, 2H), 3.75 (m, 1H), 4.08 (m, 1H). ¹³C NMR (100 MHz, CDCl₃) δ −4.7, −4.6, −4.5, 18.0, 24.5, 24.7, 25.5, 25.6, 25.8, 27.2, 27.4, 28.2, 33.2, 33.4, 61.7, 70.1, 70.2, 70.46, 70.52, 76.9, 77.0, 77.9, 81.16, 81.24, 108.08, 108.14. HRMS (FAB) calcd for C₁₈H₃₄O₄SiH⁺ [M+H]⁺: 343.2305, found 343.2305, Δ=−0.1 ppm.

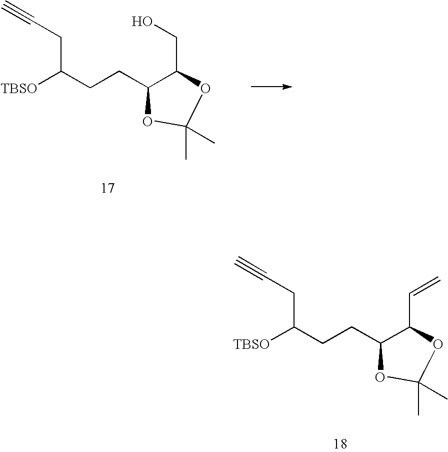

### Enzyme 18:

To a solution of 17 (87 mg, 0.254 mmol) in DMSO (1.0 mL), CH₂Cl₂ (1.0 mL) and Et₃N (1.0 mL) was added SO₃-Pyridine complex (200 mg, 2.032 mmol) at 0° C. The reaction mixture was stirred for 2 h before diluted with EtOAc and washed with HCl (0.5 N), H₂O, saturated aqueous NaHCO₃ solution and brine. The organic layers were dried with anhydrous MgSO₄, filtered, and concentrated under reduced pressure vacuum. The crude aldehyde was used directly for next step without any further purification.

To a stirring suspension of Ph₃P⁺CH₃I⁻ (204 mg, 0.505 mmol) in 3 mL THF was added KHMDS (0.5 M in toluene, 0.9 mL, 0.454 mmol) at −78° C. The solution was warmed up to 0° C. and stirred for 30 min before cooled down to −78° C. Aldehyde obtained as mention above in 2 mL THF was added via cannula and the solution was warmed up to r.t. overnight (10 h) before quenched with saturated aqueous NH₄Cl solution, extracted with EtOAc (100 mL×3). The organic layers were combined and dried with anhydrous MgSO₄, filtered, and concentrated under reduced pressure vacuum. The residue was purified on a silica gel column using petroleum ether/EtOAc (40/1) as the eluant to afford 18 as a colorless oil (73 mg, 86%). ¹H NMR (400 MHz, CDCl₃) δ −0.05 (s, 3H), 0.02 (s, 3H), 0.83 (s, 9H), 1.27 (s, 3H), 1.43 (s, 3H), 1.43-1.78 (m, 4H), 1.92 (b, 1H), 2.23-2.31 (m, 2H), 3.76 (m, 1H), 4.05-4.10 (m, 1H), 4.43-4.45 (m, 1H), 5.02-5.05 (m, 2H), 5.71-5.80 (m, 1H). ¹³C NMR (100 MHz, CDCl₃) δ −4.30, −4.11, 18.4, 26.0, 26.1, 26.2, 26.5, 26.8, 27.7, 27.8, 28.62, 28.64, 33.2, 33.6, 70.4, 70.5, 71.07, 71.13, 78.6, 78.8, 80.2, 81.78, 81.82, 108.5, 118.6, 118.7, 134.7, 134.9. HRMS (FAB) calc'd for C₁₉H₃₄O₃SiNa⁺ [M+Na]⁺: 361.2175, found 361.2175, Δ=0.0 ppm.

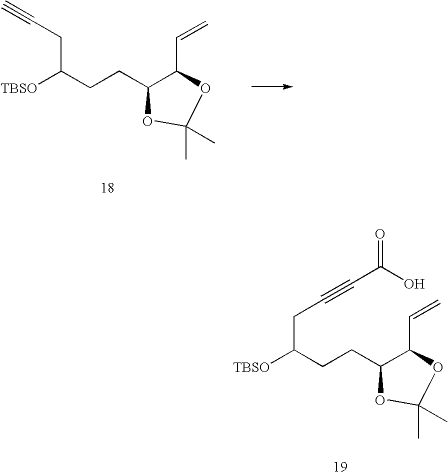

### Acid 19:

To a solution of 18 (586 mg, 1.731 mmol) in Et₂O (16 mL) was added BuLi (1.6 M in hexane, 1.817 mmol) at −78° C. and stirred for 5 min before quenched with dry ice and warmed up to r.t. The reaction mixture was washed with NaOH (0.1 M) and the aqueous layers were combined and acidified by HCl (0.1 M) until the pH=2. The aqueous layer was extracted with EtOAc and the organic layers were combined and dried with anhydrous MgSO₄, filtered, and concentrated under reduced pressure vacuum and dried on high vacuum. ¹H NMR (400 MHz, CDCl₃) δ −0.03 (s, 3H), 0.00 (s, 3H), 0.79 (s, 9H), 1.17 (s, 3H), 1.40 (s, 3H), 0.95-1.68 (m, 4H), 2.38-2.39 (m, 2H), 3.80 (b, 1H), 4.06 (m, 1H), 4.43 (m, 1H), 5.14-5.36 (m, 2H), 5.67-5.76 (m, 1H), 10.5 (bs, 1H). This crude was used directly for next step without any further purification.

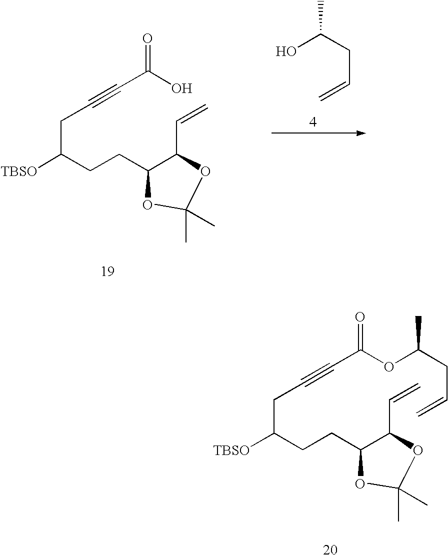

### Ester 20:

To a solution of acid 19 (249 mg, 0.651 mmol) in toluene (15 mL) was added alcohol 4 (0.081 mL, 0.781 mmol), PPh₃ (205 mg, 0.781 mmol), DIAD (0.154 mL, 0.781 mmol). The reaction mixture was stirred for 10 h and the solvent was removed under reduced pressure vacuum. The residue was purified on a silica gel column using petroleum ether/EtOAc (40/1) as the eluant to afford 20 as a colorless oil (255 mg, 85% for two steps). ¹H NMR (400 MHz, CDCl₃) δ −0.06-0.00 (m, 6H), 0.80 (s, 9H), 1.17 (d, J=6.2 Hz, 3H), 1.28 (s, 3H), 1.38 (s, 3H), 1.44-1.54 (m, 4H), 2.20-2.24 (m, 1H), 2.237-2.31 (m, 1H), 2.35-2.37 (m, 2H), 3.78-3.81 (m, 1H), 4.03-4.05 (m, 1H), 4.40-4.43 (m, 1H), 4.94-4.96 (m, 1H), 5.00-5.04 (m, 2H), 5.14 (m, 1H), 5.22 (dd, J=6.7, 7.1 Hz, 1H), 5.67-5.72 (m, 2H). ¹³C NMR (100 MHz, CDCl₃) δ −4.64, −4.58, −4.55, 0.0, 18.0, 19.3, 25.6, 25.7, 25.8, 26.1, 26.3, 27.6, 27.7, 28.2, 28.3, 33.3, 33.6, 40.0, 70.10, 70.12, 72.0, 74.9, 78.1, 78.3, 79.80, 79.82, 86.1, 108.2, 108.3, 118.1, 118.3, 118.4, 133.2, 134.2, 134.4, 153.2. HRMS (FAB) calcd for C₂₅H₄₂O₅SiH⁺ [M+H]⁺: 451.2880, found 451.2881, Δ=−0.3 ppm.

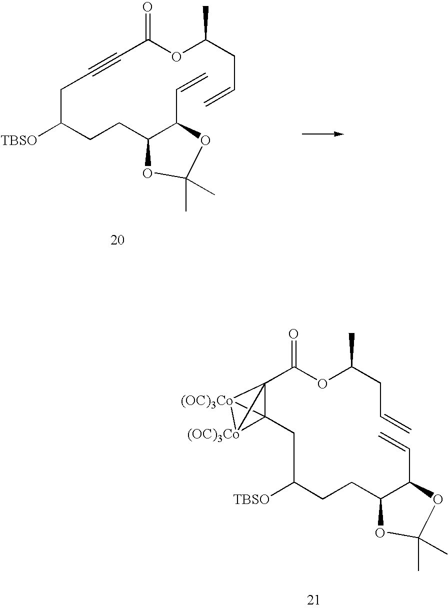

### Cobalt-Complex 21:

To a solution of 20 (20 mg, 0.044 mmol) in toluene (2.5 mil) was added Co₂(CO)_{g} (21.2 mg, 0.062 mmol). The reaction mixture was stirred for 30 min before filtered through neutral alumina and concentrated under reduced pressure vacuum. The residue was purified on preparative TLC (Whatman® Pk6F Silica Gel 60 Å 1000 μM) using Hexanes/EtOAc (20/1) as the eluant to afford 21 as a purple oil (30 mg, 94%). ¹H NMR (400 MHz, CDCl₃) δ 0.07-0.09 (m, 6H), 0.90 (s, 9H), 1.30 (d, J=8.1 Hz, 3H), 1.33 (s, 1.6H), 1.34 (s, 1.4H), 1.45 (s, 1.6H), 1.47 (s, 1.4H), 1.25-1.46 (m, 1H), 1.58-1.89 (m, 2.5H), 1.89-1.95 (m, 0.5H), 2.37-2.40 (m, 2H), 3.00-3.12 (m, 2H), 3.82-3.86 (m, 1H), 4.08-4.14 (m, 1H), 4.47-4.51 (m, 1H), 5.07-5.14 (m, 3H), 5.21 (d, J=10.4 Hz, 1H), 5.29 (d, J=17.1 Hz, 1H), 5.73-5.84 (m, 2H). ¹³C NMR (100 MHz, CDCl₃) δ 18.1, 19.4, 25.6, 25.8, 26.5, 27.0, 28.16, 28.2, 32.7, 33.0, 40.3, 41.9, 42.1, 71.9, 72.8, 73.0, 78.2, 169.0, 197. HRMS (FAB) calcd for C₃₁H₄₂Co₂O₁₁SiH⁺ [M+H]⁺: 737.1239, found 737.1240, Δ=−0.2 ppm.

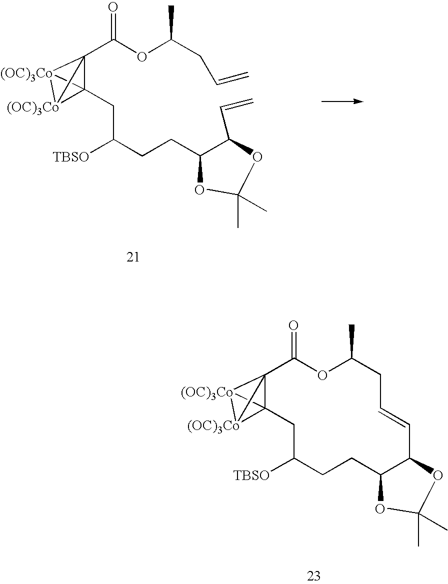

### Macrolactone 23:

To a solution of 21 (339 mg, 0.460 mmol) in CH₂Cl₂ (80 mL) was added 2^{nd} generation Grubbs catalyst (97 mg, 0.115 mol) in CH₂Cl₂ (15 mL) via cannula at r.t. The reaction mixture was stirred overnight and then the solvent was removed under reduced pressure vacuum and residue purified on preparative TLC (Whatman® Pk6F Silica Gel 60 Å 1000 μM) using Hexanes/EtOAc (10/1) as the eluant to afford 23A as a purple oil (123 mg, 38%). [α]_{D}²⁵ −44.7 (c 0.25, CHCl₃). ¹H NMR (400 MHz, CDCl₃) δ 0.08 (s, 3H), 0.09 (s, 3H), 0.91 (s, 9H), 1.31 (d, J=6.3 Hz, 3H), 1.35 (s, 3H), 1.45 (s, 3H), 1.39-1.59 (m, 2H), 1.65-1.72 (m, 1H), 1.93-1.97 (m, 1H), 2.22-2.31 (m, 1H), 2.42 (dt, J=12.9, 1.83 Hz, 1H), 3.08 (dd, J=16.0, 1.5 Hz, 1H), 3.23 (dd, J=16.0, 9.4 Hz, 1H), 3.92-3.95 (m, 1H), 4.01-4.06 (m, 1H), 4.40 (dd, J=9.4, 5.7 Hz, 1H), 5.53 (ddd, J=15.2, 10.9, 1.52 Hz, 1H), 5.72 (ddd, J=15.2, 10.7, 3.8 Hz, 1H). ¹³C NMR (100 MHz, CDCl₃) δ −4.5, −4.4, 18.5, 21.2, 23.1, 26.1, 26.2, 28.8, 30.5, 41.0, 42.7, 71.6, 71.8, 79.3, 80.0, 81.3, 92.9, 108.1, 129.1, 132.6, 169.9, 198.8. HRMS (FAB) calc'd for C₂₉H₃₈Co₂O₁₁SiH⁺ [M+H]⁺: 709.0926, found: 709.0924, Δ=0.2 ppm.

23B (136 mg, 42%). [α]_{D}²⁵ −5.9 (c 0.27, CHCl₃). ¹H NMR (400 MHz, CDCl₃) δ 0.12 (s, 3H), 0.15 (s, 3H), 0.93 (s, 9H), 1.32 (d, J=6.3 Hz, 3H), 1.35 (s, 3H), 1.42 (s, 3H), 1.11-1.54 (m, 2H), 1.67 (m, 1H), 1.88-1.93 (m, 1H), 2.24-2.33 (m, 1H), 2.41-2.45 (m, 1H), 3.05 (dd, J=14.9, 9.5 Hz, 1H), 3.18 (dd, J=14.9, 2.4 Hz, 1H), 3.54-3.59 (m, 1H), 4.03-4.09 (m, 1H), 4.42 (dd, J=9.5 Hz, 6.0 Hz, 1H), 5.28 (m, 1H), 5.53 (ddd, J=15.2, 7.9, 4.0 Hz, 1H), 5.77 (ddd, J=15.2, 10.5, 3.5 Hz, 1H). ¹³C NMR (100 MHz, CDCl₃) δ −4.3, −4.0, 18.4, 21.1, 25.8, 26.1, 26.2, 26.3, 26.9, 28.6, 32.4, 40.7, 44.6, 71.8, 73.8, 78.6, 79.8, 81.1, 93.3, 107.9, 128.6, 133.6, 169.6, 198.6. HRMS (FAB) calc'd for C₂₉H₃₈Co₂O₁₁SiH⁺ [M+H]⁺: 709.0926, found: 709.0924, Δ=0.2 ppm.

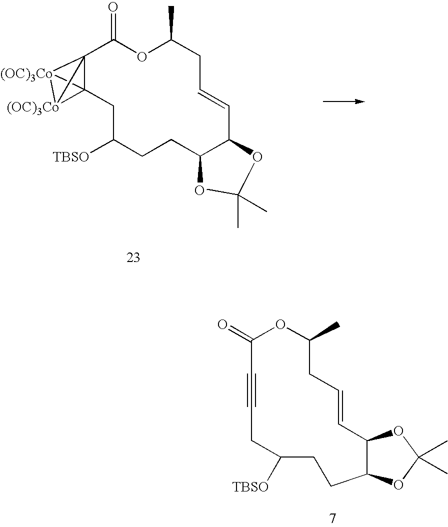

### Macrolide 7:

To a solution of 23A (123 mg, 0.174 mmol) in acetone (10 mL) was added CAN (475 mg, 0.868 mmol) at −10° C. After 20 min, the reaction mixture was filtered through neutral alumina and the solvent was removed under reduced pressure vacuum. The residue was purified on a silica gel column using petroleum ether/EtOAc (20/1) as the eluant to afford 7A as a colorless oil (69 mg, 94%). [α]_{D}²⁵ −124.6 (c 0.17, CHCl₃). ¹H NMR (400 MHz, CDCl₃) δ −0.06, (s, 3H), 0.00 (s, 3H), 0.81 (s, 9H), 1.24 (d, J=6.2 Hz, 1H), 1.33, (s, 3H), 1.40 (s, 3H), 1.59-1.81 (m, 4H), 2.18-2.22 (m, 1H), 2.28-2.31 (m, 1H), 2.34-2.45 (m, 2H), 3.90-3.93 (m, 1H), 3.98-4.01 (m, 1H), 4.33-4.36 (m, 1H), 4.83-4.87 (m, 1H), 5.42-5.53 (m, 2H). ¹³C NMR (100 MHz, CDCl₃) δ −4.8, 18.4, 20.6, 26.1, 26.3, 28.4, 28.8, 30.1, 36.5, 40.6, 70.0, 71.7, 78.9, 80.1, 88.5, 108.7, 129.7, 132.0, 153.8.

7B was prepared by same procedure from 23B. 7B: (81 mg, 95%). [α]_{D}²⁵ −173.3 (c 0.41, CHCl₃). ¹H NMR (400 MHz, CDCl₃) δ 0.00, (s, 3H), 0.01 (s, 3H), 0.81 (s, 9H), 1.26 (d, J=6.1 Hz, 3H), 1.34 (s, 3H), 1.41 (s, 3H), 1.53-1.59 (m, 2H), 1.77-1.81 (m, 2H), 2.19-2.24 (m, 1H), 2.24-2.26 (m, 1H), 2.31-2.47 (m, 2H), 3.80 (b, 1H), 3.91-3.93 (m, 1H), 4.34-4.40 (m, 1H), 4.75-4.78 (m, 1H), 4.82-4.86 (m, 1H), 5.43-5.60 (m, 2H). ¹³C NMR (100 MHz, CDCl₃) δ −5.0, −4.8, 17.9, 20.17, 20.24, 25.5, 25.6, 25.7, 25.9, 28.0, 28.2, 28.3, 28.4, 29.6, 36.0, 36.7, 40.0, 40.1, 69.5, 70.8, 71.2, 71.4, 78.5, 78.6, 79.4, 79.6, 78.8, 108.2, 129.2, 131.3, 153.0. HRMS (FAB) calc'd for C₂₅H₄₂O₅SiNa⁺[M+Na]⁺: 473.2699, found: 473.2700, Δ=0.2 ppm.

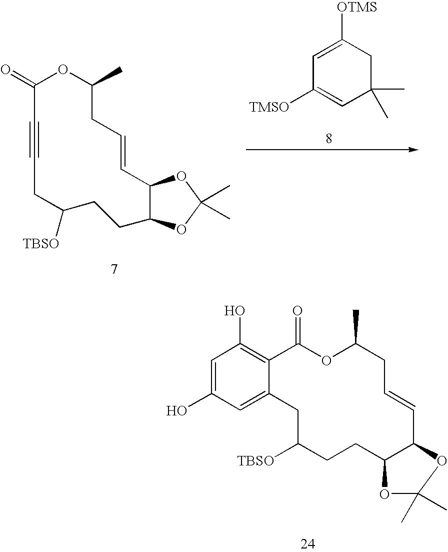

### Resorcyclic Macrolide 24:

Macrolide 7A (26 mg, 0.065 mmol) was transferred to a vial and 0.2 mL diene 8 was added. The vial was sealed and heated to 140 0° C. for 36 h. The crude mixture was purified on preparative TLC (Whatman® Pk6F Silica Gel 60 Å 1000 μM) using Hexanes/EtOAc (2/1) as the eluant to afford 24A as a colorless oil (23 mg, 74%). [α]_{D}²⁵ −99.1 (c 0.08, CHCl₃). ¹H NMR (400 MHz, CDCl₃) δ −0.15, (s, 3H), 0.09 (s, 3H), 0.89 (s, 9H), 1.36 (s, 3H), 1.38 (s, J=6.1 Hz, 1H), 1.50 (s, 3H), 1.25-1.39 (m, 4H), 1.71-1.76 (m, 1H), 2.54-2.57 (m, 2H), 2.60-2.64 (m, 1H), 3.63-3.68 (m, 2H), 4.08-4.11 (m, 1H), 4.48 (m, 1H), 5.22-5.26 (m, 1H), 5.54 (bs, 1H), 5.70-5.75 (m, 2H), 6.27 (d, J=2.6 Hz, 1H), 6.28 (d, J=2.6 Hz, 1H), 11.3 (s, 1H). ¹³C NMR (100 MHz, CDCl₃) δ −4.5, −4.1, 0.4, 14.6, 18.4, 21.3, 21.5, 24.1, 25.9, 26.3, 28.6, 32.4, 40.1, 42.4, 70.0, 73.5, 73.7, 77.7, 79.7, 101.9, 106.6, 108.7, 111.6, 130.5, 131.6, 145.7, 160.4, 165.0, 172.1. HRMS (FAB) calc'd for C₂₇H₄₂O₇SiH⁺ [M+H]⁺: 507.2778, found: 507.2777, Δ=0.2 ppm.

24B was prepared by same procedure from 7B. 24B: (81 mg, 84%). [α]_{D}²⁵ −124.2 (c 0.42, CHCl₃). ¹H NMR (400 MHz, CDCl₃) δ 0.00, (s, 3H), 0.14 (s, 3H), 0.99 (s, 9H), 1.50 (s, 3H), 1.58 (d, J=6.1 Hz, 1H), 1.40-1.67 (m, 2H), 1.64 (s, 3H), 1.87-1.92 (m, 2H), 2.64-2.72 (m, 2H), 3.09 (dd, J=3.8 Hz, 2.5 Hz, 1H), 3.45 (dd, J=13.8, 7.8 Hz, 1H), 4.02-4.04 (m, 1H), 4.20-4.22 (m, 1H), 4.70-4.74 (m, 1H), 5.50-5.52 (m, 1H), 5.76 (dd, J=15.5, 8.1 Hz, 1H), 5.93-5.95 (m, 2H), 6.42 (m, 1H), 6.48 (m, 1H), 11.62 (s, 1H). ¹³C NMR (100 MHz, CDCl₃) δ 4.3, −4.0, 14.5, 18.3, 18.4, 20.5, 21.5, 25.8, 26.2, 28.3, 28.4, 28.5, 33.1, 61.1, 72.7, 73.5, 79.1, 102.1, 107.1, 108.5, 112.7, 130.1, 130.5, 144.2, 160.7, 164.9, 171.7. LRMS (ESI) calcd for C₂₇H₄₂O₇SiNa⁺ [M+Na]⁺: 529.2, found: 529.1. LRMS (ESI) calcd for C₂₇H₄₂O₇SiCl⁻ [M+Cl]⁻: 541.3, found: 541.2.

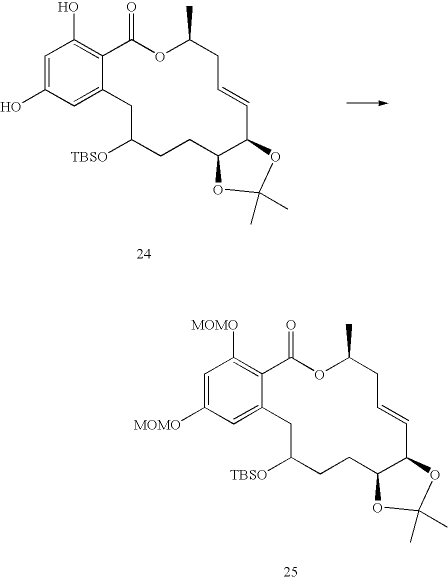

### MOM Ether 25:

To a solution of 24A (23 mg, 0.045 mmol) in CH₂Cl₂ (0.5 mL) was added diethylpropylethylamine (0.08 mL, 0.450 mmol) and MOMCl (0.018 mL, 0.227 mmol). The reaction mixture was stirred for 10 h before quenched with saturated aqueous NH₄Cl solution, extracted with EtOAC (100 mL×3). The organic layers were combined and dried with anhydrous MgSO₄, filtered, and concentrated under reduced pressure vacuum. The residue was purified on preparative TLC (Whatman® Pk6F Silica Gel 60 Å 1000 μM) using Hexanes/EtOAc (2/1) as the eluant to afford 25A as a colorless oil (21 mg, 78%). [α]_{D}²⁵ −2.86 (c 0.07, CHCl₃). ¹H NMR (400 MHz, CDCl₃) δ 0.00, (s, 3H), 0.10 (s, 3H), 0.93 (s, 9H), 1.39 (s, 3H), 1.39 (d, 3H), 1.48 (s, 3H), 1.42-1.48 (m, 1H), 1.60-1.64 (m, 2H), 1.72-1.77 (m, 1H), 2.41-2.45 (m, 2H), 2.66 (dd, J=5.6, 4.6 Hz, 1H), 2.89 (dd, J=4.5, 1.6 Hz, 1H), 3.47 (s, 3H), 3.48 (s, 3H), 3.97-3.98 (m, 1H), 4.09-4.15 (m, 1H), 4.73 (dd, J=9.0, 6.0 Hz, 1H), 5.13-5.19 (m, 4H), 5.30-5.36 (m, 1H), 5.57 (dd, J=15.4, 9.1 Hz, 1H), 5.70-5.77 (m, 1H), 6.68 (d, J=2.0 Hz, 1H), 6.86 (d, J=2.0 Hz, 1H). ¹³C NMR (100 MHz, CDCl₃) δ −4.3, −4.2, 0.5, 18.4, 21.6, 24.0, 25.9, 26.3, 28.7, 30.1, 32.3, 40.1, 40.4, 56.5, 56.6, 71.4, 71.6, 80.3, 94.7, 94.9, 101.8, 108.5, 111.4, 119.8, 130.2, 132.4, 139.4, 155.3, 158.6, 168.4.

25B was prepared by same procedure from 24B. 25B: (58 mg, 83%). [α]_{D}²⁵ −16.2 (c 0.29, CHCl₃). ¹H NMR (400 MHz, CDCl₃) δ −0.17 (s, ³H), −0.06 (s, 3H), 0.81 (s, 9H), 1.26 (s, 3H), 1.33 (d, J=6.1 Hz, 1H), 1.38 (s, 3H), 1.17-1.52 (m, 4H), 2.30-2.36 (m, 2H), 2.61 (dd, J=14.2, 6.0 Hz, 1H), 2.71 (dd, J=14.2, 6.9 Hz, 1H), 3.39 (s, 3H), 3.40 (s, 3H), 3.80-3.83 (m, 1H), 4.10-4.14 (m, 1H), 4.41 (dd, J=8.9, 6.1 Hz, 1H), 5.04-5.09 (m, 4H), 5.20-5.23 (m, 1H), 5.43 (dd, J=15.3, 9.1 Hz, 1H), 5.60-5.67 (m, 1H), 6.46 (s, 1H), 6.60 (s, 1H). ¹³C NMR (100 MHz, CDCl₃) δ 4.2, −3.8, 18.4, 21.3, 25.8, 26.2, 26.25, 26.3, 28.6, 33.0, 39.8, 40.0, 42.0, 56.5, 56.6, 71.7, 74.5, 78.6, 79.9, 94.7, 94.9, 101.5, 108.3, 111.2, 119.8, 131.0, 131.4, 139.2, 155.8, 158.9, 168.5. HRMS (FAB) calc'd for C₃₁H₅₀O₉SiH⁺ [M+H]⁺: 595.3302, found: 595.3304, Δ=−0.3 ppm.

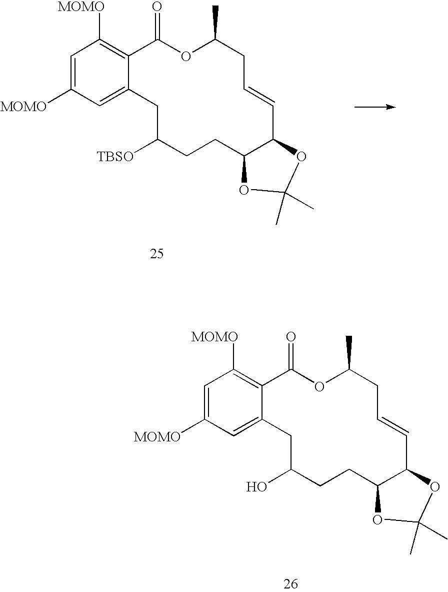

### Alcohol 26:

To a solution of 25A (21 mg, 0.035 mmol) in THF (1.4 mL) was added pyridine (0.6 mL) and HF-pyridine (30%, 0.3 mL). The reaction mixture was stirred for 10 h before quenched with MeOTf (2 mL) and stirred for 1 h. The solvent was removed under reduced pressure vacuum. The residue was purified on preparative TLC (Whatman® Pk6F Silica Gel 60 Å 1000 μM) using Hexanes/EtOAc (1/1) as the eluant to afford 26A as a colorless oil (12 mg, 78%). [α]_{D}²⁵ −105.2 (c 0.06, CHCl₃). ¹H NMR (400 MHz, CDCl₃) δ−0.05 (s, 3H), 0.05 (s, 3H), 1.35 (s, 3H), 1.38 (d, J=6.2 Hz, 3H), 1.46 (s, 3H), 1.61-1.81 (m, 4H), 2.40-2.46 (m, 2H), 2.70 (dd, J=14.1, 6.1 Hz, 1H), 2.81 (dd, J=14.1, 4.7 Hz, 1H), 3.46 (s, 6H), 3.89 (b, 1H), 4.10-4.14 (m, 1H), 4.56 (dd, J=9.1, 6.1 Hz, 1H), 5.13-5.17 (m, 4H), 5.32-5.37 (m, 1H), 5.59 (dd, J=15.4, 9.2 Hz, 1H), 5.70-5.75 (m, 1H), 6.66 (d, J=2.0 Hz, 1H), 6.70 (d, J=2.0 Hz, 1H). ¹³C NMR (100 MHz, CDCl₃) δ21.5, 25.1, 25.7, 28.6, 30.1, 32.3, 40.0, 41.6, 56.6, 56.7, 70.5, 72.0, 80.1, 94.7, 94.9, 101.9, 108.2, 111.0, 119.7, 130.6, 132.7, 138.3, 155.8, 159.1, 168.4. HRMS (FAB) calc'd for C₂₅H₃₆O₉H⁺ [M+H]⁺: 482.2438, found: 482.2437, Δ=0.1 ppm:

26B was prepared by same procedure from 25B. 26B: (20 mg, 87%). [α]_{D}²⁵ −32.0 (c 0.10, CHCl₃). ¹H NMR (500 MHz, CDCl₃) δ 1.18 (m, 2H), 1.35 (s, 3H), 1.37 (d, J=6.2 Hz, 1H), 1.42 (s, 3H), 1.55-1.69 (m, 2H), 1.98-2.06 (m, 1H), 2.42-2.48 (m, 3H), 2.78 (dd, J=13.8, 2.4 Hz, 1H), 3.41-3.50 (m, 6H), 3.62-3.67 (m, 1H), 4.19-4.25 (m, 1H), 4.49 (dd, J=9.4, 6.0 Hz, 1H), 5.11-5.18 (m, 4H), 5.36-5.42 (m, 1H), 5.56 (dd, J=15.4, 9.5 Hz, 1H), 5.65-5.72 (m, 1H), 6.57 (d, J=2.0 Hz, 1H), 6.66 (d, J=2.0 Hz, 1H). ¹³C NMR (1000 Hz, CDCl₃) δ21.6, 25.8, 26.8, 28.6, 32.0, 39.8, 42.6, 56.5, 56.7, 72.3, 74.8, 80.3, 94.7, 94.9, 101.8, 108.6, 110.7, 108.6, 110.7, 119.5, 129.9, 132.8, 138.7, 155.9, 159.0, 168.7. HRMS (FAB) calc'd for C₂₅H₃₆O₉H⁺ [M+H]⁺: 482.2438, found: 482.2437, Δ=0.1 ppm.

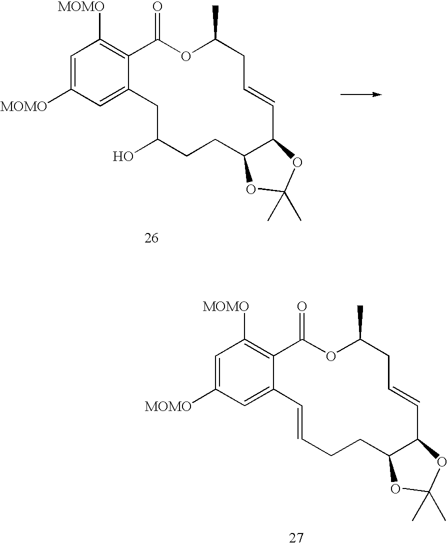

### Diene 27:

A solution of Martin's sulfurane dehydration agent (140 mg, 0.208) was added into a vial containing 26B (20 mg, 0.042) at 0° C. The reaction mixture was warmed up to r.t. over 2 h and the crude was purified on preparative TLC (Whatman® Pk6F Silica Gel 60 Å 1000 μM) using Hexanes/EtOAc (1/1) as the eluant to afford 27 as a colorless oil (16 mg, 84%). [α]_{D}²⁵ −123.8 (c 0.08, CHCl₃). ¹H NMR (500 MHz, CDCl₃) δ 1.35 (s, 3H), 1.36 (d, J=6.0 Hz, 3H), 1.46 (s, 3H), 1.49-1.55 (m, 1H), 1.80-1.85 (m, 1H), 2.07-2.11 (m, 1H), 2.29-2.32 (m, 1H), 2.45-2.55 (m, 2H), 3.41-3.50 (m, 6H), 4.16-4.21 (m, 1H), 4.56 (dd, J=9.5, 5.4 Hz, 1H, 1H), 5.10-5.20 (m, 4H), 5.32-5.36 (m, 1H), 5.59 (dd, J=15.5, 9.6 Hz, 1H), 5.70-5.77 (m, 1H), 5.15 (m, 1H), 6.24 (d, J=15.4 Hz, 1H), 6.80 (d, J=1.8 HZ, 1H), 6.68 (d, J=1.8 HZ, 1H). ¹³C NMR (125 MHz, CDCl₃) δ 21.1, 25.8, 28.6, 28.7, 29.0, 39.5, 71.6, 80.1, 84.3, 94.6, 102.6, 104.8, 108.3, 117.9, 124.8, 128.4, 129.3, 131.9, 132.3, 136.8, 155.1, 158.9, 167.3. HRMS (FAB) calcd for C₂₅H₃₄O₈H⁺ [M+H]⁺: 463.2332, found: 463.2333, Δ=−0.2 ppm.

27 could also be obtained from 26A using same procedure (90%).

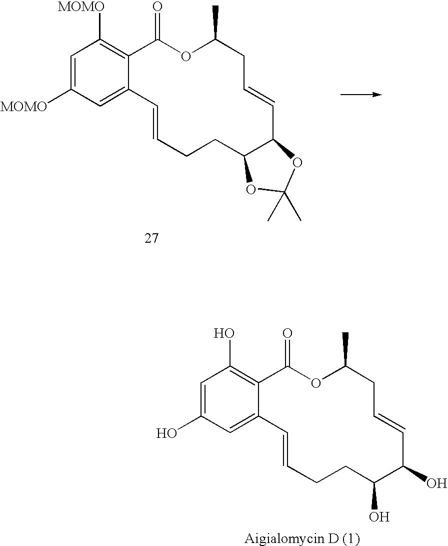

### Aigialomycin D (1):

To a solution of 27 (16 mg, 0.035) in MeOH (1.5 mL) was added HCl (1 N, 1.5 mL) and stirred for 2 d. The reaction was quenched with saturated aqueous NaHCO₃ solution and extracted with EtOAc. The organic layers were combined and dried over anhydrous MgSO₄, filtered and concentrated under reduced vacuum. The crude was purified on preparative LC (Whatman® Pk6F Silica Gel 60 Å 1000 μM) using MeOH/CH₂Cl₂ (5%) as the eluant to afford 1 as a white solid (8 mg, 69%). Mp: 84.2-86.9° C. [α]_{D}²⁵ −18.0 (c 0.03, MeOH). IR (neat) 3346, 1643, 1607, 1311, 1261, 1166, 1017, 968. ¹H NMR (500 MHz, acetone-d₆) δ 1.39 (d, J=6.4 Hz, 3H), 1.58-1.61 (m, 1H), 2.14 (m, 1H), 2.32-2.36 (m, 2H), 2.43-2.46 (m, 1H), 2.57 (ddd, J=14.5, 7.3, 3.1 Hz, 1H), 3.56 (br, 1H), 3.64 (m, 1H), 3.76 (br, 1H), 4.35 (brd, J=4.1 Hz, 1H), 5.41-5.47 (m, 1H), 5.69 (dd, J=15.6, 5.1 Hz, 1H), 5.87 (dddd, J=15.6, 7.4, 7.4, 1.4 Hz, 1H), 6.10 (ddd, J=15.9, 5.5, 5.7 Hz, 1H), 6.28 (d, J=2.3 Hz, 1H), 6.53 (d, J=2.3 Hz, 1H), 7.16 (d, J=15.9 Hz, 1H), 9.10 (bs, 1H), 11.7 (s, 1H). ¹³C NMR (125 MHz, acetone-d₆) δ 19.2, 28.1, 28.8, 38.1, 73.1, 73.4, 76.7, 102.6, 104.6, 107.9, 125.6, 130.8, 133.8, 135.9, 144.5, 163.2, 166.0, 172.3. LRMS (ESI) calcd for C₁₈H₂₂O₆Na⁺ [Na⁺H]⁺: 357.1, found: 357.3. LRMS (ESI) calcd for C₁₈H₂₁O₆⁻ [M−H]⁻: 333.1, found: 333.1. LRMS (ESI) calcd for C₁₈H₂₂O₆Cl⁻ [M+Cl]⁻: 369.1, found: 369.0. HRMS (TOF) calcd for C₁₈H₂₂O₆Na⁺[M+Na]⁺: 357.1314, found: 357.1325, Δ=3.1 ppm. All the physical data are consistent with the reported value (Isaka, M.; Suyarnsestakorn, C.; Tanticharoen, M.; Kongsaeree, P.; Thebtaranonth, Y. J. Org. Chem. 2002, 67, 1561-1566; incorporated herein by reference).

¹H NMR Data of Aigialomycin D

| position | Isolated Aigialomycin D by Isaka | Synthetic Aigailomycin D |
|---|---|---|
| 3 | 6.27 (d, 2.4) | 6.28 (d, 2.3) |
| 5 | 6.52 (d, 2.4) | 6.53 (d, 2.3) |
| 1′ | 7.14 (d, 15.9) | 7.16 (d, 15.9) |
| 2′ | 6.09 (ddd, 15.9, 5.6, 5.4) | 6.10 (ddd, 15.9, 5.7, 5.5) |
| 3′ | 2.31-2.34 (m) | 2.32-2.36 (m) |
| | 2.31-2.34 (m) | 2.32-2.36 (m) |
| 4′ | 2.14 (m) | 2.14 (m) |
| | 1.58 (m) | 1.58-1.61 (m) |
| 5′ | 3.62 (m) | 3.64 (m) |
| 6′ | 4.35 (brd, 4.3) | 4.35 (brd, 4.1) |
| 7′ | 5.68 (dd, 15.7, 5.0) | 5.69 (dd, 15.6, 5.1) |
| 8′ | 5.87 (dddd, 15.7, 7.3, 7.3, 1.2) | 5.87 |
| | | (dddd, 15.6, 7.4, 7.4, 1.4) |
| 9′ | 2.55 (ddd, 14.6, 7.5, 3.2) | 2.55 (ddd, 14.5, 7.3, 3.1) |
| | 2.42 (m) | 2.43-2.46 (m) |
| 10′ | 5.42 (m) | 5.41-5.47 (m) |
| 10′-CH₃ | 1.38 (d, 6.4) | 1.39 (d, 6.4) |
| 2-OH | 11.65 (s) | 11.7 (s) |
| 4-OH | 9.5 (br) | 9.1 (br) |
| 5′-OH | not detected | 3.56 (br) |
| 6′-OH | not detected | 3.76 (br) |

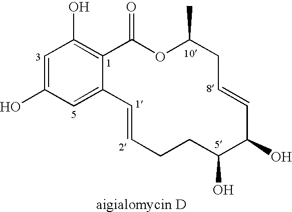

### Example 2

### Concise Route to Benzofused Macrolactones via Ynolides: Cycloproparadicicol

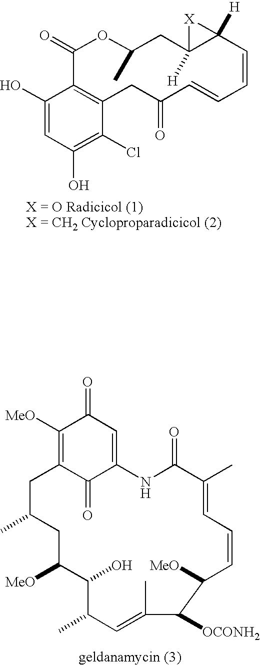

### Structures of Hsp90 Inhibitors

In this Example, we report a new approach to the broad family of resorcinylic fused macrolides. The underlying concept is captured graphically in Scheme 2-1, which is directed to our focusing target, cycloproapradicicol (2). However, as is suggested by the very facile synthesis of model compound 13 (vide infra), and has been further established in ongoing work, the method is quite general. The central element of our plan is the building of an “ynolide” intermediate and its advancement to the benzomacrolide by a Diels-Alder cycloaddition. The ynolide is constructed through olefin metathesis, enabled only by presentation of the acetylene linkage as its dicobalt hexacarbonyl cluster (see 9→10 and 14→15) (Young, D. G.; Burlison, J. A.; Peters, U. J. Org. Chem. 2003, 68, 3494; incorporated herein by reference).

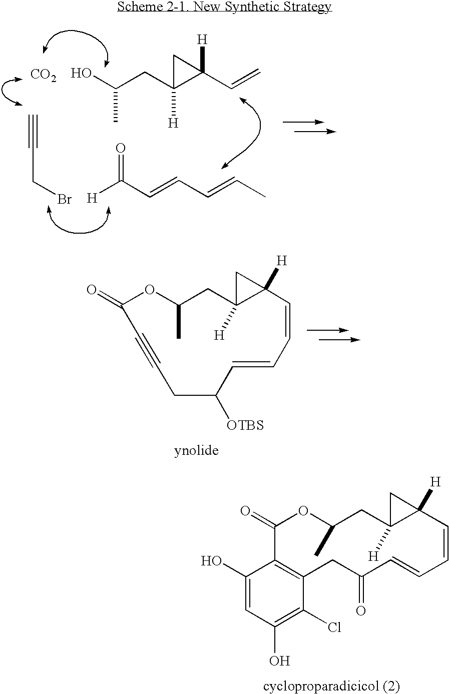

Our synthesis commenced with commercial 2,4-hexadienal (sorbaldehyde, 5, Scheme 2-2). Reformatsky-like condensation of propargyl bromide (4) with 5, followed by TBS ether protection and subsequent reaction of the lithium alkynide ion with CO₂, provided acid 6. Following reaction of racemic 6 and the known optically pure and defined alcohol 7 (Yamamoto, K.; Gabaccio, R. M.; Stachel, S. J.; Solit, D. B.; Chiosis, G., Rosen, N.; Danishefsky, S. J. Angew. Chem. Int. Ed. 2003, 42, 1280; incorporated herein by reference) under Mitsonobu conditions, ester 8 was obtained.

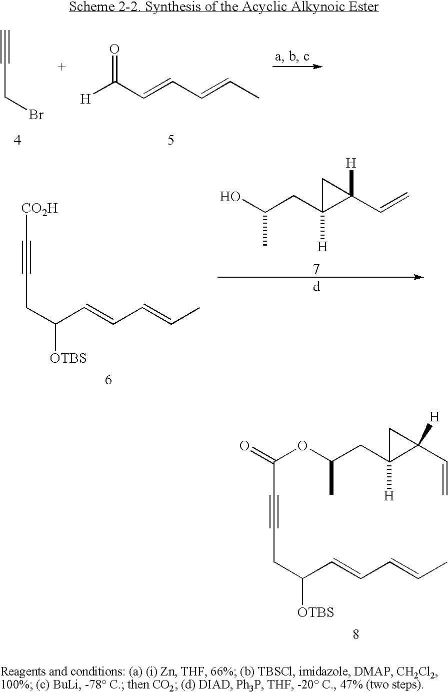

Projected ring-closing metathesis (RCM) reactions were conducted with a cyclic alkyne. Unfortunately, triene 8 failed to cyclize under a variety of RCM conditions. We took this negative finding to reflect impediments to cyclization arising from the linear character of the acetylene, possibly aggravated by rigidities associated with the trans-disubstituted cyclopropane. A more flexible model compound was prepared from acid 6 and 5-hexen-1-ol, and subjected to RCM reactions (Scheme 2-3). Again, only starting material was recovered. Aside from the constraint to cyclization imposed by linear alkyne, the cyclization could further be complicated by non productive coordination of the acetylene to the RCM catalytic machinery. It is well known that reaction of dicobalt carbonyl with acetylenes can lead to stable complexes (Greenfield, H.; Sternberg, H, W,; Friedel, R. A.; Wotiz, J. H.; Markby, R.; Wender, I. J. Am. Chem. Soc. 1956, 78, 120; Nicholas, K. M.; Pettit, R. Tetrahedron Lett. 1971, 3475; each of which is incorporated herein by reference), wherein the geometry of cobalt-complexed alkynes is distorted to approximately 140° (Dickson, R. S.; Fraser, P. J. Adv. Organomet. Chem. 1974, 12, 323; incorporated herein by reference).

In the event, cyclization of 9 proceeded smoothly under the conditions shown. Following oxidative removal of the cobalt using ammonium cerium (IV) nitrate (CAN), the desired cyclic alkynoic ester 11 was generated in high yield (Scheme 2-3).

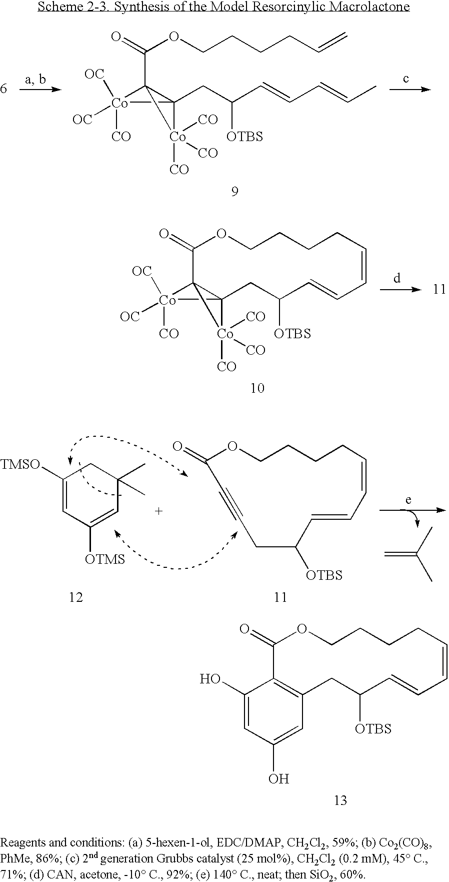

Construction of the resorcinylic skeleton called for a Diels-Alder reaction of 11 with a 1,3-bis-oxygenated diene. We found that the known dimedone-derived diene, 5,5-dimethyl-1,3-bis-trimethylsilyloxy-cyclohexa-1,3-diene (Ibuka, T.; Mori, Y.; Aoyama, T.; Inubushi, Y. Chem. Pharm. Bull. 1978, 26, 456; Langer, P.; Schneider, T.; Stoll, M. Chem. Eur. J. 2000, 6, 320; each of which is incorporated herein by reference) (12, Scheme 2-3), served our purpose best. Indeed, Diels-Alder reaction of cyclic alkyne 11 with 12 proceeded smoothly at 140° C., providing the desired aromatic product 13 in 60% yield, after concomitant retro-Diels-Alder loss of isobutene from the initial adduct, and hydrolysis of the trimethylsilyl ether groups during chromatography.

We applied this strategy to the targeted system (8). Gratifyingly, under the same RCM conditions, cyclopropane-containing cobalt complex 14 cyclized to give 15 in 57% yield, as a 2:1 mixture of two diatereomers (Scheme 2-4) (Here, we described only the conversion of the major isomer of 15 to 2. The other isomer worked equally well). In this case, removal of cobalt on 15, however, proved to be challenging, presumably due to the presence of the sensitive vinyl cyclopropane functionality. After screening a variety of conditions, we found that I₂-THF worked well (Tanaka, S.; Tsukiyama, T.; Isobe, M. Tetrahedron Lett. 1993, 34, 5757; incorporated herein by reference). The key cyclic alkyne dienophile 16 was thus obtained in 69% yield.

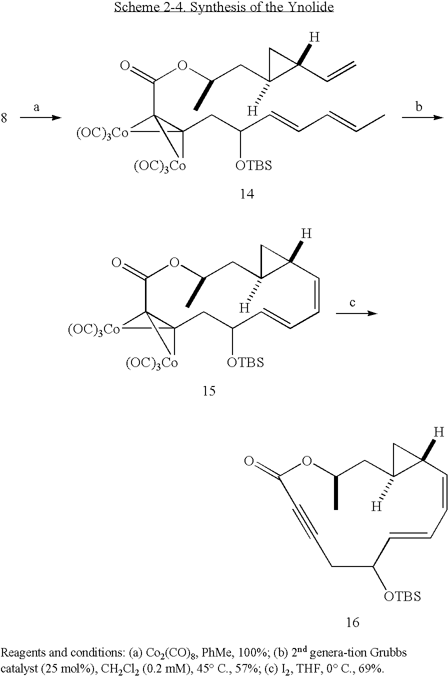

Diels-Alder reaction of 16 with diene 12 furnished the desired product 17 in 75% yield (Scheme 2-5). Transformation of 17 to the desired ketone by direct oxidation turned out to be a non-trival matter. In the end, it was accomplished following protection of the two phenolic functions, as shown, by straightford transformations to afford dechlorinated analogue 19 (Scheme 2-5). Finally, regioselective chlorination of 19 using SO₂Cl₂ in CH₂Cl₂ (Yamamoto, K.; Gabaccio, R. M.; Stachel, S. J.; Solit, D. B.; Chiosis, G., Rosen, N.; Danishefsky, S. J. Angew Chem. Int. Ed. 2003, 42, 1280; Garbaccio, R. M.; Stachel, S. J.; Baseschlin, D. K.; Danishefsky, S. J. J. Chem. Soc. 2001, 123, 1090; each of which is incorporated herein by reference), converted 19 into cycloproparadicicol (2).

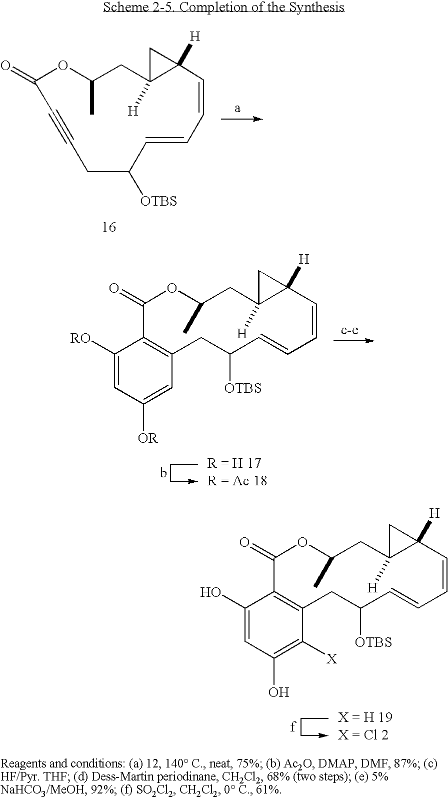

In summary, a new efficient synthetic route has been developed for a pre-clinical candidate, cycloproparadicicol (2) and, by extension, to a broad range of benzofused macrolactones.

### Experimentals:

General Methods Reagents obtained from commercial suppliers were used without further purification unless otherwise noted. THF, toluene, and methylene chloride was obtained from a dry solvent system (passed through a prepacked column of alumina) and used without further drying. All air and water sensitive reactions were performed in oven or flame-dried glassware. NMR (¹H and ¹³C) spectra were recorded on Bruker AMX-400 MHz or Bruker Advance DRX-500 MHz as noted individually, referenced to CDCl₃ (7.27 ppm for ¹H and 77.23 ppm for ¹³C). Optical rotations were obtained on a JASCO model DIP-370 digital polarimeter. Low resolution mass spectra (ESI) were determined with a PESciex AP 130 spectrometer. High resolution mass spectra (FAB) were determined at Chemistry Department of Columbia University. Flash chromatography was performed with silica gel (230-400 mesh) from EM Science as the stationary phase. Analytical thin-layer chromatography was performed on E. Merck silica gel 60 F254 plates. Compounds which were not UV active were visualized by dipping the plates in phosphomolybdic acid solution and heating. Preparative thin layer chromatography was performed using the indicated solvent on Whatman® (LK6F Silica gel 60 Å 250 μM or Pk6F Silica Gel 60 Å 1000 μM) TLC plate.

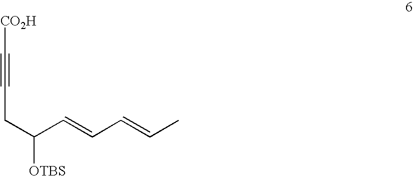

Acid 6. To a suspension of activated zinc (15 g, 230 mmol) in dry THF (50 mL) at 0° C. was added propargyl bromide 4 (19.2 mL 80 wt % in toluene, 172 mmol). The resulting mixture was stirred at 0° C. for 1 hr, and sorbaldehyde 5 (12.7 mL, 115 mmol) was added. After 1 hr at 0° C., additional zinc (4.5 g, 69 mmol) was added, and stirring was continued for 2.5 hrs at room temperature (the reaction was exothermic and ice bath was needed occasionally to keep the temperature down). The reaction was quenched by slow addition of sat. aqueous NH₄Cl (500 mL), followed by diluting with Et₂O (1 L). The layers were separated, and the organic layer was washed with H₂O (300 mL), brine (300 mL), dried (Na₂SO₄), filtered and concentrated in vacuum. The residue was dissolved in CH₂Cl₂ (750 mL) with imidazole (9.8 g, 144 mmol), t-butyldimethylsilyl chloride (19 g, 126 mmol) and 4-(dimethylamino)pyridine (1.4 g, 11.5 mmol), and stirred at room temperature for 3 hrs. Additional imidazole (4.9 g, 72 mmol) and t-butyldimethylsilyl chloride (9.5 g, 63 mmol) were added, and stirring was continued for 9 hrs. The reaction was quenched by addition of sat. aqueous NH₄Cl (200 mL). The layers were separated, and the organic layer was washed with H₂O (200 mL), brine (200 mL), dried (Na₂SO₄), filtered and concentrated in vacuum. The residue was purified by flash chromatography (silica, 0 to 10% Et₂O in hexane) to give the terminal alkyne precursor of 6 (15 g, 52%). ¹H NMR (CDCl₃, 400 MHz) δ 6.18 (dd, J=15.1, 10.5 Hz, 1H), 6.03 (ddd, J=15.0, 10.6, 1.3 Hz, 1H), 5.72 (dd, J=14.9, 6.9 Hz, 1H), 5.61 (dd, J=15.1, 6.4 Hz, 1H), 4.30 (q, J=6.3 Hz, 1H), 2.43 (ddd, J=16.5, 6.2, 2.7 Hz, 1H), 2.34 (ddd, J=16.5, 6.8, 1.7 Hz, 1H), 2.60 (t, J=2.6 Hz, 1H), 1.77 (d, J=6.8 Hz, 3H), 0.91 (s, 9H), 0.09, 0.06 (2s, 6H); ¹³C NMR (CDCl₃, 100 Hz) δ 132.5, 131.1, 130.7, 130.0, 81.7, 72.1, 70.1, 28.9, 26.1, 18.4, −2.7; IR (film) νₘₐₓ 3313, 2956, 2930, 2856, 2121, 1255, 1115, 1079, 987, 837; ESIMS m/z 273 ([M+Na⁺], C₁₅H₂₆NaOSi requires 273).

To a solution of the terminal alkyne precursor of 6 (15.0 g, 59.9 mmol) in Et₂O (270 mL) at −78° C., was added a solution of BuLi (1.6 M in hexane, 41.5 mL, 66.4 mmol). After 45 min, excess crushed dry ice was added and the reaction was allowed to warm to room temperature. The solution was acidified by addition of 0.5 M aqueous citric acid (300 mL). The layers were separated, and the aqueous layer was extracted with additional Et₂O (300 mL×2). The combined organic layers were dried (MgSO₄), filtered and concentrated in vacuum. The residue was purified by flash chromatography (silica, 50% to 100% EtOAc in hexane) to give the product as a light yellow solid (17.5 g, 99%). ¹H NMR (CDCl₃, 400 MHz) δ 6.19 (dd, J=15.1, 10.4 Hz, 1H), 6.03 (ddd, J=−14.9, 10.6, 1.3 Hz, 1H), 5.75 (dd, J=15.0, 6.8 Hz, 1H), 5.55 (dd, J=15.1, 6.4 Hz, 1H), 4.36 (q, J=6.3 Hz, 1H), 2.61-2.48 (m, 2H), 1.77 (d, J=6.8 Hz, 3H), 0.91 (s, 9H), 0.10, 0.06 (2s, 6H); ¹³C NMR (CDCl₃, 100 Hz) δ 157.6, 131.6, 131.3, 130.7, 89.7, 74.0, 71.4, 29.3, 26.0, 18.4, 18.3, −4.3, −4.7; IR (film) νₘₐₓ 2956, 2930, 2857, 2242, 1689, 1281, 1257, 1080; ESIMS m/z 317 ([M+Na⁺], Cl₆H₂₆NaO₃Si requires 317).

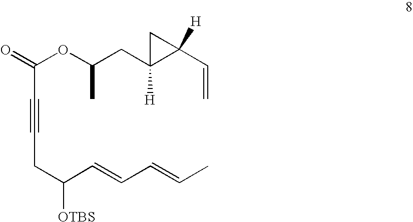

Alkynoic ester 8. To a solution of DIAD (14.7 mL, 72.9 mmol) in dry THF (350 mL) was added Ph₃P (15.8 g, 60.2 mmol), and the mixture was stirred at room temperature for one hour. At −20° C., a solution of acid 6 (13.1 g, 44.4 mmol) in 100 mL THF was added. After 15 min, a solution of alcohol 7 (4.0 g, 31.7 mmol) in 150 mL THF was added, and stirring was continued for 2 hours at −20° C. The reaction was quenched by addition of 250 mL of pH 7.2 phosphate buffer, followed by warming to room temperature and diluting with EtOAc (1.5 L). The layers were separated, and the aqueous layer was extracted with EtOAc (2×250 mL). The combined organic layers were washed with brine (250 mL), dried (Na₂SO₄), filtered and concentrated in vacuum. The residue was purified by flash chromatography (silica, 50:1→20:1 hexanes/EtOAc) to give ester 8 as a mixture of two inseparable diastereoisomers (5.9 g, 47%). ¹H NMR (CDCl₃, 400 MHz) δ 6.17 (dd, J=15.1, 10.5 Hz, 1H), 6.03 (ddd, J=12.3, 10.6, 1.4 Hz, 1H), 5.70 (dd, J=14.8, 6.8 Hz, 1H), 5.55 (dd, J=15.2, 6.4 Hz, 1H), 5.37 (ddd, J=17.1, 10.2, 8.7 Hz, 1H), 5.06 (q, J=6.4 Hz, 1H), 5.03 (dd, J=17.0, 1.5 Hz, 1H), 4.84 (dd, J=10.2, 1.6 Hz, 1H), 4.34 (q, J=6.4 Hz, 1H), 2.56-2.42 (m, 2H), 1.76 (d, J=6.8 Hz, 3H), 1.57-1.53 (m, 2H), 1.29 (d, J=6.4 Hz, 3H), 1.20-1.10 (m, 1H), 0.90 (s, 9H), 0.79-0.68 (m, 1H), 0.65-0.57 (m, 2H), 0.10, 0.05 (2s, 6H); ¹³C NMR (CDCl₃, 100 Hz) δ 154.5, 141.4, 131.8, 131.1, 130.8, 130.4, 112.1, 86.2, 74.9, 73.0, 71.5, 39.7, 29.2, 26.0, 22.4, 19.8, 18.4, 18.3, 17.2, 13.7, −4.3, −4.7; IR (film) νₘₐₓ 2955, 2930, 2856, 2238, 1710, 1253, 1068; ESIMS m/z 437 ([M+Cl⁻], C₂₄H₃₈ClO₃Si requires 437); HRMS (FAB) m/z 403.2687 ([M+H]⁺, C₂₄H₃₉O₃Si requires 403.2668).

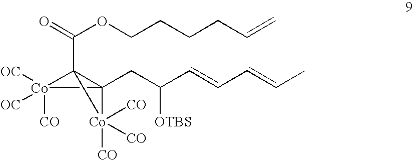

Cobalt complex 9. To a solution of acid 6 (192 mg, 0.653 mmol) and 5-hexen-1-ol (0.118 mL, 0.979 mmol) in dry CH₂Cl₂ (3 mL) was added EDCI (150 mg, 0.784 mmol) and 4-(dimethylamino)pyridine (8.0 mg, 0.065 mmol). After 3 hrs at room temperature, the reaction mixture was loaded on PTLC plates and purified (12:1 hexane/EtOAc) to give the model ester (146 mg, 59%). ¹H NMR (CDCl₃, 400 MHz) δ 6.18 (dd, J=15.1, 10.5 Hz, 1H), 6.04 (ddd, J=15.0, 10.5, 1.5 Hz, 1H), 5.79 (ddt, J=17.1, 10.3, 7.2 Hz, 1H), 5.71 (dq, J=15.0, 6.8 Hz, 1H), 5.56 (dd, J=15.1, 6.4 Hz, 1H), 5.03 (dq, J=17.1, 1.6 Hz, 1H), 4.97 (dd, J=10.2, 1.6 Hz, 1H), 4.35 (q, J=6.3 Hz, 1H), 4.16 (t, J=6.6 Hz, 2H), 2.57-2.44 (m, 2H), 2.09 (q, J=7.2 Hz, 1H), 1.77 (d, J=6.9 Hz, 3H), 1.74-1.63 (m, 2H), 1.52-1.44 (m, 2H), 0.90 (s, 9H), 0.10, 0.06 (2s, 6H); ¹³C NMR (CDCl₃, 100 Hz) δ 154.0, 138.4, 131.8, 131.1, 130.8, 130.5, 115.1, 86.6, 74.6, 71.5, 65.9, 33.4, 29.3, 28.1, 26.0, 25.3, 18.4, 18.3, −4.3, 4.7; IR (film) νₘₐₓ 2955, 2930, 2856, 2238, 1713, 1249, 1072; ESIMS m/z 399 ([M+Na⁺], C₂₂H₃₆NaO₃Si requires 399). HRMS (FAB⁺) m/z 375.2363 ([M−H]⁺, C₂₂H₃₅O₃Si requires 375.2355).

To a solution of the above alkynoic ester (77.8 mg, 0.207 mmol) in toluene (9 mL) was added Co₂(CO)₈ (99.0 mg, 0.289 mmol). The mixture was stirred at room temperature for 45 min, and then concentrated in vacuum. The dark residue was purified by PTLC (15:1 hexane/EtOAc) to give cobalt complex 9 (117.5 mg, 86%) as a red oil. ¹H NMR (CDCl₃, 400 MHz) δ 6.17 (dd, J=15.3, 10.6 Hz, 1H), 6.03 (ddd, J=15.3, 11.3 Hz, 1H), 5.68 (dd, J=14.9, 6.9 Hz, 1H), 5.61 (dd, J=15.2, 6.8 Hz, 1H), 5.38 (ddd, J=17.1, 10.1, 8.7 Hz, 1H), 5.10 (q, J=6.4 Hz, 1H), 5.04 (dd, J=17.1, 1.3 Hz, 1H), 4.85 (dd, J=10.3, 1.4 Hz, 1H), 4.41, (m, 1H), 3.20-3.15 (m, 2H), 1.76 (d, J=6.7 Hz, 3H), 1.59 (t, J=6.6 Hz, 2H), 1.32 (d, J=6.2 Hz, 3H), 1.22-1.17 (m, 1H), 0.90 (s, 9H), 0.82-0.72 (m, 1H), 0.66-0.59 (m, 2H), 0.09, 0.08 (2s, 6H); ¹³C NMR (CDCl₃, 100 Hz) δ 198.7, 169.2, 141.4, 134.3, 132.5, 132.4, 132.2, 131.0, 130.2, 128.8, 127.2, 127.1, 112.0, 93.0, 81.0, 73.7, 73.6, 73.1, 40.0, 26.1, 22.4, 19.9, 18.6, 18.4, 17.4, 13.9, 13.5, −4.2, −4.3, −4.6; IR (film) νₘₐₓ 2956, 2930, 2858, 2097, 2058, 2029, 1703, 1221, 1065; ESIMS m/z 685 ([M+Na⁺], C₂₈H₃₆Co₂NaO₉Si requires 685).

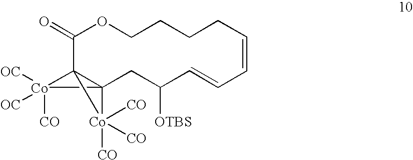

RCM product 10. To a solution of cobalt complex 9 (16 mg, 0.024 mmol) in dry CH₂Cl₂ (120 mL) was added tricyclohexyl phosphine[1,3-bis(2,4,6-trimethylphenyl)-4,5-dihydroimidazol-2-ylidene]-[bezyli-dene]ruthenium (IV) dichloride (second generation Grubbs catalyst) (6.1 mg, 0.0072 mmol). The resulting solution was heated to 45° C. for 1 hr and 10 min, then cooled to room temperature and filtered through a plug of silica gel. The solvent was removed under reduced pressure. The residue was purified by PTLC (15:1 hexane/EtOAc) to give cyclic product 10 (10.5 mg, 71%). ¹H NMR (CDCl₃, 400 MHz) δ 6.44 (dd, J=15.3, 10.8 Hz, 1H), 5.97 (t, J=10.8 Hz, 1H), 5.58 (dd, J=15.4, 7.5 Hz, 1H), 5.54 (dt, J=10.1, 4.5 Hz, 1H), 4.57-4.47 (m, 2H), 4.25-4.20 (m, 1H), 3.45-3.36 (m, 2H), 2.45-2.37 (m, 1H), 2.13-1.05 (m, 1H), 1.99-1.79 (m, 1H), 1.80-1.62 (m, 2H), 1.51-1.41 (m, 1H), 0.92 (s, 9H), 0.11, 0.08 (2s, 6H); ¹³C NMR (CDCl₃, 100 Hz) δ 199.9, 170.4, 135.2, 132.7, 129.0, 127.1, 92.8, 77.4, 73.1, 65.0, 45.2, 26.4, 26.1, 25.4, 18.5, 1.2, −4.2, 4.6; IR (film) νₘₐₓ 2955, 2930, 2857, 2098, 2059, 2027, 1702, 1213, 1057; ESIMS m/z 643 ([M+Na⁺], C₂₅H₃₀Co₂NaO₉Si requires 643).

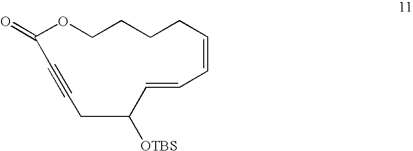

Model cyclic alkyne 11. To a solution of compound 10 (35.6 mg, 0.0574 mmol) in acetone at −10° C. was added ammonium cerium (IV) nitrate (189 mg, 0.344 mmol) portionwise. After 10 min at −10° C., the reaction was quenched by addition of diisopropylethylamine (0.18 mL, 1.03 mmol). The resulting mixture was filtered through a plug of neutral alumina, and the solvent was removed under reduced pressure. Purification by PTLC (15:1 hexane/EtOAc) afforded cyclic alkyne 11 (17.6 mg, 92%). ¹H NMR (CDCl₃, 400 MHz) δ 6.64 (dd, J=15.5, 11.1 Hz, 1H), 6.07 (t, J=11.0 Hz, 1H), 5.53 (dd, J=15.5, 7.1 Hz, 1H), 5.40 (dt, J=10.4, 4.8 Hz, 1H),), 4.41-4.25 (m, 2H), 4.06-4.01 (m, 1H), 2.69-2.62 (m, 1H), 2.56 (dd, J=17.1, 4.4 Hz, 1H), 2.46 (dd, J=17.1, 9.5 Hz, 1H), 2.26-2.21 (m, 1H), 1.75-1.61 (m, 4H), 0.89 (s, 9H), 0.08, 0.07 (2s, 6H); ¹³C NMR (CDCl₃, 100 Hz) δ 153.9, 133.2, 132.9, 128.5, 128.2, 87.7, 77.0, 72.9, 68.0, 29.0, 28.2, 26.7, 26.0, 25.6, 18.3, −4.3, −4.7; IR (film) νₘₐₓ 2954, 2929, 2857, 2238, 1716, 1245, 1110, 1075, 837; ESIMS m/z 357 ([M+Na], C₁₉H₃₀NaO₃Si requires 357). HRMS (FAB⁺) m/z 333.1888 ([M−H]⁺, Cl₉H₂₉O₃Si requires 333.1886).

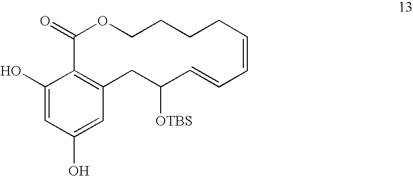

Diels-Alder product 13. Cyclic alkyne 11 (27 mg, 0.081 mmol) and excess diene 12 (0.30 mL, 0.90 mmol) were mixed and heated in a sealed vial to 140° C. for 48.5 hours. The mixture was cooled to room temperature, loaded onto a PTLC plate, and purified (4:1 hexane/EtOAc) to afford aromatic product 13 (20 mg, 60%). ¹H NMR (CDCl₃, 400 MHz) δ 11.64, (s, 1H), 6.38 (dd, J=15.4, 10.9 Hz, 1H), 6.35 (d, J=2.6 Hz, 1H), 6.30 (d, J=2.6 Hz, 1H), 6.23 (t, J=10.6 Hz, 1H), 5.95 (s, 1H), 5.78 (dd, J=15.3, 8.4 Hz, 1H), 5.60 (q, J=9.9 Hz, 1H), 4.69 (q, J=9.1 Hz, 1H), 4.12 (t, J=8.5 Hz, 2H), 3.63 (d, J=13.0 Hz, 1H), 2.62 (dd, J=13.1, 8.9 Hz, 1H), 2.50-2.40 (m, 1H), 2.12-2.05 (m, 1H), 1.87-1.76 (m, 2H), 1.56-1.44 (m, 2H), 0.78 (s, 9H), −0.20, −0.25 (2s, 6H); ¹³C NMR (CDCl₃, 100 Hz) δ 172.2, 165.7, 160.3, 144.8, 135.4, 131.0, 129.9, 126.0, 113.0, 105.3, 102.2, 78.7, 64.3, 46.6, 25.9, 24.2, 23.7, 23.0, 18.4, −4.7, −5.0; IR (film) νₘₐₓ 3380, 2954, 2929, 2856, 1648, 1620, 1254, 1169, 1106, 1061, 837; ESIMS m/z 441 ([M+Na⁺], C₂₃H₃₄NaO₅Si requires 441). HRMS (FAB⁺) m/z 418.2173 ([M]⁺, C₂₃H₃₄O₅Si requires 418.2176).

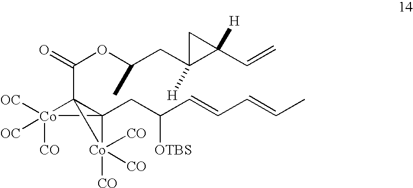

Cobalt complex 14. To a solution of alkyne 8 (526 mg, 1.31 mmol) in toluene (60 mL) was added Co₂(CO)₈ (625 mg, 1.83 mmol). The mixture was stirred at room temperature for 30 min, and the solvent was removed under reduced pressure. The dark residue was purified by flash chromatography (silica, 0 to 5% EtOAc in hexane) to give cobalt complex 14 (902 mg, 100%) as an inseparable mixture of two diastereomers. ¹H NMR (CDCl₃, 400 MHz) δ 6.17 (dd, J=15.3, 10.6 Hz, 1H), 6.03 (ddd, J=15.3, 11.3 Hz, 1H), 5.68 (dd, J=14.9, 6.9 Hz, 1H), 5.61 (dd, J=15.2, 6.8 Hz, 1H), 5.38 (ddd, J=17.1, 10.1, 8.7 Hz, 1H), 5.10 (q, J=6.4 Hz, 1H), 5.04 (dd, J=17.1, 1.3 Hz, 1H), 4.85 (dd, J=10.3, 1.4 Hz, 1H), 4.41, (m, 1H), 3.20-3.15 (m, 2H), 1.76 (d, J=6.7 Hz, 3H), 1.59 (t, J=6.6 Hz, 2H), 1.32 (d, J=6.2 Hz, 3H), 1.22-1.17 (m, 1H), 0.90 (s, 9H), 0.82-0.72 (m, 1H), 0.66-0.59 (m, 2H), 0.09, 0.08 (2s, 6H); ¹³C NMR (CDCl₃, 100 Hz) δ 198.7, 169.2, 141.4, 134.3, 132.5, 132.4, 132.2, 131.0, 130.2, 128.8, 127.2, 127.1, 112.0, 93.0, 81.0, 73.7, 73.6, 73.1, 40.0, 26.1, 22.4, 19.9, 18.6, 18.4, 17.4, 13.9, 13.5, −4.2, −4.3, −4.6; IR (film) νₘₐₓ 2956, 2930, 2858, 2097, 2058, 2029, 1703, 1221, 1065; ESIMS m/z 711 ([M+Na⁺], C₃₀H₃₈Co₂NaO₉Si requires 711).

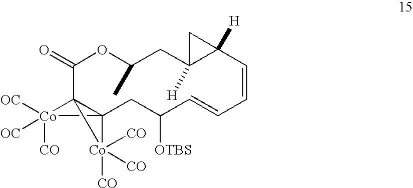

RCM product 15. To a solution of alkyne-cobalt complex 14 (67 mg, 0.097 mmol) in dry CH₂Cl₂ (485 mL) was added tricyclohexyl phosphine[1,3-bis(2,4,6-trimethylphenyl)-4,5-dihydroimidazol-2-ylidene][benzylidene]ruthenium (IV) dichloride (second generation Grubbs catalyst) (21 mg, 0.025 mmol). The resulting mixture was heated to 45° C. for 1.5 hours, and filtered through a short column of silica gel. The filtrate was concentrated in vacuum. The residue was purified by PTLC (15:1 hexanes/EtOAc) to give cyclic product 15 as a 2:1 mixture of two separable diastereomers. Major isomer (23.1 mg, 37%): ¹H NMR (CDCl₃, 400 MHz) δ 6.49 (dd, J=15.4, 10.9 Hz, 1H), 5.84 (t, J=10.6 Hz, 1H), 5.50 (dd, J=15.4, 8.5 Hz, 1H), 5.06 (dd, J=10.5, 6.9 Hz, 1H), 5.02-4.94 (m, 1H), 4.81-4.75 (m, 1H). 3.48-3.36 (m, 2H), 2.22-2.25 (m, 1H), 1.58-1.50 (m, 1H), 1.50-1.46 (m, 1H), 1.32 (d, J=6.2 Hz, 3H), 0.91 (s, 9H), 0.90-0.80 (m, 1H), 0.89 (s, 9H), 0.67-0.63 (m, 1H), 0.59-0.55 (m, 1H), 0.12, 0.09 (2s, 6H); ¹³C NMR (CDCl₃, 100 Hz) δ 199.0, 170.4, 135.7, 134.9, 129.2, 127.8, 92.0, 77.5, 72.5, 72.1, 45.5, 38.0, 26.1, 10.3, 18.4, 16.4, 16.0, −4.2, −4.6; IR (film) νₘₐₓ 2955, 2928, 2854, 2097, 2060, 2029, 1692, 1232, 1056; ESIMS m/z 669 ([M+Na⁺], C₂₇H₃₂Co₂NaO₉Si requires 669); [α]²⁵_{D} −127 (c 0.11, CHCl₃). Minor isomer (12.6 mg, 20%): ¹H NMR (CDCl₃, 500 MHz) δ 6.60 (dd, J=15.7, 9.9 Hz, 1H), 5.86 (t, J=10.3 Hz, 1H), 5.81 (dd, J=15.7, 4.4 Hz, 1H), 5.12-5.05 (m, 1H), 4.99 (t, J=9.9 Hz, 1H), 4.46-4.44 (m, 1H), 3.58 (dd, J=15.3, 9.4 Hz, 1H), 3.32 (dd, J=15.3, 4.2 Hz, 1H), 2.16 (dt, J=15.3, 4.2 Hz, 1H), 1.65-1.59 (m, 2H), 1.36 (d, J=6.5 Hz, 3H), 0.94 (s, 9H), 0.64-0.59 (m, 2H), 0.14 (s, 6H); ¹³C NMR (CDCl₃, 125 Hz) δ 198.6, 169.7, 135.9, 132.6, 126.8, 126.5, 92.0, 80.7, 73.4, 73.0, 41.8, 37.7, 26.1, 19.3, 18.5, 18.4, 15.1, 13.7, −4.5, −4.6; IR (film) νₘₐₓ 2929, 2856, 2097, 2059, 2028, 1702, 1220, 1059; ESIMS m/z 669 ([M+Na⁺], C₂₇H₃₂Co₂NaO₉Si requires 669). [α]²⁵_{D} +48 (c 0.19, CHCl₃).

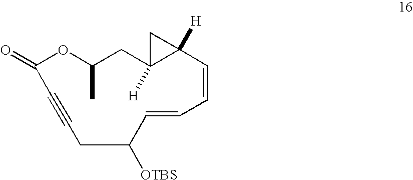

Cyclic alkyne 16. The major isomer of 15 (23.1 mg, 0.0358 mmol) was dissolved in dry THF (1 mL). At 0° C., a solution of 12 (135 mg, 0.536 mmol) in THF (5 mL) was added. After 35 minutes at 0° C., the reaction was quenched by the addition of a 2 mL 1:1 mixture of sat. aqueous Na₂S₂O₃ and NaHCO₃, followed by warming to room temperature and diluting with EtOAc (20 mL). The layers were separated, and the organic layer was washed with sat. aqueous NH₄Cl, dried (Na₂SO₄), filtered and concentrated. The residue was purified by PTLC (15:1, hexanes/EtOAc) to cyclic alkyne 16 as colorless oil (8.9 mg, 69%). ¹H NMR (CDCl₃, 400 MHz) δ 6.70 (dd, J=15.6, 11.1 Hz, 1H), 5.98 (t, J=10.8 Hz, 1H), 5.51 (dd, J=15.6, 7.7 Hz, 1H), 5.20-5.14 (m, 1H), 4.92 (t, J=10.7 Hz, 1H), 4.38-4.33 (m, 1H), 2.59 (dd, J=16.8, 4.3 Hz, 1H), 2.41 (dd, J=16.7, 10.9 Hz, 1H), 2.07 (dt, J=14.8, 1.8 Hz, 1H), 1.74-1.67 (m, 1H), 1.33 (d, J=6.5 Hz, 3H), 1.19-1.12 (m, 1H), 0.93-0.87 (m, 1H), 0.89 (s, 9H), 0.69-0.65 (m, 1H), 0.63-0.58 (m, 1H), 0.07 (s, 6H); ¹³C NMR (CDCl₃, 100 Hz) δ 153.4, 136.5, 132.5, 128.3, 125.4, 85.7, 77.4, 73.8, 72.0, 38.6, 29.2, 26.0, 20.4, 18.4, 18.2, 14.6, 12.8; IR (film) νₘₐₓ 2954, 2928, 2856, 2238, 1706, 1252, 1105, 1072, 1004; ESIMS m/z 383 ([M+Na⁺], C₂₁H₃₂NaO₃Si requires 383); HRMS (FAB⁺) m/z 360.2130 ([M]⁺, C₂₁H₃₂O₃Si requires 360.2121). [α]²⁵_{D} +56 (c 0.95, CHCl₃).

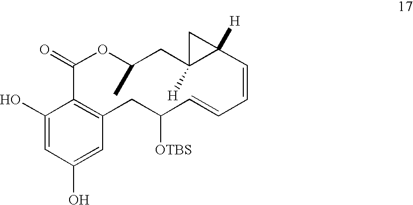

Diels-Alder product 17. Compound 16 (156 mg, 0.43 mmol) was dissolved in diene 12 (0.75 mL, 2.3 mmol) and heated to 140° C. in a sealed vial for 66 hours. The reaction mixture was cooled to room temperature and purified by PTLC (4:1, hexanes/EtOAc) to give 17 (143 mg, 75%). 7H NMR (CDCl₃, 400 MHz) δ 11.58 (s, 1H), 6.56 (dd, J=15.9, 9.3 Hz, 1H), 6.38 (d, J=2.4 Hz, 1H), 6.33 (d, J=2.4 Hz, 1H), 5.94 (t, J=9.5 Hz, 1H), 5.68 (dd, J=15.9, 6.8 Hz, 1H), 5.46-5.43 (m, 1H), 5.34 (dd, J=10.1, 4.3 Hz, 1H), 4.51 (q, J=6.7 Hz, 1H), 3.79 (dd, J=13.6, 6.2 Hz, 1H), 2.81 (dd, J=13.1, 8.9 Hz, 1H), 1.99-1.81 (m, 2H), 1.44 (d, J=6.5 Hz, 3H), 1.21-1.13 (m, 1H), 0.93-0.87 (m, 1H), 0.88 (s, 9H), 0.58-0.51 (m, 2H), −0.02 (s, 6H); ¹³C NMR (CDCl₃, 100 Hz) δ 164.8, 160.5, 143.8, 134.8, 133.7, 129.6, 128.0, 112.3, 106.1, 102.1, 75.7, 72.9, 42.6, 38.0, 26.1, 18.7, 18.4, 16.4, 16.1, 14.8, 1.4, −4.4, −4.6; IR (film) νₘₐₓ 3376, 2954, 2928, 2856, 1644, 1619, 1257, 1062, 835; ESIMS m/z 467 ([M+Na⁺], C₂₅H₃₆NaO₅Si requires 467). HRMS (FAB⁺) m/z 444.2336 ([M]⁺, C₂₅H₃₆O₅Si requires 444.2332). [α]²⁵_{D} +3.1 (c 1.3, CHCl₃).

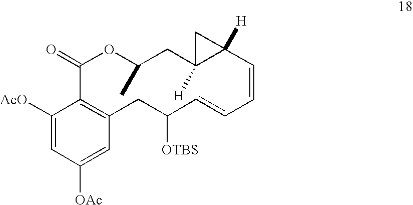

To a solution of 17 (243 mg, 0.546 mmol) in anhydrous DMF (13.5 mL) was added acetic anhydride (2.9 mL) and 4-(dimethylamino)pyridine (12.0 mg, 0.0546 mmol) sequentially. After 30 min at room temperature, the reaction was quenched by addition of 50 mL of pH 7.2 phosphate buffer. The resulting mixture was diluted with EtOAc (75 mL), separated and the aqueous layer was extracted with additional EtOAc (2×50 mL). The combined organic layers were washed with 5% aqueous NaCl (2×45 mL). The combined washings were extracted with EtOAc (2×30 mL). The combined organic layers were washed with sat. aqueous NaCl (30 mL), dried (Na₂SO₄), filtered and concentrated. Purification of the residue by PTLC (4:1 hexane/EtOAc) gave diacetate 18 (250 mg, 87%). ¹H NMR (CDCl₃, 400 MHz) δ 6.96 (d, J=2.1 Hz, 1H), 6.88 (d, J=2.1 Hz, 1H), 6.59 (dd, J=16.0, 10.5 Hz, 1H), 5.89 (t, J=10.5 Hz, 1H), 5.66 (dd, J=16.1, 4.9 Hz, 1H), 5.26-5.23 (m, 2H), 4.51 (q, J=5.8 Hz, 1H), 3.12-3.02 (m, 2H), 2.28, 2.23 (2s, 6H), 1.47 (d, J=6.2 Hz, 3H), 1.46-1.39 (m, 1H), 1.14 (ddd, J=14.9, 10.1, 2.0 Hz, 1H), 0.96-0.87 (m, 1H), 0.87 (s, 9H), 0.52-0.48 (m, 2H), −0.01, −0.02 (2s, 6H); ¹³C NMR (CDCl₃, 100 Hz) δ 168.6, 168.4, 166.4, 151.4, 148.7, 139.1, 135.9, 133.6, 129.2, 127.2, 125.6, 120.4, 114.7, 73.8, 73.2, 43.2, 40.0, 26.0, 21.3, 21.0, 19.3, 18.3, 16.9, 16.2, 17.7, −4.6, −4.7; IR (film) νₘₐₓ 2954, 2928, 2856, 1775, 1720, 1612, 1191, 1134, 1069; ESIMS m/z 551 ([M+Na⁺], C₂₉H₄₀NaO₇Si requires 551); HRMS (FAB⁺) m/z 528.2569 ([M]⁺, C₂₉H₄₀NaO₇Si requires 528.2543). [α]²⁵_{D} −38 (c 1.1, CHCl₃).

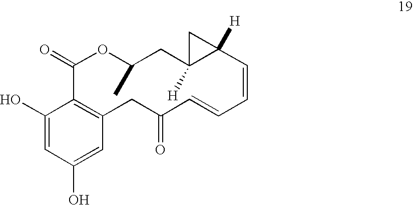

Cyclopropamonocillin 19. To a solution of diacetate 18 (250 mg, 0.473 mmol) in THF (10.2 mL) at 0° C. was added pyridine (3.4 mL) and HF-Pyridine complex (1.7 mL) sequentially. The resulting mixture was stirred at room temperature for 10.5 hrs. TMSOMe (30 mL) was added, and stirring was continued for 45 min to quench the remaining HF. The solvents were removed under reduced pressure. The alcohol isolated was dried under high vacuum, and dissolved in CH₂Cl₂ (15 mL) and cooled to 0° C. Dess-Martin periodinane (301 mg, 0.710 mmol) was added, and the resulting mixture was stirred at room temperature for 15 min. The solution was then directly loaded on PTLC plates and purified (1:1 hexane/EtOAc) to give the desired ketone (133 mg, 68%). ¹H NMR (CDCl₃, 500 MHz) δ 8.01 (dd, J=16.1, 11.3 Hz, 1H), 6.97 (d, J=1.4 Hz, 1H), 6.92 (d, J=2.0 Hz, 1H), 6.20 (t, J=10.4 Hz, 1H), 5.96 (d, J=16.0 Hz, 1H), 5.60 (dd, J=10.0, 7.2 Hz, 1H), 5.47-5.41 (m, 1H), 4.20 (d, J=13.8 Hz, 1H), 3.77 (d, J=13.8 Hz, 1H), 2.31 (dt, J=15.3, 4.4 Hz, 1H), 2.25, 2.24 (2s, 6H), 1.73-1.71 (m, 1H), 1.50 (d, J=6.5 Hz, 3H), 1.26-1.21 (m, 1H), 1.00-0.97 (m, 1H), 0.75-0.69 (m, 2H); ¹³C NMR (CDCl₃, 125 Hz) δ 198.4, 168.5, 165.0, 152.1, 149.2, 145.6, 143.7, 135.7, 129.6, 128.8, 124.6, 119.1, 115.6, 72.8, 43.0, 38.3, 21.2, 18.2, 16.7, 16.5, 15.6; IR (film) νₘₐₓ 2928, 1774, 1728, 1657, 1621, 1586, 1290, 1190, 1132, 1029; ESIMS m/z 435 ([M+Na⁺], C₂₃H₂₄NaO₇ requires 435); HRMS (FAB⁺) m/z 413.1616 ([M+H]⁺, C₂₃H₂₅O₇ requires 413.1600). [α]²⁵_{D} −269 (c 1.43, CHCl₃).

The above ketone (169 mg, 0.409 mmol) was dissolved in 26 mL 1:1 MeOH and 5% aqueous NaHCO₃, and stirred at room temperature for 14 hrs to remove the phenolic acetates. The resulting solution was diluted with sat. aqueous NH₄Cl (80 mL) and EtOAc (100 mL) and separated. The aqueous layer was extracted with additional EtOAc (3×75 mL). The combined organic layers were dried (Na₂SO₄), filtered and concentrated in vacuum. Purification by PTLC (1:1 hexane/EtOAc) provided 19 (124 mg, 92%). ¹H NMR (CDCl₃, 500 MHz) δ 11.33 (s, 1H), 8.36 (dd, J=16.1, 11.7 Hz, 1H), 6.48 (d, J=1.6 Hz, 1H), 6.40 (d, J=2.5 Hz, 1H), 6.20 (t, J=11.3 Hz, 1H), 6.01 (d, J=15.9 Hz, 1H), 5.72 (dd, J=9.8, 6.2 Hz, 1H), 5.49-5.45 (m, 1H), 5.32 (d, J=13.6 Hz, 1H), 3.51 (d, J=13.7 Hz, 1H), 2.31 (dt, J=15.8, 3.3 Hz, 1H), 1.61-1.55 (m, 1H), 1.55 (d, J=6.7 Hz, 3H), 1.32-1.28 (m, 1H), 1.03-0.97 (m, 1H), 0.74-0.72 (m, 1H), 0.64-0.63 (m, 1H); ¹³C NMR (CDCl₃, 125 Hz) δ 201.8, 170.1, 165.6, 161.6, 145.3, 139.2, 129.5, 128.5, 109.4, 104.5, 102.7, 73.5, 43.2, 27.6, 17.9, 17.2, 15.9, 13.9; IR (film) νₘₐₓ 3260, 1650, 1618, 1586, 1447, 1259, 1160, 1099, 996, 854; ESIMS m/z 351 ([M+Na⁺], C₁₉H₂₀NaO₅ requires 351); HRMS (FAB⁺) m/z 329.1382 ([M+H]⁺, C₁₉H₂₁O₅ requires 329.1389). (+)-(R, R, S)-19: [α]²⁵_{D} −189 (c 0.77, CH₂Cl₂).

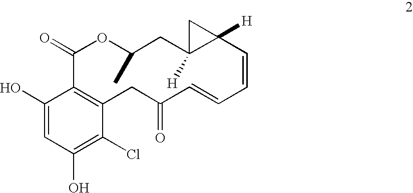

Cycloproparadicicol 2. Compound 19 (26.5 mg, 0.081 mmol) was dissolved in dry CH₂Cl₂ (5 mL) and cooled to 0° C. A solution of SO₂Cl₂ in CH₂Cl₂ (4.5 mL, diluted from 0.123 mL 1 M solution in CH₂Cl₂, 0.123 mmol) was added dropwise. After 45 minutes, the reaction was quenched by the addition of 5 mL of 5% NH₄Cl, and diluted with CH₂Cl₂. The layers were separated, and the organic phase was washed with brine, dried (MgSO₄), filtered and concentrated in vacuum. Purification of the residue by PTLC (3:1, hexanes/EtOAc) gave 2 as a white solid (17.8 mg, 61%). ¹H NMR (CDCl₃, 500 MHz) δ 10.89 (s, 1H), 8.00 (dd, J=15.9, 11.3 Hz, 1H), 6.63 (s, 1H), 6.42 (s, 1H), 6.13 (t, J=11.2 Hz, 1H), 6.04 (d, J=16.3 Hz, 1H), 5.62 (dd, J=9.9, 5.8 Hz, 1H), 5.46-5.42 (m, 1H), 4.90 (broad d, J=15.9 Hz, 1H), 3.77 (bs, 1H), 2.22 (dt, J=15.8, 3.2 Hz, 1H), 1.60-1.52 (m, 1H), 1.48 (d, J=6.7 Hz, 3H), 1.15-1.11 (m, 1H), 0.90-0.85 (m, 1H), 0.69-0.65 (m, 1H), 0.57-0.54 (m, 1H); ¹³C NMR (CDCl₃, 125 Hz) δ 199.2, 169.4, 162.7, 155.6, 143.5, 142.5, 136.9, 129.9, 128.9, 115.6, 107.8, 103.5, 74.4, 46.4, 37.4, 17.9, 17.3, 15.6, 13.6; IR (film) νₘₐₓ 3341, 1716, 1651, 1609, 1578 cm⁻¹; ESIMS m/z 385 ([M+Na⁺], C₁₉H₁₉NaO₅Cl requires 385); HRMS (ESI) m/z 385.0820 ([M+Na⁺], C₁₉H₁₉NaO₅Cl requires 385.0819). (+)-(R, R, S)-2: [α]²⁵_{D} +69 (c 0.87, CH₂Cl₂).

### Example 3

### Synthesis of Resorcinylic Macrolides via Ynolides: Establishment of Cycloproparadicicol as Synthetically Feasible Preclinical Anticancer Agent Based on Hsp90 as the Target

### Introduction

The heat shock protein 90 (Hsp90) is a molecular chaperone that mediates the stabilization and folding of various oncogenic proteins, such as Raf1 and Her2 (Banerji, U.; Judson, I.; Workman, P. Curr. Cancer Drug Targets 2003, 3, 385-390; incorporated herein by reference) Recently, Hsp90 has attracted extensive attention as a novel, potential antitumor target. The natural product family which has attracted by far the greatest attention as a potential inhibitor of the action of Hsp90 are congeners of geldanamycin (3) (Delmotte, P.; Delmotte-Plaquee, J. Nature 1953, 171, 344; Ayer, W. A.; Lee, S. P.; Tsuneda, A.; Hiratsuka, Y. Can. J. Microbiol. 1980, 26, 766-773; Roe, S. M.; Prodromou, C.; O'Brien, R.; Ladbury, J. E.; Piper, P. W.; Pearl, L. H. J. Med. Chem. 1999, 42, 260-266; each of which is incorporated herein by reference) Indeed, a derivative of geldanamycin, 17AAG (4), is the most advanced of drug candidates based on Hsp90 and is currently in phase II clinical trials (below). (Banerji, U.; Judson, I.; Workman, P. Curr. Cancer Drug Targets 2003, 3, 385-390; DeBoer, C.; Meulman, P. A.; Wnuk, R. J.; Peterson, D. H. J. Antibiot. 1970, 23, 442-447; each of which is incorporated herein by reference)

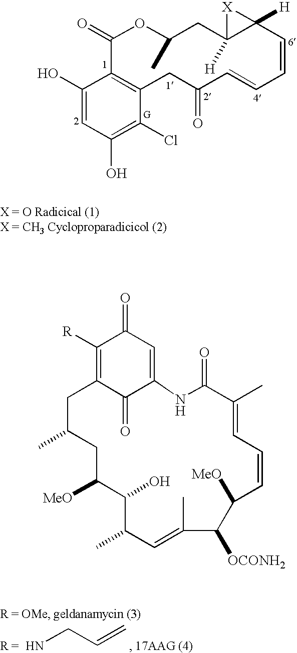

### Structures of Hsp90 Inhibitors

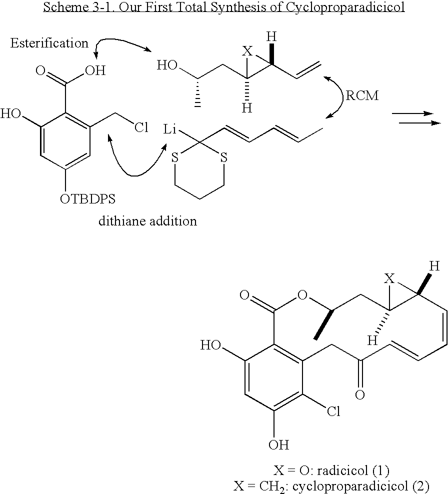

Though our laboratory has been involved in attempting to build upon 17AAG as a discovery lead centering around Hsp90 (Kuduk et al. Bioorg. Med. Chem. Len. 2000, 10, 1303-1306; Zheng et al. Cancer Res. 2000, 60, 2090-2094; Kuduk et al. Bioorg. Med. Chem. Lett. 1999, 9, 1233-1238; Chiosis et al. Curr. Cancer Drug Targets 2003, 3, 371-376; each of which is incorporated herein by reference), we hoped to evaluate the potentialities of another natural product targeting Hsp90, i.e., radicicol. We postulated that radicicol-derived drug candidates could in the long run out perform “geldanamycinoids” in that the latter would carry potential liabilities from the ansa bridged quinone substructure. Indeed, in a potentially important comparison, radicicol is significantly less hepatotoxic than 17AAG (Chiosis et al. Curr. Cancer Drug Targets 2003, 3, 371-376; incorporated herein by reference). However, radicicol was found to be ineffective in vivo animal models. We theorized that the epoxide both in radicicol and in radicicol oxime (which is active in in vivo models (Agatsuma et al. Bioorg. Med. Chem. 2002, 10, 3445-3454; incorporated herein by reference) could well be a source of nonspecific cytotoxicity, which could narrow the exploitable margin of therapeutic index. Furthermore, the potential chemical vulnerability of the dienyl epoxide raised concerns about drug shelf stability as well as pharmacokinetics. With a view to molecular editing, of the oxido function in a setting of minimal conformational perturbation of the radicicol lead, we were drawn to analogue 2 in which the epoxide linkage is replaced by a cyclopropane.

We had previously shown that compound 2 has an in vitro biological profile comparable to that of 1 (Yamamoto et al. Angew. Chem., Int. Ed. 2003, 42, 1280-1284; incorporated herein by reference). Moreover, we brought to bear an important line of evidence that 2 and 1 were closely related in their interactions with their biotargets. Thus changes in peripheral stereogenic centers in both 1 and 2 bring about the same consequences in biological function. In other words, both structures as shown are optimized from a stereo-chemical perspective. Moreover, and of considerable potential advantage for radicicol-based inhibitors, they also display cytotoxicity against Rb (retinoblastoma)-negative cells known to be resistant to 17AAG (Yamamoto et al. Angew. Chem., Int. Ed. 2003, 42, 1280-1284; incorporated herein by reference)

These promising findings inevitably raised issues as to the availability of cycloproparadicicol. Clearly, our first synthesis of this compound (Yamamoto et al. Angew. Chem., Int. Ed. 2003, 42, 1280-1284; incorporated herein by reference), while touching on several issues of academic interest in organic chemistry, did not seem promising for producing more than token amounts of the now interesting 2.

As seen, the first synthesis from our lab relied on the appropriate sequenced Mitsunobu esterification, dithiane alkylation, and ring closing olefin metathesis. While highly convergent and concise, this first generation pathway to radicicol suffered from several low yielding steps which did not improve following attempted optimization. In particular, the low yields associated with the dithiane alkylation and ring-closing metathesis (RCM) steps sharply curtailed access to cycloproparadicicol for evaluation (Yamamoto et al. Angew. Chem., Int. Ed. 2003, 42, 1280-1284; incorporated herein by reference)

Indeed, a new strategy has been formulated and reduced to practice, resulting in a much improved second generation total synthesis of cycloproparadicicol (Yang et al. J. Am. Chem. Soc. 2003, 125, 9602-9603; incorporated herein by reference). In addition, the new route shows promise of applicability to a broad range of resorcinylic macrolides (Geng et al. J. Org. Len. 2004, 6, 413-416; incorporated herein by reference). Herein, we disclose the full details of this new approach and its application to reach cycloproparadicicol as well as aigalomycin D. The synthesis and biological activities of a number of interesting analogues of cycloproparadicicol will be described. Based on the much improved route of synthesis, and based on early investigations of biological profiles, cycloproparadicicol now emerges as a candidate for full scale preclinical evaluation.

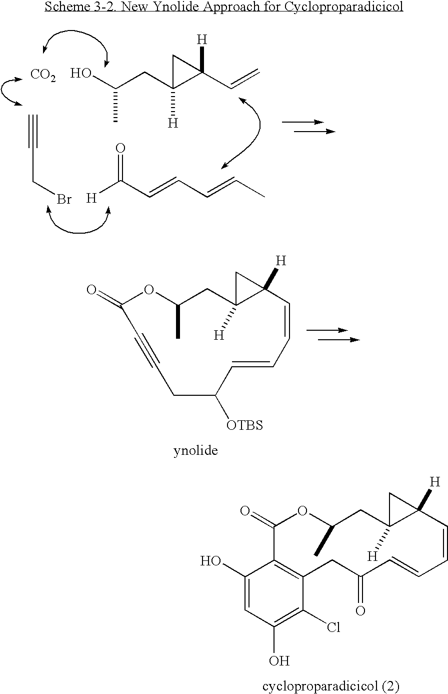

### Results and Discussions

Overall Strategy. The defining element of our second generation strategy was the building of the aromatic sector of the resorcinylic marcrolide by Diels-Alder reaction of a new type of dienophile, i.e., an “ynolide”. This cycloaddition route to the benzo-fused macrolactone represents a substantial departure from the usual mode of synthesis in which one starts with an aromatic ring and appends to it suitable arms to close the macrolactone ring (cf. Schemes 3-1 and 3-2). We hoped that the new ynolide approach, if successful, would be highly convergent and would allow for rapid access to a broad family of resorcinylic macrolides. Since it has been our experience that monoactivated acetylenic dienophiles are surprisingly weakly reactive in Diels-Alder reactions (Danishefsky et al. J. Org. Chem. 1979, 44, 4716-4717; incorporated herein by reference), the success of a projected DielsAlder cycloaddition aromatization sequence could not have been anticipated with confidence.

Synthesis of Acyclic Alkynoic Ester. To facilitate progress to the key issues of the plan, we sought to develop an efficient synthesis route to reach a seco-alkynoate ester, which, following RCM, would afford the ynolide dienophile. In practice, the synthesis commenced with commercial 2,4-hexadienal (sorbaldehyde, 6, Scheme 3-3). Reformatsky-like condensation of propargyl bromide (5) with 6 led to the expected carbinol. Following β-esterification, alkyne precursor 7 was in hand in good yield (Friedrich et al. J. Org. Chem. 1996, 61, 1082-1100; incorporated herein by reference). Treatment of 7 with n-butyllithium generated an alkynide ion, which was carboxylated with dry ice, to provide acid 8, in very high yield (Fuganti et al. Tetrahedron: Asymmetry 1994, 5, 1135-1138; incorporated herein by reference). Following reaction of racemic 8 and the known optically pure and defined alcohol 9 (Garbaccio et al. J. Am. Chem. Soc. 2001, 123, 10903-10908; incorporated herein by reference) under Mitsunobu conditions (Romo et al. J. Am. Chem. Soc. 1998, 120, 12237-12254; incorporated herein by reference), ester 10 was obtained as a diastereomeric mixture at the future C_{2′}.

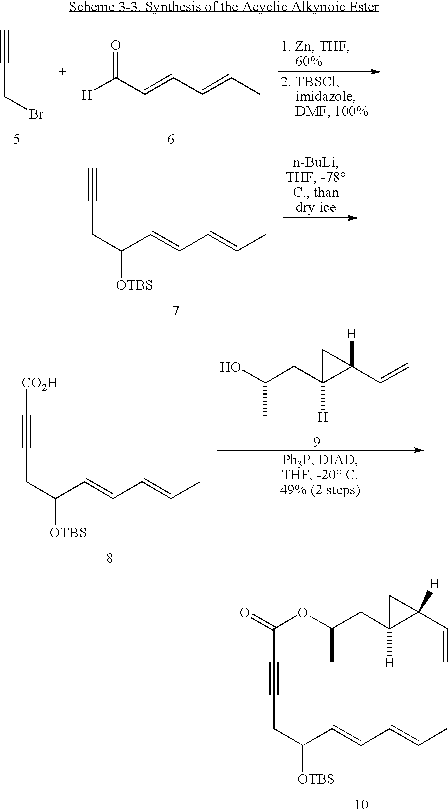

Scheme 3-4. Attempted Ring-Closing Metathesis Reactions of 10

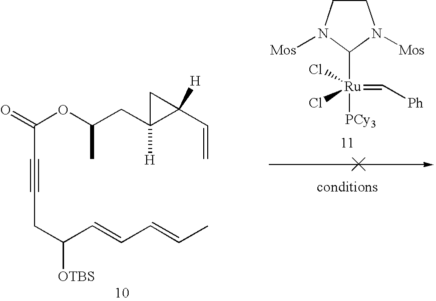

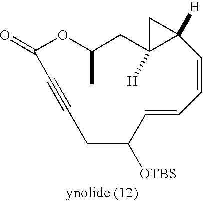

| Entry | Conditions | results |
|---|---|---|
| 1 | 0.25 equiv 11, CH2Cl2, 40° C., 0.5 mM, 2 h | recovered 10 |
| 2 | 0.25 equiv 11, toluene, 120° C., 0.5 mM, 1 h | recovered 10 |
| 3 | 1.1 equiv 11, toluene, 120° C., 0.2 mM, 10 min | 10 decomposed |
| 4 | 0.3 equiv 11, 0.3 equiv Ti(Oi-Pr)4, CH2Cl2, | recovered 10 |
| | 0.5 mM, 40° C., 2 h | |

Macrocyclization via Ring Closing Olefin Metathesis. The next key reaction was the formation of our hoped macrocyclic ynolide through ring-closing olefin metathesis. For this purpose, we made recourse to the powerful metalo carbene catalysts, innovated by Grubbs and associates, based on ligand 11 (Scholl et al. Org. Len. 1999, 1, 953-956; incorporated herein by reference). However, as shown in Scheme 3-4, all efforts to form the desired ynolide 12 using catalytic amounts of 11 provided the recovered starting ester 10. The Lewis acid, Ti(Oi-Pr)₄, was used in the hope of preventing formation of an inactive complexing locus which included the ester group. However, this attempt at reaction steering was also not successful. Attempted increase of the amounts of 11 to stoicheometric levels eventually led to decomposition of 10.

The failure of probe structure 10 to cyclize could be ascribed to conformational rigidities associated with the trans-disubstituted cyclopropane and the linear acetylene “linker.” Accordingly, a more flexible model ester 14 was prepared (in 59% yield) using standard protocols. Compound 14 was subjected to RCM conditions (Scheme 3-5). Unfortunately, no formation of cyclic product was observed.

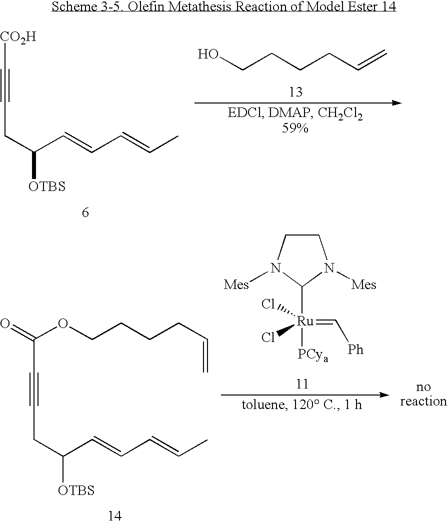

Cobalt Complexation to Promote RCM. At this stage, we reasoned that the failure of olefin metathesis might be due to the presence of the acetylene group. Aside from the constraint to cyclization imposed by its linear character, the cyclization could further be complicated by nonproductive coordination of the acetylene to the RCM catalytic machinery. The latter possibility could not be dismissed out of hand, since the enyne metathesis reactions are well established (Kinoshita et al. J. Org. Chem. 1996, 61, 8356-8357; Mori et al. J. Org. Chem. 1998, 63, 6082-6083; incorporated herein by reference). Accordingly, protection of the alkyne could well be helpful. It is well-known that reactions of dicobalt carbonyl with acetylenes can lead to stable complexes, in which the alkyne functions are, in effect, protected (Greenfield et al. J. Am. Chem. Soc. 1956, 78, 120-124; Nicholas et al. Tetrahedron Len. 1971, 37, 3475-3478; incorporated herein by reference). Moreover, the geometry of cobalt-complexed alkynes optimizes at approximately 140° (Dickson et al. J. Adv. Organomet. Chem. 1974, 12, 323-377; incorporated herein by reference). Such a departure from linearity could well favor cyclization (Recently, a similar protocol was reported: Young et al. J. Org. Chem. 2003, 68, 3494-3497; incorporated herein by reference). We thus hoped that cobalt protection of the alkyne could serve to our advantage in the problematic macrocyclization.

To probe the feasibility of this hypothesis, model ester 14 was first treated with cobalt dicarbonyl (Scheme 3-6). Complex formation proceeded readily at room temperature in toluene to provide 15 (in 87% yield) after silica gel chromatography. When complex 15 was subjected to the conditions of ring closing olefin metathesis, with catalyst, cyclization proceeded smoothly at room temperature. The cobalt-protected ynolide 16 was obtained in 71% using 25 mol % of 11 and high dilution (0.2 mM) in methylene chloride.

To retrieve the free acetylene of the ynolide, the cobalt complex had to be “demetalated.” This goal was readily achieved by treatment of 16 with excess ceric ammonium nitrate in acetone at low temperature (Magnus, P. Tetrahedron 1994, 50, 1397-1418; incorporated herein by reference). Ynolide 17 was obtained in excellent yield (Scheme 6).

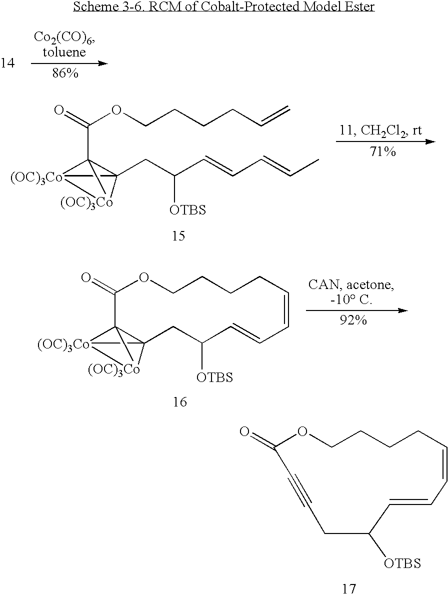

We next applied this strategy to the target system 10. Gratifyingly, as was the case with the model compound 15, RCM reaction of cobalt-complexed 18 proceeded smoothly at room temperature in CH₂Cl₂ to afford cyclic product 19 in 57% yield. Postmetathesis removal of the cobalt, however, was nontrivial. Treatment of 19 with CAN in acetone led primarily to decomposition. Other oxidizing agents such as 12 (Tanaka et al. Tetrahedron Len. 1993, 34, 5757-5760; incorporated herein by reference) or Me₃—NO (Jones et al. J. Org. Chem. 1997, 62, 9379-9381; incorporated herein by reference) improved the yields significantly but were not fully reproducible when conducted in large scales. Eventually, it was found that when the solution of 19 was buffered with 2,6-di-tert-butyl pyridine prior to the treatment with CAN, the desired product 12 was isolated in 50% yield in two steps from 18 (Scheme 3-7) (Magnus et al. J. Am. Chem. Soc. 1997, 119, 5591-5605; incorporated herein by reference). At this stage, the two stereoisomers of 12 became separable, and they were advanced individually.

Cycloaddition-Aromatization Reactions. Having achieved an efficient synthesis of the required ynolide, we directed our attentions to the next challenge, i.e., the fashioning of the desired resorcinylic macrolides using a Diels-Alder elimination sequence. We appreciated that acetylenic dienophiles, where only one of the sp carbons is activated with a typical activating group such as an ester can be rather unreactive (Danishefsky et al. J. Org. Chem. 1979, 44, 4716-4717; incorporated herein by reference). These monoactivated acetylenes must be clearly distinguished at the planning level from diactivated acetylenes such as diesters of acetylenedicarboxylic acid which are powerful dienophiles.

Scheme 3-7. Synthesis of Ynolide 12

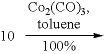

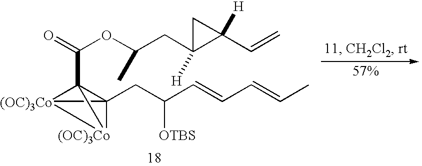

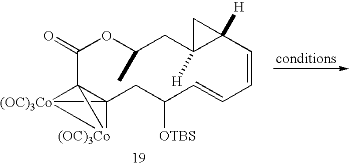

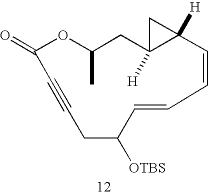

| Entry | Conditions | Yields |
|---|---|---|
| 1 | CAN, acetone, −10° C. | <10% |
| 2 | I2, THF, 0° C. | <69% |
| 3 | Me, NO, acetone/THF −78° C. tort | 66% |
| 4 | CAN, DTBP, acetone, −10° C. | 50% from 18 |

At first, trioxygen-substituted dienes of the type 20 (Yamamoto et al. Chem. Lett 1978, 649-652; incorporated herein by reference) and 21 (Danishefsky et al. J. Org. Chem. 1979, 44, 4716-4717; Banville et al. J. Chem. Soc., Perkin Trans. 1 1976, 1852-1856; Danishefsky et al. J. Org. Chem. 1978, 43, 379-380; incorporated herein by reference) seemed to be appropriate choices to attempt cycloaddition with ynolides 12. Ideally, reaction of 12 with diene 20 followed by elimination of MeOH would, upon desilylation, lead to the desired free phenol product 22. The use of diene 21 would require an additional step to deprotect the phenolic methyl ether of the product 23 (Scheme 3-8). Such deprotections had been found to be nontrivial in related systems (Garbaccio et al. J. Am. Chem. Soc. 2001, 123, 10903-10908; incorporated herein by reference).

Scheme 3-8. Attempted Diels-Alder Reactions with Acyclic Dienes OTMS

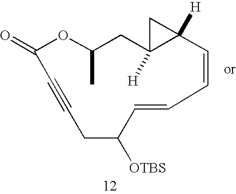

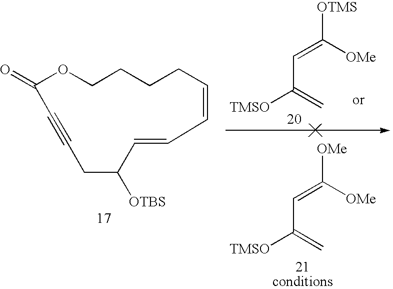

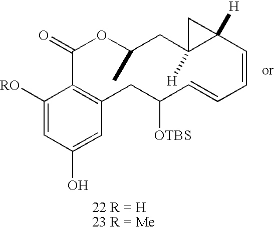

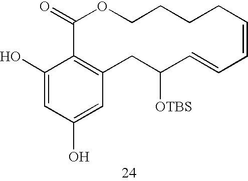

| Entry | Conditions | Results |
|---|---|---|
| 1 | 12, 20, neat, 75° C.; | recovered desilylated 12 |
| | then Et3N.HF, EtOH | |
| 2 | 12, 21, neat, 160° C.; then 0.1 N HCI | Decomposition |
| 3 | 17, 20, EuFOD, neat, 70° C.; | recovered desilylated 17 |
| | then Et3N.HF, EtOH | |
| 4 | 17, 20, Ti(Oi-Pr)4, neat, 70° C. | recovered 17 |

To our surprise, when 12 and diene 20 were heated to 70° C., no aromatic products were isolated. Desilylated 12 was recovered. Since 20 is prone to undergo a 1,5-silyl shift at temperatures above 70° C. (Dickson, R. S.; Fraser, P. J. Adv. Organomet. Chem. 1974, 12, 323-377; incorporated herein by reference), we directed our efforts to the thermally more stable diene 21. Unfortunately, even when 12 and diene 21 were heated to 160° C., no products were isolated. The ynolide 12 had decomposed. In the hope of lowering the temperature required for the cycloaddition, we turned to the use of Lewis acids. Considering the extreme acidic liability of these dienes, we chose mild reagents Eufod (Danishefsky et al. J. Org. Chem. 1984, 49, 392-393; Castellino et al. Tetrahedron Len. 1984, 25, 2307-2310; Lopez et al. Tetrahedron: Asymmetry 1991, 2, 93-96; each of which is incorporated herein by reference) in the context of projected Diels-Alder reactions of model ynolide 17 with diene 21. Unfortunately, no product formation was observed.

We next directed our attentions to the use of the dimedone derived cyclic diene 25 (Ibuka, T.; Mori, Y.; Aoyama, T.; Inubushi, Y. Chem. Pharm. Bull. 1978, 26, 456-465; Langer, P.; Schneider, T.; Stoll, M. Chem. Eur. J. 2000, 6, 3204-3214; each of which is incorporated herein by reference) (Scheme 3-9). The latter could, in principle, react with ynolide diastereomers 12 to afford desired product 22 after elimination of isobutylene (Uchiyama et al. Tetrahedron Len. 2000, 41, 10013-10017; Morrison et al. Tetrahedron Len. 2001, 42, 7367-7369; incorporated herein by reference). The possible advantages of this diene over tri-oxygen-substituted acyclic possibilities, such as 20 or 21, are that the cyclic diene could well be more reactive (due to a locking in of the s-syn conformation by the six-member ring) and more thermally stable. Happily, when diene 25 and dienophile 12 were heated to 160° C., followed by desilylation, in the course of silica gel chromatography, the desired product, 22, was obtained in 78% yield. Thus a new, highly efficient and convergent method to assemble the resorcinylic macrolide 22, needed for cycloproparadicicol, had been achieved.

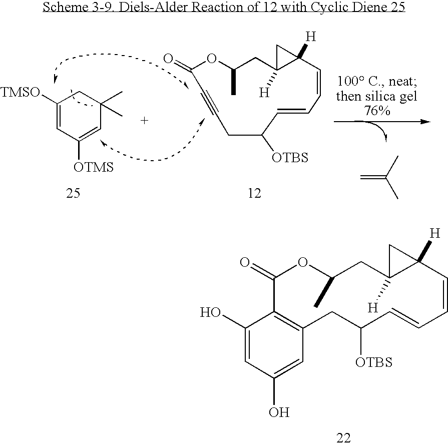

Completion of the Synthesis. To complete the synthesis of cycloproparadicicol (2), two remaining steps were required. Oxidation of the 2° alcohols to the corresponding ketone and regioselective introduction of a chlorine had to be accomplished in some as yet unspecified order. We first attempted to carry out the oxidation of the secondary homobenzylic alcohol in the presence of the free phenolic hydroxyls (Scheme 3-10). Following the removal of the TBS ether group (using HF/pyridine) in 74% yield, we surveyed a variety of oxidation conditions. Unfortunately, in the best case, we obtained only 30% yield through the use of activated MnO₂ (Gritter et al. J. Org. Chem. 1959, 24, 1051-1056; Trost et al. J. Org. Chem. 1983, 48, 3252-3265; each of which is incorporated herein by reference). The use of other oxidizing agents examined resulted in the decomposition of starting material, 22 (Huang et al. J. Org. Chem. 1976, 41, 3329-3231; Hauser et al. J. Org. Chem. 1983, 48, 1328-1333; Welch et al. Synth. Commute 1993, 23, 131-134; Tatsuta et al. Chem. Len. 2001, 172-173; Ley et al. Synthesis 1994, 639-666; each of which is incorporated herein by reference).

Scheme 3-10. Oxidation in the Presence of Free Phenols

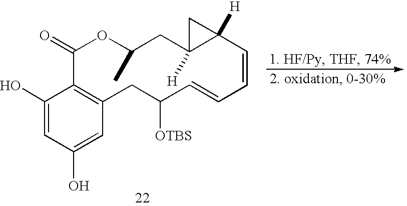

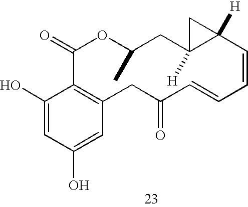

| Entry | Oxidation conditions | Yields |
|---|---|---|
| 1 | MnO₂ | <30% |
| 2 | TFAA/DMSO/Et₃N, CH₂Cl₂ | Decomposition |
| 3 | PCC, NaOAc, CH₂Cl₂ | Decomposition |
| 4 | Dess-Martin periodinane, CH₂Cl₂ | decomposition |
| 5 | TPAP, CH₂Cl₂, MS 4Å | decomposition |

Accordingly, we were obliged to protect the two free phenolic hydroxyl groups. Treatment of 22 with acetic anhydride in DMF and cat. amount of N,N-dimethylamino pyridine (DMAP) provided diacetate 24 (87% from the major isomer of 22 and 76% from the minor isomer of 22) (Scheme 11). Subsequent removal of its TBS ether gave alcohol 25. This deprotection was followed by oxidation using Dess-Martin periodinane, to afford the desired ketone intermediate in 68% yield from the major isomer of 25 (80% yield from the minor isomer). The stereochemistry of two isomers of 25 could be determined by a modified Mosher's analyses (see Experimentals for details) (Ohtani et al. J. Org. Chem. 1991, 56, 1296-1298; each of which is incorporated herein by reference). The acetate groups were cleaved under mildly basic conditions (5% NaHCO₃/MeOH, 1:1) in excellent yield (Garbaccio et al. J. Am. Chem. Soc. 2001, 123, 10903-10908; incorporated herein by reference). Finally, regioselective chlorination provided cycloproparadicicol (2) in 70% yield. Unfortunately, this product was accompanied by the formation of an isomeric chlorination product 26 in 27% yield (Garbaccio et al. J. Am. Chem. Soc. 2001, 123, 10903-10908; incorporated herein by reference). Although the issues governing such chlorinations have not necessarily been optimized, this level of regioselectivity seems to be quite general for such resorcinylic compounds (Elix et al. Aust. J. Chem. 1997, 50, 971-975; Elix et al. Aust. J. Chem. 1992, 45, 845-855; each of which is incorporated herein by reference). In summary, we have developed a new concise synthesis for cycloproparadicicol, in 6% yield following 13 steps.

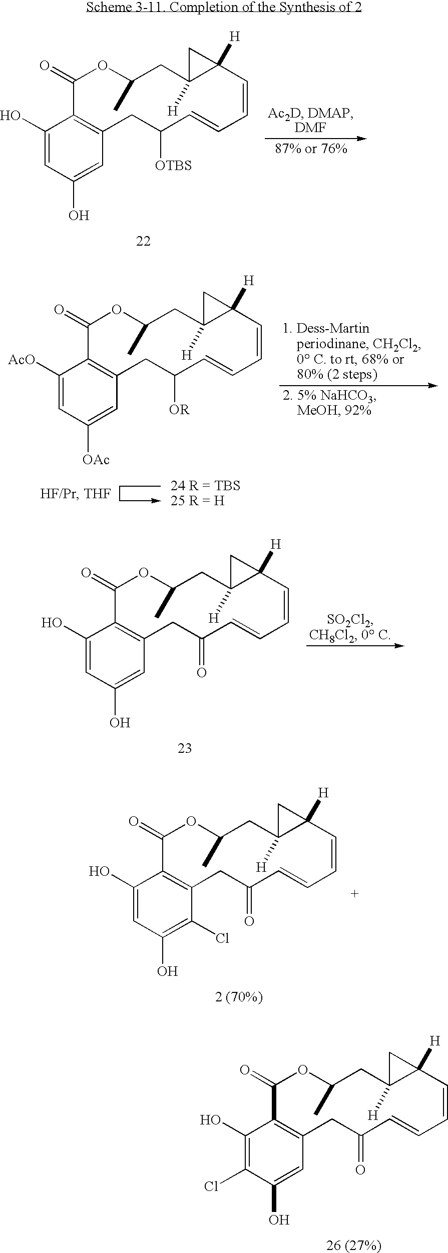

Synthesis of Analogues and Formulation of a Preliminary SAR Pattern in the Cycloproparadicicol Series. The new synthesis developed here not only allowed us to generate sufficient quantities of cycloproparadicicol to support our biological studies on the compound itself but also allowed us to fashion a series of interesting analogues to study structure-activity relationships. To evaluate the biological impact of modification of the ketone group, two alcohol isomers 27α,β were prepared (Scheme 3-12). Using SO₂Cl₂, the chloride derivative 28α was synthesized in one step from the major isomer, 27α. The methyl ether derivative of 27α, 29α was also prepared under neutral conditions (MeI/Ag₂O) (Tanis et al. J. Am. Chem. Soc. 1992, 114, 8349-8362; incorporated herein by reference). Oxime analogues of cycloproparadicicol [(Z)- and (E)-30] were synthesized as well (Agatsuma et al. Bioorg. Med. Chem. 2002, 10, 3445-3454; incorporated herein by reference).

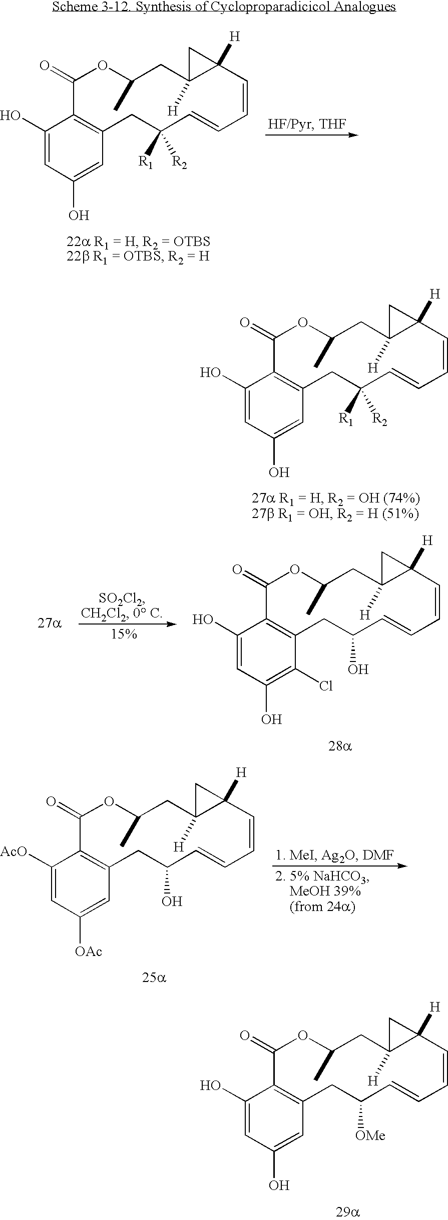

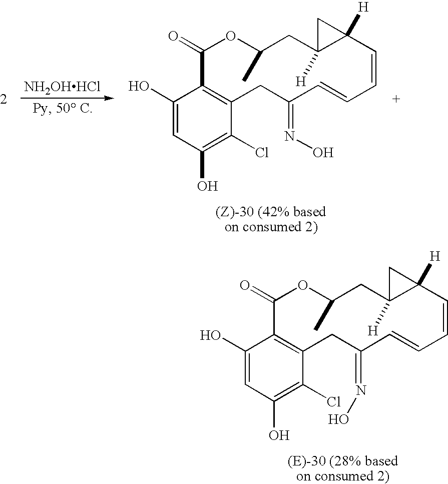

Synthesis of Difluorocycloproparadicicol. Our laboratory recently discovered that a trifluoro-derivative of epothilone exhibits a far superior in vivo profile (Chou et al. Angew. Chem., Int. Ed 2003, 42, 4762-4767; incorporated herein by reference). Accordingly, we sought to synthesize difluorocycloproparadicicol 31, hoping to identify even better druglike molecules targeting Hsp90.

The difluorocyclopropane group was introduced to the known conjugated ester 32, using reagents developed by Dolbier and co-workers, thereby affording difluoroesters 33 as a 1:1 mixture of stereoisomers (Scheme 3-13) (Tian et al. Org. Lett. 2000, 2, 563-564; incorporated herein by reference). Our goal here was to achieve the target rapidly using the methodology already described in this account. Hence, the mixture of stereoisomers was carried forward through the required synthetic steps. As outlined in Scheme 3-13, the key ynolide 36 was prepared in reasonable yields following the now familiar protocols described above. The subsequent Diels-Alder reaction with diene 25, however, proved to be very difficult, affording only 18% of product. Increasing the reaction temperature further eroded the yield, presumably due to the thermal instability of the difluorocyclopropane-containing ynolide 35. Nevertheless, using the post-Diels-Alder sequence described previously, adequate amounts of 31 and 38 were obtained to support in vitro studies. It will be noted that, after Dess-Martin oxidation, ketone 38 was isolated as a single isomer. Seemingly, this difluoroyclopropyl isomer of 38 appears to have the same cyclopropane centers (7′R, 8′R) as does cycloproparadicicol itself, based on ¹H NMR and optical rotation comparisons (see the Experimentals below for details). Presumably, the alternate trans cyclopropyl diastereomer fares poorly in terms of yield in going through the steps from 35 to the end.

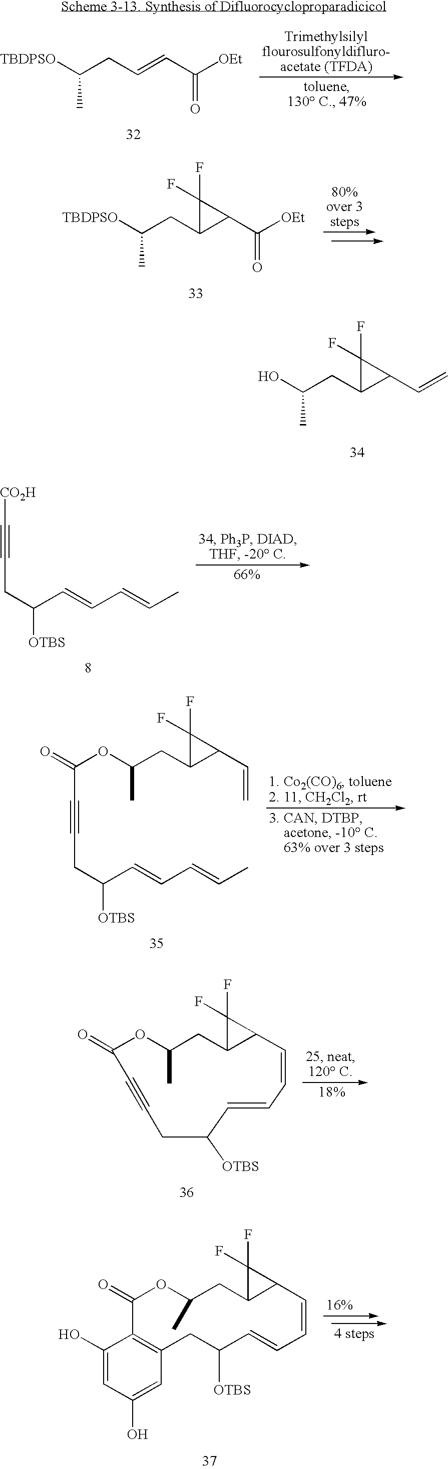

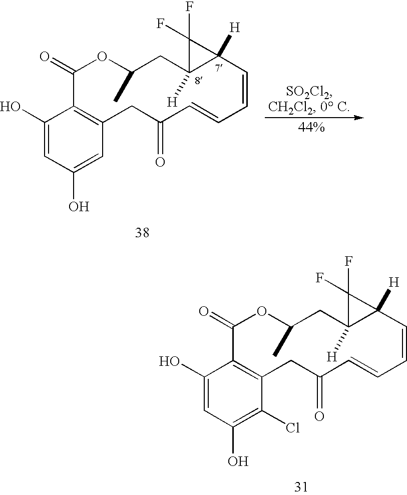

Synthetic Approach to the Cycloproparadicicol Lactam. One of the structural features of radicicol that might have contributed to its instability in vivo, in addition to the 7′-8′ epoxide, is the macrolactone. The latter could be subject to metabolic hydrolysis by esterases. With this in mind, we also set out to synthesize the lactam version of the cycloproparadicicol (see compound 39).

To introduce the functionality required to reach 39, a sequence involving Staudinger reduction of an azide was considered (Stachel et al. J. Org. Chem. 2001, 66, 4369-4378; incorporated herein by reference). However, Mitsunobu type methodology, (Mitsunobu, O, Synthesis 1981, 1-28) including Thompson's protocol (Thompson et al. J. Org. Chem. 1993, 58, 5886-5888; incorporated herein by reference) failed to deliver the corresponding azide of compound 9. This is probably due to the instability of the azide compound. Fortunately, it was found that the Nosyl approach recently developed by Fukuyama (Kurosawa et al. J. Am. Chem. Soc. 2003, 125, 8112-8113; incorporated herein by reference) afforded the desired sulfonamide 40 in 61% yield (Scheme 3-14). After the removal of the sulfonyl group, the resulting amine, generated in situ, coupled with alkynoic acid 8 to afford amide 41. Application of the protocol described above (cobalt complexation→RCM→oxidative decomplexation) to the case at hand furnished “ynelactam” 44. The Diels-Alder reaction of 44 with diene 25, however, proved to be highly problematic. The best yield realized for reaching product 45 was only around 25%, and that proved to be difficulty reproducible. It is likely that replacement of the ester group by an amide function attenuates the electronegative pull on the acetylene, thereby eroding the reactivity of this linkage as a dienophile. Currently, investigations of other types of more reactive dienes are actively underway in our laboratory to deal with the problem of poorly reactive acetylenic dienophiles. Success on this front would allow us to operate more effectively with weakly reactive dienophiles such as 44. Pending solution of the synthetic problem, studies directed to the synthesis of 39 have been deferred.

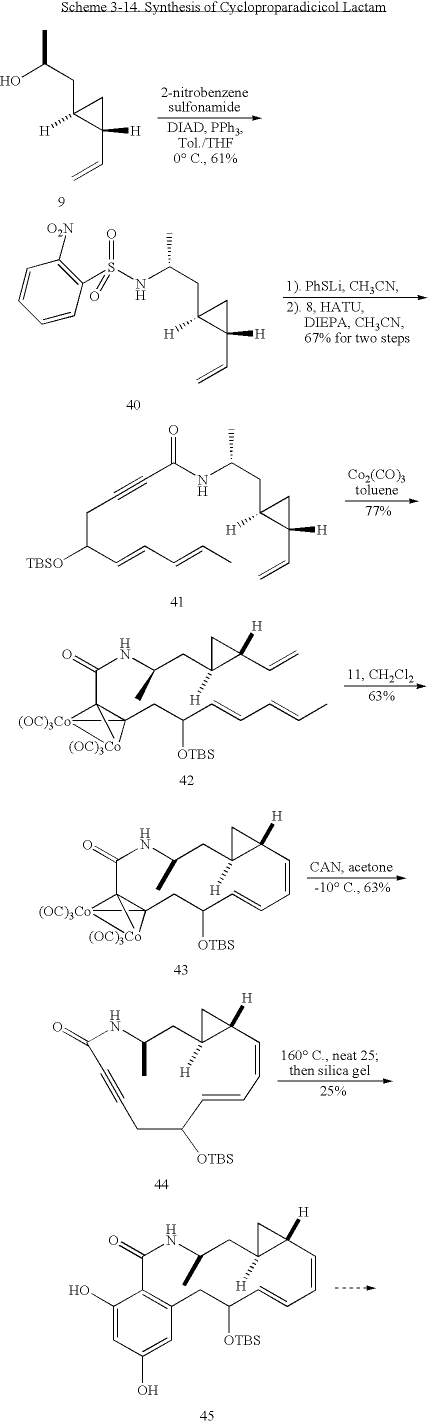

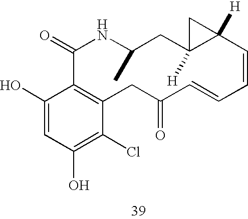

### Extension of the Ynolide Approach to the First Total Synthesis of Aigialomycin D.

Nature has provided a variety of biologically significant natural products which share the same structural motif as radicicol, i.e., a 14-member resorcinylic macrolide. We sought to test the scope and limitations of our newly developed ynolide-DA approach to such ring systems by applying it to an interesting member of this family, agialomycin D (46) (below). Recently, this compound was isolated from the marine mangrove fungus Aigialus parvus BCC5311 (Isaka et al. J. Org. Chem. 2002, 67, 1561-1566; incorporated herein by referened). It exhibited potent antimalarial activity (IC₅₀: 6.6 μg/mL against P. falciparum) and antitumor activity (IC₅₀: 3.0 μg/mL against KB cells) (Isaka et al. J. Org. Chem. 2002, 67, 1561-1566; incorporated herein by reference) We saw agialomycin D as an attractive candidate expanding upon our new “ynolide” strategy. An important issue to be overcome would be the proper emplacement of the E 1′, 2′ double bond requested to reach aigialomycin D. Also, the route must accommodate the hydroxy bearing stereogenic centers at carbons 5′ and 6′.

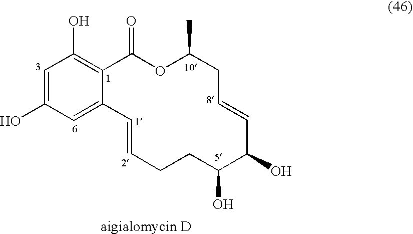

### Structure of Aigialomycin D

As shown in Scheme 3-15, the total synthesis commenced from the naturally derived D-2-deoxy-ribose 47, which already has the desired future syn 5′,6′-diol functionality of aigialomycin D in place. Compound 47 was processed forward via diol protection, Wittig olefination, hydroboration, and oxidation, to furnish key aldehyde 52, as shown in Scheme 3-15. This compound was subjected to nucleophilic propargylation followed by protection of the resultant alcohol to afford TBS ether 53. The pivaloyl group of 53 was then removed, and a vinyl group was installed through oxidation and Wittig reactions (see compound 56).

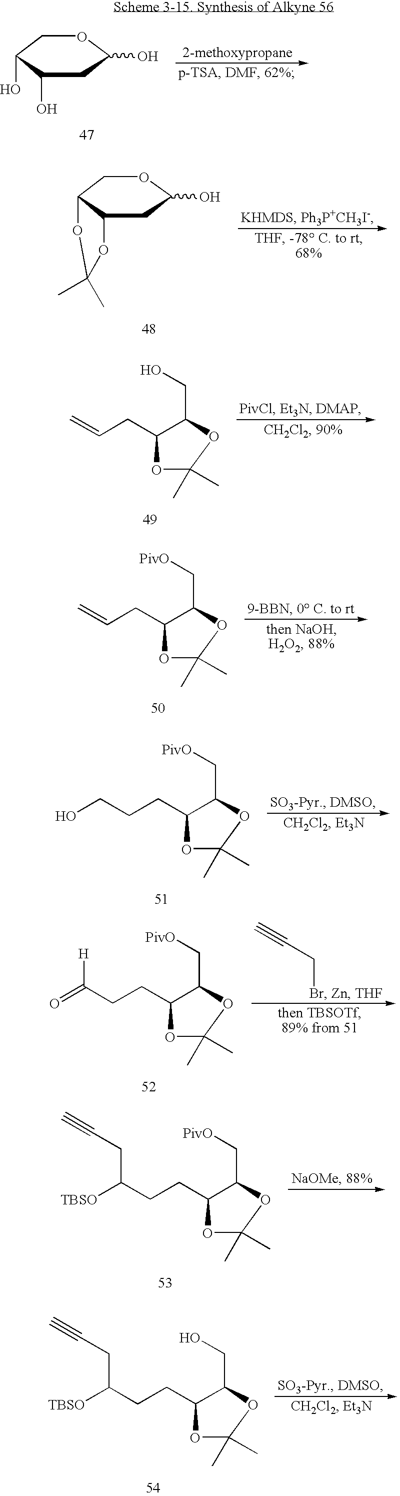

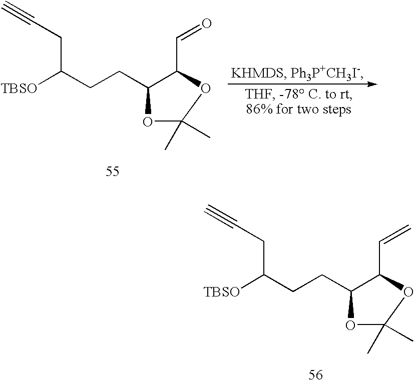

Following chemistry of the type described above, compound 56 was advanced to ester 58 through carboxylation of the alkyne function and Mitsunobu esterification using the commercially available (2R)-4-penten-2-ol. We again investigated the possibility of closure on 58, itself containing the free alkyne moiety as attempted above (see 10 and 14). Again, starting material (58) was recovered following attempted RCM. Fortunately, we found that our newly discovered protocol (cobalt complexation followed by RCM and then decomplexation) proceeded very efficiently delivering ynolide 61.

The two benzylic stereoisomers of 61 (2′-(R) and 2′-(S)) were separated by silica gel chromatography, and their absolute configurations were determined by modified Mosher's ester methodology (Ohtani et al. J. Org. Chem. 1991, 56, 1296-1298; incorporated herein by reference). As now expected, Diels-Alder reaction between 62 and diene 25 proceeded with high efficiency to afford the desired macrolide 63 in good yield. Following the protection of the phenol hydroxyl groups as the corresponding MOM ethers, the 2′-silyl group was removed and the dehydration reaction was carried out using the Martin sulfurane reagent (Martin et al. J. Am. Chem. Soc. 1971, 93, 4327-4329; incorporated herein by reference). Finally, global deprotection under acidic conditions completed the total synthesis of aigialomycin D 46. The physical data (¹H and ¹³C NMR, Mass spectrum, optical rotation, and IR) (Isalca et al. J. Org. Chem. 2002, 67, 1561-1566) in the context of the synthetic sequence and earlier spectral data serve to establish, independently, the structure of synthetic agialomycin D as shown. Moreover, the data for the final synthetic material are fully consistent with those reported for the natural product (Isaka, M.; Suyamsestakom, C.; Tanticharoen, M.; Kongsaeree, P.; Thebtaranonth, Y. J. Org. Chem. 2002, 67, 1561-1566; incorporated herein by reference).

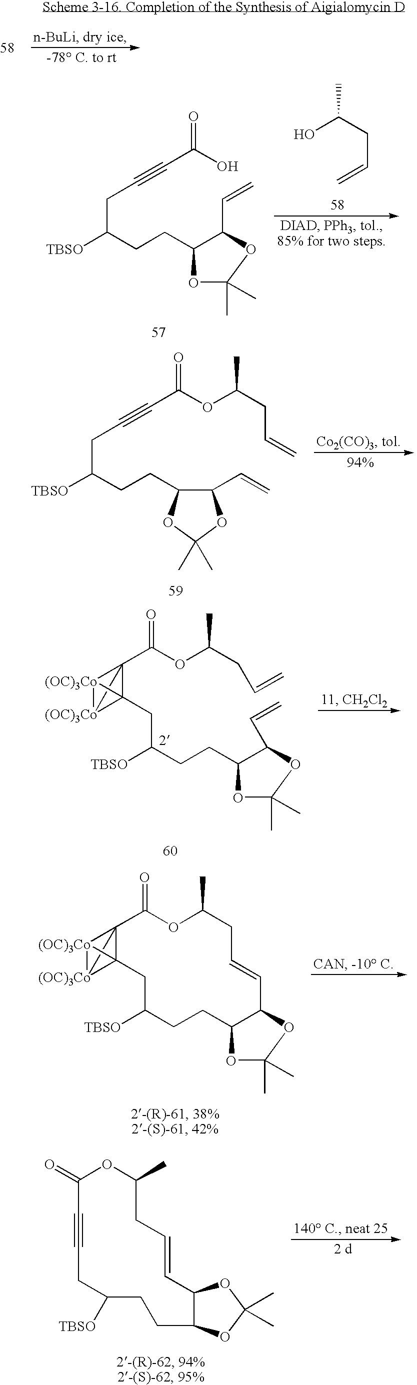

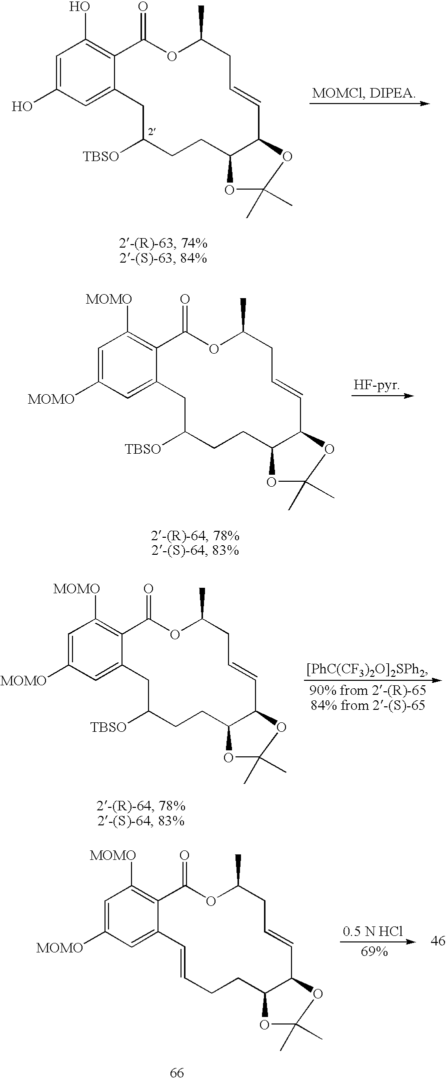

The accomplishment of the total synthesis of aigialomycin D demonstrates the generalizability of our newly developed protocol.

Biological Evaluation of Cycloproparadicicol Analogues. As discussed earlier, it appears that the inhibition of Hsp90 induces the proteasomal degradation of various oncogenic proteins, thereby leading to inhibition of tumor cell growth. We hoped to evaluate our synthetic analogues for their growth inhibition activity against MCF breast cancer cells. The IC₅₀ values of our synthetic compounds were determined after treatment of the cells with each drug listed in Table 3-1 for 72 h. Cycloproparadicicol is the most potent of the new compounds. Interestingly, chloride regioisomer 26 was found to be inactive. Of the two alcohol isomers, 27α displayed an IC₅₀ value of 150 nM, and 27β is inactive. Further modifications of 27α, however, decreased its potency. Surprisingly, introduction of the chlorine function (28α) resulted in raising the IC₅₀ to 390 nM. Compound 29a, methyl ether derivative of 27α, is completely inactive. The (Z)-oxime, (Z)-30, was found 3 times more potent that the corresponding (E)-isomer. Aigialomycin D was significantly less active than the cycloproparadicicol derivatives.

TABLE 3-1
IC50 (pM) Values of Cycloproparadicicol Analogues and Aigialomycin D

| | compound | | | | | | | | | | |
|---|---|---|---|---|---|---|---|---|---|---|---|
| | 2 | 26 | 27α | 27β | 28α | 29α | (Z)-30 | (E)-30 | 31 | 38 | 46 |
| IC50 | 54 | >500 | 150 | >500 | 390 | >10,000 | 98 | 282 | 10 000 | 3000 | >10 000 |

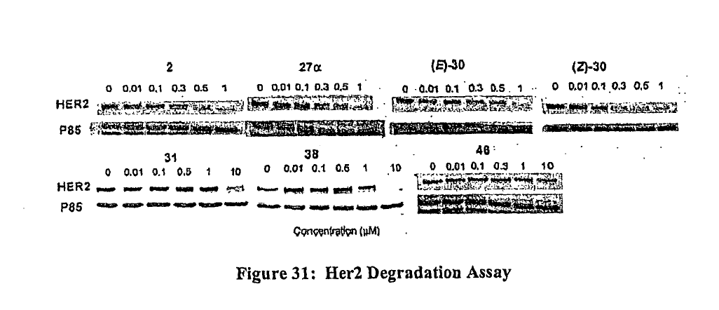

To confirm that the cytotoxicity was indeed due to the inhibition of Hsp90, we set out to investigate the degradation of Her2, one of the most sensitive client proteins of this chaperone. The expression levels of Her2 of drug-treated MCF-7 cells were analyzed using immunoblotting (FIG. 31). P85, used as a control, is not a client protein of Hsp90. As expected, the growth inhibition activities of the synthetic active analogues are reflected in their capacity to degrade Her2. The three most active compounds, 2, 27α, and (Z)-30, all were able to degrade Her2 at 0.3 μM. (E)-30 is less effective. Nonchlorinated difluoro compound 38 degraded Her2 at 1 μM, and its chlorinated counterpart 31, at 10 μM. Aigialomycin D (46) did not degrade the protein even at 10 μM.

### Conclusions and Future Directions

In summary, we have developed a new and efficient synthesis enabling the full evaluation of cycloproparadicicol as a feasible candidate for further advancement. The highlights of the new synthesis are cobalt-complexation-promoted RCM to generate ynolides, followed by Diels-Alder reaction with dimedone derived bis-siloxyl diene to assemble the benzofused macrolides. Given this pathway, we can generate grain quantities of cyloproparadicicol for biological evaluations. The generality of the synthesis plan has been demonstrated by its application to the first total synthesis of aigialomycin D.

It is, however, appropriate to point out that the sluggish dienophilicity of monoactivated acetylenes is still a problem preventing full generalizability of the method. For instance, in the work described above, i.e., in the cases of the yne lactam 44 and the ynolide 36 containing the sensitive difluorcyclopropyl group, the yields of the Diels-Alder step are low.

The biological activities of our synthetic analogues shown here, in conjunction with the earlier demonstration that cycloproparadicicol binds to Hsp90 at ca. 160 nM (Yamamoto et al. Angew. Chem., Int. Ed. 2003, 42, 1280-1284; incorporated herein by reference) support the notion of the latter as the likely target for this new group of anticancer agents. In vivo evaluations of cycloproparadicicol are in progress in various settings and will be described upon completion of full preclinical studies.

### Experimentals

General Methods: Reagents obtained from commercial suppliers were used without further purification unless otherwise noted. THF, toluene, and methylene chloride was obtained from a dry solvent system (passed through a prepacked column of alumina) and used without further drying. All air and water sensitive reactions were performed in oven or flame-dried glassware. NMR (¹H and ¹³C) spectra were recorded on Bruker AMX-400 MHz or Bruker Advance DRX-500 MHz as noted individually, referenced to CDCl₃ (7.27 ppm for ¹H and 77.23 ppm for ¹³C). Optical rotations were obtained on a JASCO model DIP-370 digital polarimeter. Low resolution mass spectra (ESI) were determined with a PESciex AP 130 spectrometer. High resolution mass spectra (FAB) were determined at Chemistry Department of Columbia University. Flash chromatography was performed with silica gel (230-400 mesh) from EM Science as the stationary phase. Analytical thin-layer chromatography was performed on E. Merck silica gel 60 F254 plates. Compounds which were not UV active were visualized by dipping the plates in phosphomolybdic acid solution and heating. Preparative thin layer chromatography was performed using the indicated solvent on Whatman® (LK6F Silica gel 60 Å 250 μM or Pk6F Silica Gel 60 Å 1000 μM) TLC plate.

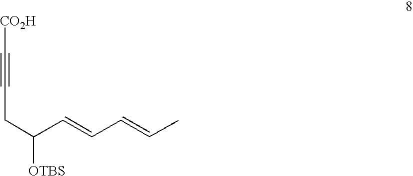

Acid 8. To a suspension of activated zinc (15 g, 230 mmol) in dry THF (50 mL) at 0° C. was added propargyl bromide 5 (19.2 mL 80 wt % in toluene, 172 mmol). The resulting mixture was stirred at 0° C. for 1 hr, and sorbaldehyde 6 (12.7 mL, 115 mmol) was added. After 1 hr at 0° C., additional zinc (4.5 g, 69 mmol) was added, and stirring was continued for 2.5 hrs at room temperature (the reaction was exothermic and ice bath was needed occasionally to keep the temperature down). The reaction was quenched by slow addition of sat. aqueous NH₄Cl (500 mL), followed by diluting with Et₂O (1 L). The layers were separated, and the organic layer was washed with H₂O (300 mL), brine (300 mL), dried (NaSO₄), filtered and concentrated in vacuum. The residue was dissolved in CH₂Cl₂ (750 mL) with imidazole (9.8 g, 144 mmol), t-butyldimethylsilyl chloride (19 g, 126 mmol) and 4-(dimethylamino)pyridine (1.4 g, 11.5 mmol), and stirred at room temperature for 3 hrs. Additional imidazole (4.9 g, 72 mmol) and t-butyldimethylsilyl chloride (9.5 g, 63 mmol) were added, and stirring was continued for 9 hrs. The reaction was quenched by addition of sat. aqueous NH₄Cl (200 mL). The layers were separated, and the organic layer was washed with H₂O (200 mL), brine (200 mL), dried (Na₂SO₄), filtered and concentrated in vacuum. The residue was purified by flash chromatography (silica, 0 to 10% Et₂O in hexane) to give terminal alkyne precursor 7(15 g, 52%). ¹H NMR (CDCl₃, 400 MHz) δ 6.18 (dd, J=15.1, 10.5 Hz, 1H), 6.03 (ddd, J=15.0, 10.6, 1.3 Hz, 1H), 5.72 (dd, J=14.9, 6.9 Hz, 1H), 5.61 (dd, J=15.1, 6.4 Hz, 1H), 4.30 (q, J=6.3 Hz, 1H), 2.43 (ddd, J=16.5, 6.2, 2.7 Hz, 1H), 2.34 (ddd, J=16.5, 6.8, 1.7 Hz, 1H), 2.60 (t, J=2.6 Hz, 1H), 1.77 (d, J=6.8 Hz, 3H), 0.91 (s, 9H), 0.09, 0.06 (2s, 6H); ¹³C NMR (CDCl₃, 100 Hz) δ 132.5, 131.1, 130.7, 130.0, 81.7, 72.1, 70.1, 28.9, 26.1, 18.4, −2.7; IR (film) vₘₐₓ 3313, 2956, 2930, 2856, 2121, 1255, 1115, 1079, 987, 837; ESIMS m/z 273 ([M+Na⁺], C₁₅H₂₆NaOSi requires 273).

To a solution of 7 (15.0 g, 59.9 mmol) in Et₂O (270 mL) at −78° C., was added a solution of BuLi (1.6 M in hexane, 41.5 mL, 66.4 mmol). After 45 min, excess crushed dry ice was added and the reaction was allowed to warm to room temperature. The solution was acidified by addition of 0.5 M aqueous citric acid (300 mL). The layers were separated, and the aqueous layer was extracted with additional Et₂O (300 mL×2). The combined organic layers were dried (MgSO₄), filtered and concentrated in vacuum. The residue was purified by flash chromatography (silica, 50% to 100% EtOAc in hexane) to give product 8 as a light yellow solid (17.5 g, 99%). ¹H NMR (CDCl₃, 400 MHz) δ 6.19 (dd, J=15.1, 10.4 Hz, 1H), 6.03 (ddd, J=14.9, 10.6, 1.3 Hz, 1H), 5.75 (dd, J=15.0, 6.8 Hz, 1H), 5.55 (dd, J=15.1, 6.4 Hz, 1H), 4.36 (q, J=6.3 Hz, 1H), 2.61-2.48 (m, 2H), 1.77 (d, J=6.8 Hz, 3H), 0.91 (s, 9H), 0.10, 0.06 (2s, 6H); ¹³C NMR (CDCl₃, 100 Hz) δ 157.6, 131.6, 131.3, 130.7, 89.7, 74.0, 71.4, 29.3, 26.0, 18.4, 18.3, −4.3, −4.7; IR (film) vₘₐₓ 2956, 2930, 2857, 2242, 1689, 1281, 1257, 1080; ESIMS m/z 317 ([M+Na⁺], C₁₆H₂₆NaO₃Si requires 317).

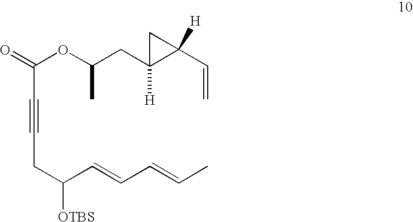

Alkynoic ester 10. To a solution of DIAD (14.7 mL, 72.9 mmol) in dry THF (350 mL) was added Ph₃P (15.8 g, 60.2 mmol), and the mixture was stirred at room temperature for one hour. At −20° C., a solution of acid 8 (13.1 g, 44.4 mmol) in 100 mL THF was added. After 15 min, a solution of alcohol 9 (4.0 g, 31.7 mmol) in 150 mL THF was added, and stirring was continued for 2 hours at −20° C. The reaction was quenched by addition of 250 mL of pH 7.2 phosphate buffer, followed by warming to room temperature and diluting with EtOAc (1.5 L). The layers were separated, and the aqueous layer was extracted with EtOAc (2×250 mL). The combined organic layers were washed with brine (250 mL), dried (Na₂SO₄), filtered and concentrated in vacuum. The residue was purified by flash chromatography (silica, 50:1→20:1 hexanes/EtOAc) to give ester 10 as a mixture of two inseparable diastereoisomers (5.9 g, 47%). ¹H NMR (CDCl₃, 400 MHz) δ 6.17 (dd, J=15.1, 10.5 Hz, 1H), 6.03 (ddd, J=12.3, 10.6, 1.4 Hz, 1H), 5.70 (dd, J=14.8, 6.8 Hz, 1H), 5.55 (dd, J=15.2, 6.4 Hz, 1H), 5.37 (ddd, J=17.1, 10.2, 8.7 Hz, 1H), 5.06 (q, J=6.4 Hz, 1H), 5.03 (dd, J=17.0, 1.5 Hz, 1H), 4.84 (dd, J=10.2, 1.6 Hz, 1H), 4.34 (q, J=6.4 Hz, 1H), 2.56-2.42 (m, 2H), 1.76 (d, J=6.8 Hz, 3H), 1.57-1.53 (m, 2H), 1.29 (d, J=6.4 Hz, 3H), 1.20-1.10 (m, 1H), 0.90 (s, 9H), 0.79-0.68 (m, 1H), 0.65-0.57 (m, 2H), 0.10, 0.05 (2s, 6H); ¹³C NMR (CDCl₃, 100 Hz) δ 154.5, 141.4, 131.8, 131.1, 130.8, 130.4, 112.1, 86.2, 74.9, 73.0, 71.5, 39.7, 29.2, 26.0, 22.4, 19.8, 18.4, 18.3, 17.2, 13.7, −4.3, −4.7; IR (film) vₘₐₓ 2955, 2930, 2856, 2238, 1710, 1253, 1068; ESIMS m/z 437 ([M+Cl], C₂₄H₃₉ClO₃Si requires 437); HRMS (FAB⁺) m/z 403.2687 ([M+H]⁺, C₂₄H₃₉O₃Si requires 403.2668).

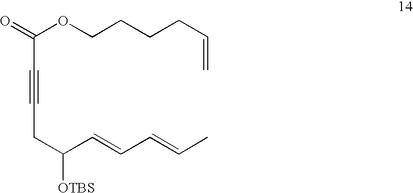

To a solution of acid 8 (192 mg, 0.653 mmol) and 5-hexen-1-ol (0.118 mL, 0.979 mmol) in dry CH₂Cl₂ (3 mL) was added EDCI (150 mg, 0.784 mmol) and 4-(dimethylamino)pyridine (8.0 mg, 0.065 mmol). After 3 hrs at room temperature, the reaction mixture was loaded on PTLC plates and purified (12:1 hexane/EtOAc) to give model ester 14 (146 mg, 59%). 0 ¹H NMR (CDCl₃, 400 MHz) δ 6.18 (dd, J=15.1, 10.5 Hz, 1H), 6.04 (ddd, J=15.0, 10.5, 1.5 Hz, 1H), 5.79 (ddt, J=17.1, 10.3, 7.2 Hz, 1H), 5.71 (dq, J=15.0, 6.8 Hz, 1H), 5.56 (dd, J=15.1, 6.4 Hz, 1H), 5.03 (dq, J=17.1, 1.6 Hz, 1H), 4.97 (dd, J=10.2, 1.6 Hz, 1H), 4.35 (q, J=6.3 Hz, 1H), 4.16 (t, J=6.6 Hz, 2H), 2.57-2.44 (m, 2H), 2.09 (q, J=7.2 Hz, 1H), 1.77 (d, J=6.9 Hz, 3H), 1.74-1.63 (m, 2H), 1.52-1.44 (m, 2H), 0.90 (s, 9H), 0.10, 0.06 (2s, 6H); ¹³C NMR (CDCl₃, 100 Hz) δ 154.0, 138.4, 131.8, 131.1, 130.8, 130.5, 115.1, 86.6, 74.6, 71.5, 65.9, 33.4, 29.3, 28.1, 26.0, 25.3, 18.4, 18.3, 4.3, −4.7; IR (film) νₘₐₓ 2955, 2930, 2856, 2238, 1713, 1249, 1072; ESIMS m/z 399 ([M+Na⁺], C₁₂H₃₆NaO₃Si requires 399). HRMS (FAB⁺) m/z 375.2363 ([M−H]⁺, C₂₂H₃₅O₃Si requires 375.2355).

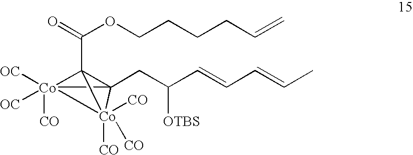

Cobalt complex 15. To a solution of ester 14 (77.8 mg, 0.207 mmol) in toluene (9 mL) was added Co₂(CO)₈ (99.0 mg, 0.289 mmol). The mixture was stirred at room temperature for 45 min, and then concentrated in vacuum. The dark residue was purified by PTLC (15:1 hexane/EtOAc) to give cobalt complex 15 (117.5 mg, 86%) as a red oil. ¹H NMR (CDCl₃, 400 MHz) 36.17 (dd, J=15.3, 10.6 Hz, 1H), 6.03 (ddd, J=15.3, 11.3 Hz, 1H), 5.68 (dd, J=14.9, 6.9 Hz, 1H), 5.61 (dd, J=15.2, 6.8 Hz, 1H), 5.38 (ddd, J=17.1, 10.1, 8.7 Hz, 1H), 5.10 (q, J=6.4 Hz, 1H), 5.04 (dd, J=17.1, 1.3 Hz, 1H), 4.85 (dd, J=10.3, 1.4 Hz, 1H), 4.41, (m, 1H), 3.20-3.15 (m, 2H), 1.76 (d, J=6.7 Hz, 3H), 1.59 (t, J=6.6 Hz, 2H), 1.32 (d, J=6.2 Hz, 3H), 1.22-1.17 (m, 1H), 0.90 (s, 9H), 0.82-0.72 (m, 1H), 0.66-0.59 (m, 2H), 0.09, 0.08 (2s, 6H); ¹³C NMR (CDCl₃, 100 Hz) 198.7, 169.2, 141.4, 134.3, 132.5, 132.4, 132.2, 131.0, 130.2, 128.8, 127.2, 127.1, 112.0, 93.0, 81.0, 73.7, 73.6, 73.1, 40.0, 26.1, 22.4, 19.9, 18.6, 18.4, 17.4, 13.9, 13.5, −4.2, −4.3, −4.6; IR (film) vₘₐₓ 2956, 2930, 2858, 2097, 2058, 2029, 1703, 1221, 1065; ESIMS m/z 685 ([M+Na⁺], C₂₈H₃₆Co₂NaO₉Si requires 685).

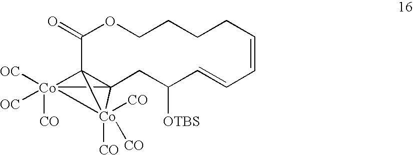

RCM product 16. To a solution of cobalt complex 15 (16 mg, 0.024 mmol) in dry CH₂Cl₂(120 mL) was added tricyclohexyl phosphine[1,3-bis(2,4,6-trimethylphenyl)-4,5-dihydroimidazol-2-ylidene]-[bezyli-dene]ruthenium (IV) dichloride (second generation Grubbs catalyst, 11) (6.1 mg, 0.0072 mmol). The resulting solution was heated to 45° C. for 1 hr and 10 min, then cooled to room temperature and filtered through a plug of silica gel. The solvent was removed under reduced pressure. The residue was purified by PTLC (15:1 hexane/EtOAc) to give cyclic product 16 (10.5 mg, 71%). ¹H NMR (CDCl₃, 400 MHz) δ 6.44 (dd, J=15.3, 10.8 Hz, 1H), 5.97 (t, J=10.8 Hz, 1H), 5.58 (dd, J=15.4, 7.5 Hz, 1H), 5.54 (dt, J=10.1, 4.5 Hz, 1H), 4.57-4.47 (m, 2H), 4.25-4.20 (m, 1H), 3.45-3.36 (m, 2H), 2.45-2.37 (m, 1H), 2.13-1.05 (m, 1H), 1.99-1.79 (m, 1H), 1.80-1.62 (m, 2H), 1.51-1.41 (m, 1H), 0.92 (s, 9H), 0.11, 0.08 (2s, 6H); ¹³C NMR (CDCl₃, 100 Hz) δ 199.9, 170.4, 135.2, 132.7, 129.0, 127.1, 92.8, 77.4, 73.1, 65.0, 45.2, 26.4, 26.1, 25.4, 18.5, 1.2, −4.2, −4.6; IR (film) vₘₐₓ 2955, 2930, 2857, 2098, 2059, 2027, 1702, 1213, 1057; ESIMS m/z 643 ([M+Na⁺], C₂₅H₃₀Co₂NaO₉Si requires 643).

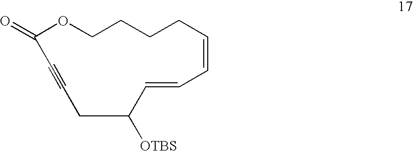

Model cyclic alkyne 11. To a solution of compound 10 (35.6 mg, 0.0574 mmol) in acetone at −10° C. was added ammonium cerium (IV) nitrate (189 mg, 0.344 mmol) portionwise. After 10 min at −10° C., the reaction was quenched by addition of diisopropylethylamine (0.18 mL, 1.03 mmol). The resulting mixture was filtered through a plug of neutral alumina, and the solvent was removed under reduced pressure. Purification by PTLC (15:1 hexane/EtOAc) afforded cyclic alkyne 11 (17.6 mg, 92%). ¹H NMR (CDCl₃, 400 MHz) δ 6.64 (dd, J=15.5, 11.1 Hz, 1H), 6.07 (t, J=11.0 Hz, 1H), 5.53 (dd, J=15.5, 7.1 Hz, 1H), 5.40 (dt, J=10.4, 4.8 Hz, 1H), 4.41-4.25 (m, 2H), 4.06-4.01 (m, 1H), 2.69-2.62 (m, 1H), 2.56 (dd, J=17.1, 4.4 Hz, 1H), 2.46 (dd, J=17.1, 9.5 Hz, 1H), 2.26-2.21 (m, 1H), 1.75-1.61 (m, 4H), 0.89 (s, 9H), 0.08, 0.07 (2s, 6H); ¹³C NMR (CDCl₃, 100 Hz) δ 153.9, 133.2, 132.9, 128.5, 128.2, 87.7, 77.0, 72.9, 68.0, 29.0, 28.2, 26.7, 26.0, 25.6, 18.3, −4.3, −4.7; IR (film) vₘₐₓ 2954, 2929, 2857, 2238, 1716, 1245, 1110, 1075, 837; ESIMS m/z 357 ([M+Na⁺], C₁₉H₃₀NaO₃Si requires 357). HRMS (FAB⁺) m/z 333.1888 ([M−H]⁺, C₁₉H₂₉O₃Si requires 333.1886).

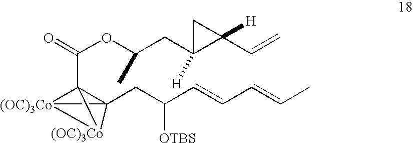

Cobalt complex 18. To a solution of alkyne 10 (526 mg, 1.31 mmol) in toluene (60 mL) was added Co₂(CO)₈ (625 mg, 1.83 mmol). The mixture was stirred at room temperature for 30 min, and the solvent was removed under reduced pressure. The dark residue was purified by flash chromatography (silica, 0 to 5% EtOAc in hexane) to give cobalt complex 18 (902 mg, 100%) as an inseparable mixture of two diastereomers. ¹H NMR (CDCl₃, 400 MHz) δ 6.17 (dd, J=15.3, 10.6 Hz, 1H), 6.03 (ddd, J=15.3, 11.3 Hz, 1H), 5.68 (dd, J=14.9, 6.9 Hz, 1H), 5.61 (dd, J=15.2, 6.8 Hz, 1H), 5.38 (ddd, J=17.1, 10.1, 8.7 Hz, 1H), 5.10 (q, J=6.4 Hz, 1H), 5.04 (dd, J=17.1, 1.3 Hz, 1H), 4.85 (dd, J=10.3, 1.4 Hz, 1H), 4.41, (m, 1H), 3.20-3.15 (m, 2H), 1.76 (d, J=6.7 Hz, 3H), 1.59 (t, J=6.6 Hz, 2H), 1.32 (d, J=6.2 Hz, 3H), 1.22-1.17 (m, 1H), 0.90 (s, 9H), 0.82-0.72 (m, 1H), 0.66-0.59 (m, 2H), 0.09, 0.08 (2s, 6H); ¹³C NMR (CDCl₃, 100 Hz) δ 198.7, 169.2, 141.4, 134.3, 132.5, 132.4, 132.2, 131.0, 130.2, 128.8, 127.2, 127.1, 112.0, 93.0, 81.0, 73.7, 73.6, 73.1, 40.0, 26.1, 22.4, 19.9, 18.6, 18.4, 17.4, 13.9, 13.5, −4.2, −4.3, 4.6; IR (film) vₘₐₓ 2956, 2930, 2858, 2097, 2058, 2029, 1703, 1221, 1065; ESIMS m/z 711 ([M+Na⁺], C₃₀H₃₈Co₂NaO₉Si requires 711).

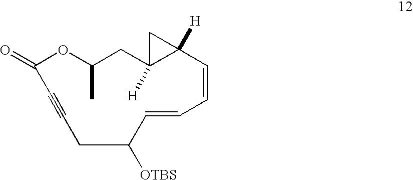

Cyclic alkyne 12. To a solution of alkyne-cobalt complex 18 (575 mg, 0.84 mmol) in dry CH₂Cl₂ (4 L) was added tricyclohexyl phosphine[1,3-bis(2,4,6-trimethylphenyl)-4,5-dihydroimidazol-2-ylidene][bezylidene]ruthenim (IV) dichloride (second generation Grubbs catalyst) (192 mg, 0.23 mmol). The resulting mixture was stirred at room temperature for 3.5 hours, and filtered through a short column of silica gel. The filtrate was concentrated in vacuum to give crude red oil product 19. To a crude 19 (1.11 g, 0.0358 mmol) in acetone (100 mL) at −10° C. was added ditertbutyl pyridine (5.8 mL, 25.8 mmol). Ceric ammonium nitrate (5.7 g, 10.3 mmol) was added in three portions. After 30 minutes at −10° C., the reaction was quenched by the addition of a diisopropylethylamine (5.4 mL, 31 mmol), followed by warming to room temperature and diluting with CH₂Cl₂ (50 mL). The mixture was filtered through a pad of neutral alumna, and washed with generous amount of CH₂Cl₂. The filtrate was concentrated in vacuum. The residue was purified by PTLC (15:1, hexanes/EtOAc) to give cyclic alkyne 12 as two separable isomers (major 245.3 mg, 40%; minor 68.8 mg, 11%). Major isomer: ¹H NMR (CDCl₃, 400 MHz) δ 6.70 (dd, J=15.6, 11.1 Hz, 1H), 5.98 (t, J=10.8 Hz, 1H), 5.51 (dd, J=15.6, 7.7 Hz, 1H), 5.20-5.14 (m, 1H), 4.92 (t, J=10.7 Hz, 1H), 4.38-4.33 (m, 1H), 2.59 (dd, J=16.8, 4.3 Hz, 1H), 2.41 (dd, J=16.7, 10.9 Hz, 1H), 2.07 (dt, J=14.8, 1.8 Hz, 1H), 1.74-1.67 (m, 1H), 1.33 (d, J=6.5 Hz, 3H), 1.19-1.12 (m, 1H), 0.93-0.87 (m, 1H), 0.89 (s, 9H), 0.69-0.65 (m, 1H), 0.63-0.58 (m, 1H), 0.07 (s, 6H); ¹³C NMR (CDCl₃, 100 Hz) δ 153.4, 136.5, 132.5, 128.3, 125.4, 85.7, 77.4, 73.8, 72.0, 38.6, 29.2, 26.0, 20.4, 18.4, 18.2, 14.6, 12.8; IR (film) vₘₐₓ 2954, 2928, 2856, 2238, 1706, 1252, 1105, 1072, 1004; ESIMS m/z 383 ([M+Na⁺], C₂₁H₂₃NaO₃Si requires a 383); HRMS (FAB⁺) m/z 360.2130 ([M]⁺, C₂₁H₃₂O₃Si requires 360.2121). [α]²⁵_{D} +56 (c 0.95, CHCl₃).

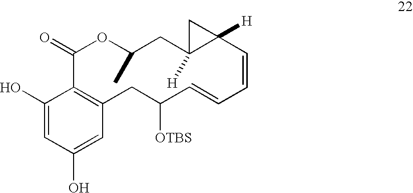

Diels-Alder product 22. Compound 16 (major isomer, 1.8 g, 5.0 mmol) was dissolved in diene 25 (14 mL, 49 mmol) and heated to 160° C. in a sealed vial for 66 hours. The reaction mixture was cooled to room temperature and purified by silica gel chromatography (9:1 hexanes/EtOAc) followed by PTLC (4:1, hexanes/EtOAc) to give 22 (2.2 g, 99%). ¹H NMR (CDCl₃, 400 MHz) δ 11.58 (s, 1H), 6.56 (dd, J=15.9, 9.3 Hz, 1H), 6.38 (d, J=2.4 Hz, 1H), 6.33 (d, J=2.4 Hz, 1H), 5.94 (t, J=9.5 Hz, 1H), 5.68 (dd, J=15.9, 6.8 Hz, 1H), 5.46-5.43 (m, 1H), 5.34 (dd, J=10.1, 4.3 Hz, 1H), 4.51 (q, J=6.7 Hz, 1H), 3.79 (dd, J=13.6, 6.2 Hz, 1H), 2.81 (dd, J=13.1, 8.9 Hz, 1H), 1.99-1.81 (m, 2H), 1.44 (d, J=6.5 Hz, 3H), 1.21-1.13 (m, 1H), 0.93-0.87 (m, 1H), 0.88 (s, 9H), 0.58-0.51 (m, 2H), −0.02 (s, 6H); ¹³C NMR (CDCl₃, 100 Hz) δ 164.8, 160.5, 143.8, 134.8, 133.7, 129.6, 128.0, 112.3, 106.1, 102.1, 75.7, 72.9, 42.6, 38.0, 26.1, 18.7, 18.4, 16.4, 16.1, 14.8, 1.4, −4.4, −4.6; IR (film) vₘₐₓ 3376, 2954, 2928, 2856, 1644, 1619, 1257, 1062, 835; ESIMS m/z 467 ([M+Na⁺], C₂₅H₃₆NaO₅Si requires 467). HRMS (FAB⁺) m/z 444.2336 ([M]⁺, C₂₅H₃₆O₃Si requires 444.2332). [α]²⁵_{D} +3.1 (c 1.3, CHCl₃).

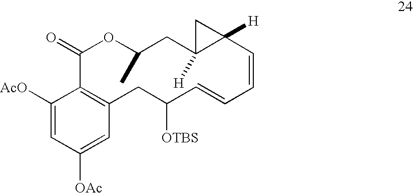

To a solution of 22 (major isomer, 243 mg, 0.546 mmol) in anhydrous DMF (13.5 mL) was added acetic anhydride (2.9 mL) and 4-(dimethylamino)pyridine (12.0 mg, 0.0546 mmol) sequentially. After 30 min at room temperature, the reaction was quenched by addition of 50 mL of pH 7.2 phosphate buffer. The resulting mixture was diluted with EtOAc (75 mL), separated and the aqueous layer was extracted with additional EtOAc (2×50 mL). The combined organic layers were washed with 5% aqueous NaCl (2×45 mL). The combined washings were extracted with EtOAc (2×30 mL). The combined organic layers were washed with sat. aqueous NaCl (30 mL), dried (Na₂SO₄), filtered and concentrated. Purification of the residue by PTLC (4:1 hexane/EtOAc) gave diacetate 18 (250 mg, 87%). ¹H NMR (CDCl, 400 MHz) δ 6.96 (d, J=2.1 Hz, 1H), 6.88 (d, J=2.1 Hz, 1H), 6.59 (dd, J=16.0, 10.5 Hz, 1H), 5.89 (t, J=10.5 Hz, 1H), 5.66 (dd, J=16.1, 4.9 Hz, 1H), 5.26-5.23 (m, 2H), 4.51 (q, J=5.8 Hz, 1H), 3.12-3.02 (m, 2H), 2.28, 2.23 (2s, 6H), 1.47 (d, J=6.2 Hz, 3H), 1.46-1.39 (m, 1H), 1.14 (ddd, J=14.9, 10.1, 2.0 Hz, 1H), 0.96-0.87 (m, 1H), 0.87 (s, 9H), 0.52-0.48 (m, 2H), −0.01, −0.02 (2s, 6H); ¹³C NMR (CDCl₃, 100 Hz) δ 168.6, 168.4, 166.4, 151.4, 148.7, 139.1, 135.9, 133.6, 129.2, 127.2, 125.6, 120.4, 114.7, 73.8, 73.2, 43.2, 40.0, 26.0, 21.3, 21.0, 19.3, 18.3, 16.9, 16.2, 17.7, −4.6, −4.7; IR (film) vₘₐₓ 2954, 2928, 2856, 1775, 1720, 1612, 1191, 1134, 1069; ESIMS m/z 551 ([M+Na⁺], C₂₉H₄₀NaO₇Si requires 551); HRMS (FAB⁺) m/z 528.2569 ([M]⁺, C₂₉H₄₀O₇Si requires 528.2543). [α]²⁵_{D} −38 (c 1.1, CHCl₃).

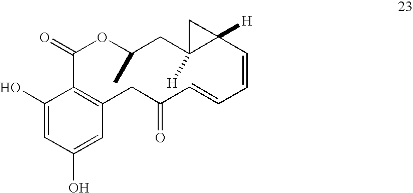

Cyclopropamonocillin 23. To a solution of diacetate 24 (250 mg, 0.473 mmol) in THF (10.2 mL) at 0° C. was added pyridine (3.4 mL) and HF-Pyridine complex (1.7 mL) sequentially. The resulting mixture was stirred at room temperature for 10.5 hrs. TMSOMe (30 mL) was added, and stirring was continued for 45 min to quench the remaining HF. The solvents were removed under reduced pressure. The alcohol isolated was dried under high vacuum, and dissolved in CH₂Cl₂ (15 mL) and cooled to 0° C. Dess-Martin periodinane (301 mg, 0.710 mmol) was added, and the resulting mixture was stirred at room temperature for 15 min. The solution was then directly loaded on PTLC plates and purified (1:1 hexane/EtOAc) to give the desired ketone (133 mg, 68%). ¹H NMR (CDCl₃, 500 MHz) δ 8.01 (dd, J=16.1, 11.3 Hz, 1H), 6.97 (d, J=1.4 Hz, 1H), 6.92 (d, J=2.0 Hz, 1H), 6.20 (t, J=10.4 Hz, 1H), 5.96 (d, J=16.0 Hz, 1H), 5.60 (dd, J=10.0, 7.2 Hz, 1H), 5.47-5.41 (m, 1H), 4.20 (d, J=13.8 Hz, 1H), 3.77 (d, J=13.8 Hz, 1H), 2.31 (dt, J=15.3, 4.4 Hz, 1H), 2.25, 2.24 (2s, 6H), 1.73-1.71 (m, 1H), 1.50 (d, J=6.5 Hz, 3H), 1.26-1.21 (m, 1H), 1.00-0.97 (m, 1H), 0.75-0.69 (m, 2H); ¹³C NMR (CDCl₃, 125 Hz) δ 198.4, 168.5, 165.0, 152.1, 149.2, 145.6, 143.7, 135.7, 129.6, 128.8, 124.6, 119.1, 115.6, 72.8, 43.0, 38.3, 21.2, 18.2, 16.7, 16.5, 15.6; IR (film) vₘₐₓ 2928, 1774, 1728, 1657, 1621, 1586, 1290, 1190, 1132, 1029; ESIMS m/z 435 ([M+Na⁺], C₂₃H₂₄NaO₇ requires 435); HRMS (FAB⁺) m/z 413.1616 ([M+H]⁺, C₂₃H₂₅O₇ requires 413.1600). [α]²⁵_{D} −269 (c 1.43, CHCl₃).

The above ketone (169 mg, 0.409 mmol) was dissolved in 26 mL 1:1 MeOH and 5% aqueous NaHCO₃, and stirred at room temperature for 14 hrs to remove the phenolic acetates. The resulting solution was diluted with sat. aqueous NH₄Cl (80 mL) and EtOAc (100 mL) and separated. The aqueous layer was extracted with additional EtOAc (3×75 mL). The combined organic layers were dried (Na₂SO₄), filtered and concentrated in vacuum. Purification by PTLC (1:1 hexane/EtOAc) provided 23 (124 mg, 92%). ¹H NMR (CDCl₃, 500 MHz) δ 11.33 (s, 1H), 8.36 (dd, J=16.1, 11.7 Hz, 1H), 6.48 (d, J=1.6 Hz, 1H), 6.40 (d, J=2.5 Hz, 1H), 6.20 (t, J=11.3 Hz, 1H), 6.01 (d, J=15.9 Hz, 1H), 5.72 (dd, J=9.8, 6.2 Hz, 1H), 5.49-5.45 (m, 1H), 5.32 (d, J=13.6 Hz, 1H), 3.51 (d, J=13.7 Hz, 1H), 2.31 (dt, J=15.8, 3.3 Hz, 1H), 1.61-1.55 (m, 1H), 1.55 (d, J=6.7 Hz, 3H), 1.32-1.28 (m, 1H), 1.03-0.97 (m, 1H), 0.74-0.72 (m, 1H), 0.64-0.63 (m, 1H); ¹C NMR (CDCl₃, 125 Hz) δ 201.8, 170.1, 165.6, 161.6, 145.3, 139.2, 129.5, 128.5, 109.4, 104.5, 102.7, 73.5, 43.2, 27.6, 17.9, 17.2, 15.9, 13.9; IR (film) vₘₐₓ 3260, 1650, 1618, 1586, 1447, 1259, 1160, 1099, 996, 854; ESIMS m/z 351 ([M+Na⁺], C₁₉H₂₀NaO₅ requires 351); HRMS (FAB⁺) m/z 329.1382 ([M+H⁺], C₁₉H₂₁O₅ requires 329.1389). (+)-(R, R, S)-19: [α]²⁵_{D} −189 (c 0.77, CH₂Cl₂).

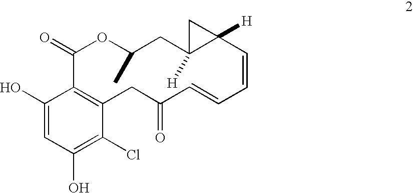

Cycloproparadicicol 2. Compound 23 (90.4 mg, 0.275 mmol) was dissolved in dry CH₂Cl₂ (17 mL) and cooled to 0° C. A solution of SO₂Cl₂ (0.035 mL, 0.44 mmol)) in CH₂Cl₂ (3.2 mL) was added dropwise. After 1 hour, the reaction was quenched by the addition of 20 mL of 5% NH₄Cl, and diluted with CH₂Cl₂. The layers were separated, and the organic phase was washed with brine, dried (MgSO₄), filtered and concentrated in vacuum. Purification of the residue by PTLC (3:1, hexanes/EtOAc) gave 2 as a white solid (68.8 mg, 70%), along with regioisomer 26 (26.8 mg, 27%). ¹H NMR (CDCl₃, 500 MHz) δ 10.89 (s, 1H), 8.00 (dd, J=15.9, 11.3 Hz, 1H), 6.63 (s, 1H), 6.42 (s, 1H), 6.13 (t, J=11.2 Hz, 1H), 6.04 (d, J=16.3 Hz, 1H), 5.62 (dd, J=9.9, 5.8 Hz, 1H), 5.46-5.42 (m, 1H), 4.90 (broad d, J=15.9 Hz, 1H), 3.77 (bs, 1H), 2.22 (dt, J=15.8, 3.2 Hz, 1H), 1.60-1.52 (m, 1H), 1.48 (d, J=6.7 Hz, 3H), 1.15-1.11 (m, 1H), 0.90-0.85 (m, 1H), 0.69-0.65 (m, 1H), 0.57-0.54 (m, 1H); ¹³C NMR (CDCl₃, 125 Hz) δ 199.2, 169.4, 162.7, 155.6, 143.5, 142.5, 136.9, 129.9, 128.9, 115.6, 107.8, 103.5, 74.4, 46.4, 37.4, 17.9, 17.3, 15.6, 13.6; IR (film) vₘₐₓ 3341, 1716, 1651, 1609, 1578 cm⁻¹; ESIMS m/z 385 ([M+Na⁺], C₁₉H₁₉NaO₅Cl requires 385); HRMS (ESI) m/z 385.0820 ([M+Na⁺], C₁₉H₁₉NaO₅Cl requires 385.0819). (+)-(R, R, S)-2: [α]²⁵_{D} +69 (c 0.87, CH₂Cl₂).

Regioisomer 26: ¹H NMR (500 MHz, CDCl₃): δ 12.02 (s, 1H), 8.25 (dd, J=15.9, 11.4 Hz, 1H), 7.56 (s, 1H), 6.63 (s, 1H), 6.20 (d, J=11.3, 11.2 Hz, 1H), 6.02 (d, J=16.0 Hz, 1H), 5.70 (dd, J=9.8, 6.2 Hz, 1H), 5.52-5.48 (m, 1H), 5.26 (d, J=13.8 Hz, 1H), 3.50 (d, J=13.8 Hz, 1H), 2.33 (dt, J=15.9, 3.2 Hz, 1H), 1.56 (d, J=6.7 Hz, 3H), 1.30-1.25 (m, 1H), 1.01-0.92 (m, 1H), 0.74-0.71 (m, 1H), 0.65-0.62 (m, 1H); ¹³C NMR (125 MHz, CDCl₃): δ 200.8, 170.3, 160.6, 157.0, 145.2, 144.9, 137.5, 129.7, 129.0, 117.8, 109.1, 74.6, 73.8, 43.4, 37.8, 18.1, 17.4, 15.8, 14.1; ESIMS m/z 384.9 ([M+Na⁺], C₁₉H₁₉ClNaO₅ requires 385.1).

Determination of the stereochemistry at the 2′ position of 25α:

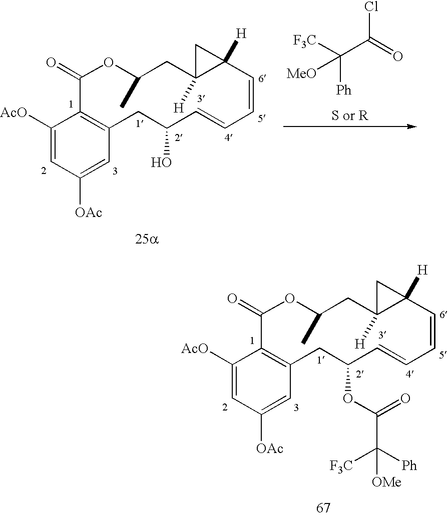

The stereochemistry of 2′ of 25α was determined according to the protocol reported by Ikuko Ohtani et al. (Ohtani, I.; Kusumi, T.; Kashman, Y.; Kakisawa, H. J. Org. Chem. 1991, 56, 1296-1298; incorporated herein by reference)/

To a solution of 25α (7 mg, 0.017 mmol) in CH₂Cl₂ (0.15 mL) was added triethylamine (0.033 mL, 0.238 mmol), DMAP (0.5 mg, 0.004 mmol) and (S)- or (R)-methoxytrifluorophenyl acetic chloride (0.025 mL, 0.136 mmol). The reaction mixture was stirred overnight before loaded onto PTLC (250 μm) and eluted by Hex/EtOAc (4/1). The product (R) or (S) ester was isolated as colorless oil (7.5 mg, 70%).

Δδ=δ_{S}−δ_{R}.

Δδ_{3′}=δ_{S3′}−δ_{R3′}=5.55−5.68=−0.13 ppm;

Δδ_{4′}=δ_{S4′}−δ_{R4′}=6.12−6.14=−0.02 ppm;

Δδ_{5′}=δ_{S5′}−δ_{R5′}=5.85−5.91=−0.06 ppm;

Δδ_{6′}=δ_{S6′}−δ_{R6′}=5.16−5.18=−0.02 ppm;

Δδ_{1′a}=δ_{S1′a}−δ_{R1′a}=3.36−3.25=0.01 ppm;

Δδ_{1′b}=δ_{S1′b}−δ_{R1′b}=3.23−3.13=0.10 ppm;

Δδ₅=δ_{S5}−δ_{R5}=6.93−6.92=0.01 ppm;

Δδ₃=δ_{S3}−δ_{R3}=7.16−7.14=0.02 ppm.

Therefore, the 2′ position should have a R configuration as shown above.

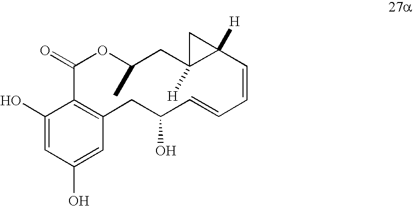

Alcohol 27α (major isomer). ¹H NMR (500 MHz, CDCl₃): δ 11.6 (s, 1H), 6.81 (dd, J=15.8, 9.4 Hz, 1H), 6.39 (d, J=2.4 Hz, 1H), 6.33 (d, J=2.3 Hz, 1H), 5.94 (dd, J=9.8, 9.7 Hz, 1H), 5.81 (dd, J=15.8, 6.2 Hz, 1H), 5.42-5.38 (m, 1H), 5.33 (dd, J=10.1, 5.0 Hz, 1H), 4.64 (m, 1H), 3.93 (dd, J=13.8, 8.1 Hz, 1H), 2.93 (dd, J=13.8, 5.9 Hz, 1H), 2.03 (dt, J=15.7, 5.0 Hz, 1H), 1.88-1.80 (m, 1H), 1.49 (d, J=6.6 Hz, 3H), 1.22-1.15 (m, 1H), 0.59-0.56 (m, 1H), 0.55-0.52 (m, 1H); ¹³C NMR (125 MHz, CDCl₃): δ 171.0, 165.3, 160.8, 143.2, 135.0, 132.6, 129.7, 129.4, 110.8, 106.1, 102.4, 74.4, 73.1, 40.5, 37.8, 18.7, 16.5, 16.1, 14.4; ESIMS m/z 353.0 ([M+Na⁺], C₁₉H₂₂NaO₅ requires 353.1).

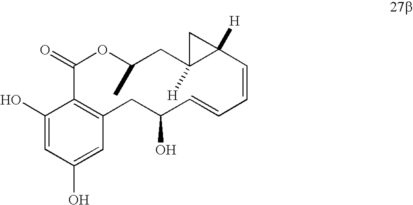

Alcohol 27β (minor). ¹H NMR (500 MHz, CDCl₃): δ 10.83 (s, 1H), 6.66 (dd, J=16.3, 8.6 Hz, 1H), 6.52 (d, J=2.6 Hz, 1H), 6.31 (d, J=2.6 Hz, 1H), 5.89 (dd, J=8.9, 7.6 Hz, 1H), 5.31-5.22 (m, 1H), 5.81 (dd, J=15.8, 6.2 Hz, 1H), 5.26 (dd, J=10.5, 5.9 Hz, 1H), 4.58-4.55 (m, 1H), 3.57-3.48 (m, 2H), 2.01-1.98 (m, 2H), 1.47 (d, J=6.5 Hz, 3H), 0.97-0.92 (m, 1H), 0.64-0.51 (m, 2H); ¹³C NMR (125 MHz, CDCl₃): δ 169.8, 164.0, 160.2, 143.8, 134.7, 133.1, 128.1, 127.8, 111.3, 107.9, 102.4, 72.6, 72.4, 39.9, 37.6, 19.4, 16.7, 16.3, 14.7; ESIMS m/z 353.0 ([M+Na⁺], C₁₉H₂₂NaO₅ requires 353.1).

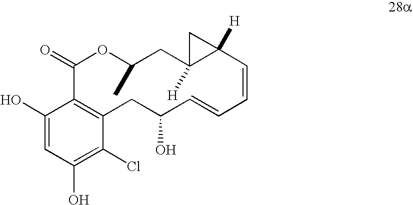

¹H NMR (500 MHz, CDCl₃): δ 11.4 (s, 1H), 7.14 (dd, J=15.3, 11.1 Hz, 1H), 6.56 (s, 1H), 6.53-5.96 (m, 4H), 5.78 (dd, J=15.6, 3.8 Hz), 5.38-5.33 (m, 1H), 4.55 (dd, J=14.8, 6.7 Hz, 1H), 3.72 (dd, J=14.6, 8.6 Hz, 1H), 3.57 (bs, 1H), 2.31-2.27 (m, 1H), 1.59-1.55 (m, 1H), 1.49 (d, J=6.6 Hz, 3H), 0.91-0.88 (m, 1H), 0.58-0.54 (m, 1H), 0.43-0.38 (m, 1H); ESIMS m/z 387.0 ([M+Na⁺], C₁₉H₂₁NaClO₅ requires 387.1).

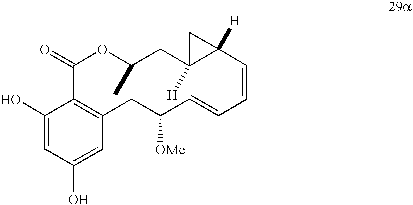

Methylether 29α. To a solution of crude 25a (˜0.033 mmol) in DMF (0.5 mL) was added CH₃I (0.0062 mL, 0.099 mmol), followed by the addition of Ag₂O (11.5 mg, 0.050 mmol). The mixture was stirred at rt for 21.5 h, and filtered through Celite. The solvent and then removed, and the resulting residue was dissolved in 2 mL of 1:1 mixture of MeOH and 5% NaHCO₃. After stirring at rt for 18.5 h, work-up and purification as described for 23 provided 29α (4.4 mg, 39% over 3 steps). ¹H NMR (500 MHz, CDCl₃): δ 11.2 (s, 1H), 6.17 (dd, J=15.9, 10.6 Hz, 1H), 6.24 (d, J=1.6 Hz, 1H), 6.16 (d, J=1.8 Hz, 1H), 5.77 (dd, J=10.6, 10.5 Hz, 1H), 5.64 (dd, J=15.9, 3.5 Hz, 1H), 5.15-5.10 (m, 1H), 4.99 (dd, J=10.2, 6.6 Hz, 1H), 4.58 (m, 1H), 3.63 (s, 3H), 2.85 (dd, J=14.8, 5.3 Hz, 1H), 2.73 (dd, J=14.7, 9.5 Hz, 1H), 2.17 (dt, J=15.2, 4.4 Hz, 1H), 1.36 (d, J=6.3 Hz, 3H), 1.05-1.00 (m, 1H), 0.78-0.73 (m, 1H), 0.44-0.39 (m, 2H); ¹³C NMR (125 MHz, CDCl₃): δ 169.1, 158.9, 157.9, 137.1, 134.7, 134.2, 129.7, 127.6, 116.5, 106.5, 97.4, 73.0, 70.5, 55.8, 39.6, 39.2, 17.9, 16.2, 14.7; ESIMS m/z 367.2 ([M+Na⁺], C₂₀H₂₄NaO₅ requires 367.2).

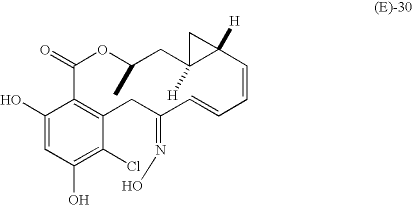

E-Oxime. Cycloproparadicicol 2 (4.6 mg, 0.0127 mmol) and hydroxylamine hydrochloride (2.2 mg, 0.032 mmol) were dissolved in pyridine (0.22 mL). The mixture was heated to 50° C. for 7 h. The pyridine was then removed in vacuum, and the residue was purified by PTLC (1:1, Hexanes/EtOAc) to afford two separable isomers along with recovered 2 (E-isomer: 1.0 mg, 28%; Z-isomer: 1.5 mg, 42% based on consumed starting material). ¹H NMR (400 MHz, CDCl₃) (E-isomer): δ 8.61 (d, J=4.2 Hz, 1H), 7.46 (dd, J=16.2, 11.2 Hz, 1H), 6.56 (s, 1H), 6.12 (d, J=16.2 Hz, 1H), 6.04 (dd, J=11.2, 9.8 Hz, 1H), 5.41-5.37 (m, 1H), 5.31 (dd, J=9.8, 5.3 Hz, 1H), 4.62 (d, J=16.3 Hz, 1H), 4.26 (d, J=16.3 Hz, 1H), 2.17 (dt, J=15.8, 2.8 Hz, 1H), 1.53-1.45 (m, 1H), 1.45 (d, J=7.1 Hz, 3H), 1.03-0.97 (m, 1H), 0.79-0.71 (m, 1H), 0.58-0.53 (m, 1H), 0.45-0.42 (m, 1H); ¹³C NMR (100 MHz, CDCl₃): δ 169.4, 162.5, 157.3, 156.3, 138.3, 137.9, 135.6, 131.1, 124.4, 114.1, 108.9, 103.3, 74.3, 37.8, 28.3, 17.9, 17.3, 15.4, 13.0; ESIMS m/z 400.0 ([M+Na⁺], C₁₉H₂₀ClNNaO₅ requires 400.1).

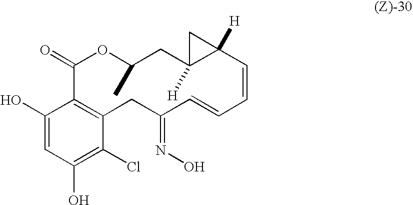

Z-Oxime. ¹H NMR (400 MHz, CDCl₃) (Z-isomer): δ 8.60 (d, J=4.3 Hz, 1H), 7.56 (dd, J=15.5, 11.7 Hz, 1H), 6.75 (d, J=16.3 Hz, 1H), 6.57 (s, 1H), 6.08 (dd, J=10.8, 10.3 Hz, 1H), 5.43-5.39 (m, 2H), 4.78 (d, J=14.7 Hz, 1H), 3.8 (m, 1H), 2.17 (d, J=15.7, 1H), 1.55 (dd, J=11.9, 11.5 Hz, 1H), 1.46 (d, J=6.6 Hz, 3H), 0.87-0.80 (m, 2H), 0.61-0.56 (m, 1H), 0.49-0.46 (m, 1H); ¹³C NMR (100 MHz, CDCl₃): δ 169.3, 162.0, 156.5, 140.5, 137.3, 131.0, 117.3, 115.7, 108.7, 103.5, 74.2, 35.4, 18.2, 17.4, 15.6, 13.4; ESIMS m/z 400.2 ([M+Na⁺], C₁₉H₂₀ClNNaO₅ requires 400.1).

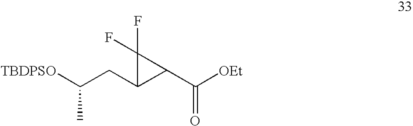

Difluorocyclopropanation products. To a solution of 32 (1.58 g, 3.98 mmol) in toluene (1 mL) was added NaF (25 mg, 0.58 mmol). Trimethylsilyl fluorosulfonyldifluoroacetate (TFDA) (6.5 mL, 32.9 mmol) was added dropwise via a syringe pump at a rate of 1 mL/h. After 6.5 h, the reaction mixture was cooled to rt and filtered. The solvent was removed in vacuum. The residue was purified by PTLC (9:1 Hexanes/EtOAc) to give product as a mixture of diastereomers (830 mg, 47%, ratio 1/0.8). Major isomer: ¹H NMR (400 MHz, CDCl₃): δ 7.75-7.72 (m, 5H), 7.49-7.27 (m, 5H), 4.26-4.19 (m, 2H), 4.06 (m, 1H), 2.55-2.49 (m, 1H), 2.13 (dd, J=13.3, 7.5 Hz, 1H), 1.76-1.68 (m, 2H), 1.34-1.28 (m, 3H), 1.32 (d, J=7.1 Hz, 3H), 1.15 (d, J=6.6 Hz, 3H), 1.12 (s, 9H). ¹³C NMR (100 MHz, CDCl₃): δ 166.9, 136.0, 134.5, 129.9, 127.9, 68.1, 61.5, 35.2, 34.9, 31.8, 31.4, 27.1, 26.1, 23.6, 19.3, 14.3. ESIMS m/z 469.1 ([M+Na⁺], C₂₅H₃₂F₂NaO₃Si requires 469.2).

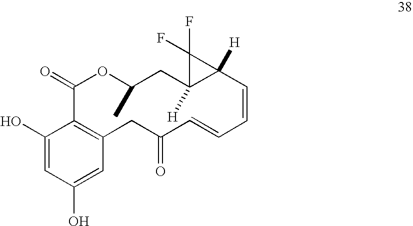

Difluorocyclopropamonocilin. ¹H NMR (400 MHz, CDCl₃): δ 11.2 (s, 1H), 8.13 (dd, J=16.0, 11.6 Hz, 1H), 7.50 (bs, 1H), 6.42 (d, J=2.2 Hz, 1H), 6.40 (d, J=2.2 Hz, 1H), 6.38 (t, J=10.3 Hz, 1H), 6.02 (d, J=16.0 Hz, 1H), 5.93 (dd, J=10.0, 6.0 Hz, 1H), 5.59-5.56 (m, 1H), 5.24 (d, J=13.7 Hz, 1H), 3.54 (d, J=13.7 Hz, 1H), 2.18 (m, 1H), 1.01-1.99 (m, 1H), 1.65-1.55 (m, 2H), 1.49 (d, J=6.9 Hz, 3H); [α]_{D}²⁵ −17.1 (c 0.42, CH₂Cl₂); ESIMS m/z 386.9 ([M+Na⁺], C₁₉H₁₈F₂NaO₅ requires 387.1).

We assigned the stereo configuration at the difluorocyclopropyl group as same as that in cycloproparadicicol itself, based on the sign of known optical rotation of cycloproparadicicol ([α]_{D}²⁵ −123.7 (c 0.70, CH₂Cl₂)) and its C7′, 8′-epimer ([α]_{D}²⁵ +221.3 (c 1.10, CH₂Cl₂) (Yamamoto, K.; Garbaccio, R. M.; Stachel, S. J.; Solit, D. B.; Chiosis, G.; Rosen, N.; Danishefsky, S. J. Angew. Chem. Int. Ed. 2003, 42, 1280-1284; incorporated herein by reference). Also, in the ¹H spectrum of 38, H6′ (5.93 ppm) has a coupling constant of 6.0 Hz with H7′. In the ¹H of cycloproparadicicol, a very similar coupling constant is observed between H6′ and H7′, e.g. 6.19 Hz (Yamamoto et al. Angew. Chem. Int. Ed. 2003, 42, 1280-1284; incorporated herein by reference). Of course, we realized that we will have to determine absolute stereochemistry of 38 by X-ray in the future when the material can be available after an improved synthesis is achieved.

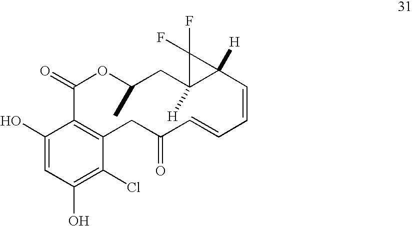

Difluorocycloproparadicicol. ¹H NMR (400 MHz, CDCl₃): δ 11.2 (s, 1H), 7.69 (dd, J=15.9, 10.7 Hz, 1H), 7.05 (bs, 1H), 6.38 (s, 1H), 6.38 (dd, J=10.6, 9.7 Hz, 1H), 6.02 (d, J=16.3 Hz, 1H), 5.93 (dd, J=10.4, 5.6 Hz, 1H), 5.55 (m, 1H), 4.74 (d, J=16.8 Hz, 1H), 3.92 (d, J=17.0 Hz, 1H), 2.18 (m, 1H), 1.01-1.99 (m, 1H), 1.65-1.55 (m, 2H), 1.49 (d, J=6.9 Hz, 3H); ESIMS m/z 433.0 ([M+Cl⁻], C₁₉H₁₇Cl₂F₂O₅ requires 433.1).

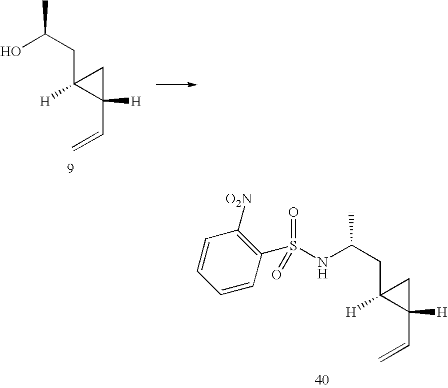

### Sulfonamide 40:

To a mixture of 9 (11 mg, 0.087 mmol), 2-nitrobenzenesulfonamide (44 mg, 0.218 mmol), triphenylphosphine (30 mg, 0.113 mmol) in toluene (0.4 mL) and THF (0.2 mL) was added diisopropylazadicarboxylate (DIAD, 0.022 mL, 0.113 mmol) at 0° C. The reaction mixture was warmed up to r.t. and stirred for 2 hours. The reaction mixture was purified by PTLC (eluant: Hex/EtOAc=3/1) to afford sulfonamide 40 as a colorless oil (17 mg, 61%). ¹H NMR (400 MHz, CDCl₃) δ0.11-0.40 (m, 1H), 0.41-0.47 (m, 1H), 0.57 (m, 1H), 1.03-1.10 (m, 1H), 1.11 (d, J=6.6 Hz, 1H), 1.19-1.32 (m, 1H), 1.43-1.47 (m, 1H), 3.50-3.55 (m, 1H), 4.69 (dd, J=10.3, 1.4 Hz, 1H), 4.88 (dd, J=10.3, 7.0 Hz, 1H), 5.11-5.18 (m, 1H), 5.22 (d, J=9.7 Hz, 1H), 7.50-7.69 (m, 2H), 7.75-7.78 (m, 1H), 8.05-8.08 (m, 1H). ¹³C NMR (100 MHz, CDCl₃) δ 13.6, 17.6, 21.9, 26.5, 41.7, 51.7, 12.3, 125.7, 131.1, 133.2, 133.9, 141.4, 148.2, 171.6. LRMS (ESI) calcd for C₁₄H₁₈N₂O₄SNa⁺[M+Na]⁺: 333.1, found 333.0. LRMS (ESI) calc'd for Cl₄H₁₈N₂O₄SCl⁻[M+Cl]⁻: 345.1, found 345.0.

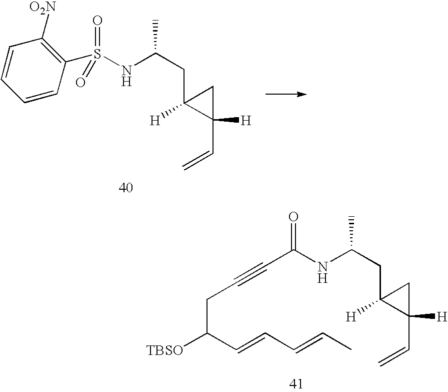

### Amide 41:

To a solution of 40 (211 mg, 0.680 mmol) in CH₃CN (15 mL) was added PhSLi (1.0 M in THF, 1.1 mL, 1.02 mmol) and stirred for 1 h. Then a pre-mixed solution of acid 8 (460 mg, 1.56 mmol), DIEA (0.28 mL, 1.63 mmol), HATU (647 mg, 1.7 mmol) in CH₃CN (4 mL) was added to the reaction mixture. After 0.5 h, the reaction mixture was concentrated and purified on a silica gel column (eluant: Hex/EtOAc=4/1) to afford amid 41 as a colorless oil (182 mg, 67% for two steps). ¹H NMR (400 MHz, CDCl₃) δ 0.00 (s, 3H), 0.03 (s, 3H), 0.49-0.52 (m, 1H), 0.57-0.60 (m, 1H), 0.68-0.69 (m, 1H), 0.85 (s, 9H), 1.12 (d, J=7.3 Hz, 1H), 1.32-1.37 (m, 1H), 1.41-1.46 (m, 1H), 1.70 (d, J=6.7 Hz, 1H), 2.34-2.39 (dd, J=16.7, 6.4 Hz, 2H), 4.02-4.08 (m, 1H), 4.24-4.30 (m, 1H), 4.80 (d, J=10.3 Hz, 1H), 5.00 (d, J=7.0 Hz, 1H), 5.28-5.36 (m, 1H), 5.48-5.53 (m, 2H), 5.61-5.69 (m, 1H), 5.69-5.61 (m, 1H), 6.09-6.14 (m, 1H). ¹³C NMR (100 MHz, CDCl₃) δ −5.9, −5.5, 0.0, 12.2, 16.2, 17.1, 17.2, 19.1, 21.7, 24.8, 27.8, 39.0, 45.0, 70.4, 82.7, 110.9, 127.4, 129.2, 129.6, 129.8, 130.7, 140.2, 151.5. HRMS (FAB) calc'd for C₂₄H₃₉NO₂SiNa⁺[M+H]⁺: 424.2648, found: 424.2648, Δ=−0.1 ppm.

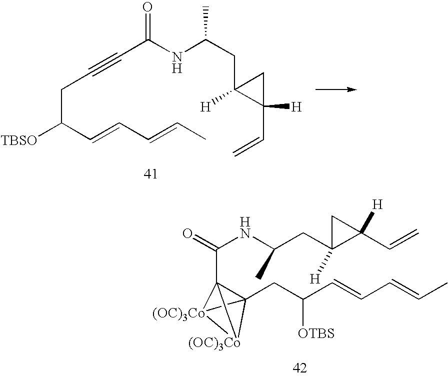

### Cobalt Complex 42:

To a solution of 41 (669 mg, 1.66 mmol) in toluene (50 mL) was added Co₂(CO)₈(797 mg, 2.33 mmol). After 0.5 h, the reaction mixture was concentrated and purified on a silica gel column (eluant: Hex/EtOAc=20/1) to afford Cobalt complex 42 as a purple oil (934 mg, 85%). ¹H NMR (400 MHz, CDCl₃) δ 0.00 (s, 3H), 0.02 (s, 3H), 0.48-0.50 (m, 1H), 0.52-0.56 (m, 1H), 0.66-0.68 (m, 1H), 0.81 (s, 9H), 1.07-1.10 (m, 1H), 1.15 (d, J=6.0 Hz, 1H), 1.21 (m, 2H), 1.65 (d, J=6.7 Hz, 1H), 3.21 (m, 2H), 3.99-4.04 (m, 1H), 4.33-4.40 (m, 1H), 4.75 (d, J=10.1 Hz, 1H), 4.93 (d, J=16.9 Hz, 1H), 5.26-5.33 (m, 1H), 5.51-5.65 (m, 2H), 5.66 (m, 1H), 5.90-5.96 (m, 1H), 6.05-6.10 (m, 1H). ¹³C NMR (100 MHz, CDCl₃) δ −4.8, −4.6, 13.5, 17.5, 18.1, 18.4, 20.4, 22.3, 25.9, 40.4, 43.0, 46.8, 73.2, 73.4, 85.1, 93.1, 111.8, 129.8, 130.9, 131.9, 132.2, 132.3, 166.6, 198.7. LRMS (ESI) calc'd for C₃₀H₃₉Co₂NO₈SNa⁺[M+Na]⁺: 710.1, found 710.2.

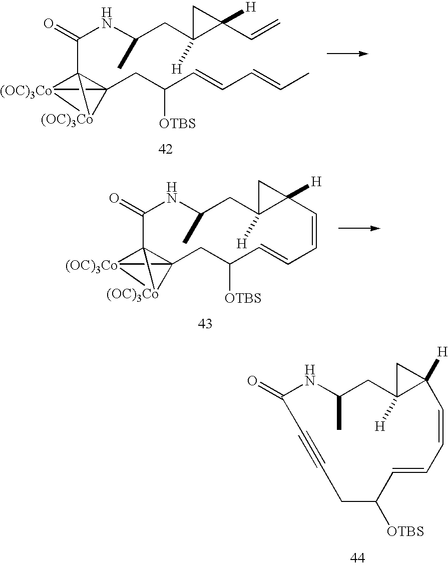

### Macrolactam 44:

To a solution of 42 (902 mg, 1.31 mmol) in CH₂Cl₂ (6.6 L) was added 11 (2^{nd} generation Grubbs' catalyst, 446 mg, 0.52 mmol). The reaction mixture was stirred for 5 h and filtered through silical gel and washed with CH₂Cl₂ and then concentrated. To the residue was then added acetone (80 mL) and di-tert-butylpyridine (4.4 mL, 19.7 mmol) and cooled to 0° C. To this mixture at 0° C. was added CAN (4.2 g, 7.87 mmol). After 0.5 h, DIEA (4.1 mL, 23.6 mmol) was added to quench the reaction and the mixture was filtered by neutral alumina and concentrated. The crude was purified by PTLC (eluant: Hex/EtOAc=3/1) to afford two isomer of 44. Major isomer of 44: (176 mg, 37%). ¹H NMR (400 MHz, CDCl₃) δ 0.00 (s, 3H), 0.01 (s, 3H), 0.60-0.61 (m, 1H), 0.65-0.69 (m, 1H), 0.73 (m, 1H), 0.82 (s, 9H), 0.85-0.98 (m, 1H), 1.13 (d, J=6.8 Hz, 1H), 1.55-1.61 (m, 2H), 1.97-2.01 (m, 1H), 2.25 (dd, J=16.4, 10.9 Hz, 1H), 2.51 (dd, J=16.4, 4.3, Hz, 1H), 4.24-4.32 (m, 2H), 4.79-4.84 (m, 1H), 5.50 (dd, J=15.4, 7.8 Hz, 1H), 5.92-5.98 (m, 1H), 6.56 (dd, J=15.4, 11.2 Hz, 1H). ¹³C NMR (100 MHz, CDCl₃) δ −4.5, −4.2, 1.0, 12.9, 15.1, 18.5, 19.8, 20.1, 26.2, 29.1, 38.3, 44.8, 74.0, 79.4, 84.1, 126.2, 127.0, 134.7, 135.4, 152.5. LRMS (ESI) calcd for C₂₁H₃₃NO₂SiNa⁺ [M+Na]⁺: 382.2, found 382.2.

Minor isomer of 44 (108 mg, 23%). ¹H NMR (400 MHz, CDCl₃) δ −0.02 (s, 3H), 0.00 (s, 3H), 0.61-0.63 (m, 1H), 0.64-0.70 (m, 2H), 0.84 (s, 9H), 0.92-0.99 (m, 1H), 1.12 (d, J=6.8 Hz, 1H), 1.57-1.62 (m, 1H), 1.97-2.02 (m, 1H), 2.28 (dd, J=16.9, 2.3 Hz, 1H), 2.57 (dd, J=16.9, 4.4, Hz, 1H), 4.03-4.07 (m, 1H), 4.48-4.49 (m, 1H), 4.78 (t, J=10.7 Hz, 1H), 5.63 (dd, J=15.0, 3.9 Hz, 1H), 5.98 (t, J=11.0 Hz, 1H), 6.07 (bd, J=9.0 Hz, 1H), 6.95 (dd, J=15.0, 11.4 Hz, 1H). ¹³C NMR (100 MHz, CDCl₃) δ −6.1, −5.8, 0.0, 11.5, 13.7, 17.2, 18.3, 18.5, 24.7, 24.8, 27.3, 36.8, 43.4, 66.7, 77.4, 82.3, 125.3, 125.6, 131.7, 133.3, 151.5. HRMS (FAB) calcd for C₂₁H₃₃NO₂SiNa⁺ [M+Na]⁺: 382.2178, found 382.2178; Δ=−0.1 ppm.

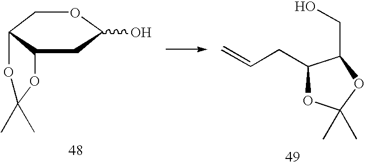

### (2R,3S)-Hex-5-ene-1,2,3-triol 2,3-acetonide (49):

To a stirring suspension of Ph₃P⁺CH₃I⁺ (11.2 g, 27.7 mmol) in 30 mL THF was added KHMDS (0.5 M in toluene, 46.0 mL, 23.0 mmol) at −78° C. The solution was warmed up to 0° C. and stirred for 30 min before cooled down to −78° C. Acetonide 48 (Barbat, J.; Gelas, J.; Horton, D. Carbohydr. Res. 1983, 116, 312-316; incorporated herein by reference) (1.6 g, 9.2 mmol) in 5 mL THF was added via cannula and the solution was warmed up to r.t. overnight (10 h) before quenched with saturated aqueous NH₄Cl solution, extracted with EtOAC (100 mL×3). The organic layers were combined and dried with anhydrous MgSO₄, filtered, and concentrated under reduced pressure vacuum. The residue was purified on a silica gel column using petroleum ether/EtOAc (4/1) as the eluant to afford 49 as a colorless oil (1.02 g, 68%). [α]_{D}²⁵ 54.8 (c 0.26, CHCl₃). ¹H NMR (400 MHz, CDCl₃) δ 1.34 (s, 3H), 1.46 (s, 3H), 2.02 (b, 1H), 2.25-2.32 (m, 1H), 2.37-2.44 (m, 1H), 3.65 (m, 1H), 4.16-4.21 (m, 1H), 4.24-4.28 (m, 1H), 5.10-5.18 (m, 2H), 5.79-5.89 (m, 1H). ¹³C NMR (100 MHz, CDCl₃) δ 25.4, 28.1, 33.6, 61.6, 76.2, 77.8, 108.3, 117.3, 134.2. LRMS (ESI) calcd for C₉H₁₆O₃Na⁺ [M+Na]⁺: 195.1, found 194.9. LRMS (ESI)calcd for C₉H₁₆O₃Cl⁻ [M+Cl]: 207.1, found 207.1.

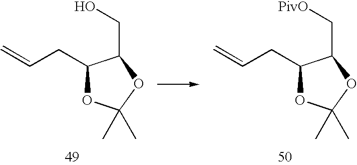

### Pivalate (50):

To a solution of 49 (752 mg, 4.37 mmol), DMAP (106 mg, 0.874 mmol) and triethylamine (2.5 ml, 17.5 mmol) in CH₂Cl₂ (8 ml) was added PivCl (1.1 ml, 8.74 mmol) at 0° C. The reaction mixture was warmed up to r.t. overnight (10 h) before quenched with saturated aqueous NaHCO₃ solution, extracted with EtOAC (100 mL×3). The organic layers were combined and dried with anhydrous MgSO₄, filtered, and concentrated under reduced pressure vacuum. The residue was purified on a silica gel column using hexanes/EtOAc (4/1) as the eluant to afford 50 as a colorless oil (984 mg, 90%). [α]_{D}²⁵ 15.6 (c 0.32, CHCl₃). ¹H NMR (400 MHz, CDCl₃) δ 1.22 (s, 9H), 1.33 (s, 3H), 1.47 (s, 3H), 2.28-2.41 (m, 2H), 4.10-4.13 (m, 2H), 4.22-4.29 (m, 2H), 5.10-5.17 (m, 2H), 5.81-5.91 (m, 1H). ¹³C NMR (100 MHz, CDCl₃) δ 25.9, 27.5, 28.4, 39.1, 63.2, 75.5, 77.8, 108.8, 117.7, 134.6, 178.5. LRMS (ESI) calcd for C₁₄H₂₄O₄Na⁺ [M+Na]⁺ 279.2, found 278.9.

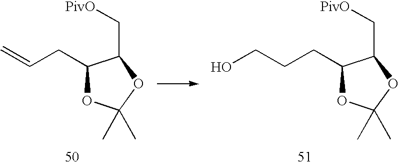

### Alcohol 51:

To a solution of 50 (166 mg, 0.648 mmol) in THF (1.5 mL) was added 9-BBN (0.5 M in THF, 2.8 mL, 1.425 mmol) at 0° C. The reaction mixture was warmed up to r.t. over 4 h and H₂O (0.1 mL), NaOH (3 M, 0.7 mL) and H₂O₂ (30%, 0.2 mL) were added. The reaction mixture was diluted with H₂O 2.5 h later and acidified with citric acid (5%) till pH=7. The mixture was extracted with EtOAc, washed with saturated aqueous Na₂S₂O₃, H₂O and brine. The organic layers were combined and dried with anhydrous MgSO₄, filtered, and concentrated under reduced pressure vacuum. The residue was purified on a silica gel column using petroleum ether/EtOAc (1/1) as the eluant to afford 51 as a colorless oil (156 mg, 88%). [α]_{D}²⁵ 82.4 (c 0.07, CHCl₃). ¹H NMR (400 MHz, CDCl₃) δ 1.21 (s, 9H), 1.36 (s, 3H), 1.46 (s, 3H), 1.59-1.80 (m, 4H), 2.30 (b, 1H), 3.54 (m, 2H), 4.08 (dd, J=6.1, 11.5 Hz, 1H), 4.12 (dd, J=5.6, 11.5 Hz, 1H), 4.19 (m, 1H), 4.25 (m, 1H). ¹³C NMR (100 MHz, CDCl₃) δ 25.5, 25.9, 27.1, 28.0, 29.9, 38.7, 62.3, 62.9, 75.3, 77.0, 108.3, 178.2. HRMS (ESI) calcd for C₁₄H₂₆O₅Na⁺ [M+Na]⁺ 297.1678, found 297.1660, Δ=−6.0 ppm.

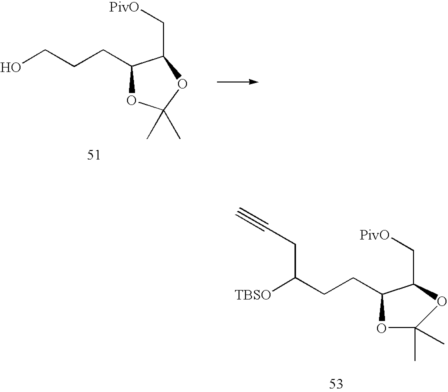

### Alkyne 53:

To a solution of 51 (682 mg, 2.485 mmol) in CH₂Cl₂ (5 mL), DMSO (5 mL) and Et₃N (3 mL) was added SO₃-pyridine complex (1.6 g, 10.2 mmol) at 0° C. The reaction mixture was stirred for 1 h before diluted with EtOAc and washed with HCl (0.5 N), H₂O, saturated aqueous NaHCO₃ solution and brine. The organic layers were dried with anhydrous MgSO₄, filtered, and concentrated under reduced pressure vacuum. The crude aldehyde 52 was used directly for next step without any further purification. To a suspension of Zn (Nano-size power, pre-activated, 390 mg, 5.949 mmol) in THF (10 mL) was added propargyl bromide (80% in toluene, 0.53 mL, 4.759 mmol) at 0° C. The reaction mixture was stirred for 1 h at 0° C. and a solution of aldehyde 52 thus obtained in THF (5 mL) was added and reaction mixture was warmed up to r.t. over 2 h before quenched with saturated aqueous NH₄Cl solution and extracted with EtOAc. The organic layers were dried with anhydrous MgSO₄, filtered, and concentrated under vacuum. To a solution of crude alcohol thus obtained and 2,6-lutidine (0.6 mL, 4.76 mmol) in CH₂Cl₂ (8 mL) was added TBSOTf (0.82 mL, 3.57 mmol) and the reaction mixture was stirred for 10 h before quenched with saturated aqueous NH₄Cl solution, extracted with EtOAc (100 mL×3). The organic layers were combined and dried with anhydrous MgSO₄, filtered, and concentrated under reduced pressure vacuum. The residue was purified on a silica gel column using petroleum ether/EtOAc (20/1) as the eluant to afford 53 as a colorless oil (896 mg, 89% from 51). ¹H NMR (400 MHz, CDCl₃) δ −0.02-0.05 (m, 6H), 0.80 (s, 9H), 1.13 (s, 9H), 1.26 (s, 3H), 1.36 (s, 3H), 1.42-1.74 (m, 4H), 1.81 (m, 1H), 2.22-2.29 (m, 2H), 3.78 (m, 1H), 4.01-4.08 (m, 3H), 4.12-4.17 (m, 1H). ¹³C NMR (100 MHz, CDCl₃) δ 18.0, 18.1, 24.3, 24.7, 25.6, 25.8, 27.0, 27.1, 27.2, 28.06, 28.07, 33.2, 33.3, 38.7, 62.8, 62.9, 70.1, 70.2, 70.4, 70.5, 75.2, 76.1, 77.1, 81.2, 108.20, 108.24, 178.1. HRMS (FAB) calcd for C₂₃H₄₂O₅SiH⁺ [M+H]⁺: 427.2880, found 427.2880, Δ=−0.1 ppm.

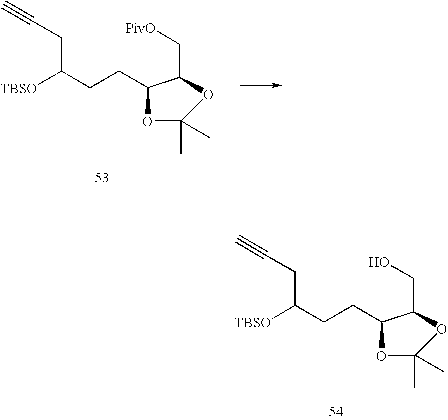

### Alcohol 54:

To a solution of 53 (124 mg, 0.291 mmol) in MeOH (6 mL) was added NaOMe/MeOH (25%, 0.2 mL) and the reaction mixture was stirred for 10 h before quenched with saturated aqueous NH₄Cl solution, extracted with EtOAc (100 mL×3). The organic layers were combined and dried with anhydrous MgSO₄, filtered, and concentrated under reduced pressure vacuum. The residue was purified on a silica gel column using petroleum ether/EtOAc (8/1) as the eluant to afford 54 as a colorless oil (87 mg, 88%). ¹H NMR (400 MHz, CDCl₃) δ −0.02-0.00 (m, 6H), 0.80 (s, 9H), 1.32 (s, 3H), 1.38 (s, 3H), 1.32-1.91 (m, 4H), 1.90 (m, 1H), 1.96 (b, 1H), 2.23-2.31 (m, 2H), 3.53 (m, 2H), 3.75 (m, 1H), 4.08 (m, 1H). ¹³C NMR (100 MHz, CDCl₃) δ −4.7, −4.6, −4.5, 18.0, 24.5, 24.7, 25.5, 25.6, 25.8, 27.2, 27.4, 28.2, 33.2, 33.4, 61.7, 70.1, 70.2, 70.46, 70.52, 76.9, 77.0, 77.9, 81.16, 81.24, 108.08, 108.14. HRMS (FAB) calcd for C₁₈H₃₄O₄SiH⁺ [M+H]⁺: 343.2305, found 343.2305, Δ=−0.1 ppm.

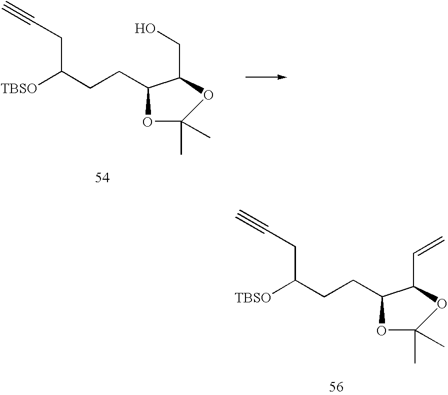

### Enyne 56:

To a solution of 54 (87 mg, 0.254 mmol) in DMSO (1.0 mL), CH₂Cl₂ (1.0 mL) and Et₃N (1.0 mL) was added SO₃-Pyrdine complex (200 mg, 2.032 mmol) at 0° C. The reaction mixture was stirred for 2 h before diluted with EtOAc and washed with HCl (0.5 N), H₂O, saturated aqueous NaHCO₃ solution and brine. The organic layers were dried with anhydrous MgSO₄, filtered, and concentrated under reduced pressure vacuum. The crude aldehyde 55 was used directly for next step without any further purification. To a stirring suspension of Ph₃P⁺CH₃I⁻ (204 mg, 0.505 mmol) in 3 mL THF was added KHMDS (0.5 M in toluene, 0.9 mL, 0.454 mmol) at −78° C. The solution was warmed up to 0° C. and stirred for 30 min before cooled down to −78° C. Aldehyde obtained as mention above in 2 mL THF was added via cannula and the solution was warmed up to r.t. overnight (10 h) before quenched with saturated aqueous NH₄Cl solution, extracted with EtOAc (100 mL×3). The organic layers were combined and dried with anhydrous MgSO₄, filtered, and concentrated under reduced pressure vacuum. The residue was purified on a silica gel column using petroleum ether/EtOAc (40/1) as the eluant to afford 56 as a colorless oil (73 mg, 86%). ¹H NMR (400 MHz, CDCl₃) δ −0.05 (s, 3H), 0.02 (s, 3H), 0.83 (s, 9H), 1.27 (s, 3H), 1.43 (s, 3H), 1.43-1.78 (m, 4H), 1.92 (b, 1H), 2.23-2.31 (m, 2H), 3.76 (m, 1H), 4.05-4.10 (m, 1H), 4.43-4.45 (m, 1H), 5.02-5.05 (m, 2H), 5.71-5.80 (m, 1H). ¹³C NMR (100 MHz, CDCl₃) δ −4.30, −4.11, 18.4, 26.0, 26.1, 26.2, 26.5, 26.8, 27.7, 27.8, 28.62, 28.64, 33.2, 33.6, 70.4, 70.5, 71.07, 71.13, 78.6, 78.8, 80.2, 81.78, 81.82, 108.5, 118.6, 118.7, 134.7, 134.9. HRMS (FAB) calcd for C₁₉H₃₄O₃SiNa⁺ [M+Na]⁺: 361.2175, found 361.2175, Δ=0.0 ppm.

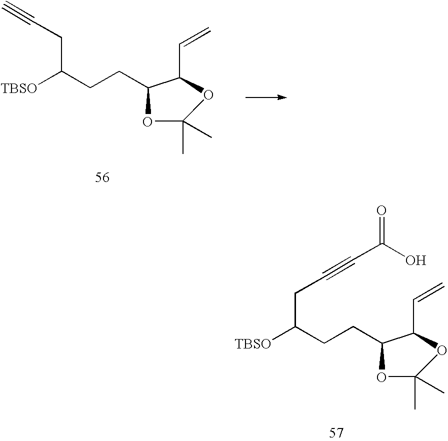

### Acid 57:

To a solution of 56 (586 mg, 1.731 mmol) in Et₂O (16 mL) was added BuLi (1.6 M in hexane, 1.817 mmol) at −78° C. and stirred for 5 min before quenched with dry ice and warmed up to r.t. The reaction mixture was washed with NaOH (0.1 M) and the aqueous layers were combined and acidified by HCl (0.1 M) until the pH=2. The aqueous layer was extracted with EtOAc and the organic layers were combined and dried with anhydrous MgSO₄, filtered, and concentrated under reduced pressure vacuum and dried on high vacuum. ¹H NMR (400 MHz, CDCl₃) δ −0.03 (s, 3H), 0.00 (s, 3H), 0.79 (s, 9H), 1.17 (s, 3H), 1.40 (s, 3H), 0.95-1.68 (m, 4H), 2.38-2.39 (m, 2H), 3.80 (b, 1H), 4.06 (m, 1H), 4.43 (m, 1H), 5.14-5.36 (m, 2H), 5.67-5.76 (m, 1H), 10.5 (bs, 1H). This crude was used directly for next step without any further purification.

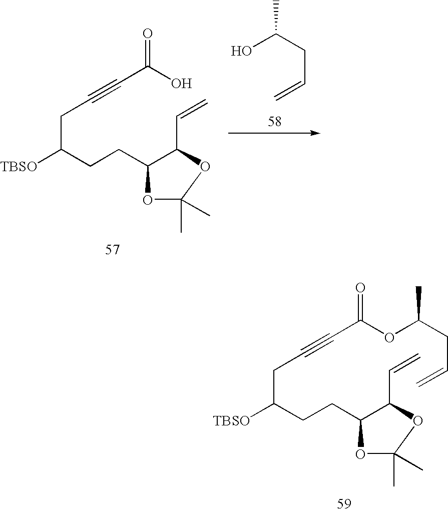

### Ester 59:

To a solution of acid 57 (249 mg, 0.651 mmol) in toluene (15 mL) was added alcohol 58 (0.081 mL, 0.781 mmol), PPh₃ (205 mg, 0.781 mmol), DIAD (0.154 mL, 0.781 mmol). The reaction mixture was stirred for 10 h and the solvent was removed under reduced pressure vacuum. The residue was purified on a silica gel column using petroleum ether/EtOAc (40/1) as the eluant to afford 59 as a colorless oil (255 mg, 85% for two steps). ¹H NMR (400 MHz, CDCl₃) δ −0.06-0.00 (m, 6H), 0.80 (s, 9H), 1.17 (d, J=6.2 Hz, 3H), 1.28 (s, 3H), 1.38 (s, 3H), 1.44-1.54 (m, 4H), 2.20-2.24 (m, 1H), 2.237-2.31 (m, 1H), 2.35-2.37 (m, 2H), 3.78-3.81 (m, 1H), 4.03-4.05 (m, 1H), 4.40-4.43 (m, 1H), 4.94-4.96 (m, 1H), 5.00-5.04 (m, 2H), 5.14 (m, 1H), 5.22 (dd, J=6.7, 7.1 Hz, 1H), 5.67-5.72 (m, 2H). ¹³C NMR (100 MHz, CDCl₃) δ −4.64, −4.58, −4.55, 0.0, 18.0, 19.3, 25.6, 25.7, 25.8, 26.1, 26.3, 27.6, 27.7, 28.2, 28.3, 33.3, 33.6, 40.0, 70.10, 70.12, 72.0, 74.9, 78.1, 78.3, 79.80, 79.82, 86.1, 108.2, 108.3, 118.1, 118.3, 118.4, 133.2, 134.2, 134.4, 153.2. HRMS (FAB) calcd for C₂₅H₄₂O₅SiH⁺ [M+H]⁺: 451.2880, found 451.2881, Δ=−0.3 ppm.

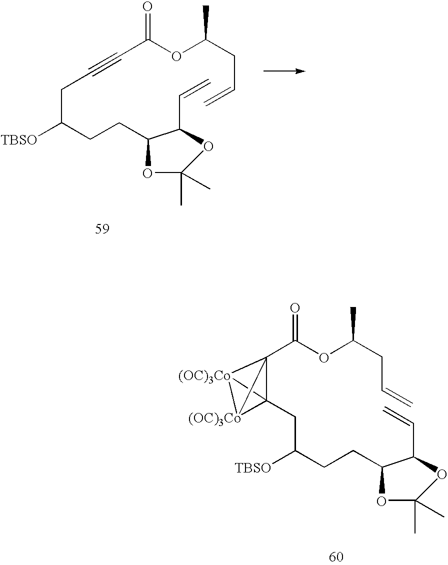

### Cobalt-Complex 60:

To a solution of 59 (20 mg, 0.044 mmol) in toluene (2.5 mL) was added Co₂(CO)₈ (21.2 mg, 0.062 mmol). The reaction mixture was stirred for 30 min before filtered through neutral alumina and concentrated under reduced pressure vacuum. The residue was purified on preparative TLC (Whatman® Pk6F Silica Gel 60 Å 1000 μM) using Hexanes/EtOAc (20/1) as the eluant to afford 60 as a purple oil (30 mg, 94%). ¹H NMR (400 MHz, CDCl₃) δ 0.07-0.09 (m, 6H), 0.90 (s, 9H), 1.30 (d, J=8.1 Hz, 3H), 1.33 (s, 1.6H), 1.34 (s, 1.4H), 1.45 (s, 1.6H), 1.47 (s, 1.4H), 1.25-1.46 (m, 1H), 1.58-1.89 (m, 2.5H), 1.89-1.95 (m, 0.5H), 2.37-2.40 (m, 2H), 3.00-3.12 (m, 2H), 3.82-3.86 (m, 1H), 4.08-4.14 (m, 1H), 4.47-4.51 (m, 1H), 5.07-5.14 (m, 3H), 5.21 (d, J=10.4 Hz, 1H), 5.29 (d, J=17.1 Hz, 1H), 5.73-5.84 (m, 2H). ¹³C NMR (100 MHz, CDCl₃) δ 18.1, 19.4, 25.6, 25.8, 26.5, 27.0, 28.16, 28.2, 32.7, 33.0, 40.3, 41.9, 42.1, 71.9, 72.8, 73.0, 78.2, 169.0, 197. HRMS (FAB) calcd for C₃₁H₄₂Co₂O₁₁SiH⁺ [M+H]⁺: 737.1239, found 737.1240, Δ=−0.2 ppm.

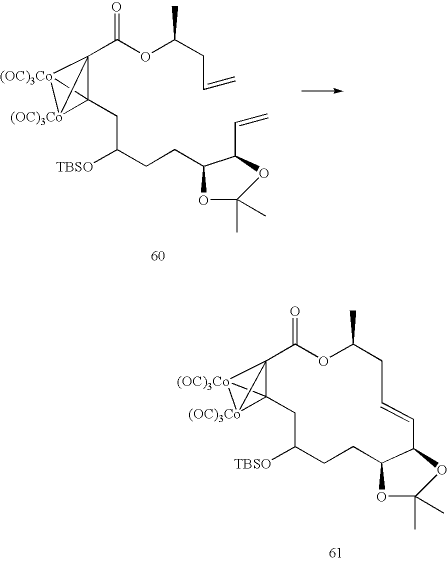

### Macrolactone 61:

To a solution of 60 (339 mg, 0.460 mmol) in CH₂Cl₂ (80 mL) was added 2^{nd} generation Grubbs catalyst (97 mg, 0.115 mol) in CH₂Cl₂ (15 mL) via cannula at r.t. The reaction mixture was stirred overnight and then the solvent was removed under reduced pressure vacuum and residue purified on preparative TLC (Whatman® Pk6F Silica Gel 60 Å 1000 μM) using Hexanes/EtOAc (10/1) as the eluant to afford (2′R)-61 as a purple oil (123 mg, 38%). [α]_{D}²⁵ −44.7 (c 0.25, CHCl₃). ¹H NMR (400 MHz, CDCl₃) δ 0.08 (s, 3H), 0.09 (s, 3H), 0.91 (s, 9H), 1.31 (d, J=6.3 Hz, 3H), 1.35 (s, 3H), 1.45 (s, 3H), 1.39-1.59 (m, 2H), 1.65-1.72 (m, 1H), 1.93-1.97 (m, 1H), 2.22-2.31 (m, 1H), 2.42 (dt, J=12.9, 1.83 Hz, 1H), 3.08 (dd, J=16.0, 1.5 Hz, 1H), 3.23 (dd, J=16.0, 9.4 Hz, 1H), 3.92-3.95 (m, 1H), 4.01-4.06 (m, 1H), 4.40 (dd, J=9.4, 5.7 Hz, 1H), 5.53 (ddd, J=15.2, 10.9, 1.52 Hz, 1H), 5.72 (ddd, J=15.2, 10.7, 3.8 Hz, 1H). ¹³C NMR (100 MHz, CDCl₃) δ 4.5, −4.4, 18.5, 21.2, 23.1, 26.1, 26.2, 28.8, 30.5, 41.0, 42.7, 71.6, 71.8, 79.3, 80.0, 81.3, 92.9, 108.1, 129.1, 132.6, 169.9, 198.8. HRMS (FAB) calcd for C₂₉H₃₈Co₂O₁₁SiH⁺ [M+H]⁺: 709.0926, found: 709.0924, Δ=0.2 ppm. (2′R)-61 (136 mg, 42%). [α]_{D}²⁵ −5.9 (c 0.27, CHCl₃). ¹H NMR (400 MHz, CDCl₃) δ 0.12 (s, 3H), 0.15 (s, 3H), 0.93 (s, 9H), 1.32 (d, J=6.3 Hz, 3H), 1.35 (s, 3H), 1.42 (s, 3H), 1.11-1.54 (m, 2H), 1.67 (m, 1H), 1.88-1.93 (m, 1H), 2.24-2.33 (m, 1H), 2.41-2.45 (m, 1H), 3.05 (dd, J=14.9, 9.5 Hz, 1H), 3.18 (dd, J=14.9, 2.4 Hz, 1H), 3.54-3.59 (m, 1H), 4.03-4.09 (m, 1H), 4.42 (dd, J=9.5 Hz, 6.0 Hz, 1H), 5.28 (m, 1H), 5.53 (ddd, J=15.2, 7.9, 4.0 Hz, 1H), 5.77 (ddd, J=15.2, 10.5, 3.5 Hz, 1H). ¹³C NMR (100 MHz, CDCl₃) δ −4.3, −4.0, 18.4, 21.1, 25.8, 26.1, 26.2, 26.3, 26.9, 28.6, 32.4, 40.7, 44.6, 71.8, 73.8, 78.6, 79.8, 81.1, 93.3, 107.9, 128.6, 133.6, 169.6, 198.6. HRMS (FAB) calcd for C₂₉H₃₈Co₂O₁₁SiH⁺ [M+H]⁺: 709.0926, found: 709.0924, Δ=0.2 ppm.

Note: the absolute configuration of 2′-OH was determined in compound 65.

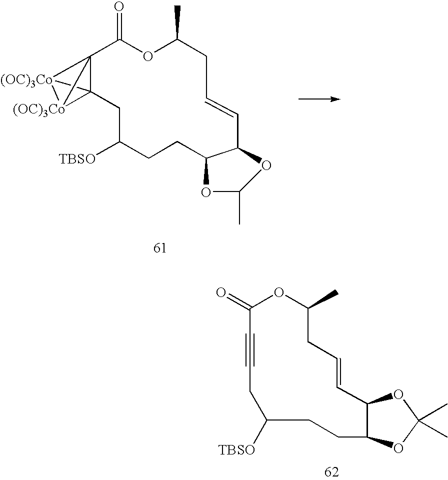

### Macrolide 62:

To a solution of (2′R)-61 (123 mg, 0.174 mmol) in acetone (10 mL) was added CAN (475 mg, 0.868 mmol) at −10° C. After 20 min, the reaction mixture was filtered through neutral alumina and the solvent was removed under reduced pressure vacuum. The residue was purified on a silica gel column using petroleum ether/EtOAc (20/1) as the eluant to afford (2′R)-62 as a colorless oil (69 mg, 94%). [α]_{D}²⁵ −124.6 (c 0.17, CHCl₃). ¹H NMR (400 MHz, CDCl₃) δ −0.06, (s, 3H), 0.00 (s, 3H), 0.81 (s, 9H), 1.24 (d, J=6.2 Hz, 1H), 1.33, (s, 3H), 1.40 (s, 3H), 1.59-1.81 (m, 4H), 2.18-2.22 (m, 1H), 2.28-2.31 (m, 1H), 2.34-2.45 (m, 2H), 3.90-3.93 (m, 1H), 3.98-4.01 (m, 1H), 4.33-4.36 (m, 1H), 4.83-4.87 (m, 1H), 5.42-5.53 (m, 2H). ¹³C NMR (100 MHz, CDCl₃) δ −4.8, 18.4, 20.6, 26.1, 26.3, 28.4, 28.8, 30.1, 36.5, 40.6, 70.0, 71.7, 78.9, 80.1, 88.5, 108.7, 129.7, 132.0, 153.8.

(2′S)-62 was prepared by same procedure from (2′S)-61. (2′S)-62: (81 mg, 95%). [α]_{D}²⁵ −173.3 (c 0.41, CHCl₃). ¹H NMR (400 MHz, CDCl₃) δ 0.00, (s, 3H), 0.01 (s, 3H), 0.81 (s, 9H), 1.26 (d, J'2 6.1 Hz, 3H), 1.34 (s, 3H), 1.41 (s, 3H), 1.53-1.59 (m, 2H), 1.77-1.81 (m, 2H), 2.19-2.24 (m, 1H), 2.24-2.26 (m, 1H), 2.31-2.47 (m, 2H), 3.80 (b, 1H), 3.91-3.93 (m, 1H), 4.34-4.40 (m, 1H), 4.75-4.78 (m, 1H), 4.82-4.86 (m, 1H), 5.43-5.60 (m, 2H). ¹³C NMR (100 MHz, CDCl₃) δ −5.0, −4.8, 17.9, 20.17, 20.24, 25.5, 25.6, 25.7, 25.9, 28.0, 28.2, 28.3, 28.4, 29.6, 36.0, 36.7, 40.0, 40.1, 69.5, 70.8, 71.2, 71.4, 78.5, 78.6, 79.4, 79.6, 78.8, 108.2, 129.2, 131.3, 153.0. HRMS (FAB) calcd for C₂₅H₄₂O₅SiNa⁺ [M+Na]⁺: 473.2699, found: 473.2700, Δ=0.2 ppm.

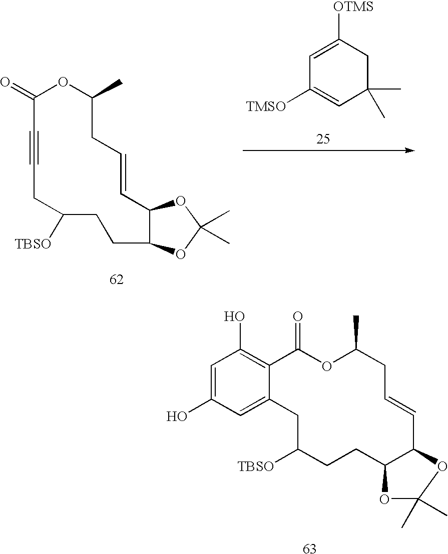

### Resorcyclic Macrolide 63:

Macrolide (2′R)-62 (26 mg, 0.065 mmol) was transferred to a vial and 0.2 mL diene 25 was added. The vial was sealed and heated to 140 0° C. for 36 h. The crude mixture was purified on preparative TLC (Whatman® Pk6F Silica Gel 60 Å 1000 μM) using Hexanes/EtOAc (2/1) as the eluant to afford (2′R)-63 as a colorless oil (23 mg, 74%). [α]_{D}²⁵ −99.1 (c 0.08, CHCl₃). ¹H NMR (400 MHz, CDCl₃) δ −0.15, (s, 3H), 0.09 (s, 3H), 0.89 (s, 9H), 1.36 (s, 3H), 1.38 (s, J=6.1 Hz, 1H), 1.50 (s, 3H), 1.25-1.39 (m, 4H), 1.71-1.76 (m, 1H), 2.54-2.57 (m, 2H), 2.60-2.64 (m, 1H), 3.63-3.68 (m, 2H), 4.08-4.11 (m, 1H), 4.48 (m, 1H), 5.22-5.26 (m, 1H), 5.54 (bs, 1H), 5.70-5.75 (m, 2H), 6.27 (d, J=2.6 Hz, 1H), 6.28 (d, J=2.6 Hz, 1H), 11.3 (s, 1H). ¹³C NMR (100 MHz, CDCl₃) δ −4.5, −4.1, 0.4, 14.6, 18.4, 21.3, 21.5, 24.1, 25.9, 26.3, 28.6, 32.4, 40.1, 42.4, 70.0, 73.5, 73.7, 77.7, 79.7, 101.9, 106.6, 108.7, 111.6, 130.5, 131.6, 145.7, 160.4, 165.0, 172.1. HRMS (FAB) calcd for C₂₇H₄₂O₇SiH⁺ [M+H]⁺: 507.2778, found: 507.2777, Δ=0.2 ppm.

(2′S)-63 was prepared by same procedure from (2′S)-62. (2′S)-63: (81 mg, 84%). [α]_{D}²⁵ −124.2 (c 0.42, CHCl₃). ¹H NMR (400 MHz, CDCl₃) δ 0.00, (s, 3H), 0.14 (s, 3H), 0.99 (s, 9H), 1.50 (s, 3H), 1.58 (d, J=6.1 Hz, 1H), 1.40-1.67 (m, 2H), 1.64 (s, 3H), 1.87-1.92 (m, 2H), 2.64-2.72 (m, 2H), 3.09 (dd, J=3.8 Hz, 2.5 Hz, 1H), 3.45 (dd, J=13.8, 7.8 Hz, 1H), 4.02-4.04 (m, 1H), 4.20-4.22 (m, 1H), 4.70-4.74 (m, 1H), 5.50-5.52 (m, 1H), 5.76 (dd, J=15.5, 8.1 Hz, 1H), 5.93-5.95 (m, 2H), 6.42 (m, 1H), 6.48 (m, 1H), 11.62 (s, 1H). ¹³C NMR (100 MHz, CDCl₃) δ −4.3, −4.0, 14.5, 18.3, 18.4, 20.5, 21.5, 25.8, 26.2, 28.3, 28.4, 28.5, 33.1, 61.1, 72.7, 73.5, 79.1, 102.1, 107.1, 108.5, 112.7, 130.1, 130.5, 144.2, 160.7, 164.9, 171.7. LRMS (ESI) calcd for C₂₇H₄₂O₇SiNa⁺ [M+Na]⁺: 529.2, found: 529.1. LRMS (ESI) calcd for C₂₇H₄₂O₇SiCl⁻ [M+Cl]: 541.3, found: 541.2.

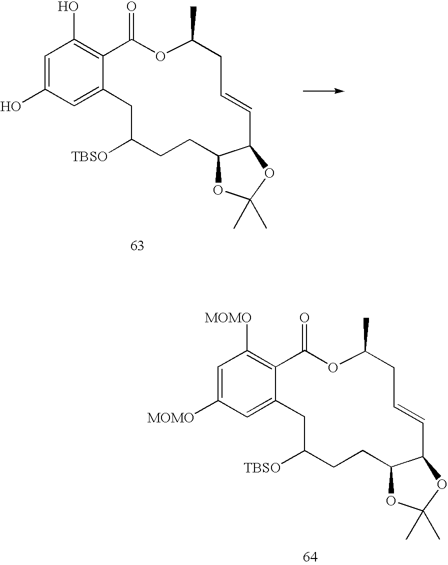

### MOM Ether 64:

To a solution of (2′R)-63 (23 mg, 0.045 mmol) in CH₂Cl₂ (0.5 mL) was added diethylpropylethylamine (0.08 mL, 0.450 mmol) and MOMCl (0.018 mL, 0.227 mmol). The reaction mixture was stirred for 10 h before quenched with saturated aqueous NH₄Cl solution, extracted with EtOAC (100 mL×3). The organic layers were combined and dried with anhydrous MgSO₄, filtered, and concentrated under reduced pressure vacuum. The residue was purified on preparative TLC (Whatman® Pk6F Silica Gel 60 Å 1000 μM) using Hexanes/EtOAc (2/1) as the eluant to afford (2′R)-64 as a colorless oil (21 mg, 78%). [α]_{D}²⁵ −2.86 (c 0.07, CHCl₃).). ¹H NMR (400 MHz, CDCl₃) δ 0.00, (s, 3H), 0.10 (s, 3H), 0.93 (s, 9H), 1.39 (s, 3H), 1.39 (d, 3H), 1.48 (s, 3H), 1.42-1.48 (m, 1H), 1.60-1.64 (m, 2H), 1.72-1.77 (m, 1H), 2.41-2.45 (m, 2H), 2.66 (dd, J=5.6, 4.6 Hz, 1H), 2.89 (dd, J=4.5, 1.6 Hz, 1H), 3.47 (s, 3H), 3.48 (s, 3H), 3.97-3.98 (m, 1H), 4.09-4.15 (m, 1H), 4.73 (dd, J=9.0, 6.0 Hz, 1H), 5.13-5.19 (m, 4H), 5.30-5.36 (m, 1H), 5.57 (dd, J=15.4, 9.1 Hz, 1H), 5.70-5.77 (m, 1H), 6.68 (d, J=2.0 Hz, 1H), 6.86 (d, J=2.0 Hz, 1H). ¹³C NMR (100 MHz, CDCl₃) δ −4.3, −4.2, 0.5, 18.4, 21.6, 24.0, 25.9, 26.3, 28.7, 30.1, 32.3, 40.1, 40.4, 56.5, 56.6, 71.4, 71.6, 80.3, 94.7, 94.9, 101.8, 108.5, 111.4, 119.8, 130.2, 132.4, 139.4, 155.3, 158.6, 168.4.

(2′S)-64 was prepared by same procedure from (2′S)-63. (2′S)-64: (58 mg, 83%). [α]_{D}²⁵ −16.2 (c 0.29, CHCl₃). ¹H NMR (400 MHz, CDCl₃) δ −0.17 (s, 3H), −0.06 (s, 3H), 0.81 (s, 9H), 1.26 (s, 3H), 1.33 (d, J=6.1 Hz, 1H), 1.38 (s, 3H), 1.17-1.52 (m, 4H), 2.30-2.36 (m, 2H), 2.61 (dd, J=14.2, 6.0 Hz, 1H), 2.71 (dd, J=14.2, 6.9 Hz, 1H), 3.39 (s, 3H), 3.40 (s, 3H), 3.80-3.83 (m, 1H), 4.10-4.14 (m, 1H), 4.41 (dd, J=8.9, 6.1 Hz, 1H), 5.04-5.09 (m, 4H), 5.20-5.23 (m, 1H), 5.43 (dd, J=15.3, 9.1 Hz, 1H), 5.60-5.67 (m, 1H), 6.46 (s, 1H), 6.60 (s, 1H). ¹³C NMR (100 MHz, CDCl₃) δ −4.2, −3.8, 18.4, 21.3, 25.8, 26.2, 26.25, 26.3, 28.6, 33.0, 39.8, 40.0, 42.0, 56.5, 56.6, 71.7, 74.5, 78.6, 79.9, 94.7, 94.9, 101.5, 108.3, 111.2, 119.8, 131.0, 131.4, 139.2, 155.8, 158.9, 168.5. HRMS (FAB) calcd for C₃₁H₅₀O₉SiH⁺ [M+H]⁺: 595.3302, found: 595.3304, Δ=−0.3 ppm.

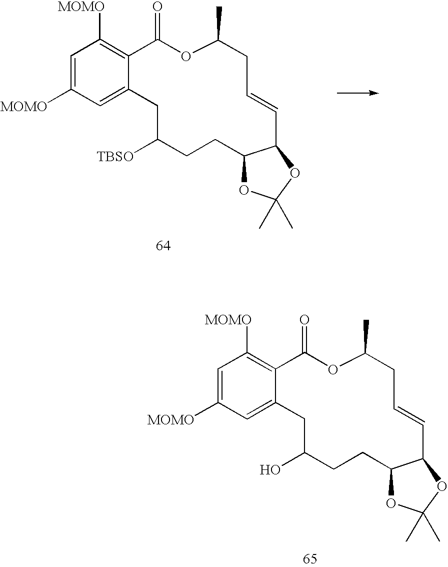

### Alcohol 65:

To a solution of (2′R)-64 (21 mg, 0.035 mmol) in THF (1.4 mL) was added pyridine (0.6 mL) and HF-pyridine (30%, 0.3 mL). The reaction mixture was stirred for 10 h before quenched with MeOTf (2 mL) and stirred for 1 h. The solvent was removed under reduced pressure vacuum. The residue was purified on preparative TLC (Whatman® Pk6F Silica Gel 60 Å 1000 μM) using Hexanes/EtOAc (1/1) as the eluant to afford (2′R)-65 as a colorless oil (12 mg, 78%). [α]_{D}²⁵ −105.2 (c 0.06, CHCl₃). ¹H NMR (400 MHz, CDCl₃) δ −0.05 (s, 3H), 0.05 (s, 3H), 1.35 (s, 3H), 1.38 (d, J=6.2 Hz, 3H), 1.46 (s, 3H), 1.61-1.81 (m, 4H), 2.40-2.46 (m, 2H), 2.70 (dd, J=14.1, 6.1 Hz, 1H), 2.81 (dd, J=14.1, 4.7 Hz, 1H), 3.46 (s, 6H), 3.89 (b, 1H), 4.10-4.14 (m, 1H), 4.56 (dd, J=9.1, 6.1 Hz, 1H), 5.13-5.17 (m, 4H), 5.32-5.37 (m, 1H), 5.59 (dd, J=15.4, 9.2 Hz, 1H), 5.70-5.75 (m, 1H), 6.66 (d, J=2.0 Hz, 1H), 6.70 (d, J=2.0 Hz, 1H). ¹³C NMR (100 MHz, CDCl₃) δ 21.5, 25.1, 25.7, 28.6, 30.1, 32.3, 40.0, 41.6, 56.6, 56.7, 70.5, 72.0, 80.1, 94.7, 94.9, 101.9, 108.2, 111.0, 119.7, 130.6, 132.7, 138.3, 155.8, 159.1, 168.4. HRMS (FAB) calcd for C₂₅H₃₆O₉H⁺ [M+H]⁺: 482.2438, found: 482.2437, Δ=0.1 ppm.

(2′S)-65 was prepared by same procedure from (2′S)-64. (2′S)-65: (20 mg, 87%). [α]_{D}²⁵ −32.0 (c 0.10, CHCl₃). ¹H NMR (500 MHz, CDCl₃) δ 1.18 (m, 2H), 1.35 (s, 3H), 1.37 (d, J=6.2 Hz, 1H), 1.42 (s, 3H), 1.55-1.69 (m, 2H), 1.98-2.06 (m, 1H), 2.42-2.48 (m, 3H), 2.78 (dd, J=13.8, 2.4 Hz, 1H), 3.41-3.50 (m, 6H), 3.62-3.67 (m, 1H), 4.19-4.25 (m, 1H), 4.49 (dd, J=9.4, 6.0 Hz, 1H), 5.11-5.18 (m, 4H), 5.36-5.42 (m, 1H), 5.56 (dd, J=15.4, 9.5 Hz, 1H), 5.65-5.72 (m, 1H), 6.57 (d, J=2.0 Hz, 1H), 6.66 (d, J=2.0 Hz, 1H). ¹³C NMR (100 MHz, CDCl₃) δ 21.6, 25.8, 26.8, 28.6, 32.0, 39.8, 42.6, 56.5, 56.7, 72.3, 74.8, 80.3, 94.7, 94.9, 101.8, 108.6, 110.7, 108.6, 110.7, 119.5, 129.9, 132.8, 138.7, 155.9, 159.0, 168.7. HRMS (FAB) calcd for C₂₅H₃₆O₉H⁺ [M+H]⁺: 482.2438, found: 482.2437, Δ=0.1 ppm.

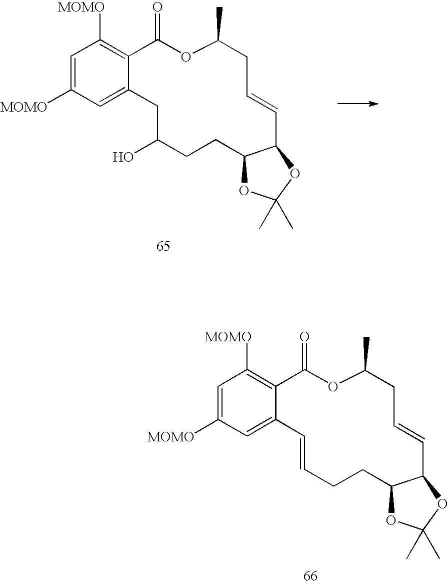

### Diene 66:

A solution of Martin's sulfurane dehydration agent (140 mg, 0.208) was added into a vial containing (2′R)-65 (20 mg, 0.042) at 0° C. The reaction mixture was warmed up to r.t. over 2 h and the crude was purified on preparative TLC (Whatman® Pk6F Silica Gel 60 Å 1000 μM) using Hexanes/EtOAc (1/1) as the eluant to afford 66 as a colorless oil (16 mg, 84%). [α]_{D}²⁵ −123.8 (c 0.08, CHCl₃). ¹H NMR (500 MHz, CDCl₃) δ 1.35 (s, 3H), 1.36 (d, J=6.0 Hz, 3H), 1.46 (s, 3H), 1.49-1.55 (m, 1H), 1.80-1.85 (m, 1H), 2.07-2.11 (m, 1H), 2.29-2.32 (m, 1H), 2.45-2.55 (m, 2H), 3.41-3.50 (m, 6H), 4.16-4.21 (m, 1H), 4.56 (dd, J=9.5, 5.4 Hz, 1H, 1H), 5.10-5.20 (m, 4H), 5.32-5.36 (m, 1H), 5.59 (dd, J=115.5, 9.6 Hz, 1H), 5.70-5.77 (m, 1H), 5.15 (m, 1H), 6.24 (d, J=15.4 Hz, 1H), 6.80 (d, J=1.8 HZ, 1H), 6.68 (d, J=1.8 HZ, 1H). ¹³C NMR (125 MHz, CDCl₃) δ 21.1, 25.8, 28.6, 28.7, 29.0, 39.5, 71.6, 80.1, 84.3, 94.6, 102.6, 104.8, 108.3, 117.9, 124.8, 128.4, 129.3, 131.9, 132.3, 136.8, 155.1, 158.9, 167.3. HRMS (FAB) calcd for C₂₅H₃₄O₈H⁺ [M+H]⁺: 463.2332, found: 463.2333, Δ=−0.2 ppm.

66 could also be obtained from (2′S)-65 using same procedure (90%).

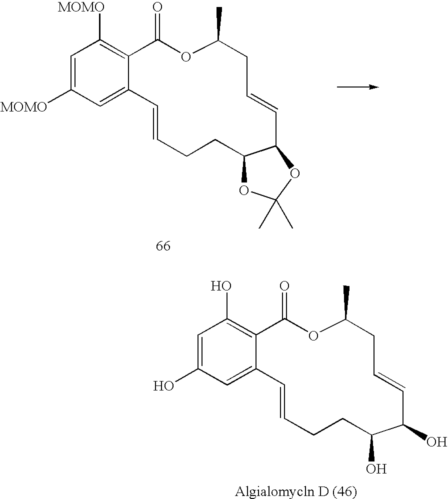

### Aigialomycin D (46):

To a solution of 66 (16 mg, 0.035) in MeOH (1.5 mL) was added HCl (1 N, 1.5 mL) and stirred for 2 d. The reaction was quenched with saturated aqueous NaHCO₃ solution and extracted with EtOAc. The organic layers were combined and dried over anhydrous MgSO₄, filtered and concentrated under reduced vacuum. The crude was purified on preparative TLC (Whatman® Pk6F Silica Gel 60 Å1000 μM) using MeOH/CH₂Cl₂ (5%) as the eluant to afford 46 as a white solid (8 mg, 69%). Mp: 84.2-86.9° C. [α]_{D}²⁵ −18.0 (c 0.03, MeOH). IR (neat) 3346, 1643, 1607, 1311, 1261, 1166, 1017, 968. ¹H NMR (500 MHz, acetone-d₆) δ 1.39 (d, J=6.4 Hz, 3H), 1.58-1.61 (m, 1H), 2.14 (m, 1H), 2.32-2.36 (m, 2H), 2.43-2.46 (m, 1H), 2.57 (ddd, J=14.5, 7.3, 3.1 Hz, 1H), 3.56 (br, 1H), 3.64 (m, 1H), 3.76 (br, 1H), 4.35 (brd, J=4.1 Hz, 1H), 5.41-5.47 (m, 1H), 5.69 (dd, J=15.6, 5.1 Hz, 1H), 5.87 (dddd, J=15.6, 7.4, 7.4, 1.4 Hz, 1H), 6.10 (ddd, J=15.9, 5.5, 5.7 Hz, 1H), 6.28 (d, J=2.3 Hz, 1H), 6.53 (d, J=2.3 Hz, 1H), 7.16 (d, J=15.9 Hz, 1H), 9.10 (bs, 1H), 11.7 (s, 1H). ¹³C NMR (125 MHz, acetone-d₆) δ 19.2, 28.1, 28.8, 38.1, 73.1, 73.4, 76.7, 102.6, 104.6, 107.9, 125.6, 130.8, 133.8, 135.9, 144.5, 163.2, 166.0, 172.3. LRMS (ESI) calcd for C₁₈H₂₂O₆Na⁺ [Na⁺H]⁺: 357.1, found: 357.3. LRMS (ESI) calcd for C₁₈H₂₁O₆ [M−H]⁻: 333.1, found: 333.1. LRMS (ESI) calcd for C₁₈H₂₂O₆Cl⁻−[M+Cl]: 369.1, found: 369.0. HRMS (TOF) calcd for C₁₈H₂₂O₆Na⁺ [M+Na]⁺: 357.1314, found: 357.1325, Δ=3.1 ppm. All the physical data are consistent with the reported value (Isaka et al. J. Org. Chem. 2002, 67, 1561-1566; incorporated herein by reference).

Determination of the stereochemistry at the 2′ position of 65:

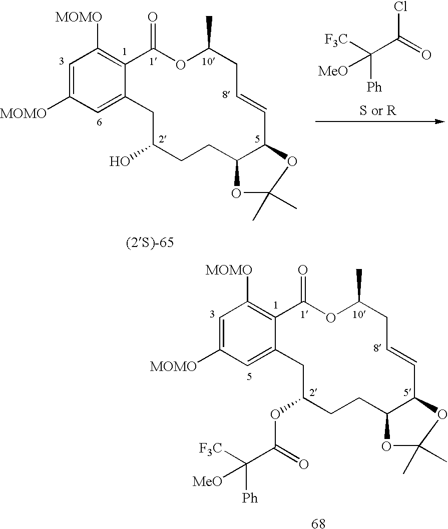

The stereochemistry of 2′ of (2′S)-65 was determined according to the protocol reported by Ikuko Ohtani et al. (Org. Chem. 1991, 56, 1296-1298; incorporated herein by reference).

To a solution of XX (1.5 mg, 0.003 mmol) in CH₂Cl₂ (0.1 mL) was added triethylamine (0.007 mL, 0.042 mmol), DMAP (0.1 mg, 0.001 mmol) and (S)- or (R)-methoxytrifluorophenyl acetic chloride (0.005 mL, 0.024 mmol). The reaction mixture was stirred overnight before loaded onto PTLC (250 μm) and eluted by Hex/EtOAc (4/1). The product (R) or (S) ester 68 was isolated as colorless oil (1.7 mg, 80%).

### Δδ=δ_{S}−δ_{R}.

### Δδ_{3′}=δ_{S5′}−δ_{R5′}=−0.05 ppm;

Δδ_{4′}=δ_{S6′}−δ_{R6′}=−0.13 ppm;

### ε_{5′}=δ_{S7′}−δ_{R7′}=−0.02 ppm;

### Δδ_{6′}=δ_{S8′}−δ_{R8′}=−0.05 ppm;

Δδ_{1′}=δ_{S1′}−δ_{R1′}=0.11 ppm;

Δδ₅=δ_{S5}−δ_{R5}=0.05 ppm;

### Δδ₃=δ_{S3}−δ_{R3}=0.01 ppm.

Therefore, the 2′ position should have a S configuration as shown above.

Cell Culture. The human cancer line MCF7 was obtained from American Type Culture Collection (Manassas, Va., USA) and maintained in 1:1 mixture of DME:F12 supplemented with 10% heat-inactivated fetal bovine serum, 2 mM glutamine, 50 units/ml of penicillin and streptomycin, and incubated in a humidified 5% CO₂/air atmosphere at 37° C. For the Alamar blue proliferation assay, 2×10³ cells were plated in 96-well plates. Twenty four hours later, cells were treated with the compound for 72 or 120 hours. On day 3 or 5, Alamar Blue viability assay (AccuMe Westlake, Ohio) was performed as described elsewhere (White, M. J.; DiCaprio, M. J Greenberg, D. A. J. Neurosci. Methods 1996, 70, 195-200; incorporated herein by reference). IC₅₀s were calculated as the doses of the compound required to inhibit cell growth by 50% of control values.

Immunoblotting. For immunoblotting, cells in culture were washed and harvested in PBS, and then lysed in NP-40 buffer (50 mMTris, pH 7.5, 1% Nonidet P-40, 150 mM NaCl, 2.5 mM Na₃VO₄, 10 mM phenylmethylsulfonyl fluoride, and 10 μM each leupeptin, aprotinin, and soybean trypsin inhibitor). Cell lysates were cleared by centrifugation at 14,000×g for 10 minutes, and supernatants were collected as the experimental samples. Protein concentrations were determined using BCA reagent (Pierce Chemical Co., Rockford, Ill.). Lysates were added to sample buffer [0.3125 M Tris-HCl (pH 6.8), 10% SDS, 50% glycerol, and 77.5 mg/ml DTT], and equal amounts of protein were resolved by 7% SDS-PAGE and transferred to nitrocellulose membranes. Blots were blocked in 5% nonfat milk in Tris-buffered saline [0.1% Tween 20, 10 mM Tris (pH 7.4), and 150 mM NaCl] and subsequently probed with antibody (HER2 rabbit polyclonal IgG, Santa Cruz Biotechnology, Santa Cruz, Calif.; and PI3 kinase P85, Upstate Cell Signaling Solutions, Lake Placid, N.Y.). After incubation with horseradish peroxidase-conjugated secondary antibodies, proteins were visualized by chemiluminescence using the ECL detection reagents (Amersham Corp., Piscataway, N.J.). To prepare lysate from xenograft tumors, tumor tissue was homogenized in 2% SDS lysis buffer for 30 s then processed as above.

### Example 4

### In Vitro and In Vivo Testing

### Cell Culture Experimental:

The human cancer cell lines MCF7, BT474 and N417 were obtained from the American Type Culture Collection (Manassas, Va., USA) and maintained in a 1:1 mixture of DME:F12 supplemented with 2 mM glutamine, 50 U/mL penicillin, 50 U/mL streptomycin and 5% heat inactivated fetal bovie serum (Gemini Bioproducts) and incubated at 37° C. in 5% CO₂.

Also, cytoxicity assay methods are described in Chou et al. Proc. Natl. Acad. Sci. USA 98:813-18, 2001; incorporated herein by reference.

### Protein Assays:

Cells were grown to 60-70% confluence and exposed to drugs or DMSO vehicle for the indicated time periods. Lysates were prepared using 50 mM Tris pH 7.4, 2% SDS and 10% glycerol lysis buffer. Protein concentration was determined using the BCA kit (Pierce Chemical Co.) according to the manufacturer's instructions. Clarified protein lysates (20-50 μg) were electrophoretically resolved on denaturing SDS-PAGE, transferred to nitrocellulose and probed with the following primary antibodies: anti-Her2 (C-18).

### Antiproliferative Index:

Growth assays were performed by seeding 10000 cells (MCF7, BT474 or N417) per well in 6-welldishes and incubating for 24 h before drug treatment. Drugs or vehicle were administered as outlined for each experiment, and cells were incubated for the time periods depicted and then the number quantified by a Coulter counter.

### Flow Cytometry:

Cell cycle distribution was assayed according to Nusse et al. with a Becton Dickinson fluorescence-activated cell sorter and analyzed by a Cell Cycle Multi-cycle system (Phoenix Flow System, San Diego, Calif., USA).

### In Vivo Activity:

Although a variety of methods known in the art can be utilized, one exemplary method by which the in vivo activity of the inventive compounds is determined is by subcutaneously transplanting a desired tumor mass in mice. Drug treatment is then initiated when tumor mass reaches approximately 100 mm³ after transplantation of the tumor mass. A suitable composition, comprising any one inventive compounds described above, including classes thereof, subclasses thereof, or species thereof, optionally further comprising a pharmaceutically acceptable carrier and optionally further comprising an additional therapeutic agent has, is then administered to the mice, preferably in saline and also administered once a day at doses in the range of 0.001 mg/kg, to about 50 mg/kg, although it will be appreciated that other doses can also be administered, as described herein (e.g., 0.01 mg/kg to about 25 mg/kg of body weight, or 0.1 mg/kg to about 10 mg/kg of body weight), or, in some embodiments, at dosages in the range of about 50 mg/kg to about 100 mg/kg, or dosages below 0.001 mg/kg. Body weight and tumor size are then measured daily and changes in percent ratio to initial values are plotted. In cases where the transplanted tumor ulcerates, the weight loss exceeds 25-30% of control weight loss, the tumor weight reaches 10% of the body weight of the cancer-bearing mouse, or the cancer-bearing mouse is dying, the animal is sacrificed in accordance with NIH guidelines for animal welfare.

In addition, methods for studying the therapeutic effect of compounds in vivo in nude mice are described in Chou et al. Proc. Natl. Acad. Sci. USA 98:8113-18, 2001.

### Results:

The methods for studying the cytotoxicity of the radicicol and cycloproparadicicol compounds in vitro for CCRF-CEM, CCRF-CEM/Vinblastine, and CCRF-CEM/Taxol cells are described in Chou et al. Proc. Natl. Acad. Sci. USA 98:8113-8118, 2001. The method of studying the therapeutical effect of these compounds in vivo is also described in Chou et al. Proc. Natl. Acad. Sci. USA 98:8113-8118, 2001.

The tested radicicol and cycloproparadicicol compounds suppressed xenograft tumor growth in nude mice about 60-88%. These effects are not dose dependent. All of the test compound that were tested in vivo are relatively non-toxic to normal tissues. The compounds do not show cross-resistance with vinblastine. Therefore, these compounds do not seem to be substrates for the multi-drug resistant (MDR) P-glycoprotein (P-gp). The compounds tested were also not found to be cross-resistant with Taxol. These compound may be particularly useful in conjunction with other anti-cancer agents. The radicicol and cycloproparadicicol compounds may increase the therapeutic effect of the combination without increasing the toxicity of the combination.

What is claimed is:

1. A compound having the structure: 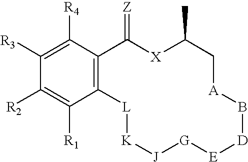
wherein
R₁ is hydrogen, halogen, cyano, —OR_{A}, —N(R_{A})₂, —SR_{A}, —O(C═O)R_{A}, —N(R_{A})(C═O)(R_{A}),
—C(O)R_{A}, —C(O)OR_{A}, —CON(R_{A})₂, —OCO₂R_{A}, or an aliphatic, heteroaliphatic, aryl, heteroaryl, alkylaryl, or alkylheteroaryl moiety, wherein each occurrence of R_{A} is independently hydrogen, a protecting group, or an aliphatic, heteroaliphatic, aryl, heteroaryl, alkylaryl, or alkylheteroaryl moiety;
R₂ is hydrogen, halogen, cyano, —OR_{B}, —N(R_{B})₂, —SR_{B}, —O(C═O)R_{B}, —N(R_{B})(C═O)(R_{B}),
—C(O)R_{B}, —C(O)OR_{B}, —CON(R_{B})₂, —OCO₂R_{B}, or an aliphatic, heteroaliphatic, aryl, heteroaryl, alkylaryl, or alkylheteroaryl moiety, wherein each occurrence of R_{B} is independently hydrogen, a protecting group or an aliphatic, heteroaliphatic, aryl, heteroaryl, alkylaryl, or alkylheteroaryl moiety;
R₃ is hydrogen, halogen, cyano, —OR_{C}, —N(R_{C})₂, —SR_{C}, —O(C═O)R_{C}, —N(R_{C})(C═O)(R_{C}),
—C(O)R_{C}, —C(O)OR_{C}, —CON(R_{C})₂, —OCO₂R_{C}, or an aliphatic, heteroaliphatic, aryl, heteroaryl, alkylaryl, or alkylheteroaryl moiety, wherein each occurrence of R_{C} is independently hydrogen, a protecting group, or an aliphatic, heteroaliphatic, aryl, heteroaryl, alkylaryl, or alkylheteroaryl moiety;
R₄ is hydrogen, halogen, cyano, —OR_{D}, —N(R_{D})₂, —SR_{D}, —O(C═O)R_{D}, —N(R_{D})(C═O)(R_{D}),
—C(O)R_{D}, —C(O)OR_{D}, —CON(R_{D})₂, —OCO₂R_{D}, or an aliphatic, heteroaliphatic, aryl, heteroaryl, alkylaryl, or alkylheteroaryl moiety, wherein each occurrence of R_{D} is independently hydrogen, a protecting group or an aliphatic, heteroaliphatic, aryl, heteroaryl, alkylaryl, or alkylheteroaryl moiety;
Z is O, S, or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;
X is O, S or NR_{G}, wherein R_{G} is hydrogen or lower alkyl;
A and B together represent 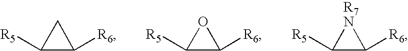
—CHR₅—CHR₆—, —CR₅═CR₆—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring;
D and E together represent 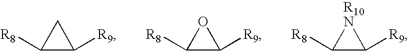
—CHR₉—CHR₉—, —CR₉═CR₉—, wherein R₈ and R₉ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₀ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₈—CHR₉—, R₉ and R₉ taken together represent a substituted or unsubstituted 3-7 membered aliphatic, heteroaliphatic, aryl or heteroaryl ring;
G and J together represent 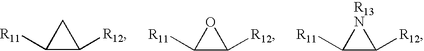
—CHR₁₁—CHR₁₂—, —CR₁₁ ═CR₁₂—, wherein R₁₁ and R₁₂ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₃ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₁—CHR₁₂—, R₁₁ and R₁₂ taken together represent a substituted or unsubstituted 3-7 membered aliphatic, heteroaliphatic, aryl or heteroaryl ring;
K and L together represent 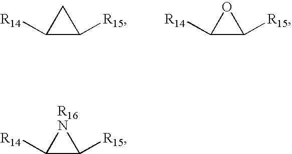
—CHR₁₄—CHR₁₅—, —CR₁₄═CR₁₅—, wherein R₁₄ and R₁₅ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₆ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₄—CHR₁₅—, R₁₄ and R₁₅ taken together represent a substituted or unsubstituted 3-7 membered aliphatic, heteroaliphatic, aryl or heteroaryl ring;
whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted; and
pharmaceutically acceptable derivatives thereof.

2. The compound of claim 1, wherein Z and X are each 0, and the compound has the structure:

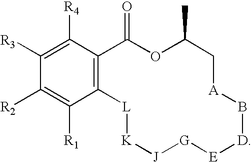

3. The compound of claim 1, wherein Z is O and X is NR_{G}, and the compound has the structure:

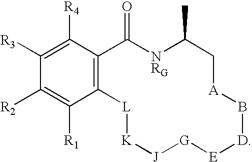

4. The compound of claim 3, wherein R^{G} is H.

5. The compound of claim 1, wherein G and J together represent —CH₂—CH₂— and the compound has the structure:

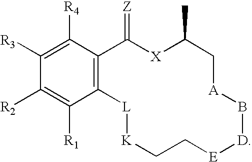

6. The compound of claim 1, wherein A-B is —CH═CH— and the compound has the structure:

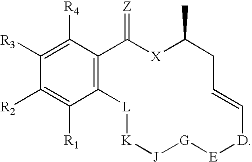

7. The compound of claim 1, wherein K and L together represent —CH═CH— and the compound has the structure:

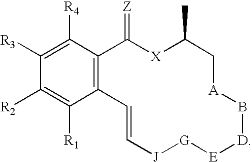

8. The compound of claim 1, wherein D and E together represent —CHOH═CHOH— and the compound has the structure:

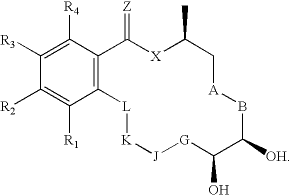

9. The compound of claim 1, wherein A, B, D, E, G, J, K, and L are as represented in the structure:

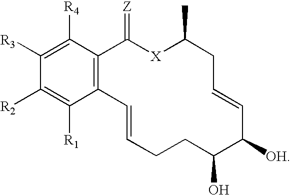

10. A compound of structure:

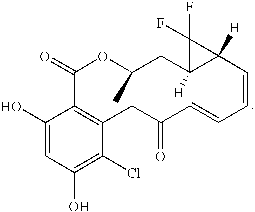

11. A pharmaceutical composition comprising a compound of claim 1 and a pharmaceutically acceptable carrier.

12. The pharmaceutical composition of claim 11, further comprising one or more additional therapeutic agents.

13. The pharmaceutical composition of claim 12, wherein the one or more additional therapeutic agents comprises an anticancer agent.

14. A method for treating cancer comprising:
administering a therapeutically effective amount of a compound of claim 1 to a subject in need thereof.

15. The method of claim 14, wherein the therapeutically effective amount is in the range of 0.001 mg/kg to 50 mg/kg of body weight.

16. The method of claim 14, wherein the therapeutically effective amount is in the range of 0.01 mg/kg to about 25 mg/kg of body weight.

17. The method of claim 14, said method further comprising administering one or more additional therapeutic agents in combination with the compound.

18. The method of claim 17, wherein the one or more additional therapeutic agents comprises an anticancer agent.

19. A method for inhibiting the growth of or killing cancer cells, said method comprising:
contacting the cancer cells with an amount of a compound of claim 1 effective to inhibit the growth of or kill cancer cells.

20. A method for the synthesis of a compound having the structure (I): 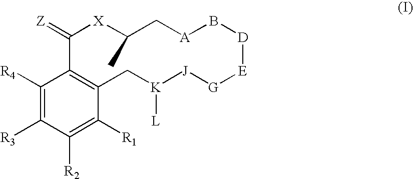
wherein
R¹ is hydrogen, halogen, an aliphatic, heteroaliphatic, aryl, heteroaryl, alkylaryl, or alkylheteroaryl moiety, or N(R_{A})₂, wherein each occurrence of R_{A} is independently hydrogen, a protecting group, or an aliphatic, heteroaliphatic, aryl, heteroaryl, alkylaryl, or alkylheteroaryl moiety;
R₂ is hydrogen, halogen, cyano, —OR_{B}, —N(R_{B})₂, —SR_{B}, —O(C═O)R_{B}, —N(R_{B})(C═O)(R_{B}),
—C(O)R_{B}, —C(O)OR_{B}, —CON(R_{B})₂, —OCO₂R_{B}, or an aliphatic, heteroaliphatic, aryl, heteroaryl, alkylaryl, or alkylheteroaryl moiety, wherein each occurrence of R_{B} is independently hydrogen, a protecting group or an aliphatic, heteroaliphatic, aryl, heteroaryl, alkylaryl, or alkylheteroaryl moiety;
R₃ is hydrogen, halogen, an aliphatic, heteroaliphatic, aryl, heteroaryl, alkylaryl, or alkylheteroaryl moiety, or —N(R_{C})₂, wherein each occurrence of R_{C} is independently hydrogen, a protecting group, or an aliphatic, heteroaliphatic, aryl, heteroaryl, alkylaryl, or alkylheteroaryl moiety;
R₄ is hydrogen, halogen, cyano, —OR_{D}, —N(R_{D})₂, —SR_{D}, —O(C═O)R_{D}, —N(R_{D})(C═O)(R_{D}),
—C(O)R_{D}, —C(O)OR_{D}, —CON(R_{D})₂, —OCO₂R_{D}, or an aliphatic, heteroaliphatic, aryl, heteroaryl, alkylaryl, or alkylheteroaryl moiety, wherein each occurrence of R_{D} is independently hydrogen, a protecting group or an aliphatic, heteroaliphatic, aryl, heteroaryl, alkylaryl, or alkylheteroaryl moiety;
Z is O, S or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;
X is O, S or NR_{G}, wherein R_{G} is hydrogen or lower alkyl;
A and B together represent 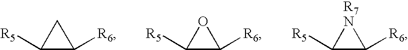
—CHR₅—CHR₆—, —CR₅═CR—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₅ taken together represent a substituted or unsubstituted 3-7 membered aliphatic, heteroaliphatic, aryl or heteroaryl ring;
D and E together represent —CHR₈—CHR₉—, —CR₈═CR₉—, wherein R₉ and R₉ are each independently hydrogen or lower alkyl;
G and J together represent —CHR₁₀—CHR₁₁—, —CR₁₀═CR₁₁—, wherein R₁₀ and R₁₁ are each independently hydrogen or lower alkyl;
K and L together represent C═O, C═S, CH—CH₃, CH—CH(R_{L})₂, C═C(R_{L})₂, —CH₂—,
—C(—S(CH₂)₃S—)—, CH—OR_{L}, CH—SR_{L}, CH—N(R_{L})₂, CH—N(R_{L})(C═O)(R_{L}), C═N—O—R_{L}, CH—N═O, C═C(R_{L})—N(R_{L})₂, C═N—R_{L}, C═N—N(R_{L})₂, or, if the dotted line - - - represents a bond, whereby a double bond is present, then K and L together represent C—N(R_{L})₂, wherein each occurrence of R_{L} is independently hydrogen, a protecting group, an aliphatic, heteroaliphatic, aryl, heteroaryl, alkylaryl, or alkylheteroaryl moiety, or two occurrences of R_{L} taken together represent a 3 to 7-membered cyclic aliphatic, heteroaliphatic, aromatic or heteroaromatic moiety;
whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted;
wherein one or any two of R₁, R_{A}, R₂, R_{B}, R₃, R_{C}, R₄, R_{D}, R₅, R₆, R_{J}, or R_{L} are optionally a linker covalently bonded to a compound selected from the group consisting of radicicol, monocillin, analogues of radicicol and monocillin, geldanamycin, analogues of geldanamycin, and steroids,
said method comprising:
(1) reacting an acidic component having the structure: 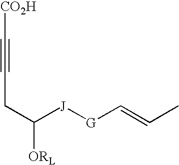
wherein R_{L}, J, and G are as defined above, with a chiral component having the structure: 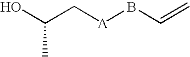
wherein A and B are as defined above, in the presence of an esterification reagent to generate an intermediate having the structure: 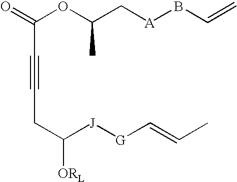
(2) complexing the intermediate with a cobalt, such as dicobalt hexcarbonyl, to yield a structure: 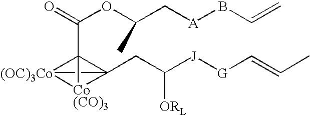
(3) cyclizing the combalt complex in the presence of an olefin metathesis catalyst to generate the compound: 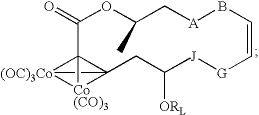
(4) removing the cobalt to form a ynolide;
(5) reacting the alkyne moiety of the ynolide with a diene under cyclcoaddition conditions to generate the compound: 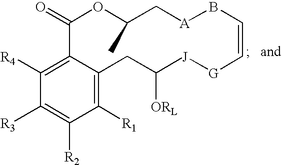
(6) optionally further reacting the macrocycle with one or more reagents to diversify and optionally deprotecting the macrocycle to generate a compound having the formula (I).

21. The method of claim 20, wherein the step of esterification is performed using diethylazodicarboxylate (DIAD) in the presence of triphenylphosphine or trifurylphosphine.

22. The method of claim 20, wherein the step of olefin metathesis is performed using an olefin metathesis catalyst.

23. The method of claim 20, wherein the step of olefin metathesis is performed using a ruthenium-based olefin metathesis catalyst.

24. The method of claim 23, wherein the step of olefin metathesis is performed using Ru(1,3-dimesityl-4,5-dihydro-imidazol-2-ylidene)(═CHCH═C(CH₃)₂)PCp₃Cl₂.

25. A method for synthesis of a macrocycle having the structure (IIa): 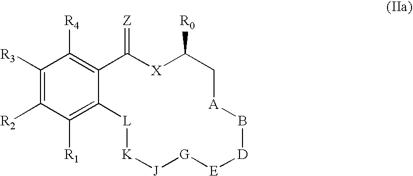
wherein
R₀ is hydrogen, halogen, cyano, —OR_{Z}, —N(R_{Z})₂, —SR_{Z}, —O(C═O)R_{Z}, —N(R_{Z})(C═O)(R_{Z}), —C(O)R_{Z}, —C(O)OR_{Z}, —CON(R_{Z})₂, —OCO₂R_{Z}, or an aliphatic, heteroaliphatic, aryl, heteroaryl, alkylaryl, or alkylheteroaryl moiety, wherein each occurrence of R_{Z} is independently hydrogen, a protecting group, or an aliphatic, heteroaliphatic, aryl, heteroaryl, alkylaryl, or alkylheteroaryl moiety
R₁ is hydrogen, halogen, cyano, —OR_{A}, —N(R_{A})₂, —SR_{A}, —O(C═O)R_{A}, —N(R_{A})(C═O)(R_{A}),
—C(O)R_{A}, —C(O)OR_{A}, —CON(R_{A})₂, —OCO₂R_{A}, or an aliphatic, heteroaliphatic, aryl, heteroaryl, alkylaryl, or alkylheteroaryl moiety, wherein each occurrence of R_{A} is independently hydrogen, a protecting group, or an aliphatic, heteroaliphatic, aryl, heteroaryl, alkylaryl, or alkylheteroaryl moiety;
R₂ is hydrogen, halogen, cyano, —OR_{B}, —N(R_{B})₂, —SR_{B}, —O(C═O)R_{B}, —N(R_{B})(C═O)(R_{B}),
—C(O)R_{B}, —C(O)OR_{B}, —CON(R_{B})₂, —OCO₂R_{B}, or an aliphatic, heteroaliphatic, aryl, heteroaryl, alkylaryl, or alkylheteroaryl moiety, wherein each occurrence of R_{B} is independently hydrogen, a protecting group or an aliphatic, heteroaliphatic, aryl, heteroaryl, alkylaryl, or alkylheteroaryl moiety;
R₃ is hydrogen, halogen, cyano, —OR_{C}, —N(R_{C})₂, —SR_{C}, —O(C═O)R_{C}, —N(R_{C})(C═O)(R_{C}),
—C(O)R_{C}, —C(O)OR_{C}, —CON(R_{C})₂, —OCO₂R_{C}, or an aliphatic, heteroaliphatic, aryl, heteroaryl, alkylaryl, or alkylheteroaryl moiety, wherein each occurrence of R_{C} is independently hydrogen, a protecting group, or an aliphatic, heteroaliphatic, aryl, heteroaryl, alkylaryl, or alkylheteroaryl moiety;
R₄ is hydrogen, halogen, cyano, —OR_{D}, —N(R_{D})₂, —SR_{D}, —O(C═O)R_{D}, —N(R_{D})(C═O)(R_{D}),
—C(O)R_{D}, —C(O)OR_{D}, —CON(R_{D})₂, —OCO₂R_{D}, or an aliphatic, heteroaliphatic, aryl, heteroaryl, alkylaryl, or alkylheteroaryl moiety, wherein each occurrence of R_{D} is independently hydrogen, a protecting group or an aliphatic, heteroaliphatic, aryl, heteroaryl, alkylaryl, or alkylheteroaryl moiety;
Z is O, S, or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;
X is O, S or NR_{G}, wherein R_{G} is hydrogen or lower alkyl;
A and B together represent 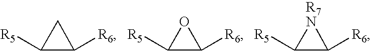
—CHR₅—CHR₆—, —CR₅═CR₆—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K} or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring;
D and E together represent 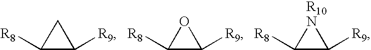
—CHR₈—CHR₉—, —CR₈═CR₉—, wherein R₈ and R₉ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J}, —CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₀ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₈—CHR₉—, R₉ and R₉ taken together represent a substituted or unsubstituted 3-7 membered aliphatic, heteroaliphatic, aryl or heteroaryl ring:
G and J together represent 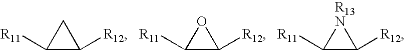
—CHR₁₁—CHR₁₂—, —CR₁₁═CR₁₂—, wherein R₁₁ and R₁₂ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J}, —CON(R)_{K}, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₃ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K} or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₁—CHR₁₂—, R₁₁ and R₁₂ taken together represent a substituted or unsubstituted 3-7 membered aliphatic, heteroaliphatic, aryl or heteroaryl ring;
K and L together represent 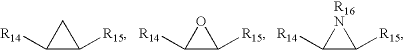
—CHR₁₄—CHR₁₅—, —CR₁₄═CR₁₅—, wherein R₁₄ and R₁₅ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(—O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₆ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K} or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₄—CHR₁₅—, R₁₄ and R₁₅ taken together represent a substituted or unsubstituted 3-7 membered aliphatic, heteroaliphatic, aryl or heteroaryl ring;
whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted,
said method comprising:
(1) reacting a component having the structure: 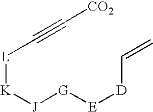
wherein R_{L}, J, and G are as defined above, with a chiral component having the structure: 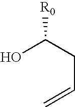
wherein A, B, D, E, G, J, K, and L are as defined above, in the presence of an esterification reagent to generate an intermediate having the structure: 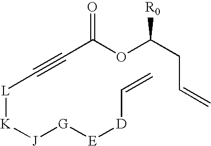
(2) complexing the intermediate with a cobalt, such as dicobalt hexcarbonyl, to yield a structure: 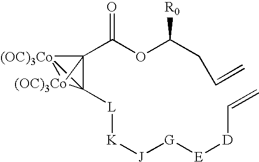
(3) cyclizing the combalt complex in the presence of an olefin metathesis catalyst to generate the compound: 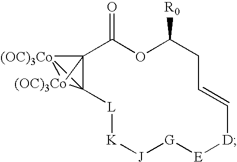
(4) removing the cobalt to form a ynolide;
(5) reacting the alkyne moiety of the ynolide with a diene under cyclcoaddition conditions to generate the compound: 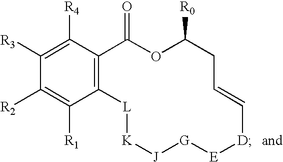
(6) optionally further reacting the macrocycle with one or more reagents to diversify and optionally deprotecting the macrocycle to generate a compound having the formula (IIa).

26. The method of claim 25, wherein the method further comprises further diversifying the macrocycle to generate a compound having the structure (II) as defined herein.

27. The method of claim 25, wherein the step of olefin metathesis is performed using an olefin metathesis catalyst.

28. The method of claim 25, wherein the step of olefin metathesis is performed using a ruthenium-based olefin metathesis catalyst.

29. The method of claim 28, wherein the step of olefin metathesis is performed using Ru(1,3-dimesityl-4,5-dihydro-imidazol-2-ylidene)(═CHCH═C(CH₃)₂)PCp₃Cl₂.

30. A compound of the formula: 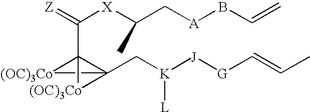
wherein
Z is O, S or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;
X is O, S or NR_{G}, wherein R_{G} is hydrogen or lower alkyl;
A and B together represent 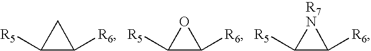
—CHR₅—CHR₆—, —CR₅═C₆—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring,
G and J together represent —CHR₁₀—CHR₁₁—, —CR₁₀ ═CR₁₁—, wherein R₁₀ and R₁₁ are each independently hydrogen or lower alkyl;
K and L together represent C═O, C═S, CH—CH₃, CH—CH(R_{L})₂, C═C(R_{L})₂, —CH₂—C(—S(CH₂)₃S—)—, CH—OR_{L}, CH—SR_{L}, CH—N(R_{L})₂, CH—N(R_{L})(C═O)(R_{L}), C═N—O—R_{L}, CH—N═O, C═C(R_{L})—N(R_{L})₂, C═N—R_{L}, C═N—N(R_{L})₂, or, if the dotted line - - - represents a bond, whereby a double bond is present, then K and L together represent C—N(R_{L})₂, wherein each occurrence of R_{L} is independently hydrogen, a protecting group, an aliphatic, heteroaliphatic, aryl, heteroaryl, alkylaryl, or alkylheteroaryl moiety, or two occurrences of R_{L} taken together represent a 3 to 7-membered cyclic aliphatic, heteroaliphatic, aromatic or heteroaromatic moiety;
whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted;
wherein one or any two of R_{A}, R_{B}, R_{C}, R_{D}, R_{S}, R_{S}, R_{J}, or R_{L} are optionally a linker covalently bonded to a compound selected from the group consisting of radicicol, monocillin, analogues of radicicol and monocillin, geldanamycin, analogues of geldanamycin, and steroids.

31. A compound of the formula: 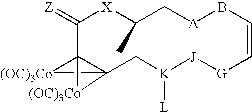
wherein
Z is O, S or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;
X is O, S or NR_{G}, wherein R_{G} is hydrogen or lower alkyl;
A and B together represent 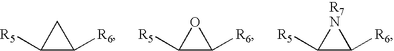
—CHR₅—CHR₆—, —CR₅═CR₆—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring,
G and J together represent —CHR₁₀—CHR₁₁—, —CR₁₀ ═CR₁₁—, wherein R₁₀ and R₁₁ are each independently hydrogen or lower alkyl;
K and L together represent C═O, C═S, CH—CH₃, CH—CH(R_{L})₂, C═C(R_{L})₂, —CH₂, —C(—S(CH₂)₃S—)—, CH—OR_{L}, CH—SR_{L}, CH—N(R_{L})₂, CH—N(R_{L})(C═O)(R_{L}), C═N—O—R_{L}, CH—N═O, C═C(R_{L})—N(R_{L})₂, C═N—R_{L}, C═N—N(R_{L})₂, or, if the dotted line - - - represents a bond, whereby a double bond is present, then K and L together represent C—N(R_{L})₂, wherein each occurrence of R_{L} is independently hydrogen, a protecting group, an aliphatic, heteroaliphatic, aryl, heteroaryl, alkylaryl, or alkylheteroaryl moiety, or two occurrences of R_{L} taken together represent a 3 to 7-membered cyclic aliphatic, heteroaliphatic, aromatic or heteroaromatic moiety;
whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted;
wherein one or any two of R_{A}, R_{B}, R_{C}, R_{D}, R₅, R₆, R_{J}, or R_{L} are optionally a linker covalently bonded to a compound selected from the group consisting of radicicol, monocillin, analogues of radicicol and monocillin, geldanamycin, analogues of geldanamycin, and steroids.

32. A compound of the formula: 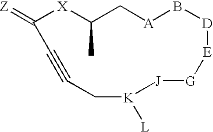
wherein
Z is O, S or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;
X is O, S or NR_{G}, wherein R_{G} is hydrogen or lower alkyl;
A and B together represent 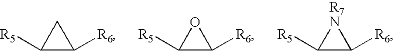
—CHR₅—CHR₆—, —CR₅—CR₆—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R^{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring;
D and E together represent —CHR₈—CHR₉—, —CR₈═CR₉—, wherein R₈ and R₉ are each independently hydrogen or lower alkyl;
G and J together represent —CHR₁₀—CHR₁₁—, —CR₁₀ ═CR₁₁—, wherein R₁₀ and R₁₁ are each independently hydrogen or lower alkyl;
K and L together represent C═O, C═S, CH—CH₃, CH—CH(R_{L})₂, C═C(R_{L})₂, —CH₂, —C(—S(CH₂)₃S—)—, CH—OR_{L}, CH—SR_{L}, CH—N(R_{L})₂, CH—N(R_{L})(C═O)(R_{L}), C═N—O—R_{L}, CH—N═O, C═C(R_{L})—N(R_{L})₂, C═N—R_{L}, C═N—N(R_{L})₂, or, if the dotted line - - - represents a bond, whereby a double bond is present, then K and L together represent C—N(R_{L})₂, wherein each occurrence of R_{L} is independently hydrogen, a protecting group, an aliphatic, heteroaliphatic, aryl, heteroaryl, alkylaryl, or alkylheteroaryl moiety, or two occurrences of R_{L} taken together represent a 3 to 7-membered cyclic aliphatic, heteroaliphatic, aromatic or heteroaromatic moiety;
whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted;
wherein one or any two of R_{A}, R_{B}, R_{C}, R_{D}, R₅, R₆, R^{J}, or R_{L} are optionally a linker covalently bonded to a compound selected from the group consisting of radicicol, monocillin, analogues of radicicol and monocillin, geldanamycin, analogues of geldanamycin, and steroids.

33. The compound of claim 32, wherein D and E together represent —CR₈═CR₉—.

34. A compound of formula: 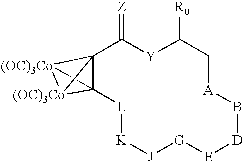
wherein
R₀ is hydrogen, cyano, —OR_{Z}, —N(R^{Z})₂, —SR_{Z}, —O(C═O)R_{Z}, —N(R_{Z})(C═O)(R_{Z}), —C(O)R_{Z}, —C(O)OR_{Z}, —CON(R_{Z})₂, —OCO₂R_{Z}, or an aliphatic, heteroaliphatic, aryl, heteroaryl, alkylaryl, or alkylheteroaryl moiety, wherein each occurrence of R_{Z} is independently hydrogen, a protecting group, or an aliphatic, heteroaliphatic, aryl, heteroaryl, alkylaryl, or alkylheteroaryl moiety
Z is O, S, or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;
X is O, S or NR_{G}, wherein R_{G} is hydrogen or lower alkyl;
A and B together represent 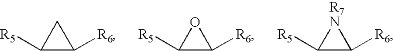
—CHR₅—CHR₆—, —CR₅═CR₆—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(—O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R), —C(O)R_{K}, —C(O)OR_{K}, —CON(R_{K})₂, —OCO₂R_{Y} or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring;
D and E together represent 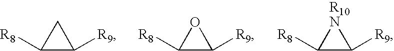
—CHR₈—CHR₉—, —CR₈═CR₉—, wherein R₈ and R₉ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₀ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₈—CHR₉—, R₉ and R₉ taken together represent a substituted or unsubstituted 3-7 membered aliphatic, heteroaliphatic, aryl or heteroaryl ring;
G and J together represent 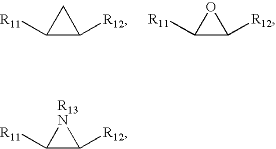
—CHR₁₁—CHR₁₂—, —CR₁₁═CR₁₂—, wherein R₁₁ and R₁₂ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₃ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₁—CHR₁₂—, R₁₁ and R₁₂ taken together represent a substituted or unsubstituted 3-7 membered aliphatic, heteroaliphatic, aryl or heteroaryl ring;
K and L together represent 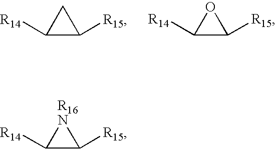
—CHR₁₄—CHR₁₅—, —CR₁₄═CR₁₅—, wherein R₁₄ and R₁₅ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₆ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₄—CHR₁₅—, R₁₄ and R₁₅ taken together represent a substituted or unsubstituted 3-7 membered aliphatic, heteroaliphatic, aryl or heteroaryl ring;
whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted.

35. The compound of claim 34, wherein A and B together represent —CR₅═CR₆—.

36. A compound of the formula: 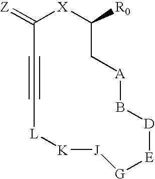
wherein
R₀ is hydrogen, cyano, —OR_{Z}, —N(R_{Z})₂, —SR_{Z}, —O(C═O)R_{Z}, —N(R_{Z})(C═O)(R_{Z}), —C(O)R_{Z}, —C(O)OR_{Z}, —CON(R_{Z})₂, —OCO₂R_{Z}, or an aliphatic, heteroaliphatic, aryl, heteroaryl, alkylaryl, or alkylheteroaryl moiety, wherein each occurrence of R_{Z} is independently hydrogen, a protecting group, or an aliphatic, heteroaliphatic, aryl, heteroaryl, alkylaryl, or alkylheteroaryl moiety
Z is O, S, or NR_{E}, wherein R_{E} is hydrogen, a protecting group, an aliphatic, heteroaliphatic, aryl, heteroaryl, alkylaryl, or alkylheteroaryl moiety, or OR_{F}, wherein R_{F} is hydrogen, a protecting group, an aliphatic, heteroaliphatic, aryl, heteroaryl, alkylaryl, or alkylheteroaryl moiety;
X is O, S or NR_{G}, wherein R_{G} is hydrogen or lower alkyl;
A and B together represent 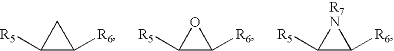
—CHR₅—CHR₆—, —CR₅═CR₆—, wherein R₅ and R₆ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₇ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K} or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₅—CHR₆—, R₅ and R₆ taken together represent a substituted or unsubstituted 3-7 membered aliphatic, heteroaliphatic, aryl or heteroaryl ring;
D and E together represent 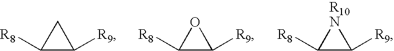
—CHR₈—CHR₉—, —CR═CR₉—, wherein R₈ and R₉ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(—O)OR_{J},
—CON(R_{J})₂, —OCO₂R_{J}, —OS(—O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₀ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₈—CHR₉—, R₉ and R₉ taken together represent a substituted or unsubstituted 3-7 membered aliphatic, heteroaliphatic, aryl or heteroaryl ring;
G and J together represent 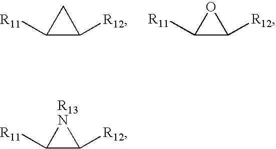
—CHR₁₁—CHR₁₂—, —CR₁₁═CR₁₂—, wherein R₁₁ and R₁₂ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR_{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₃ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₁—CHR₁₂—, R₁₁ and R₁₂ taken together represent a substituted or unsubstituted 3-7 membered aliphatic, heteroaliphatic, aryl or heteroaryl ring;
K and L together represent 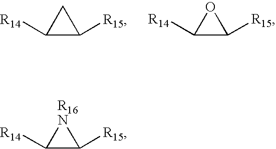
—CHR₁₄—CHR₁₅—, —CR₁₄═CR₁₅—, wherein R₁₄ and R₁₅ are each independently hydrogen, halogen, cyano, —OR_{J}, —N(R_{J})₂, —SR_{J}, —O(C═O)R_{J}, —O(S═O)R_{J}, —N(R_{J})(C═O)(R_{J}), —C(═O)R_{J}, —C(═O)OR_{J},
—CON(R_{J})₂, —OCO₂R_{J}, —OS(═O)OR^{J} or an aliphatic, heteroaliphatic, aryl, heteroaryl, alkylaryl, or alkylheteroaryl moiety, wherein each occurrence of R_{J} is independently hydrogen, a protecting group, or an aliphatic, heteroaliphatic, aryl, heteroaryl, alkylaryl, or alkylheteroaryl moiety, and wherein R₁₆ is hydrogen, a protecting group, —OR_{K}, —SR_{K}, —C(O)OR_{K}, —C(O)NR_{K}, —S(O)₂R_{K}, —O(C═O)R_{K}, —N(R_{K})(C═O)(R_{K}), —C(O)R_{K}, —C(O)OR_{K}, —CON(R_{K})₂, —OCO₂R_{K}, or an aliphatic, heteroaliphatic, aryl, heteroaryl, alkylaryl, or alkylheteroaryl moiety, wherein each occurrence of R_{K} is independently hydrogen, a protecting group or an aliphatic, heteroaliphatic, aryl, heteroaryl, alkylaryl, or alkylheteroaryl moiety, or when A and B together represent —CHR₁₄—CHR₁₅—, R₁₄ and R₁₅ taken together represent a substituted or unsubstituted 3-7 membered aliphatic, heteroaliphatic, aryl or heteroaryl ring;
whereby each of the foregoing aliphatic and heteroaliphatic moieties may independently be substituted or unsubstituted, cyclic or acyclic, or branched or unbranched, and each aryl, heteroaryl, alkylaryl, and alkylheteroaryl moiety may be substituted or unsubstituted.

37. The compound of claim 36, wherein A and B together represent —CR₅═CR₆—.

38. The compound of claim 36, wherein the compound has the formula: [chemical structure]
